(19) United States
(12) Patent Application Publication
(10) Pub. No.: US 2022/0238815 A1
(43) Pub. Date: Jul. 28, 2022

(54) ORGANIC ELECTROLUMINESCENT ELEMENT, COMPOUND, AND ELECTRONIC APPLIANCE

(71) Applicant: IDEMITSU KOSAN CO.,LTD., Chiyoda-ku, Tokyo (JP)

(72) Inventors: Takushi SHIOMI, Sodegaura-shi (JP); Hiromi NAKANO, Sodegaura-shi (JP)

(73) Assignee: IDEMITSU KOSAN CO.,LTD., Chiyoda-ku, Tokyo (JP)

(21) Appl. No.: 17/612,293

(22) PCT Filed: May 19, 2020
(86) PCT No.: PCT/JP2020/019779

§ 371 Date: Nov. 18, 2021

(30) Foreign Application Priority Data
May 20, 2019 (JP) 2019-094653

(51) Int. Cl. H01L 51/00

(52) U.S. Cl. CPC H01L 51/0072; H01L 51/0056; H01L 51/5016; H01L 51/0073; H01L 51/0074; H01L 51/0094

(57) ABSTRACT

An organic electroluminescence device includes: an anode; a cathode; an emitting layer provided between the anode and the cathode, in which the emitting layer contains a delayed fluorescent compound M2 and a compound M3 represented by a formula (3), and a singlet energy S₁(M2) of the compound M2 and a singlet energy S₁(M3) of the compound M3 satisfy the relationship S₁(M3)>S₁(M2). In the formula (3), A₃₀ is a group represented by a formula (3a) or the like.

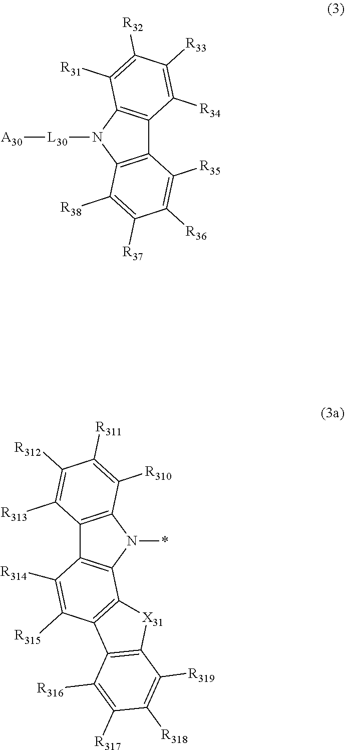

### TECHNICAL FIELD

The present invention relates to an organic electroluminescence device, a compound, and an electronic device.

### BACKGROUND ART

When a voltage is applied to an organic electroluminescence device (hereinafter, occasionally referred to as “organic EL device”), holes are injected from an anode and electrons are injected from a cathode into an emitting layer. The injected electrons and holes are recombined in the emitting layer to form excitons. Specifically, according to the electron spin statistics theory, singlet excitons and triplet excitons are generated at a ratio of 25%:75%.

A fluorescent organic EL device using light emission from singlet excitons has been applied to a full-color display such as a mobile phone and a television set, but an internal quantum efficiency is said to be at a limit of 25%. Accordingly, studies has been made to improve a performance of the organic EL device.

For instance, it is expected to further efficiently emit the organic EL device using triplet excitons in addition to singlet excitons. In view of the above, a highly efficient fluorescent organic EL device using thermally activated delayed fluorescence (hereinafter, sometimes simply referred to as “delayed fluorescence”) has been proposed and studied.

A TADF (Thermally Activated Delayed Fluorescence) mechanism uses such a phenomenon that inverse intersystem crossing from triplet excitons to singlet excitons thermally occurs when a material having a small energy difference (ΔST) between singlet energy level and triplet energy level is used. Thermally activated delayed fluorescence is explained in “Yuki Hando-tai no Debaisu Bussei (Device Physics of Organic Semiconductors)” (edited by ADACHI, Chihaya, published by Kodansha, issued on Apr. 1, 2012, on pages 261-268).

As a compound exhibiting TADF properties (hereinafter also referred to as a TADF compound), for example, a compound in which a donor moiety and an acceptor moiety are bonded in a molecule is known.

Examples of literatures relating to an organic EL device include Patent Literatures 1, 2 and 3 and Non-Patent Literature 1.

### CITATION LIST

### Patent Literature(s)

Patent Literature 1: Chinese Patent Application Publication No. 107793398
Patent Literature 2: International Publication No. WO2014/166585
Patent Literature 3: Korean Patent Publication No. 2014-143618

### Non-Patent Literature(s)

Non-Patent Literature 1: Nature Communications, 2014, 5, 4016

### SUMMARY OF THE INVENTION

### Problems to be Solved by the Invention

In order to improve performance of an electronic device such as a display, an organic EL device has been required to be highly improved in performance.

An object of the invention is to provide a compound and an organic electroluminescence device capable of achieving higher performance, particularly an improved luminous efficiency, and an electronic device including the organic electroluminescence device.

### Means for Solving the Problem(s)

According to an aspect of the invention, an organic electroluminescence device includes: an anode; a cathode; an emitting layer provided between the anode and the cathode, in which the emitting layer contains a delayed fluorescent compound M2 and a compound M3 represented by a formula (3) below, and a singlet energy S₁(M2) of the compound M2 and a singlet energy S₁(M3) of the compound M3 satisfy a relationship of a numerical formula (Numerical Formula 1) below,

S₁(M3)>S₁(M2) (Numerical Formula 1).

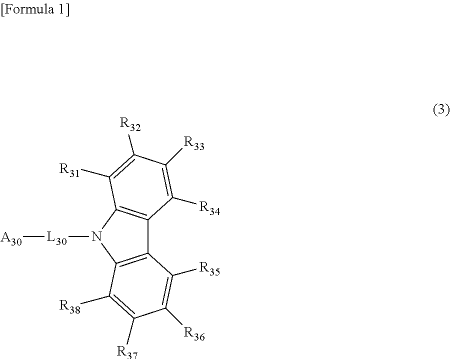

In the formula (3): A₃₀ is a group represented by a formula (3a), (3b), (3c), (3d), (3e) or (3f) below.

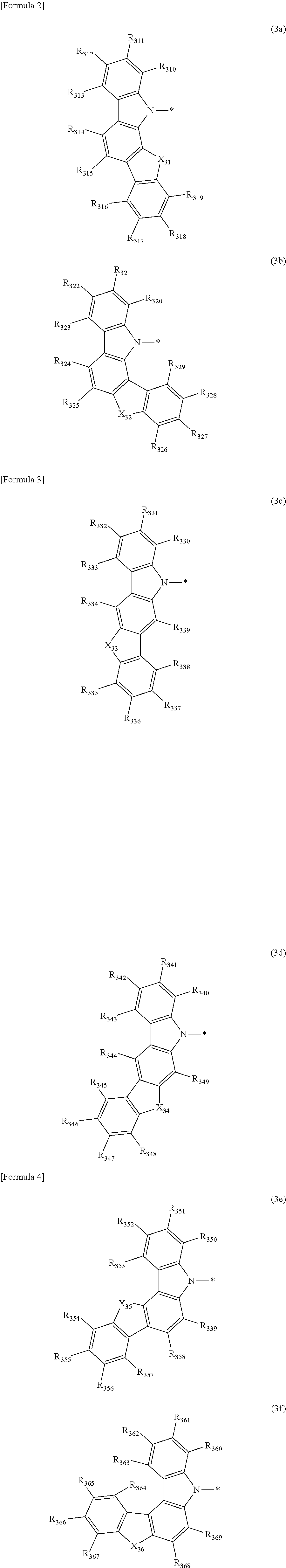

In the formulae (3a), (3b), (3c), (3d), (3e) and (3f)

X₃₁, X₃₂, X₃₃, X₃₄, X₃₅ and X₃₆ are each independently an oxygen atom, a sulfur atom, NR₃₇₁ or SiR₃₇₂R₃₇₃;

R₃₇₁, R₃₇₂ and R₃₇₃ are each independently a hydrogen atom or a substituent;

R₃₁₀ to R₃₁₉ are each independently a hydrogen atom or a substituent, or at least one pair of a pair of R₃₁₀ and R₃₁₁, a pair of R₃₁₁ and R₃₁₂, a pair of R₃₁₂ and R₃₁₃, a pair of R₃₁₄ and R₃₁₅, a pair of R₃₁₆ and R₃₁₇, a pair of R₃₁₇ and R₃₁₈, or a pair of R₃₁₈ and R₃₁₉ are mutually bonded to form a ring;

R₃₂₀ to R₃₂₉ are each independently a hydrogen atom or a substituent, or at least one pair of a pair of R₃₂₀ and R₃₂₁, a pair of R₃₂₁ and R₃₂₂, a pair of R₃₂₂ and R₃₂₃, a pair of R₃₂₄ and R₃₂₅, a pair of R₃₂₆ and R₃₂₇, a pair of R₃₂₇ and R₃₂₈, or a pair of R₃₂₈ and R₃₂₉ are mutually bonded to form a ring;

R₃₃₀ to R₃₃₉ are each independently a hydrogen atom or a substituent, or at least one pair of a pair of R₃₃₀ and R₃₃₁, a pair of R₃₃₁ and R₃₃₂, a pair of R₃₃₂ and R₃₃₃, a pair of R₃₃₅ and R₃₃₆, a pair of R₃₃₆ and R₃₃₇, or a pair of R₃₃₇ and R₃₃₈ are mutually bonded to form a ring;

R₃₄₀ to R₃₄₉ are each independently a hydrogen atom or a substituent, or at least one pair of a pair of R₃₄₀ and R₃₄₁, a pair of R₃₄₁ and R₃₄₂, a pair of R₃₄₂ and R₃₄₃, a pair of R₃₄₅ and R₃₄₆, a pair of R₃₄₆ and R₃₄₇, or a pair of R₃₄₇ and R₃₄₈ are mutually bonded to form a ring;

R₃₅₀ to R₃₅₉ are each independently a hydrogen atom or a substituent, or at least one pair of a pair of R₃₅₀ and R₃₅₁, a pair of R₃₅₁ and R₃₅₂, a pair of R₃₅₂ and R₃₅₃, a pair of R₃₅₄ and R₃₅₅, a pair of R₃₅₅ and R₃₅₆, a pair of R₃₅₆ and R₃₅₇, or a pair of R₃₅₈ and R₃₅₉ are mutually bonded to form a ring;

R₃₆₀ to R₃₆₉ are each independently a hydrogen atom or a substituent, or at least one pair of a pair of R₃₆₀ and R₃₆₁, a pair of R₃₆₁ and R₃₆₂, a pair of R₃₆₂ and R₃₆₃, a pair of R₃₆₄ and R₃₆₅, a pair of R₃₆₅ and R₃₆₆, a pair of R₃₆₆ and R₃₆₇, or a pair of R₃₆₈ and R₃₆₉ are mutually bonded to form a ring;

R₃₇₁, R₃₇₂ and R₃₇₃ as a substituent and R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

* represents a bonding position to L₃₀.

In the formula (3):

L₃₀ is a substituted or unsubstituted arylene group having 6 to 22 ring carbon atoms, a group formed by bonding two substituted or unsubstituted arylene groups having 6 to 22 ring carbon atoms, or a group formed by bonding three substituted or unsubstituted arylene groups having 6 to 22 ring carbon atoms;

R₃₁ to R₃₈ are each independently a hydrogen atom or a substituent, or at least one pair of a pair of R₃₁ and R₃₂, a pair of R₃₂ and R₃₃, a pair of R₃₃ and R₃₄, a pair of R₃₅ and R₃₆, a pair of R₃₆ and R₃₇, or a pair of R₃₇ and R₃₈ are mutually bonded to form a ring; and

a substituent for substituting L₃₀, and R₃₁ to R₃₈ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms.

According to another aspect of the invention, a compound represented by a formula (301) below is provided.

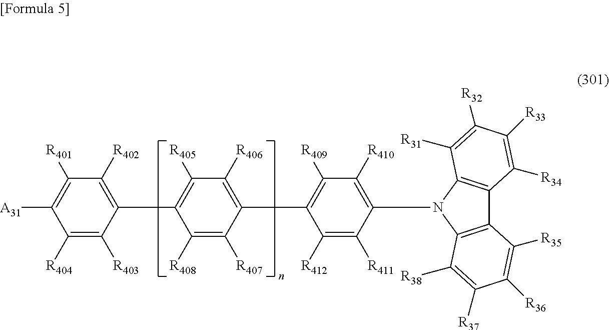

In the formula (301): A₃₁ is a group represented by a formula (31a), (31b), (31c), (31d), (31e) or (31f) below.

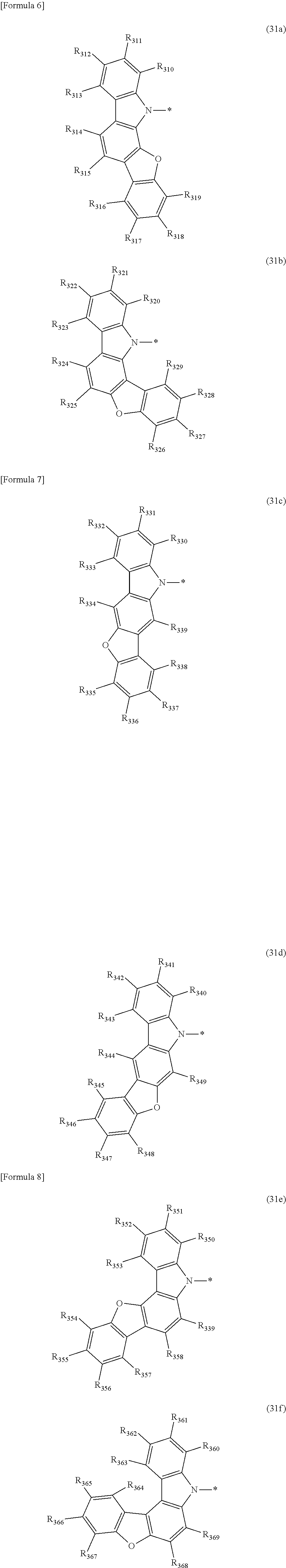

In the formulae (31a), (31b), (31c), (31d), (31e) and (31f):

R₃₁₀ to R₃₁₉ are each independently a hydrogen atom or a substituent;

R₃₂₀ to R₃₂₉ are each independently a hydrogen atom or a substituent;

R₃₃₀ to R₃₃₉ are each independently a hydrogen atom or a substituent;

R₃₄₀ to R₃₄₉ are each independently a hydrogen atom or a substituent;

R₃₅₀ to R₃₅₉ are each independently a hydrogen atom or a substituent;

R₃₆₀ to R₃₆₉ are each independently a hydrogen atom or a substituent;

R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ as a substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

* represents a bonding position to a benzene ring bonded to R₄₀₁ to R₄₀₄.

In the formula (301):

R₃₁ to R₃₈ are each independently a hydrogen atom or a substituent;

R₄₀₁ to R₄₁₂ are each independently a hydrogen atom or a substituent;

R₃₁ to R₃₈ as a substituent and R₄₀₁ to R₄₁₂ as a substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms;

n is 0, 1, 2 or 3;

when a plurality of R₄₀₅ are present, the plurality of R₄₀₅ are mutually the same or different;

when a plurality of R₄₀₆ are present, the plurality of R₄₀₆ are mutually the same or different;

when a plurality of R₄₀₇ are present, the plurality of R₄₀₇ are mutually the same or different; and

when a plurality of R₄₀₈ are present, the plurality of R₄₀₈ are mutually the same or different.

According to still another aspect of the invention, a compound represented by a formula (310) below is provided.

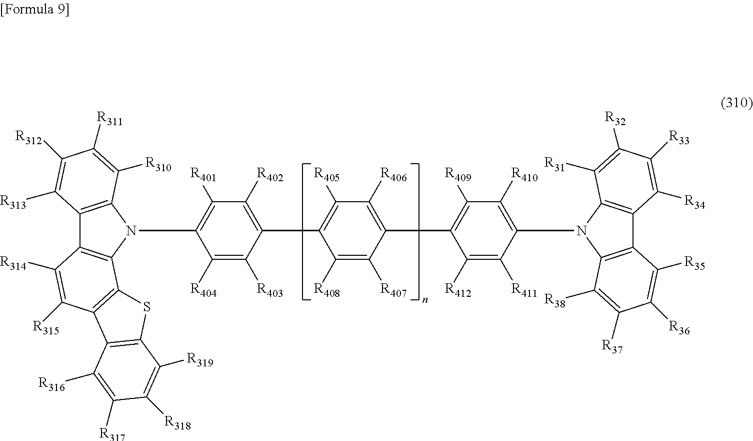

In the formula (310):

R₃₁₀ to R₃₁₉ are each independently a hydrogen atom or a substituent;

R₃₁ to R₃₈ are each independently a hydrogen atom or a substituent;

R₄₀₁ to R₄₁₂ are each independently a hydrogen atom or a substituent;

R₃₁₀ to R₃₁₉ as a substituent, R₃₁ to R₃₈ as a substituent and R₄₀₁ to R₄₁₂ as a substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms;

n is 0, 1, 2 or 3;

when a plurality of R₄₀₅ are present, the plurality of R₄₀₅ are mutually the same or different;

when a plurality of R₄₀₆ are present, the plurality of R₄₀₆ are mutually the same or different;

when a plurality of R₄₀₇ are present, the plurality of R₄₀₇ are mutually the same or different; and

when a plurality of R₄₀₈ are present, the plurality of R₄₀₈ are mutually the same or different.

According to a further aspect of the invention, an organic electroluminescence device includes: an anode; a cathode; an emitting layer provided between the anode and the cathode, in which the emitting layer contains a delayed fluorescent compound M2 and the compound according to any one of the above aspects of the invention.

According to a still further aspect of the invention, an electronic device including the organic electroluminescence device according to any one of the above aspects of the invention is provided.

According to the above aspects of the invention, a compound and an organic electroluminescence device capable of achieving higher performance, particularly an improved luminous efficiency, and an electronic device including the organic electroluminescence device can be provided.

### BRIEF DESCRIPTION OF DRAWING(S)

FIG. 1 schematically shows an exemplary arrangement of an organic electroluminescence device according to a first exemplary embodiment of the invention.

FIG. 2 schematically shows a device of measuring transient PL.

FIG. 3 shows an example of decay curves of the transient PL.

FIG. 4 schematically shows a relationship in energy level and energy transfer between a compound M3 and a compound M2 in an emitting layer of an exemplary organic electroluminescence device according to the first exemplary embodiment of the invention.

FIG. 5 schematically shows a relationship in energy level and energy transfer between the compound M3, the compound M2, and a compound M1 in an emitting layer of an exemplary organic electroluminescence device according to a second exemplary embodiment of the invention.

### DESCRIPTION OF EMBODIMENT(S)

### First Exemplary Embodiment

An arrangement of an organic EL device according to a first exemplary embodiment of the invention will be described below.

The organic EL device includes an anode, a cathode, and an organic layer between the anode and the cathode. This organic layer includes at least one layer formed of an organic compound(s). Alternatively, the organic layer includes a plurality of layers formed of an organic compound(s). The organic layer may further include an inorganic compound. In the organic EL device according to the exemplary embodiment, at least one layer of the organic layer is an emitting layer. Accordingly, the organic layer may, for instance, be provided by a single emitting layer, or include a layer(s) usable for an organic EL device. Examples of the layer(s) usable for the organic EL device, which are not particularly limited, include at least one layer selected from the group consisting of a hole injecting layer, hole transporting layer, electron injecting layer, electron transporting layer, and blocking layer.

The organic EL device according to the exemplary embodiment includes an emitting layer between the anode and the cathode.

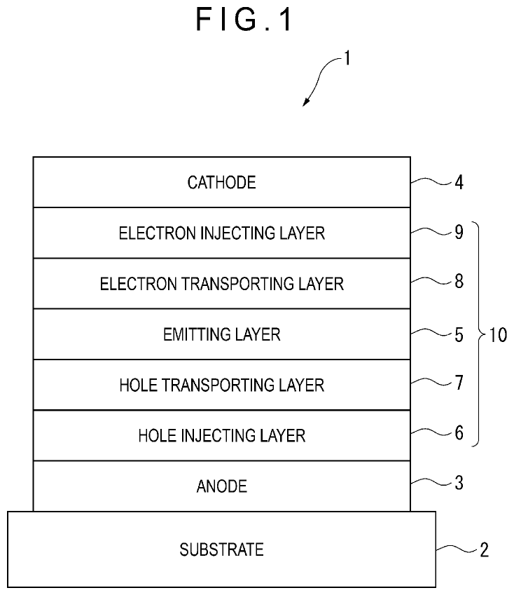

FIG. 1 schematically shows an exemplary arrangement of an organic EL device according to the first exemplary embodiment.

An organic EL device 1 includes a light-transmissive substrate 2, an anode 3, a cathode 4, and an organic layer 10 provided between the anode 3 and the cathode 4. The organic layer 10 includes a hole injecting layer 6, a hole transporting layer 7, an emitting layer 5, an electron transporting layer 8, and an electron injecting layer 9, which are sequentially laminated on the anode 3.

In an exemplary arrangement of the exemplary embodiment, the emitting layer may contain a metal complex.

In an exemplary arrangement of the exemplary embodiment, the emitting layer preferably does not contain a phosphorescent material (dopant material).

In an exemplary arrangement of the exemplary embodiment, the emitting layer preferably does not contain a heavy-metal complex and a phosphorescent rare earth metal complex. Examples of the heavy-metal complex herein include iridium complex, osmium complex, and platinum complex.

In an exemplary arrangement of the exemplary embodiment, the emitting layer also preferably does not contain a metal complex.

In the organic EL device according to the exemplary embodiment, the emitting layer contains a delayed fluorescent compound M2 and a compound M3 represented by a formula (3) below.

In this arrangement, the compound M2 is preferably a dopant material (also referred to as a guest material, emitter or luminescent material), and the compound M3 is preferably a host material (also referred to as a matrix material).

### Emitting Layer

### Compound M3

The emitting layer of the exemplary embodiment contains the compound M3 represented by the formula (3).

The compound M3 of the exemplary embodiment may be a thermally activated delayed fluorescent compound or a compound exhibiting no thermally activated delayed fluorescence. However, the compound M3 is preferably a compound exhibiting no thermally activated delayed fluorescence.

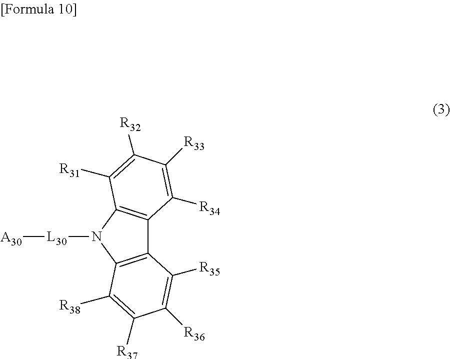

In the formula (3), A₃₀ is a group represented by a formula (3a), (3b), (3c), (3d), (3e) or (3f) below.

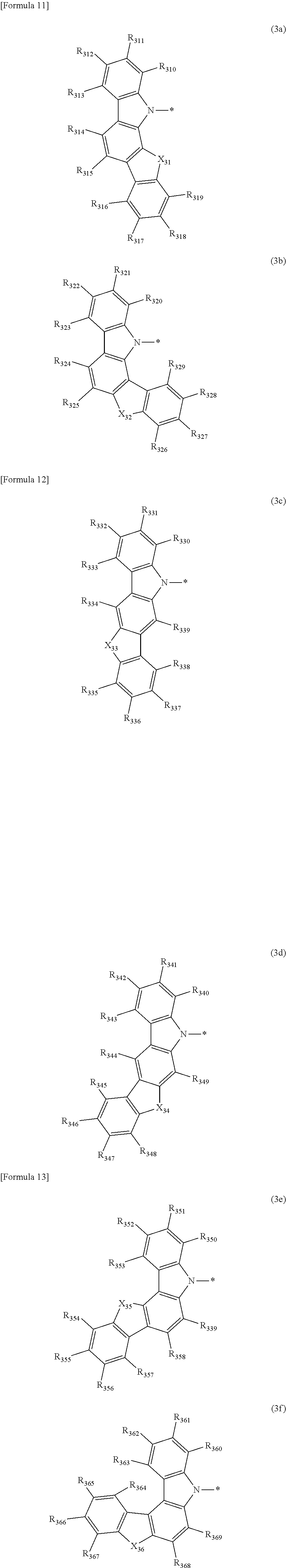

In the formulae (3a), (3b), (3c), (3d), (3e) and (3f):

X₃₁, X₃₂, X₃₃, X₃₄, X₃₅ and X₃₆ are each independently an oxygen atom, a sulfur atom, NR₃₇₁ or SiR₃₇₂R₃₇₃;

R₃₇₁, R₃₇₂ and R₃₇₃ are each independently a hydrogen atom or a substituent;

R₃₁₀ to R₃₁₉ are each independently a hydrogen atom or a substituent, or at least one pair of a pair of R₃₁₀ and R₃₁₁, a pair of R₃₁₁ and R₃₁₂, a pair of R₃₁₂ and R₃₁₃, a pair of R₃₁₄ and R₃₁₅, a pair of R₃₁₆ and R₃₁₇, a pair of R₃₁₇ and R₃₁₈, or a pair of R₃₁₈ and R₃₁₉ are mutually bonded to form a ring;

R₃₂₀ to R₃₂₉ are each independently a hydrogen atom or a substituent, or at least one pair of a pair of R₃₂₀ and R₃₂₁, a pair of R₃₂₁ and R₃₂₂, a pair of R₃₂₂ and R₃₂₃, a pair of R₃₂₄ and R₃₂₅, a pair of R₃₂₆ and R₃₂₇, a pair of R₃₂₇ and R₃₂₈, or a pair of R₃₂₈ and R₃₂₉ are mutually bonded to form a ring;

R₃₃₀ to R₃₃₉ are each independently a hydrogen atom or a substituent, or at least one pair of a pair of R₃₃₀ and R₃₃₁, a pair of R₃₃₁ and R₃₃₂, a pair of R₃₃₂ and R₃₃₃, a pair of R₃₃₅ and R₃₃₆, a pair of R₃₃₆ and R₃₃₇, or a pair of R₃₃₇ and R₃₃₈ are mutually bonded to form a ring;

R₃₄₀ to R₃₄₉ are each independently a hydrogen atom or a substituent, or at least one pair of a pair of R₃₄₀ and R₃₄₁, a pair of R₃₄₁ and R₃₄₂, a pair of R₃₄₂ and R₃₄₃, a pair of R₃₄₅ and R₃₄₆, a pair of R₃₄₆ and R₃₄₇, or a pair of R₃₄₇ and R₃₄₈ are mutually bonded to form a ring;

R₃₅₀ to R₃₅₉ are each independently a hydrogen atom or a substituent, or at least one pair of a pair of R₃₅₀ and R₃₅₁, a pair of R₃₅₁ and R₃₅₂, a pair of R₃₅₂ and R₃₅₃, a pair of R₃₅₄ and R₃₅₅, a pair of R₃₅₅ and R₃₅₆, a pair of R₃₅₆ and R₃₅₇, or a pair of R₃₅₈ and R₃₅₉ are mutually bonded to form a ring;

R₃₆₀ to R₃₆₉ are each independently a hydrogen atom or a substituent, or at least one pair of a pair of R₃₆₀ and R₃₆₁, a pair of R₃₆₁ and R₃₆₂, a pair of R₃₆₂ and R₃₆₃, a pair of R₃₆₄ and R₃₆₅, a pair of R₃₆₅ and R₃₆₆, a pair of R₃₆₆ and R₃₆₇, or a pair of R₃₆₈ and R₃₆₉ are mutually bonded to form a ring;

R₃₇₁, R₃₇₂ and R₃₇₃ as a substituent and R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

* represents a bonding position to L₃₀.

In the formula (3):

L₃₀ is a substituted or unsubstituted arylene group having 6 to 22 ring carbon atoms, a group formed by bonding two substituted or unsubstituted arylene groups having 6 to 22 ring carbon atoms, or a group formed by bonding three substituted or unsubstituted arylene groups having 6 to 22 ring carbon atoms;

R₃₁ to R₃₈ are each independently a hydrogen atom or a substituent, or at least one pair of a pair of R₃₁ and R₃₂, a pair of R₃₂ and R₃₃, a pair of R₃₃ and R₃₄, a pair of R₃₅ and R₃₆, a pair of R₃₆ and R₃₇, or a pair of R₃₇ and R₃₈ are mutually bonded to form a ring; and

a substituent for substituting L₃₀, and R₃₁ to R₃₈ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms.

In the compound M3, A₃₀ is preferably the group represented by the formula (3a) or (3d).

In the compound M3, A₃₀ is more preferably the group represented by the formula (3a). When A₃₀ is represented by the formula (3a), the compound M3 is represented by a formula (3A) below.

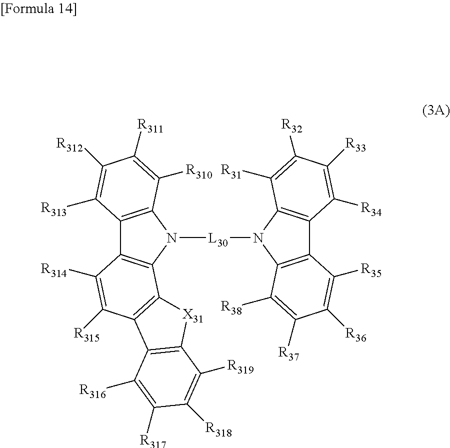

R₃₁ to R₃₈, L₃₀, X₃₁ and R₃₁₀ to R₃₁₉ in the formula (3A) respectively represent the same as R₃₁ to R₃₈, L₃₀, X₃₁ and R₃₁₀ to R₃₁₉ in the formula (3) or (3a).

In the compound M3, it is also preferable that a pair of R₃₁ and R₃₂, a pair of R₃₂ and R₃₃, a pair of R₃₃ and R₃₄, a pair of R₃₅ and R₃₆, a pair of R₃₆ and R₃₇, and a pair of R₃₇ and R₃₈ are not mutually bonded. In other words, in the compound M3, R₃₁ to R₃₈ are also preferably each independently a hydrogen atom or a substituent.

In the compound M3, it is also preferable that at least one pair of a pair of R₃₁ and R₃₂, a pair of R₃₂ and R₃₃, a pair of R₃₃ and R₃₄, a pair of R₃₅ and R₃₆, a pair of R₃₆ and R₃₇, or a pair of R₃₇ and R₃₈ are mutually bonded to form a ring.

The compound M3 is also preferably represented by a formula (31), (32), (33), (34), (35) or (36) below.

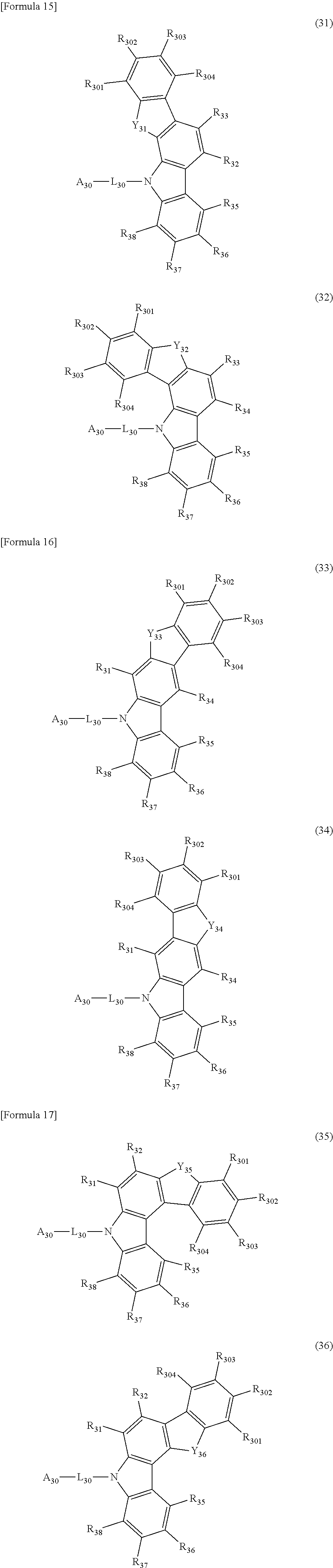

In the formulae (31) to (36):

Y₃₁, Y₃₂, Y₃₃, Y₃₄, Y₃₅ and Y₃₆ are each independently an oxygen atom, a sulfur atom, NR₃₇₄ or SiR₃₇₅R₃₇₆;

R₃₇₄, R₃₇₅ and R₃₇₆ are each independently a hydrogen atom or a substituent;

A₃₀, L₃₀ and R₃₁ to R₃₈ respectively represent the same as A₃₀, L₃₀ and R₃₁ to R₃₈ in the formula (3);

R₃₀₁ to R₃₀₄ are each independently a hydrogen atom or a substituent, or at least one pair of a pair of R₃₀₁ and R₃₀₂, a pair of R₃₀₂ and R₃₀₃, or a pair of R₃₀₃ and R₃₀₄ are mutually bonded to form a ring; and

R₃₀₁ to R₃₀₄ as a substituent and R₃₇₄, R₃₇₅ and R₃₇₆ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms.

In the compound M3, it is preferable that none of a pair of R₃₀₁ and R₃₀₂, a pair of R₃₀₂ and R₃₀₃, and a pair of R₃₀₃ and R₃₀₄ are mutually bonded.

In the compound M3, R₃₀₁ to R₃₀₄ are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound M3, R₃₀₁ to R₃₀₄ are more preferably each independently a hydrogen atom, a substituted or unsubstituted aryl group having 6 to 30 ring carbon atoms, or a substituted or unsubstituted heterocyclic group having 5 to 30 ring atoms.

In the compound M3, R₃₀₁ to R₃₀₄ are further preferably each independently a hydrogen atom or a substituted or unsubstituted aryl group having 6 to 30 ring carbon atoms.

In the compound M3, R₃₀₁ to R₃₀₄ are further more preferably a hydrogen atom.

In the compound M3, Y₃₁, Y₃₂, Y₃₃, Y₃₄, Y₃₅ and Y₃₆ are preferably each independently an oxygen atom or a sulfur atom.

In the compound M3, R₃₁ to R₃₈ are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound M3, R₃₁ to R₃₈ are more preferably each independently a hydrogen atom, a substituted or unsubstituted aryl group having 6 to 30 ring carbon atoms, or a substituted or unsubstituted heterocyclic group having 5 to 30 ring atoms.

In the compound M3, R₃₁ to R₃₈ are further preferably each independently a hydrogen atom or a substituted or unsubstituted aryl group having 6 to 30 ring carbon atoms.

In the compound M3, R₃₁ to R₃₈ are further more preferably a hydrogen atom.

In the compound M3, R₃₁ to R₃₈ and R₃₀₁ to R₃₀₄ are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound M3, R₃₁ to R₃₈ and R₃₀₁ to R₃₀₄ are more preferably each independently a hydrogen atom, a substituted or unsubstituted aryl group having 6 to 30 ring carbon atoms, or a substituted or unsubstituted heterocyclic group having 5 to 30 ring atoms.

In the compound M3, R₃₁ to R₃₈ and R₃₀₁ to R₃₀₄ are further preferably each independently a hydrogen atom or a substituted or unsubstituted aryl group having 6 to 30 ring carbon atoms.

In the compound M3, R₃₁ to R₃₈ and R₃₀₁ to R₃₀₄ are further more preferably a hydrogen atom.

In the compound M3, L₃₀ is preferably a substituted or unsubstituted phenylene group, a substituted or unsubstituted biphenylene group, or a substituted or unsubstituted terphenylene group.

In the compound M3, L₃₀ is more preferably a substituted or unsubstituted biphenylene group.

In the compound M3, it is preferable that a substituent for substituting L₃₀ (this substituent is sometimes referred to as a substituent F_{L}) is a substituted or unsubstituted aryl group having 6 to 30 ring carbon atoms or a substituted or unsubstituted alkyl group having 1 to 30 carbon atoms.

In the compound M3, L₃₀ is also preferably at least one group selected from the group consisting of groups represented by formulae (L30-1) to (L30-4), (L30-4-2) and (L30-5) to (L30-11) below.

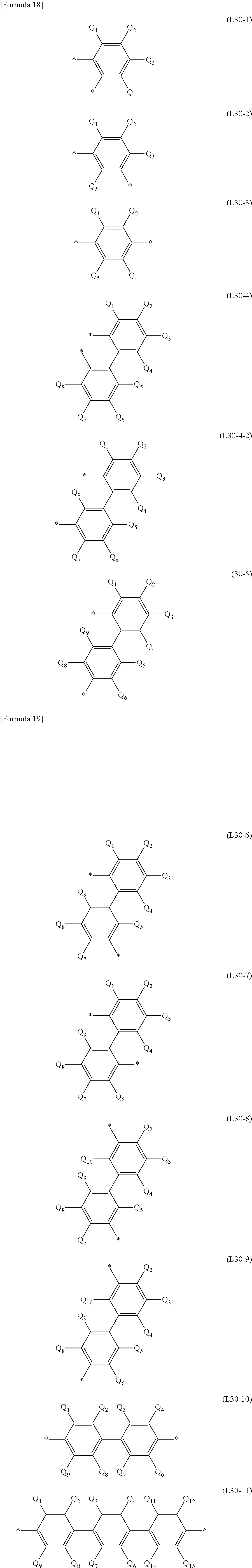

In the formulae (L30-1) to (L30-4), (L30-4-2) and (L30-5) to (L30-11):

Q₁ to Q₁₄ are each independently a hydrogen atom or a substituent; and

Q₁ to Q₁₄ as a substituent are each independently a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms.

Q₁ to Q₁₄ as a substituent are preferably each independently a hydrogen atom, a substituted or unsubstituted aryl group having 6 to 30 ring carbon atoms, or a substituted or unsubstituted alkyl group having 1 to 30 carbon atoms.

In the compound M3, X₃₁, X₃₂, X₃₃, X₃₄, X₃₅ and X₃₆ are also preferably each independently an oxygen atom, NR₃₀₁ or SiR₃₀₂R₃₀₃.

In the compound M3, X₃₁, X₃₂, X₃₃, X₃₄, X₃₅ and X₃₆ are also preferably each independently an oxygen atom or a sulfur atom.

In the compound M3, X₃₁, X₃₂, X₃₃, X₃₄, X₃₅ and X₃₆ are also preferably an oxygen atom.

In the compound M3: the formula (3a) is represented by, for instance, a formula (3a-1) below when X₃₁ is an oxygen atom; the formula (3a) is represented by, for instance, a formula (3a-2) below when X₃₁ is a sulfur atom; the formula (3a) is represented by, for instance, a formula (3a-3) below when X₃₁ is NR₃₇₁; and the formula (3a) is represented by, for instance, a formula (3a-4) when X₃₁ is SiR₃₇₂R₃₇₃.

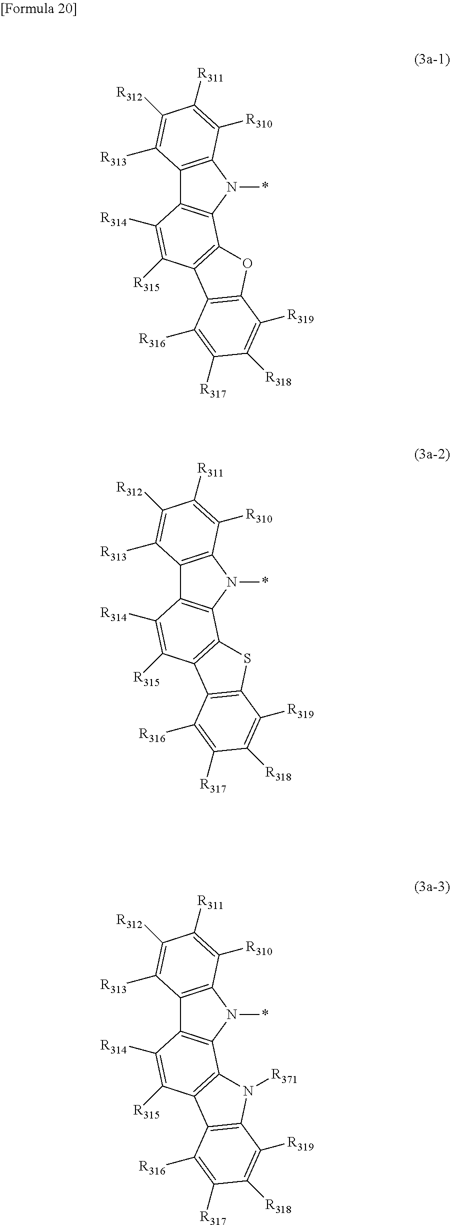

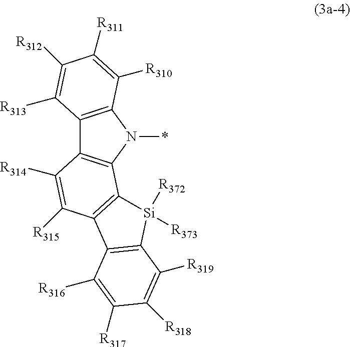

In the compound M3, it is preferable that: a pair of R₃₁₀ and R₃₁₁, a pair of R₃₁₁ and R₃₁₂, a pair of R₃₁₂ and R₃₁₃, a pair of R₃₁₄ and R₃₁₅, a pair of R₃₁₆ and R₃₁₇, a pair of R₃₁₇ and R₃₁₈, and a pair of R₃₁₈ and R₃₁₉ are not mutually bonded; a pair of R₃₂₀ and R₃₂₁, a pair of R₃₂₁ and R₃₂₂, a pair of R₃₂₂ and R₃₂₃, a pair of R₃₂₄ and R₃₂₅, a pair of R₃₂₆ and R₃₂₇, a pair of R₃₂₇ and R₃₂₈, and a pair of R₃₂₈ and R₃₂₉ are not mutually bonded; a pair of R₃₃₀ and R₃₃₁, a pair of R₃₃₁ and R₃₃₂, a pair of R₃₃₂ and R₃₃₃, a pair of R₃₃₅ and R₃₃₆, a pair of R₃₃₆ and R₃₃₇, and a pair of R₃₃₇ and R₃₃₈ are not mutually bonded; a pair of R₃₄₀ and R₃₄₁, a pair of R₃₄₁ and R₃₄₂, a pair of R₃₄₂ and R₃₄₃, a pair of R₃₄₅ and R₃₄₆, a pair of R₃₄₆ and R₃₄₇, and a pair of R₃₄₇ and R₃₄₈ are not mutually bonded; a pair of R₃₅₀ and R₃₅₁, a pair of R₃₅₁ and R₃₅₂, a pair of R₃₅₂ and R₃₅₃, a pair of R₃₅₄ and R₃₅₅, a pair of R₃₅₅ and R₃₅₆, a pair of R₃₅₆ and R₃₅₇, and a pair of R₃₅₈ and R₃₅₉ are not mutually bonded; and a pair of R₃₆₀ and R₃₆₁, a pair of R₃₆₁ and R₃₆₂, a pair of R₃₆₂ and R₃₆₃, a pair of R₃₆₄ and R₃₆₅, a pair of R₃₆₅ and R₃₆₆, a pair of R₃₆₆ and R₃₆₇, and a pair of R₃₆₈ and R₃₆₉ are not mutually bonded.

In the compound M3, R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound M3, R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ are more preferably each independently a hydrogen atom, a substituted or unsubstituted aryl group having 6 to 30 ring carbon atoms, or a substituted or unsubstituted heterocyclic group having 5 to 30 ring atoms.

In the compound M3, R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ are further preferably each independently a hydrogen atom or a substituted or unsubstituted aryl group having 6 to 30 ring carbon atoms.

In the compound M3, R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ are further more preferably a hydrogen atom.

### Manufacturing Method of Compound M3 According to Exemplary Embodiment

The compound M3 of the exemplary embodiment can be manufactured, for instance, by a method described later in Examples. The compound M3 of the exemplary embodiment can be manufactured, for instance, by application of known substitution reactions and/or materials depending on a target compound according to reactions described later in Examples.

### Specific Examples of Compound M3

Specific examples of the compound M3 of the exemplary embodiment include compounds below. It should however be noted that the invention is not limited by the specific examples of the compound.

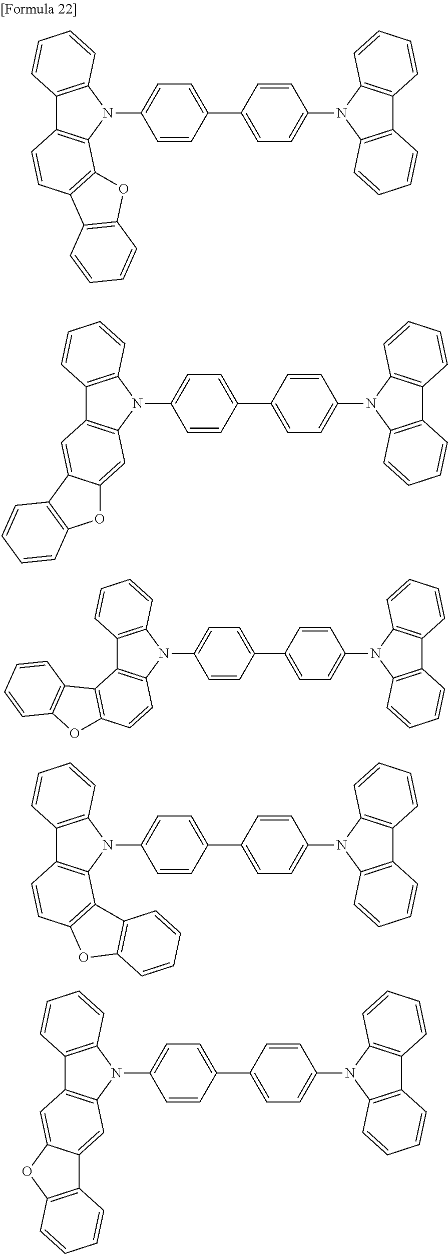

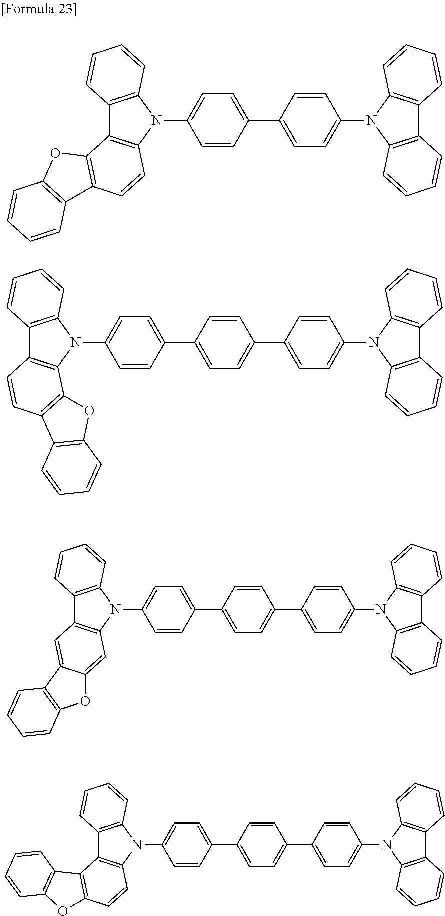

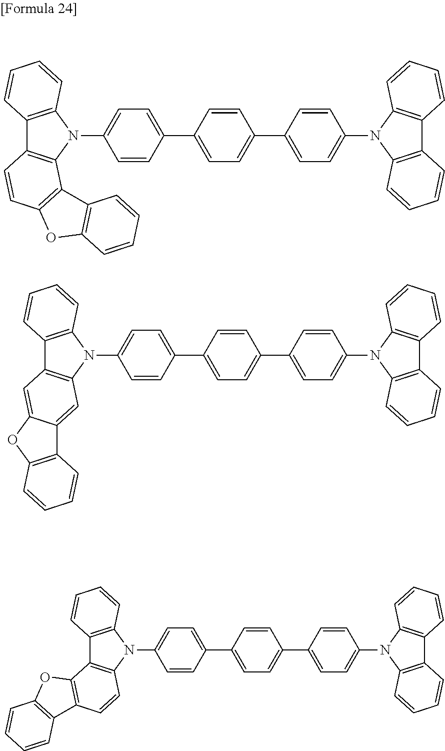 [SMILES: c1ccc2c(c1)oc1ccc3c4ccccc4n(-c4ccc(-c5ccc(-c6ccc(-n7c8ccccc8c8ccccc87)cc6)cc5)cc4)c3c12 ; c1ccc2c(c1)oc1cc3c4ccccc4n(-c4ccc(-c5ccc(-c6ccc(-n7c8ccccc8c8ccccc87)cc6)cc5)cc4)c3cc12 ; c1ccc2c(c1)oc1c2ccc2c1c1ccccc1n2-c1ccc(-c2ccc(-c3ccc(-n4c5ccccc5c5ccccc54)cc3)cc2)cc1]

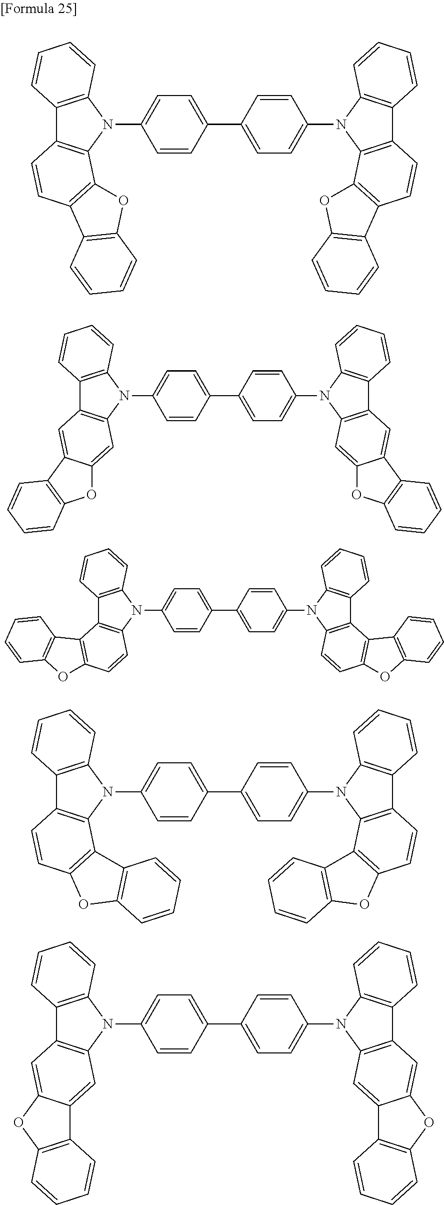

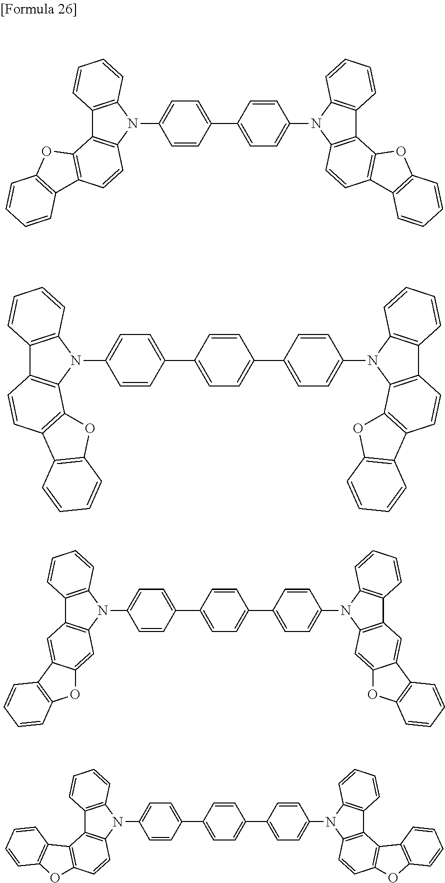 [SMILES: c1ccc2c(c1)oc1c2ccc2c1c1ccccc1n2-c1ccc(-c2ccc(-n3c4ccccc4c4c5oc6ccccc6c5ccc43)cc2)cc1 ; c1ccc2c(c1)oc1c2ccc2c3ccccc3n(-c3ccc(-c4ccc(-c5ccc(-n6c7ccccc7c7ccc8c9ccccc9oc8c76)cc5)cc4)cc3)c21 ; c1ccc2c(c1)oc1cc3c(cc12)c1ccccc1n3-c1ccc(-c2ccc(-c3ccc(-n4c5ccccc5c5cc6c(cc54)oc4ccccc46)cc3)cc2)cc1 ; c1ccc2c(c1)oc1ccc3c(c4ccccc4n3-c3ccc(-c4ccc(-c5ccc(-n6c7ccccc7c7c8c(ccc76)oc6ccccc68)cc5)cc4)cc3)c12]

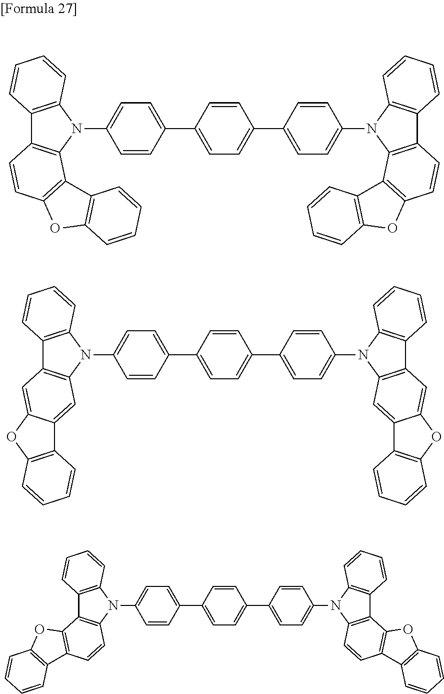 [SMILES: c1ccc2c(c1)oc1ccc3c4ccccc4n(-c4ccc(-c5ccc(-c6ccc(-n7c8ccccc8c8ccc9oc%10ccccc%10c9c87)cc6)cc5)cc4)c3c12 ; c1ccc2c(c1)oc1cc3c4ccccc4n(-c4ccc(-c5ccc(-c6ccc(-n7c8ccccc8c8cc9oc%10ccccc%10c9cc87)cc6)cc5)cc4)c3cc12 ; c1ccc2c(c1)oc1c2ccc2c1c1ccccc1n2-c1ccc(-c2ccc(-c3ccc(-n4c5ccccc5c5c6oc7ccccc7c6ccc54)cc3)cc2)cc1]

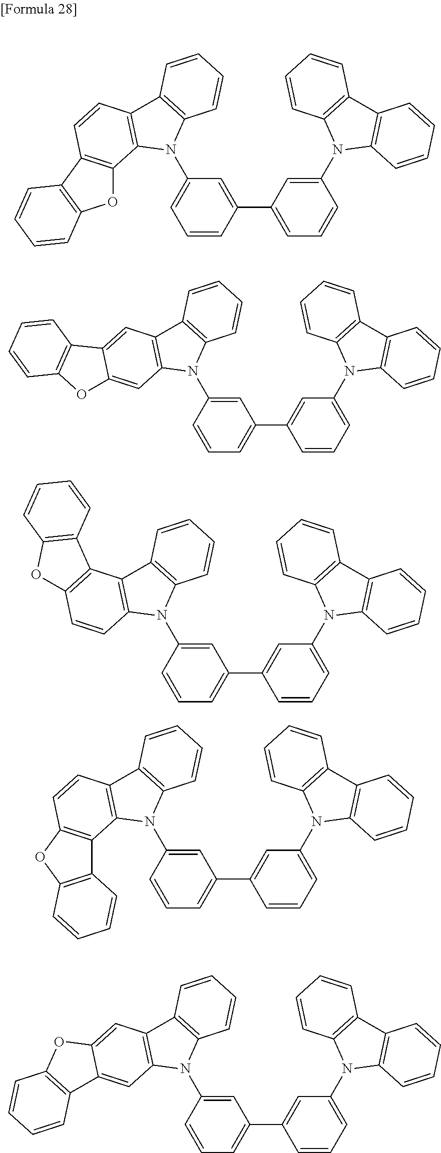

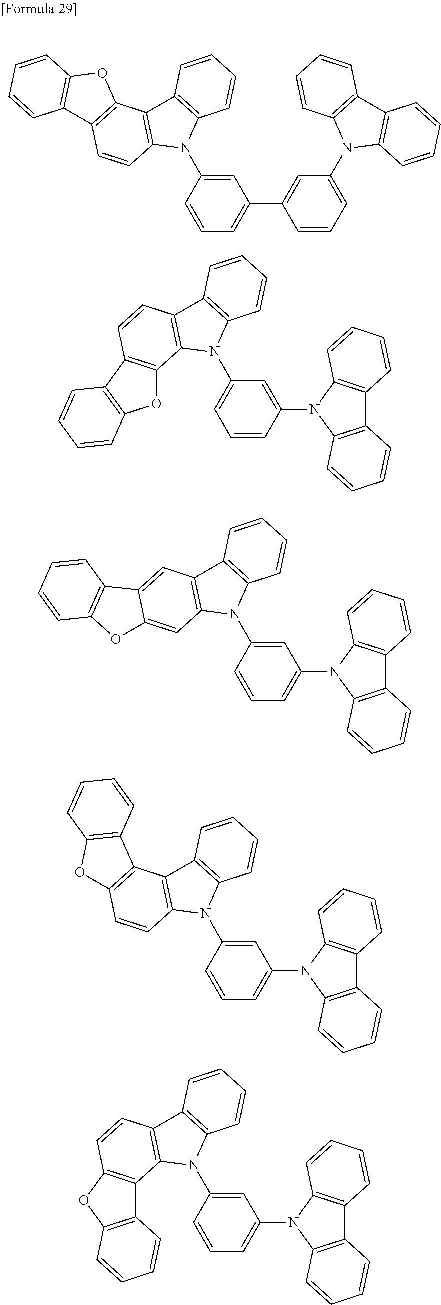

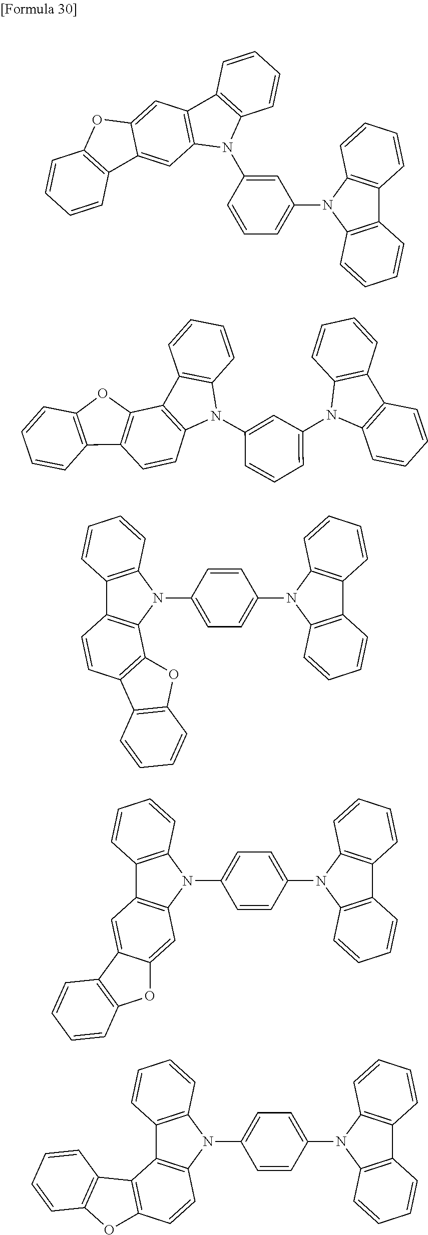

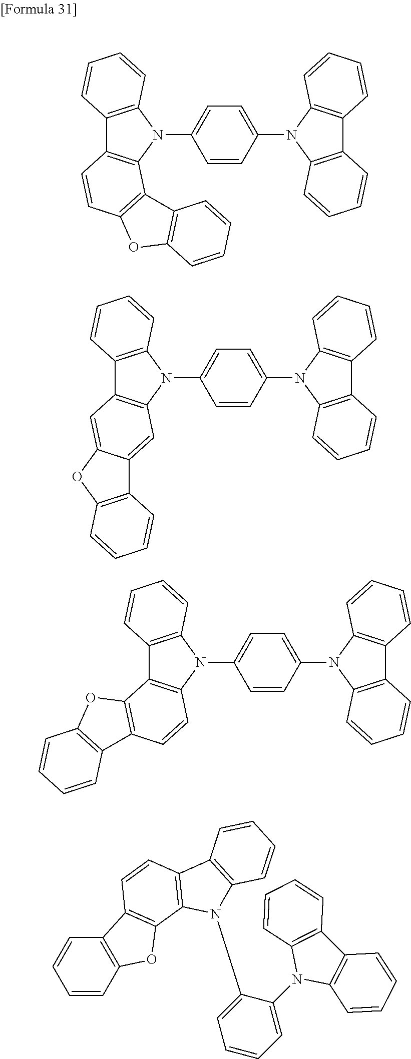

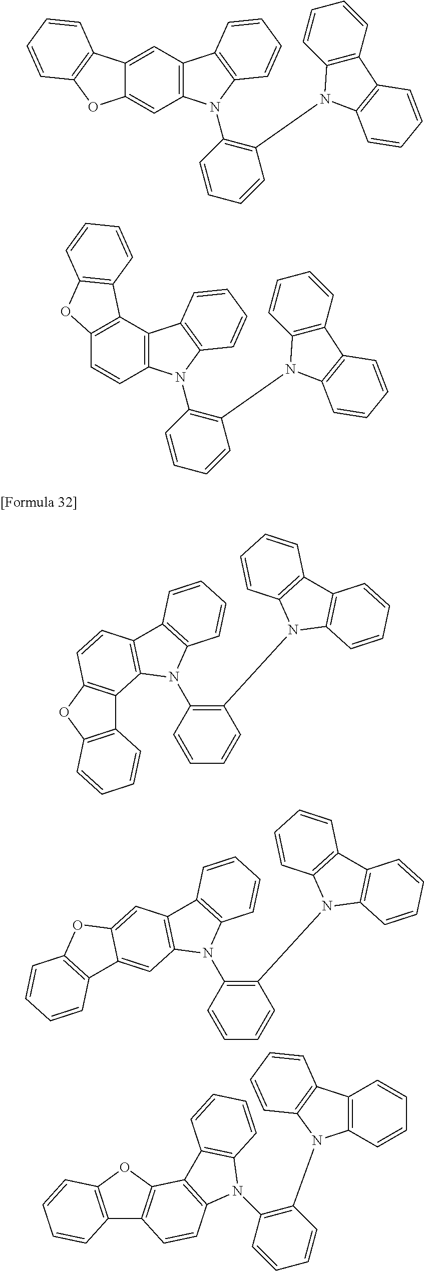

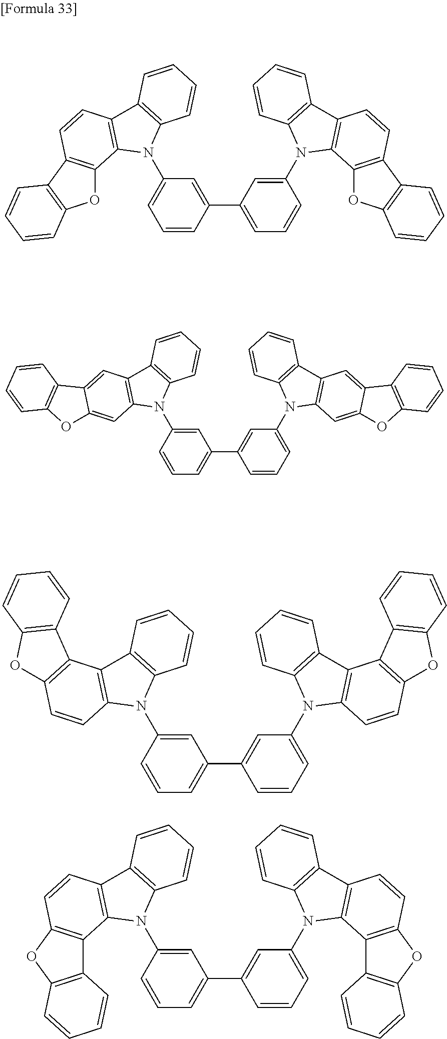

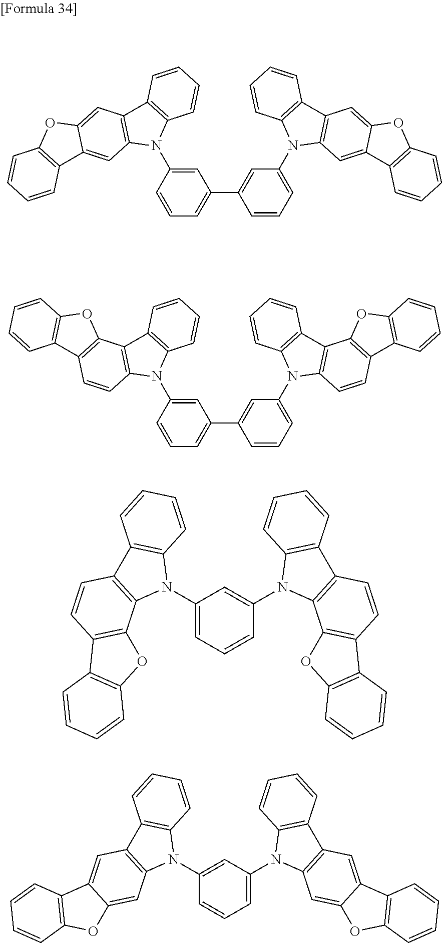 [SMILES: c1cc(-c2cccc(-n3c4ccccc4c4cc5oc6ccccc6c5cc43)c2)cc(-n2c3ccccc3c3cc4oc5ccccc5c4cc32)c1 ; c1cc(-c2cccc(-n3c4ccccc4c4c5oc6ccccc6c5ccc43)c2)cc(-n2c3ccccc3c3c4oc5ccccc5c4ccc32)c1 ; c1cc(-n2c3ccccc3c3ccc4c5ccccc5oc4c32)cc(-n2c3ccccc3c3ccc4c5ccccc5oc4c32)c1 ; c1cc(-n2c3ccccc3c3cc4c(cc32)oc2ccccc24)cc(-n2c3ccccc3c3cc4c(cc32)oc2ccccc24)c1]

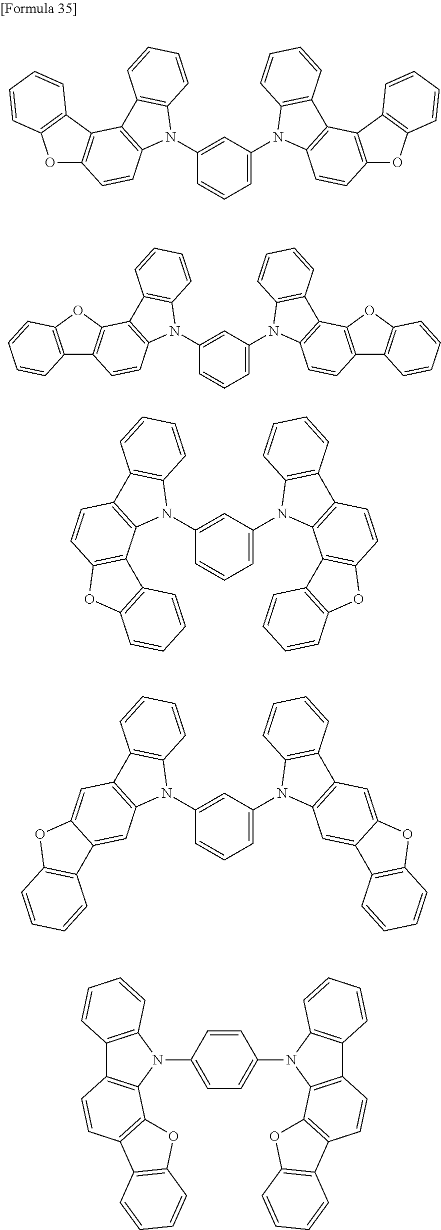 [SMILES: c1cc(-n2c3ccccc3c3c4c(ccc32)oc2ccccc24)cc(-n2c3ccccc3c3c4c(ccc32)oc2ccccc24)c1 ; c1cc(-n2c3ccccc3c3c4oc5ccccc5c4ccc32)cc(-n2c3ccccc3c3c4oc5ccccc5c4ccc32)c1 ; c1cc(-n2c3ccccc3c3ccc4oc5ccccc5c4c32)cc(-n2c3ccccc3c3ccc4oc5ccccc5c4c32)c1 ; c1cc(-n2c3ccccc3c3cc4oc5ccccc5c4cc32)cc(-n2c3ccccc3c3cc4oc5ccccc5c4cc32)c1 ; c1ccc2c(c1)oc1c2ccc2c3ccccc3n(-c3ccc(-n4c5ccccc5c5ccc6c7ccccc7oc6c54)cc3)c21]

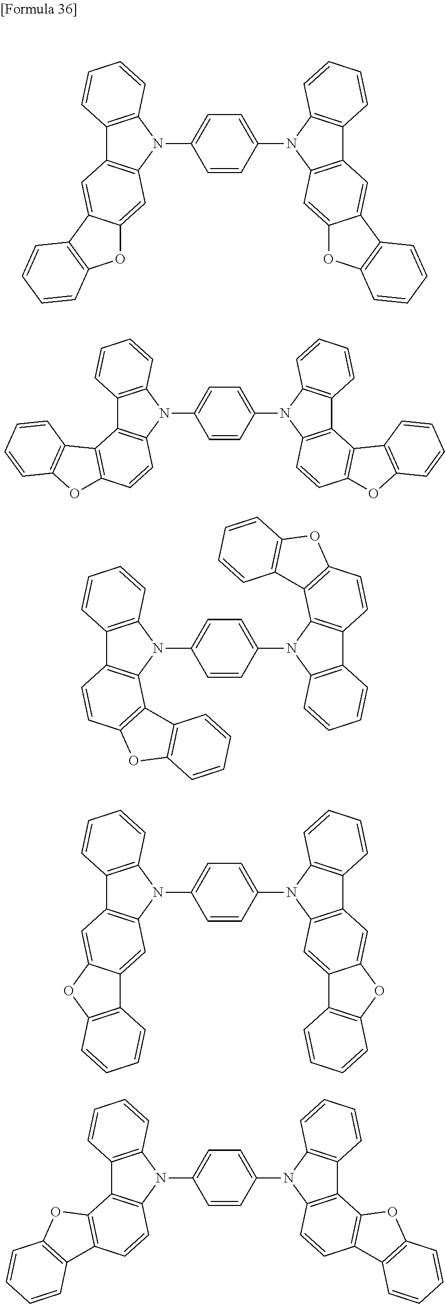

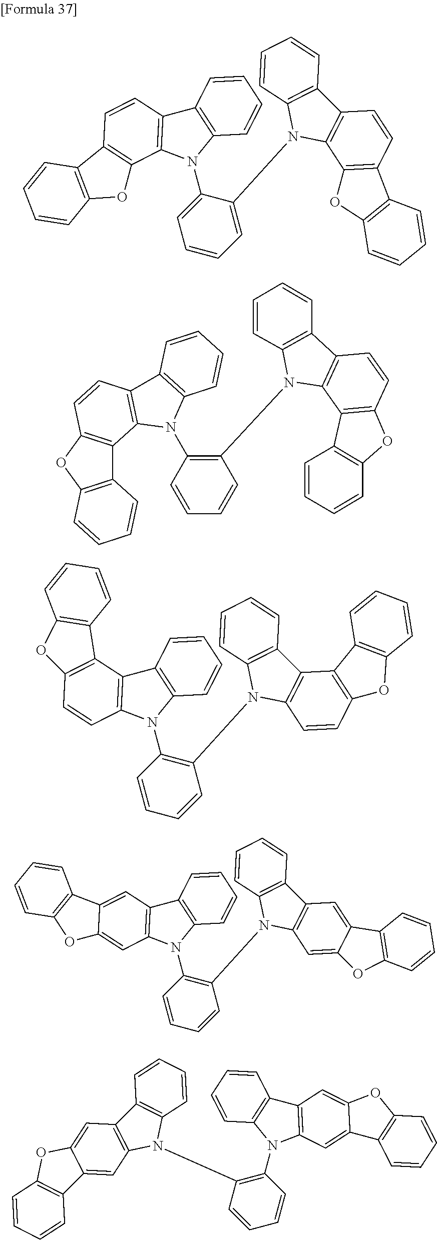

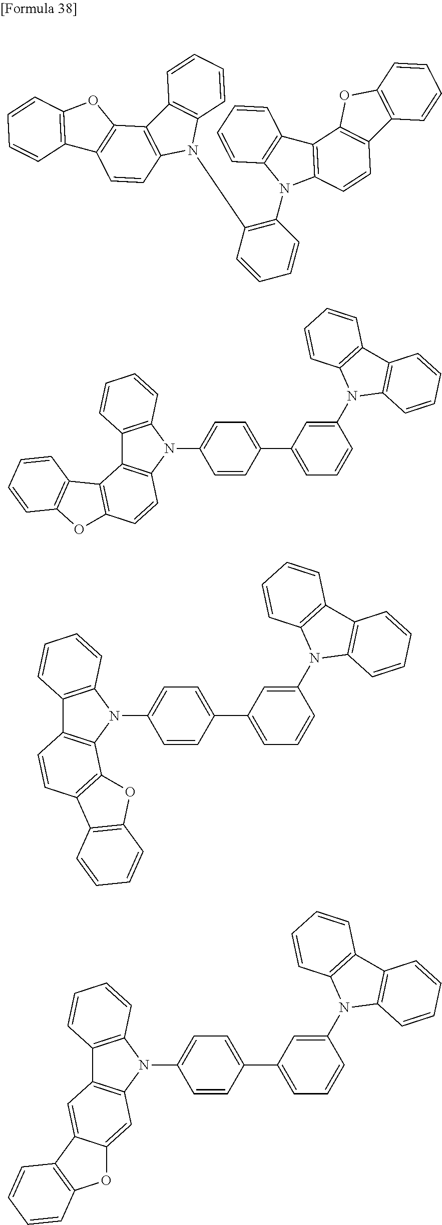 [SMILES: c1ccc(-n2c3ccccc3c3c4oc5ccccc5c4ccc32)c(-n2c3ccccc3c3c4oc5ccccc5c4ccc32)c1 ; c1cc(-c2ccc(-n3c4ccccc4c4c5c(ccc43)oc3ccccc35)cc2)cc(-n2c3ccccc3c3ccccc32)c1 ; c1cc(-c2ccc(-n3c4ccccc4c4ccc5c6ccccc6oc5c43)cc2)cc(-n2c3ccccc3c3ccccc32)c1 ; c1cc(-c2ccc(-n3c4ccccc4c4cc5c(cc43)oc3ccccc35)cc2)cc(-n2c3ccccc3c3ccccc32)c1]

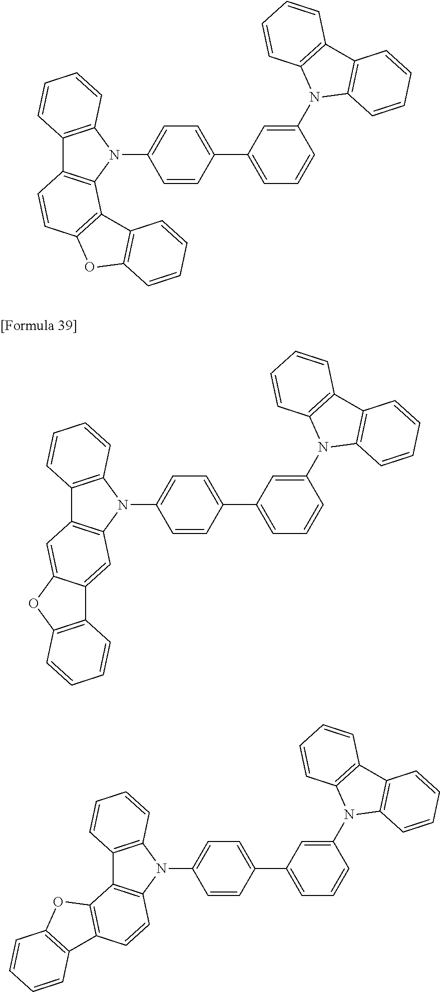 [SMILES: c1cc(-c2ccc(-n3c4ccccc4c4ccc5oc6ccccc6c5c43)cc2)cc(-n2c3ccccc3c3ccccc32)c1 ; c1cc(-c2ccc(-n3c4ccccc4c4cc5oc6ccccc6c5cc43)cc2)cc(-n2c3ccccc3c3ccccc32)c1 ; c1cc(-c2ccc(-n3c4ccccc4c4c5oc6ccccc6c5ccc43)cc2)cc(-n2c3ccccc3c3ccccc32)c1]

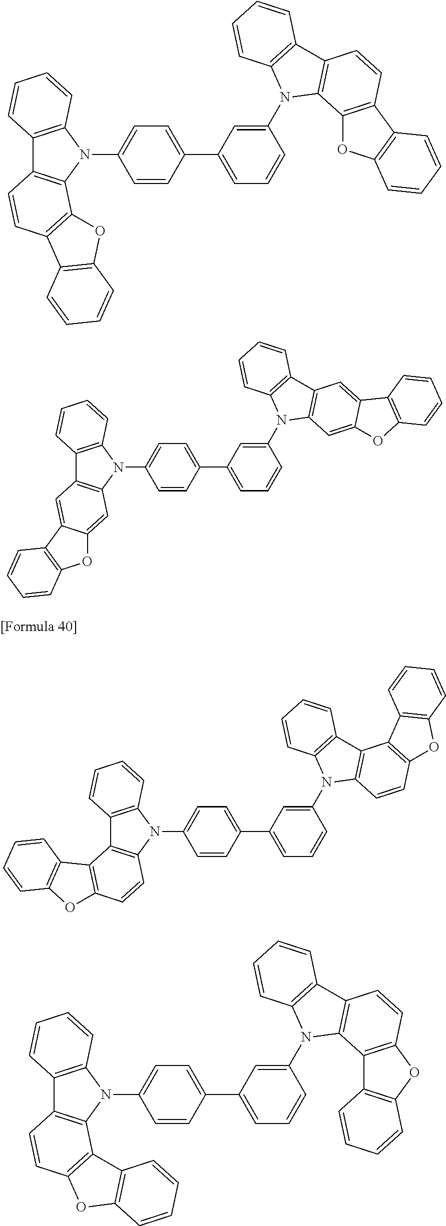 [SMILES: c1cc(-c2ccc(-n3c4ccccc4c4ccc5c6ccccc6oc5c43)cc2)cc(-n2c3ccccc3c3ccc4c5ccccc5oc4c32)c1 ; c1cc(-c2ccc(-n3c4ccccc4c4cc5c(cc43)oc3ccccc35)cc2)cc(-n2c3ccccc3c3cc4c(cc32)oc2ccccc24)c1 ; c1cc(-c2ccc(-n3c4ccccc4c4c5c(ccc43)oc3ccccc35)cc2)cc(-n2c3ccccc3c3c4c(ccc32)oc2ccccc24)c1 ; c1cc(-c2ccc(-n3c4ccccc4c4ccc5oc6ccccc6c5c43)cc2)cc(-n2c3ccccc3c3ccc4oc5ccccc5c4c32)c1]

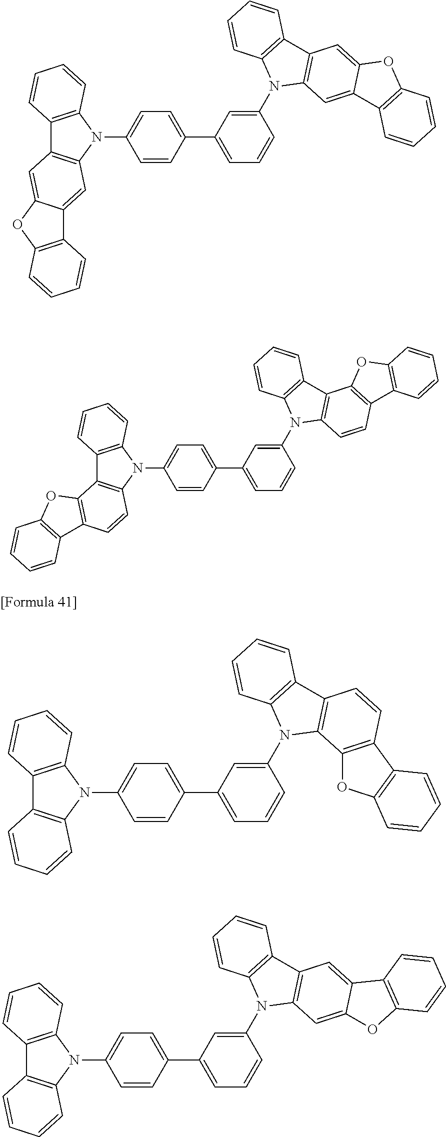 [SMILES: c1cc(-c2ccc(-n3c4ccccc4c4cc5oc6ccccc6c5cc43)cc2)cc(-n2c3ccccc3c3cc4oc5ccccc5c4cc32)c1 ; c1cc(-c2ccc(-n3c4ccccc4c4c5oc6ccccc6c5ccc43)cc2)cc(-n2c3ccccc3c3c4oc5ccccc5c4ccc32)c1 ; c1cc(-c2ccc(-n3c4ccccc4c4ccccc43)cc2)cc(-n2c3ccccc3c3ccc4c5ccccc5oc4c32)c1 ; c1cc(-c2ccc(-n3c4ccccc4c4ccccc43)cc2)cc(-n2c3ccccc3c3cc4c(cc32)oc2ccccc24)c1]

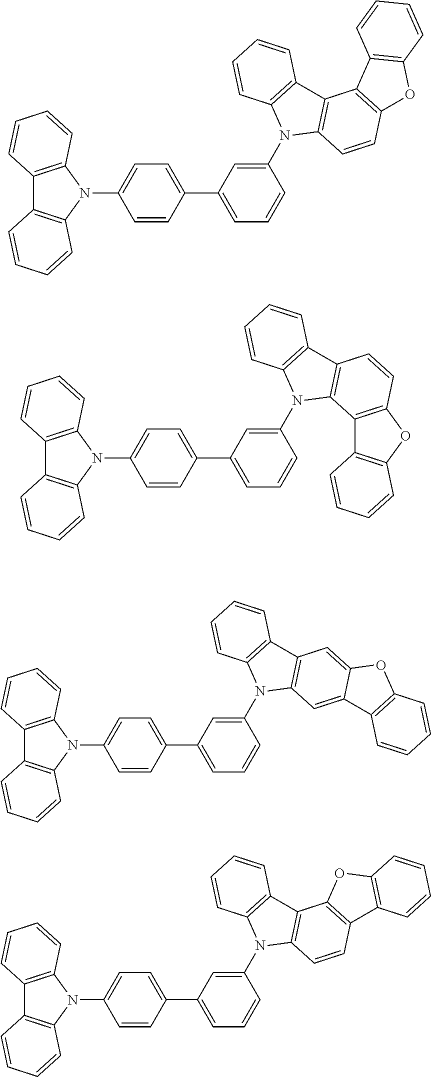 [SMILES: c1cc(-c2ccc(-n3c4ccccc4c4ccccc43)cc2)cc(-n2c3ccccc3c3c4c(ccc32)oc2ccccc24)c1 ; c1cc(-c2ccc(-n3c4ccccc4c4ccccc43)cc2)cc(-n2c3ccccc3c3ccc4oc5ccccc5c4c32)c1 ; c1cc(-c2ccc(-n3c4ccccc4c4ccccc43)cc2)cc(-n2c3ccccc3c3cc4oc5ccccc5c4cc32)c1 ; c1cc(-c2ccc(-n3c4ccccc4c4ccccc43)cc2)cc(-n2c3ccccc3c3c4oc5ccccc5c4ccc32)c1]

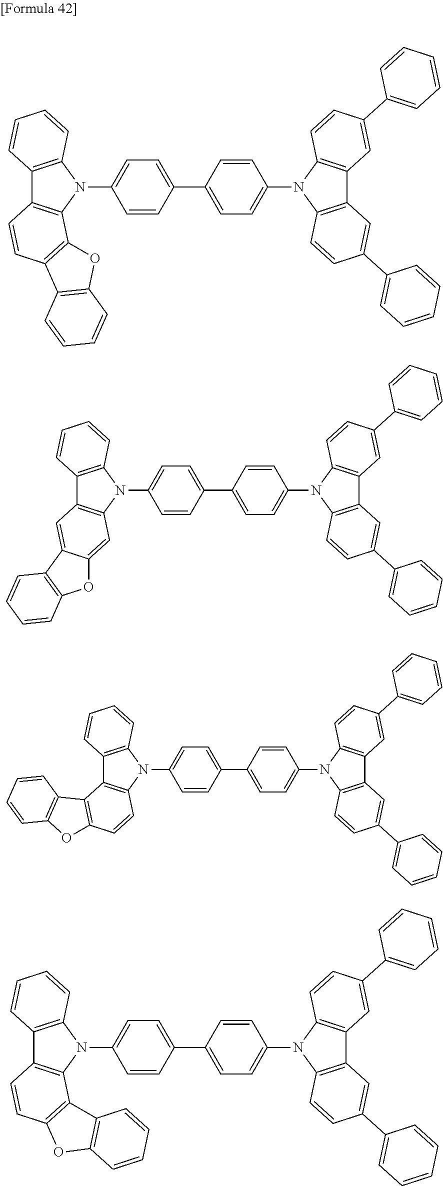

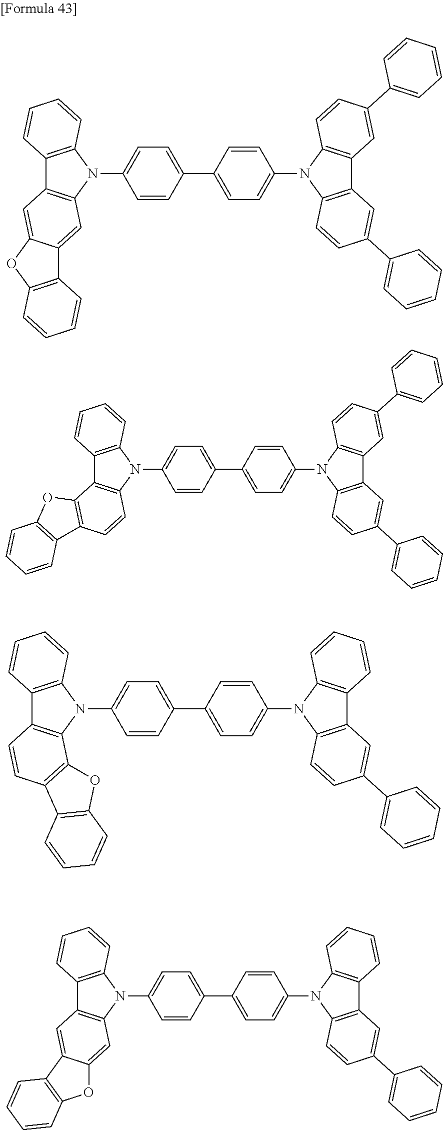 [SMILES: c1ccc(-c2ccc3c(c2)c2cc(-c4ccccc4)ccc2n3-c2ccc(-c3ccc(-n4c5ccccc5c5cc6oc7ccccc7c6cc54)cc3)cc2)cc1 ; c1ccc(-c2ccc3c(c2)c2cc(-c4ccccc4)ccc2n3-c2ccc(-c3ccc(-n4c5ccccc5c5c6oc7ccccc7c6ccc54)cc3)cc2)cc1 ; c1ccc(-c2ccc3c(c2)c2ccccc2n3-c2ccc(-c3ccc(-n4c5ccccc5c5ccc6c7ccccc7oc6c54)cc3)cc2)cc1 ; c1ccc(-c2ccc3c(c2)c2ccccc2n3-c2ccc(-c3ccc(-n4c5ccccc5c5cc6c(cc54)oc4ccccc46)cc3)cc2)cc1]

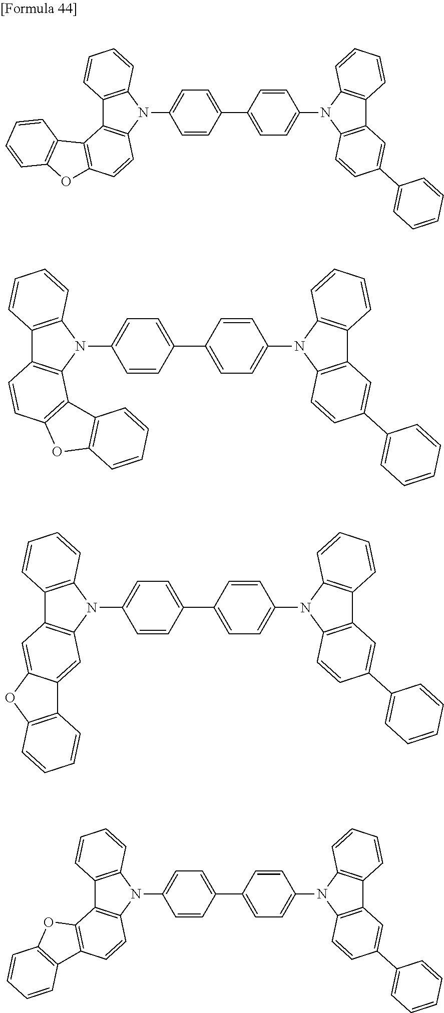 [SMILES: c1ccc(-c2ccc3c(c2)c2ccccc2n3-c2ccc(-c3ccc(-n4c5ccccc5c5c6c(ccc54)oc4ccccc46)cc3)cc2)cc1 ; c1ccc(-c2ccc3c(c2)c2ccccc2n3-c2ccc(-c3ccc(-n4c5ccccc5c5ccc6oc7ccccc7c6c54)cc3)cc2)cc1 ; c1ccc(-c2ccc3c(c2)c2ccccc2n3-c2ccc(-c3ccc(-n4c5ccccc5c5cc6oc7ccccc7c6cc54)cc3)cc2)cc1 ; c1ccc(-c2ccc3c(c2)c2ccccc2n3-c2ccc(-c3ccc(-n4c5ccccc5c5c6oc7ccccc7c6ccc54)cc3)cc2)cc1]

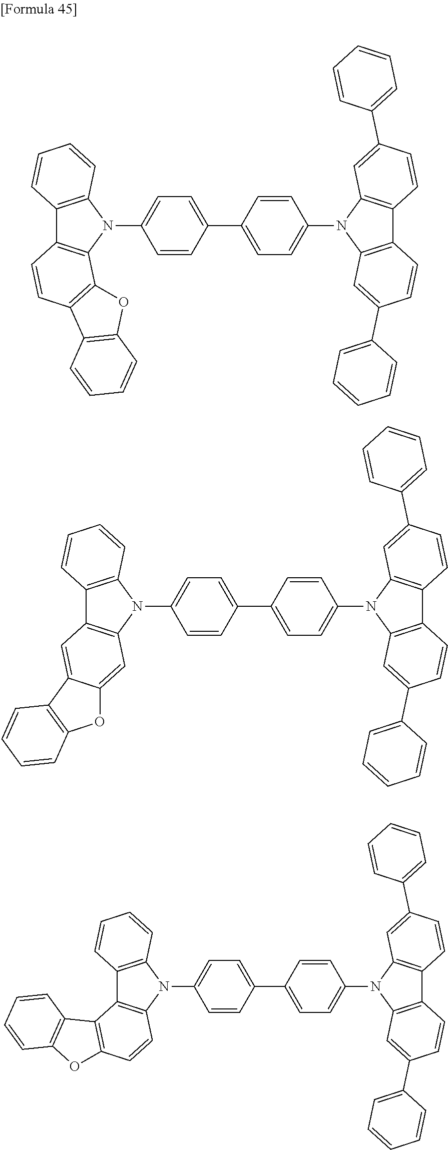

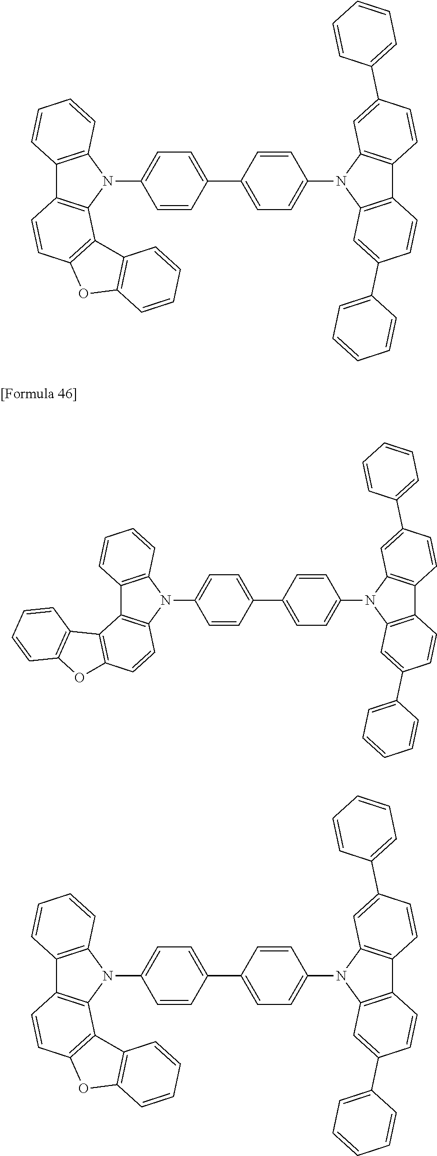 [SMILES: c1ccc(-c2ccc3c4ccc(-c5ccccc5)cc4n(-c4ccc(-c5ccc(-n6c7ccccc7c7ccc8oc9ccccc9c8c76)cc5)cc4)c3c2)cc1 ; c1ccc(-c2ccc3c4ccc(-c5ccccc5)cc4n(-c4ccc(-c5ccc(-n6c7ccccc7c7c8c(ccc76)oc6ccccc68)cc5)cc4)c3c2)cc1 ; c1ccc(-c2ccc3c4ccc(-c5ccccc5)cc4n(-c4ccc(-c5ccc(-n6c7ccccc7c7ccc8oc9ccccc9c8c76)cc5)cc4)c3c2)cc1]

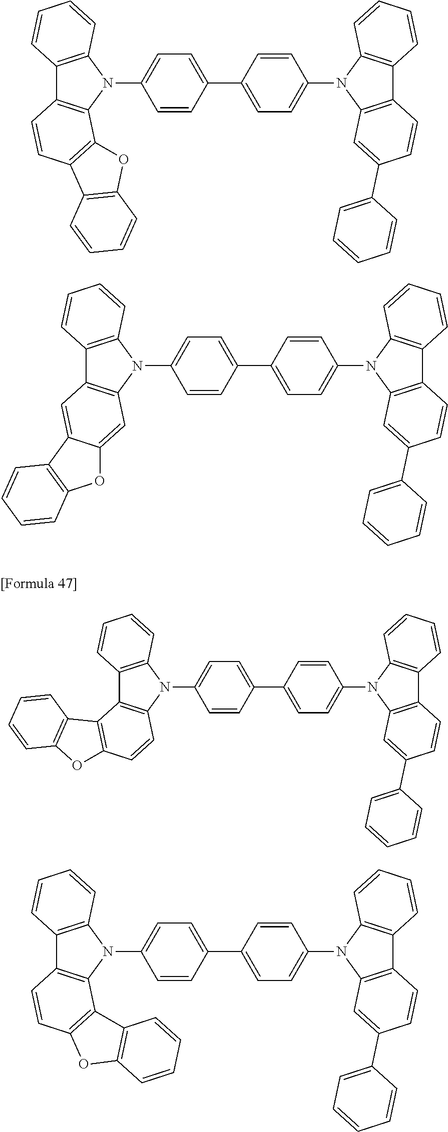

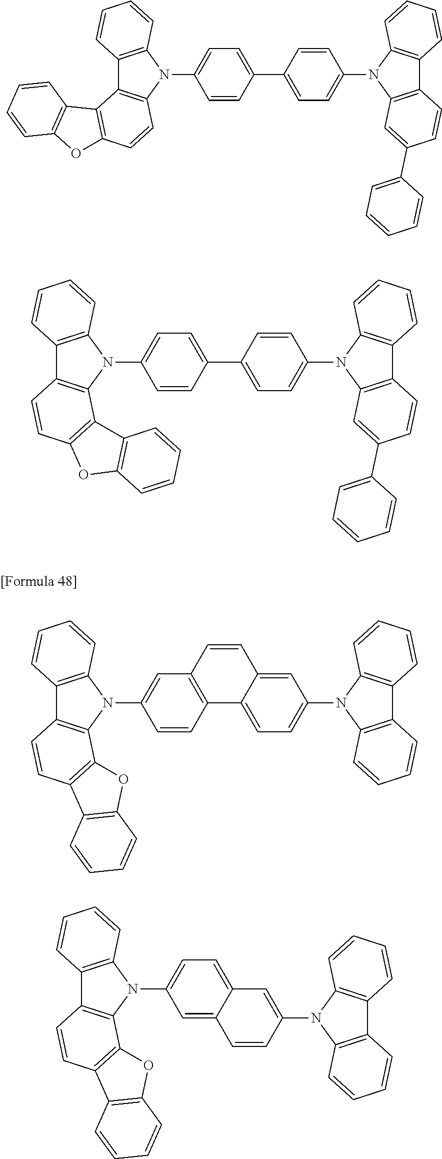

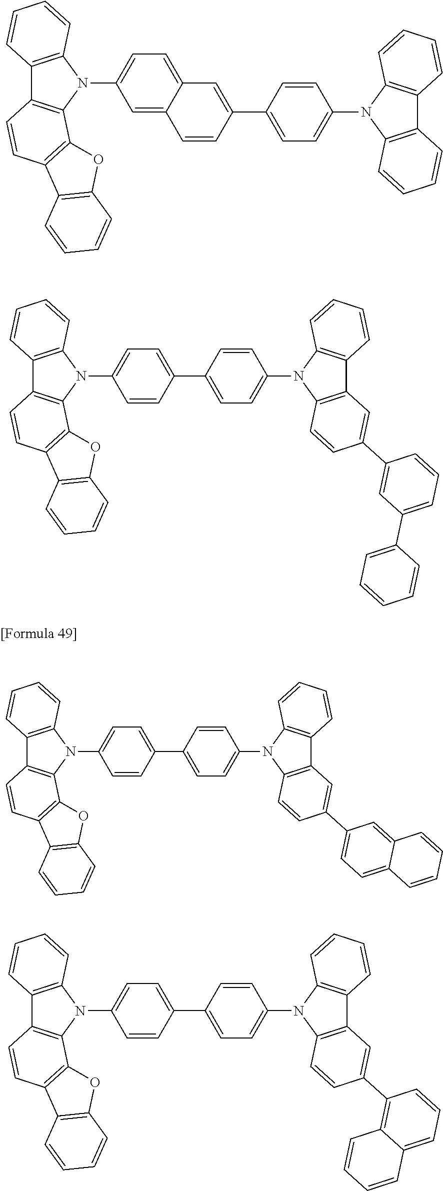

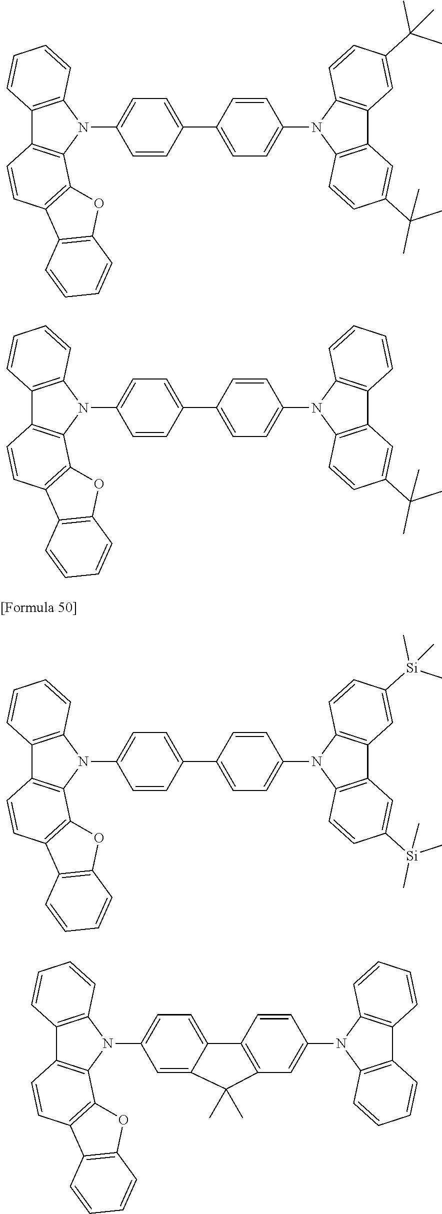

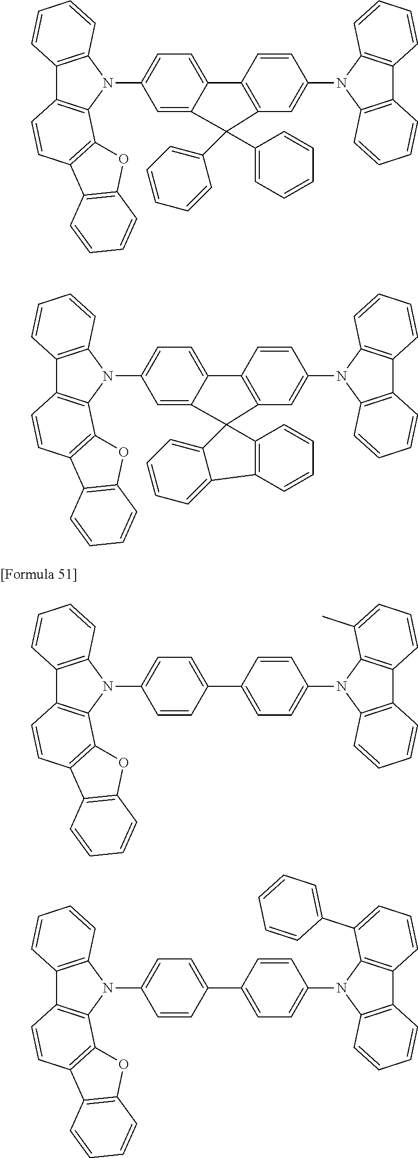

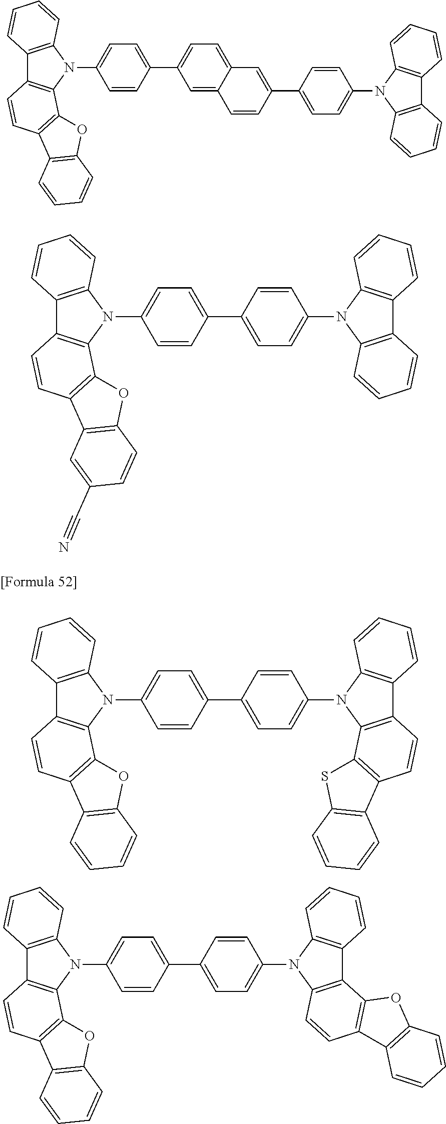

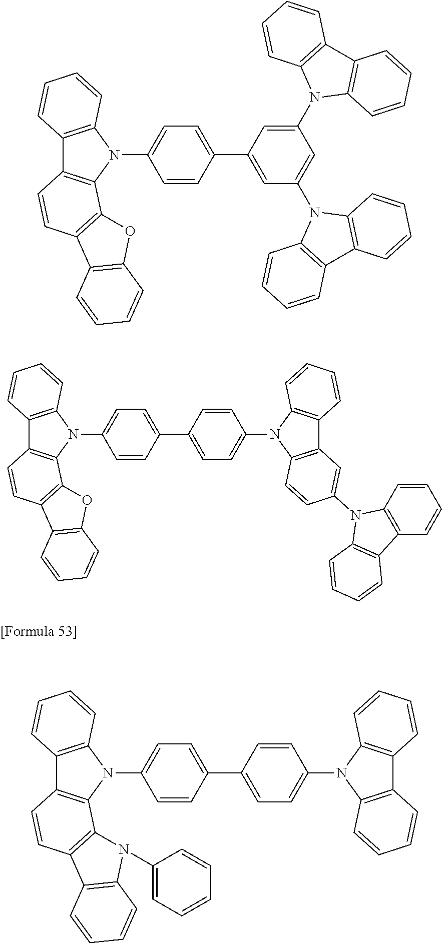 [SMILES: c1ccc2c(c1)oc1c2ccc2c3ccccc3n(-c3ccc(-c4cc(-n5c6ccccc6c6ccccc65)cc(-n5c6ccccc6c6ccccc65)c4)cc3)c21 ; c1ccc2c(c1)oc1c2ccc2c3ccccc3n(-c3ccc(-c4ccc(-n5c6ccccc6c6cc(-n7c8ccccc8c8ccccc87)ccc65)cc4)cc3)c21 ; c1ccc(-n2c3ccccc3c3ccc4c5ccccc5n(-c5ccc(-c6ccc(-n7c8ccccc8c8ccccc87)cc6)cc5)c4c32)cc1]

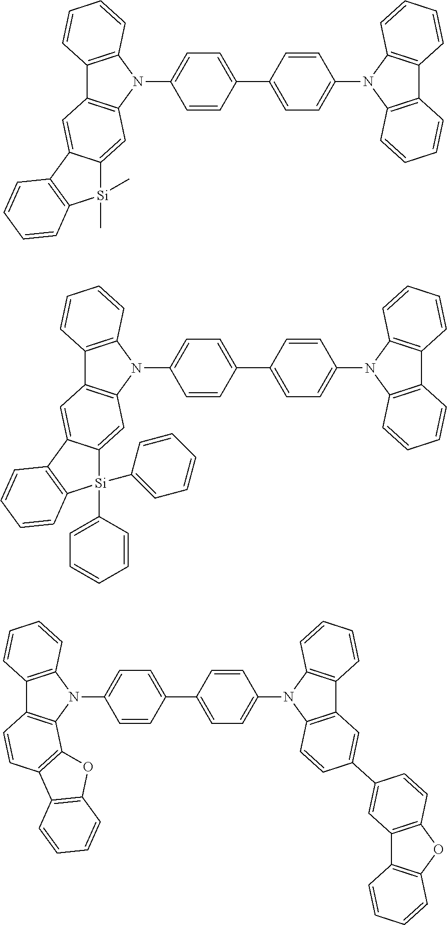 [SMILES: C[Si]1(C)c2ccccc2-c2cc3c4ccccc4n(-c4ccc(-c5ccc(-n6c7ccccc7c7ccccc76)cc5)cc4)c3cc21 ; c1ccc([Si]2(c3ccccc3)c3ccccc3-c3cc4c5ccccc5n(-c5ccc(-c6ccc(-n7c8ccccc8c8ccccc87)cc6)cc5)c4cc32)cc1 ; c1ccc2c(c1)oc1ccc(-c3ccc4c(c3)c3ccccc3n4-c3ccc(-c4ccc(-n5c6ccccc6c6ccc7c8ccccc8oc7c65)cc4)cc3)cc12]

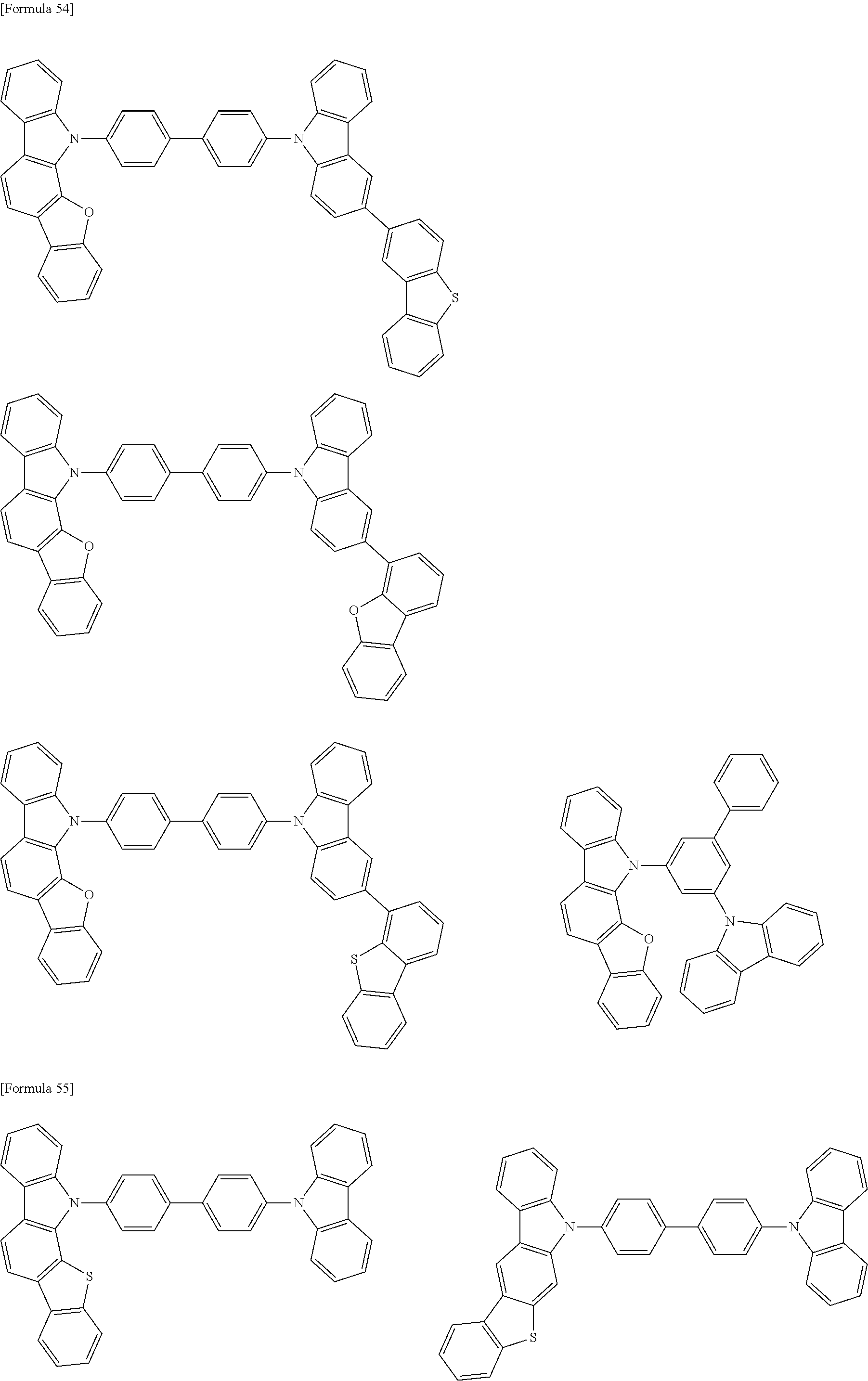

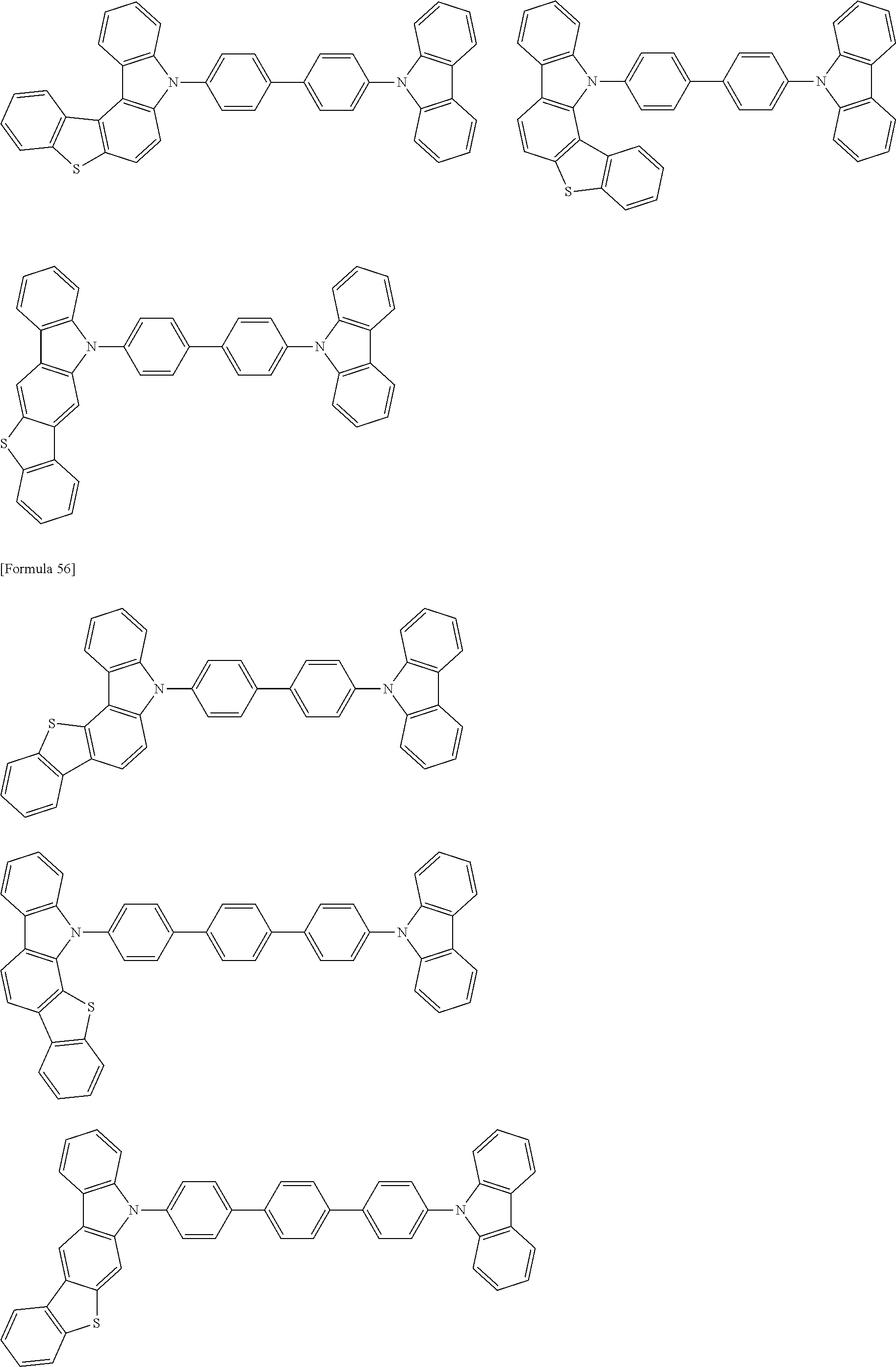

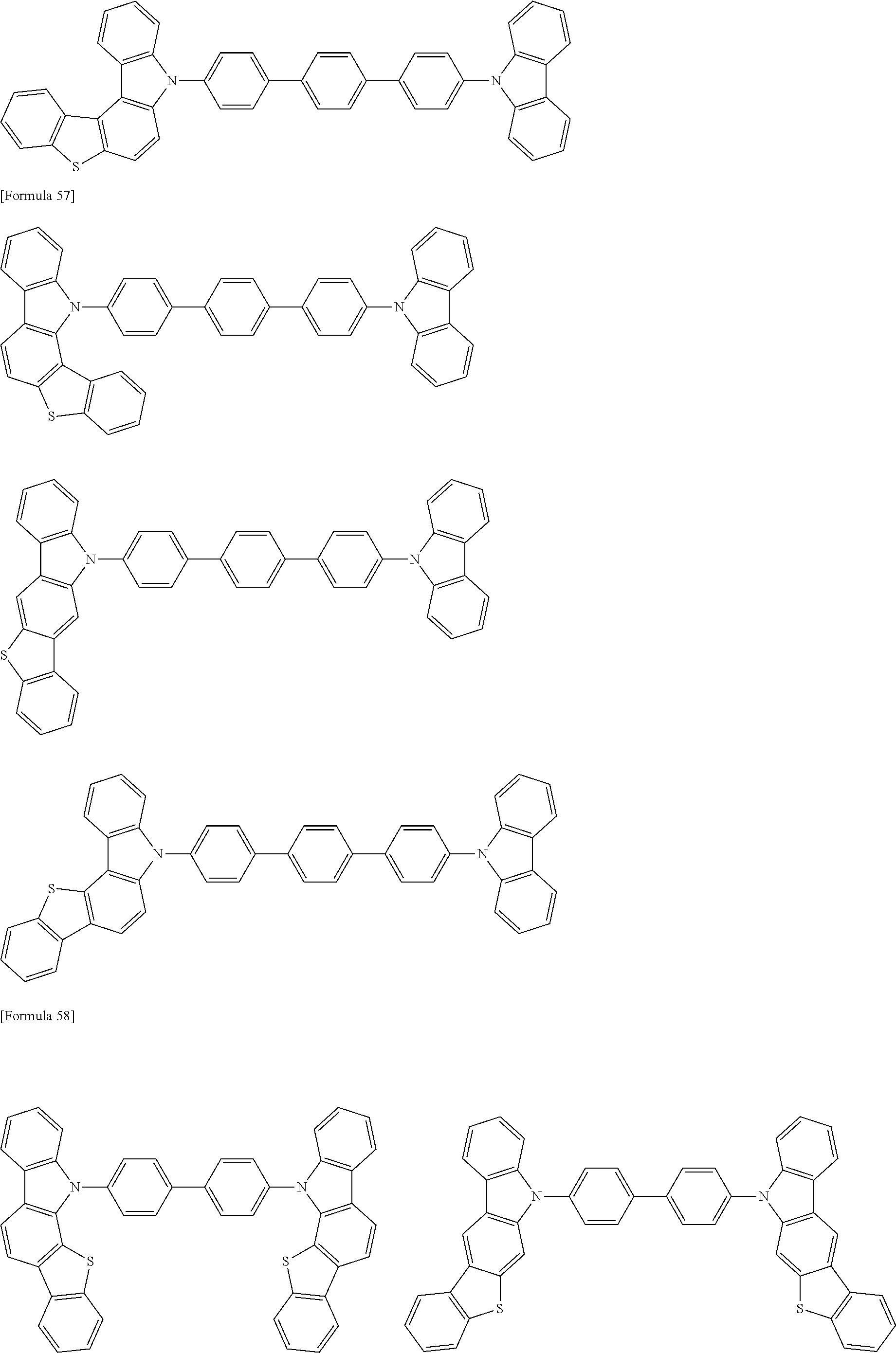

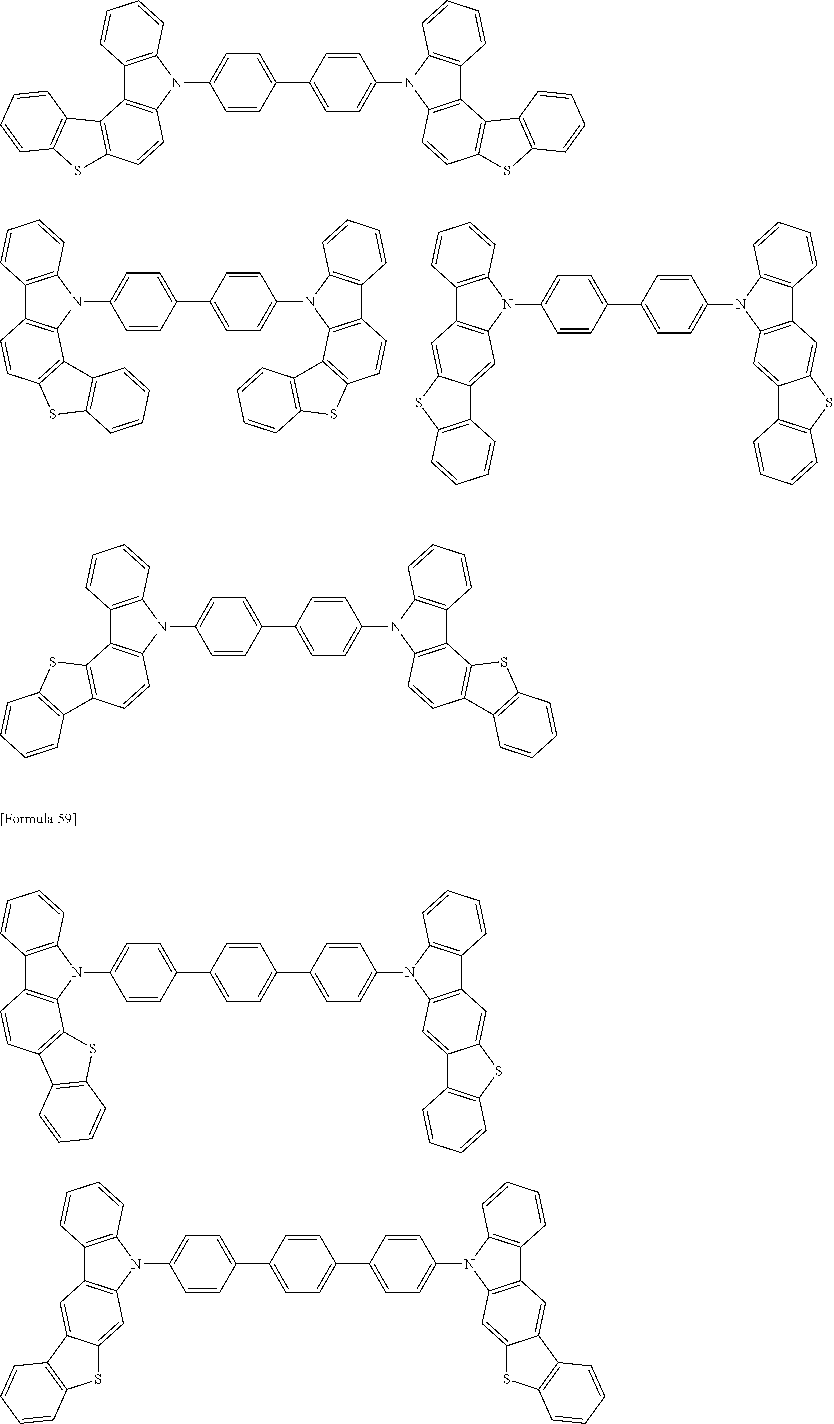

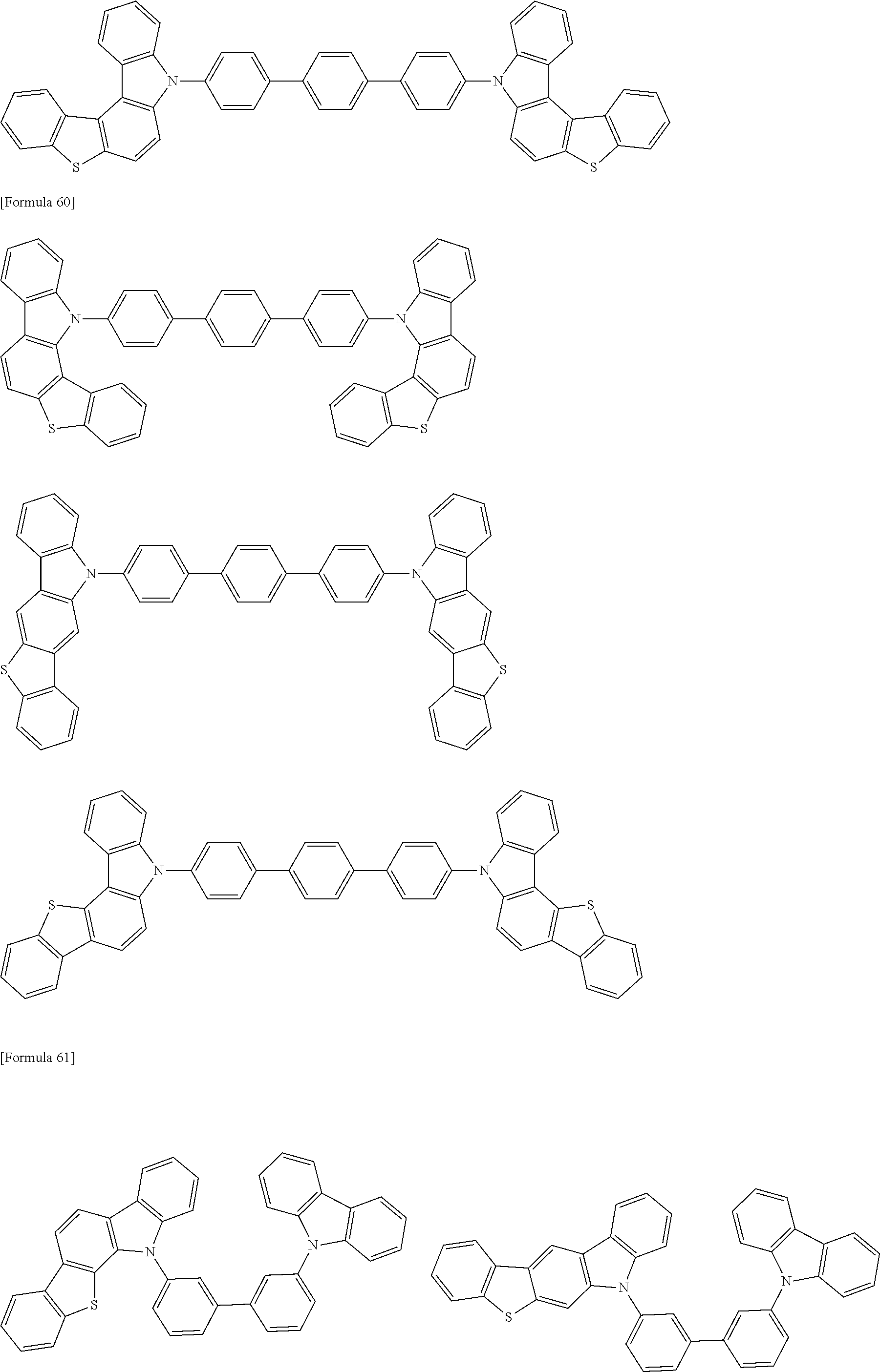

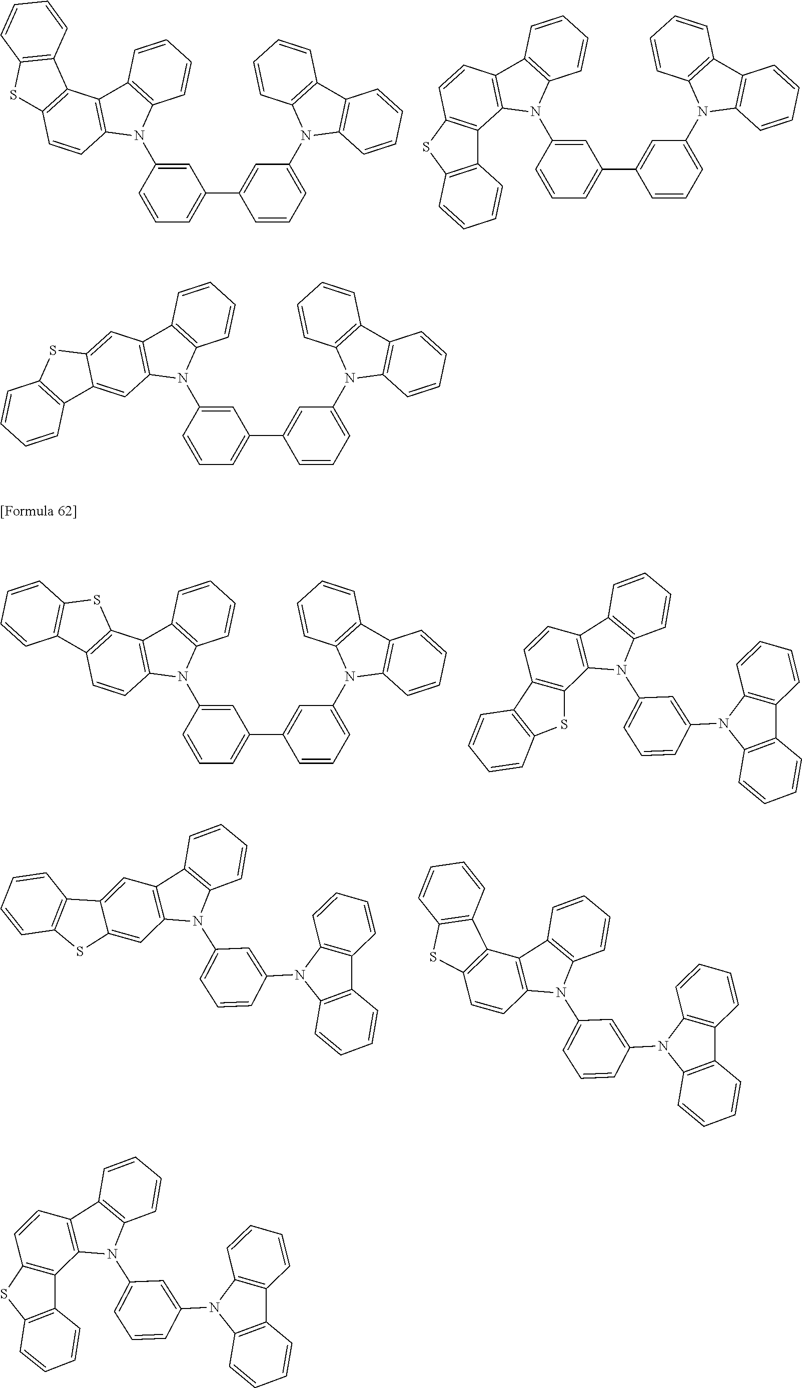

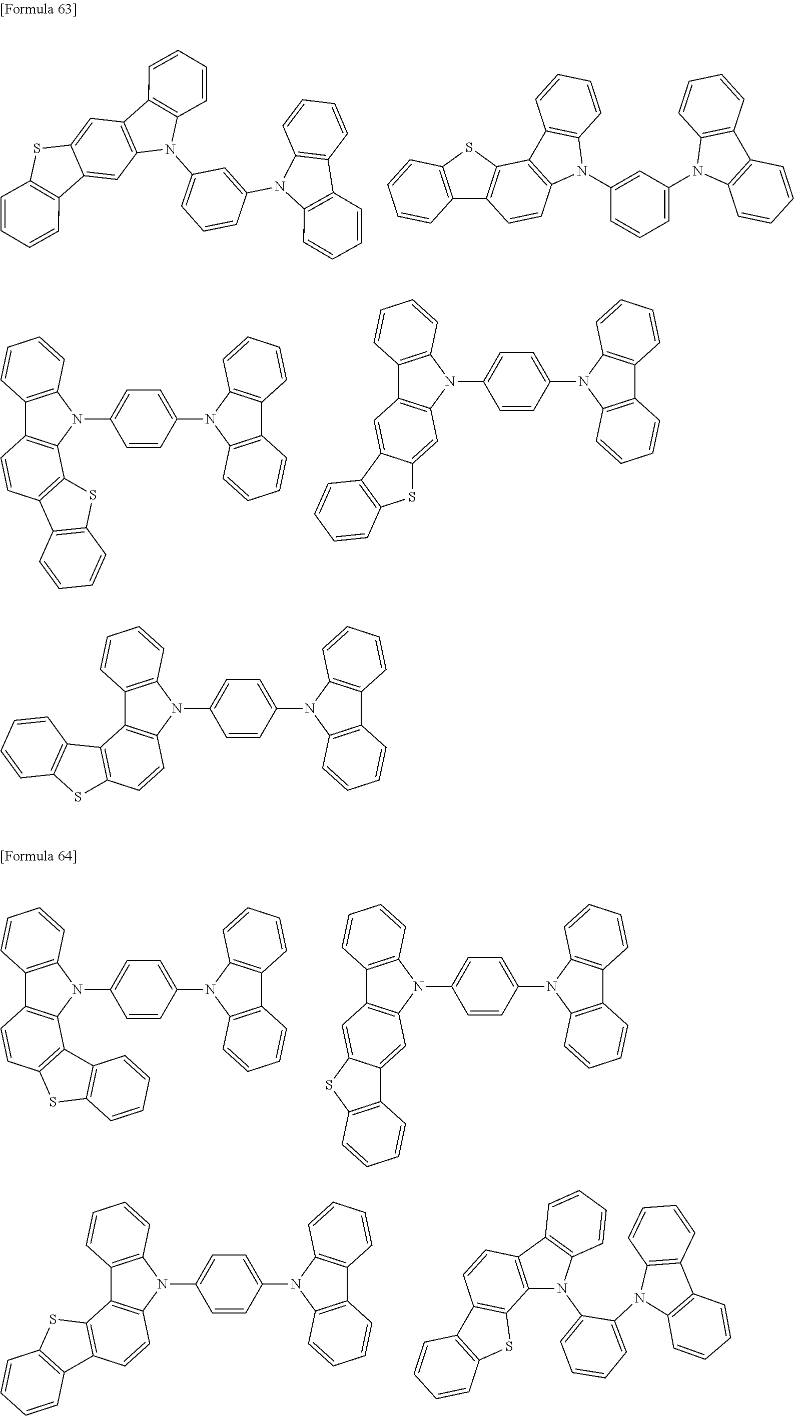

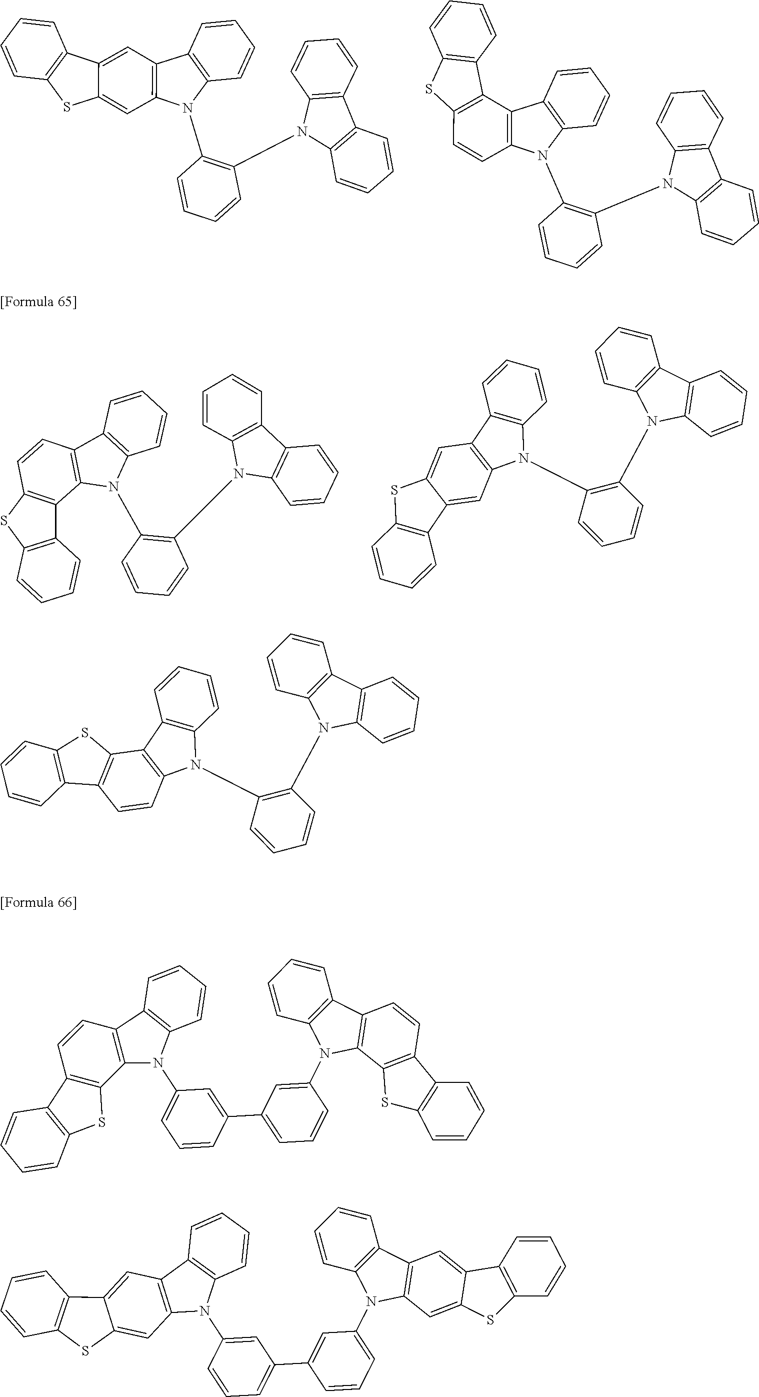

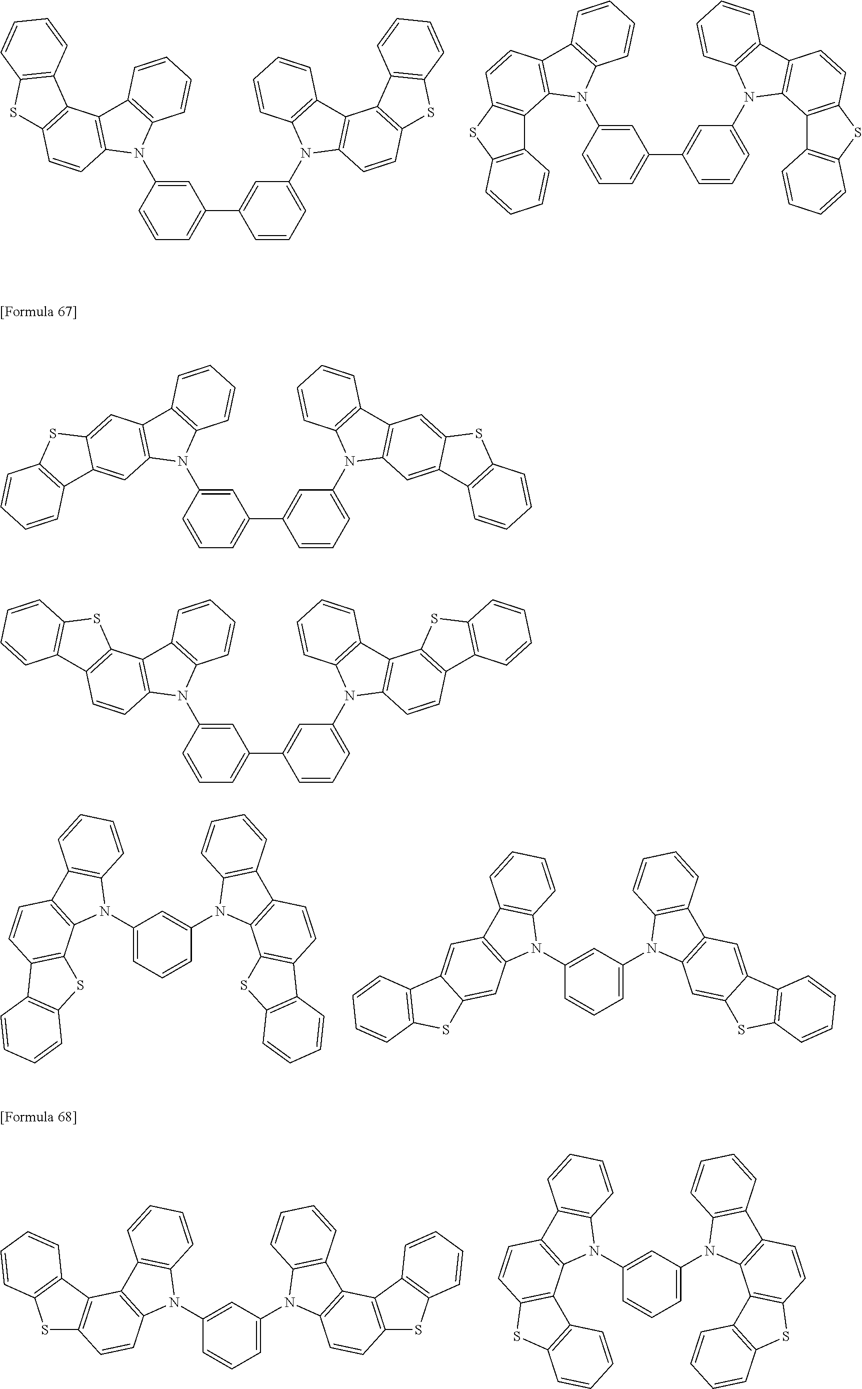

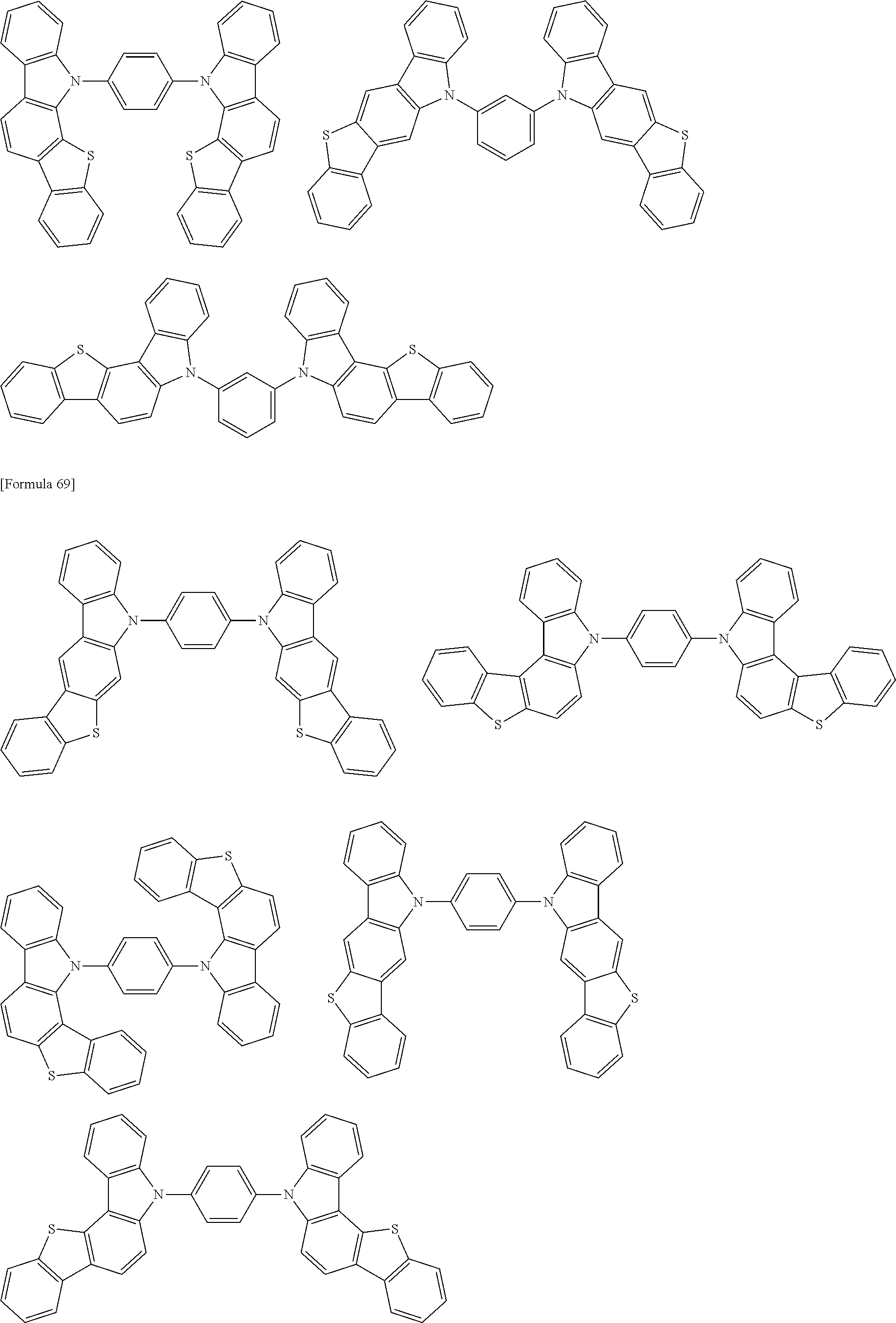

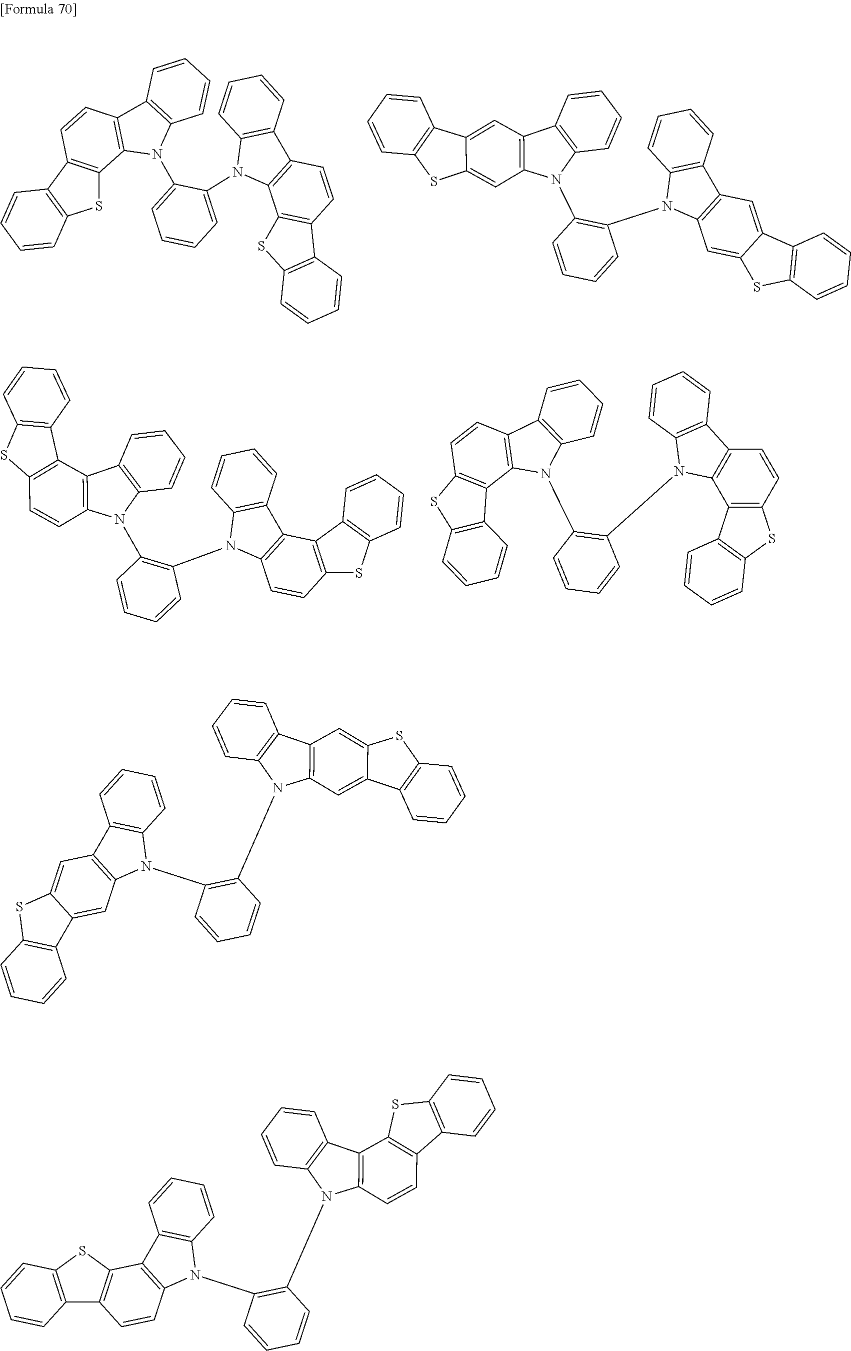

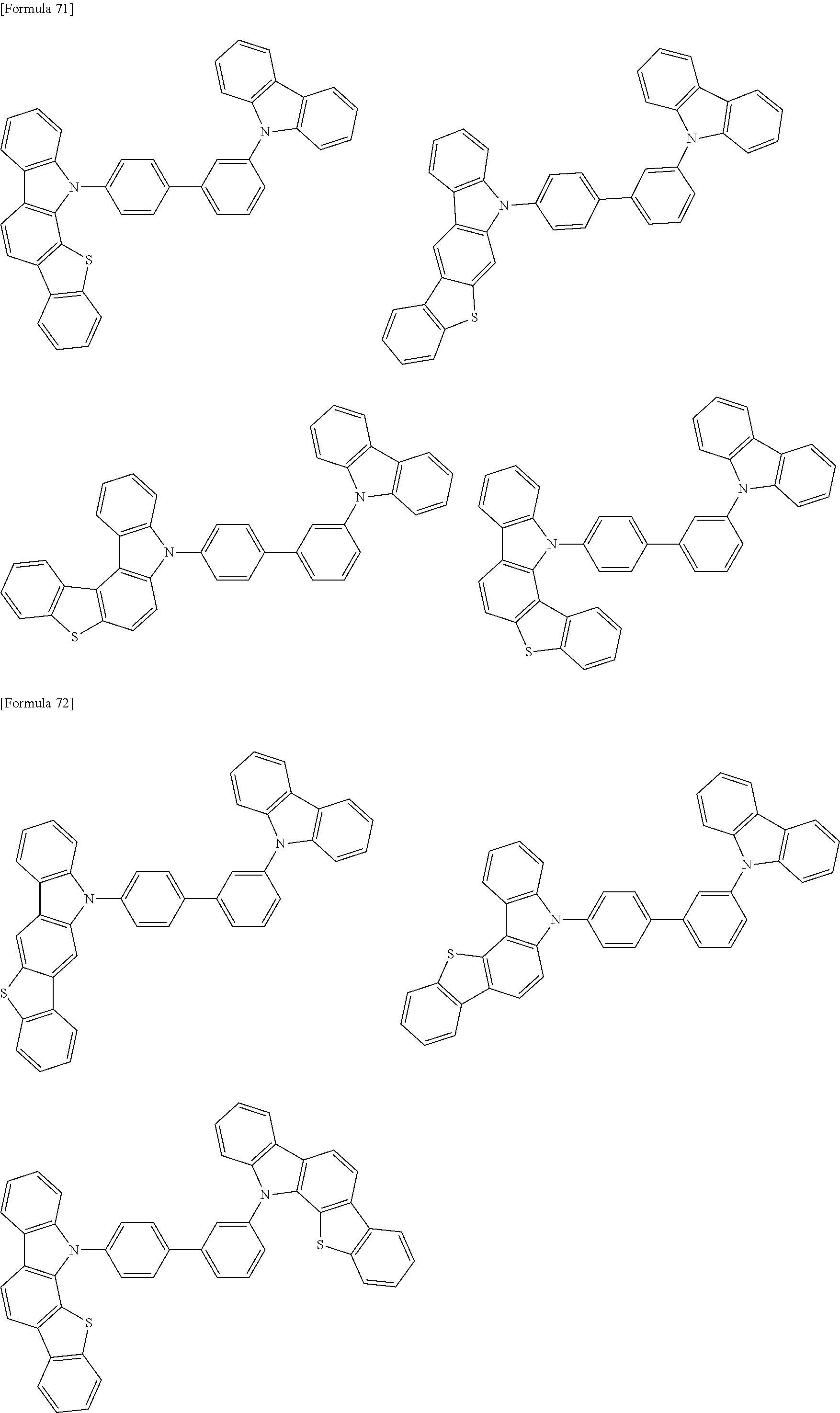

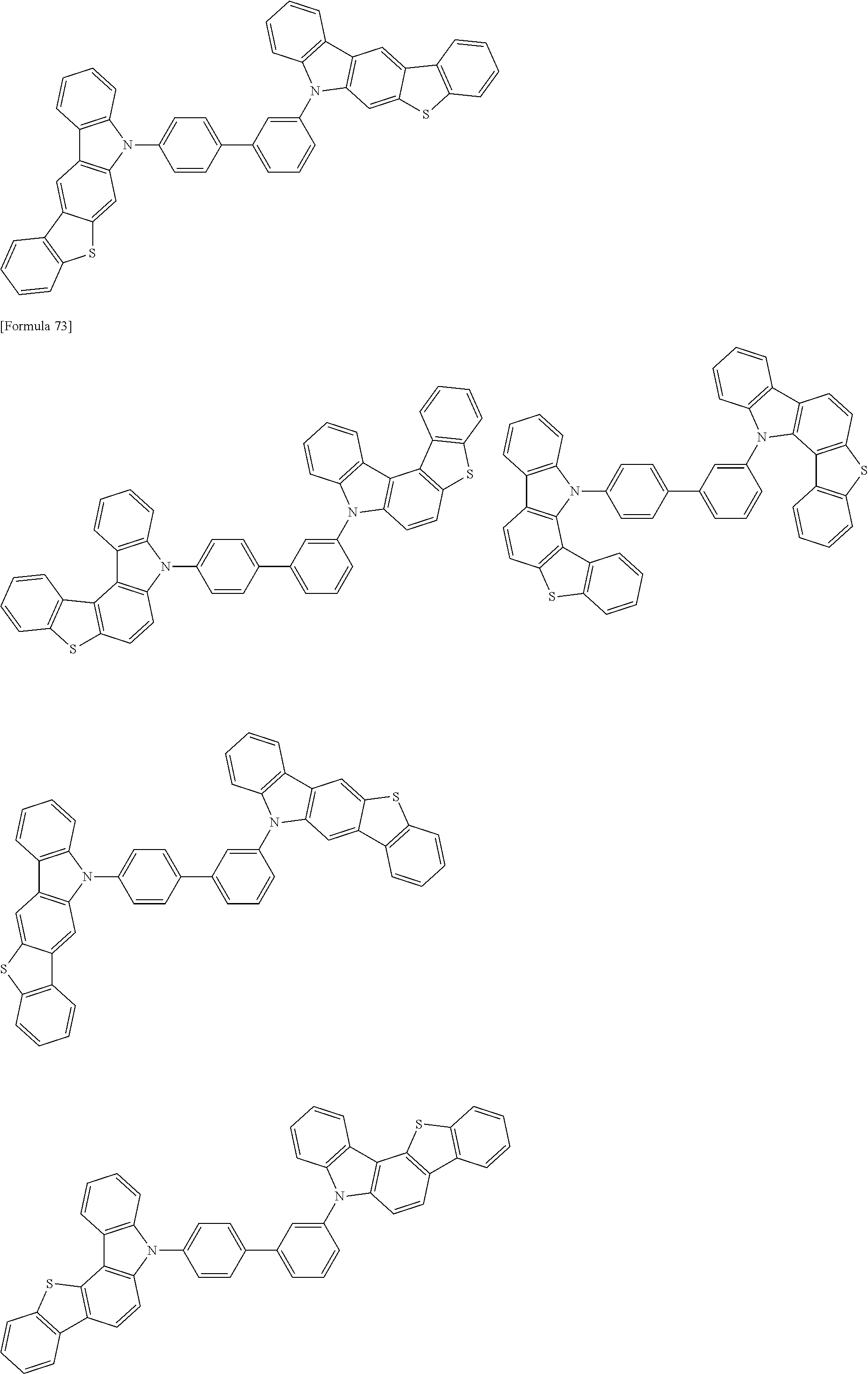

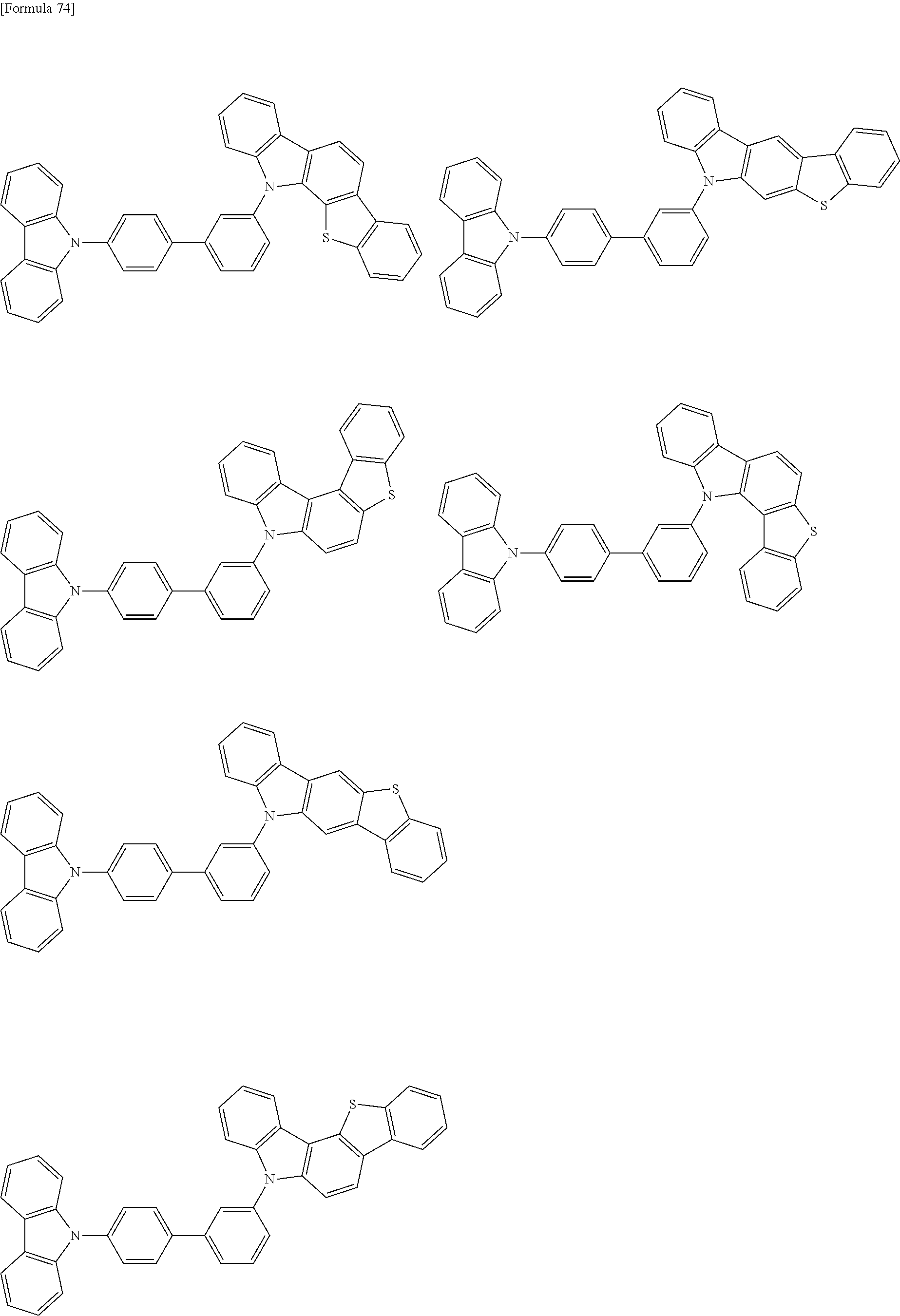

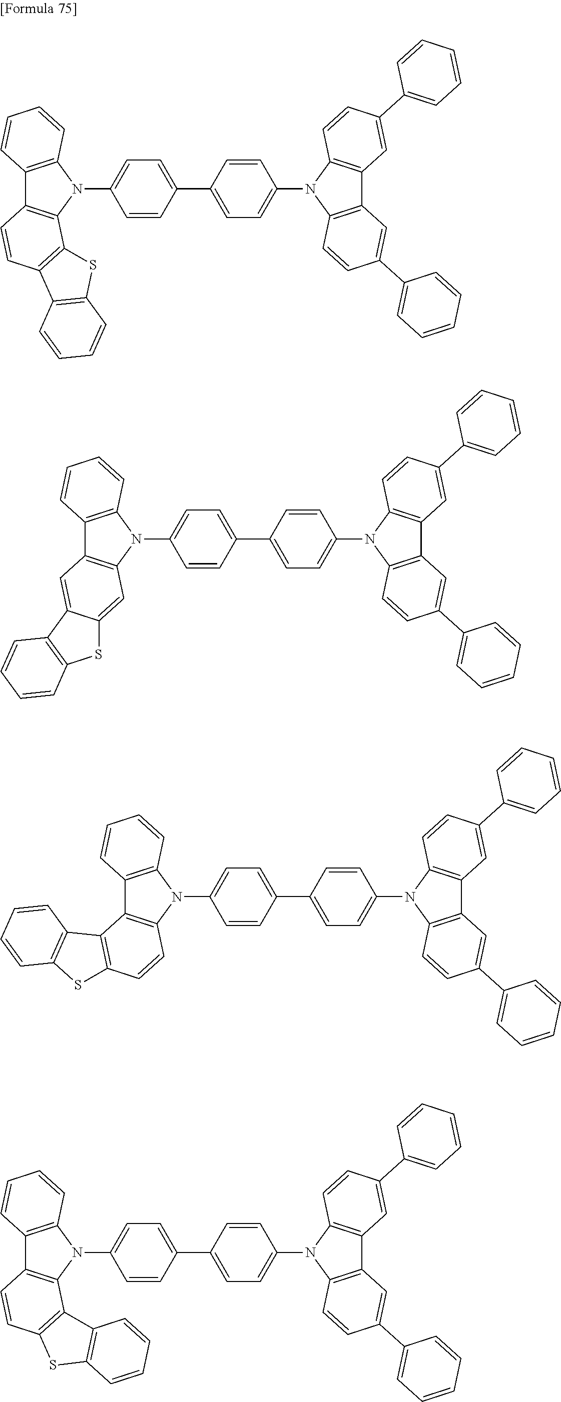 [SMILES: c1ccc(-c2ccc3c(c2)c2cc(-c4ccccc4)ccc2n3-c2ccc(-c3ccc(-n4c5ccccc5c5ccc6c7ccccc7sc6c54)cc3)cc2)cc1 ; c1ccc(-c2ccc3c(c2)c2cc(-c4ccccc4)ccc2n3-c2ccc(-c3ccc(-n4c5ccccc5c5cc6c(cc54)sc4ccccc46)cc3)cc2)cc1 ; c1ccc(-c2ccc3c(c2)c2cc(-c4ccccc4)ccc2n3-c2ccc(-c3ccc(-n4c5ccccc5c5c6c(ccc54)sc4ccccc46)cc3)cc2)cc1 ; c1ccc(-c2ccc3c(c2)c2cc(-c4ccccc4)ccc2n3-c2ccc(-c3ccc(-n4c5ccccc5c5ccc6sc7ccccc7c6c54)cc3)cc2)cc1]

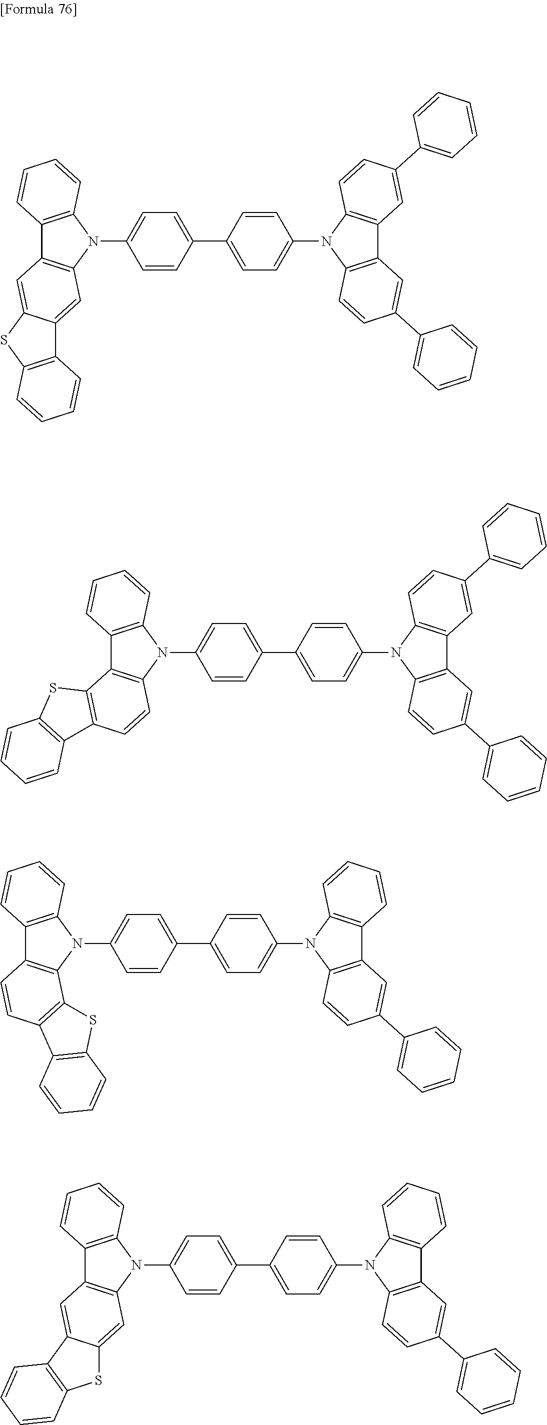 [SMILES: c1ccc(-c2ccc3c(c2)c2cc(-c4ccccc4)ccc2n3-c2ccc(-c3ccc(-n4c5ccccc5c5cc6sc7ccccc7c6cc54)cc3)cc2)cc1 ; c1ccc(-c2ccc3c(c2)c2cc(-c4ccccc4)ccc2n3-c2ccc(-c3ccc(-n4c5ccccc5c5c6sc7ccccc7c6ccc54)cc3)cc2)cc1 ; c1ccc(-c2ccc3c(c2)c2ccccc2n3-c2ccc(-c3ccc(-n4c5ccccc5c5ccc6c7ccccc7sc6c54)cc3)cc2)cc1 ; c1ccc(-c2ccc3c(c2)c2ccccc2n3-c2ccc(-c3ccc(-n4c5ccccc5c5cc6c(cc54)sc4ccccc46)cc3)cc2)cc1]

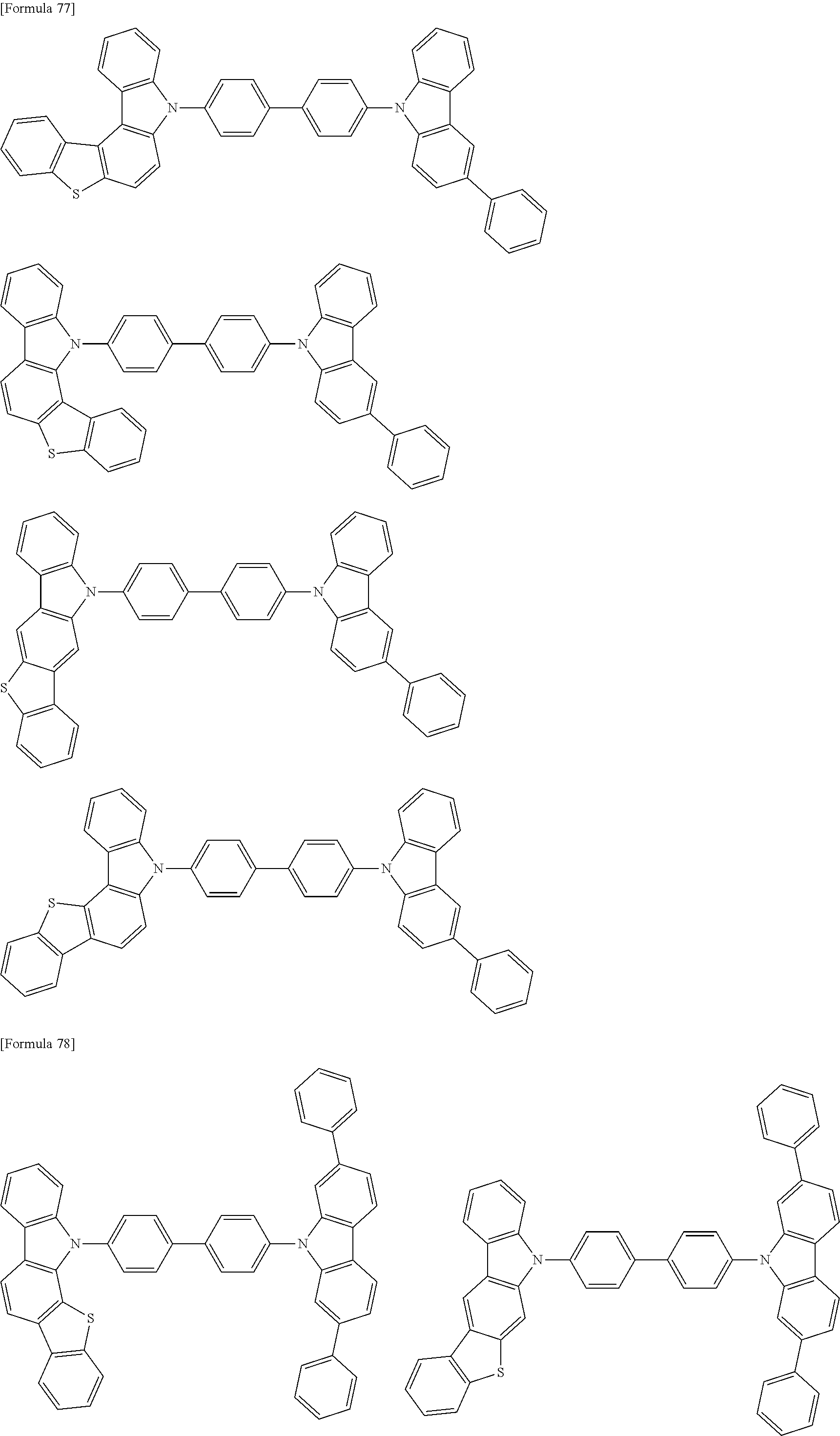

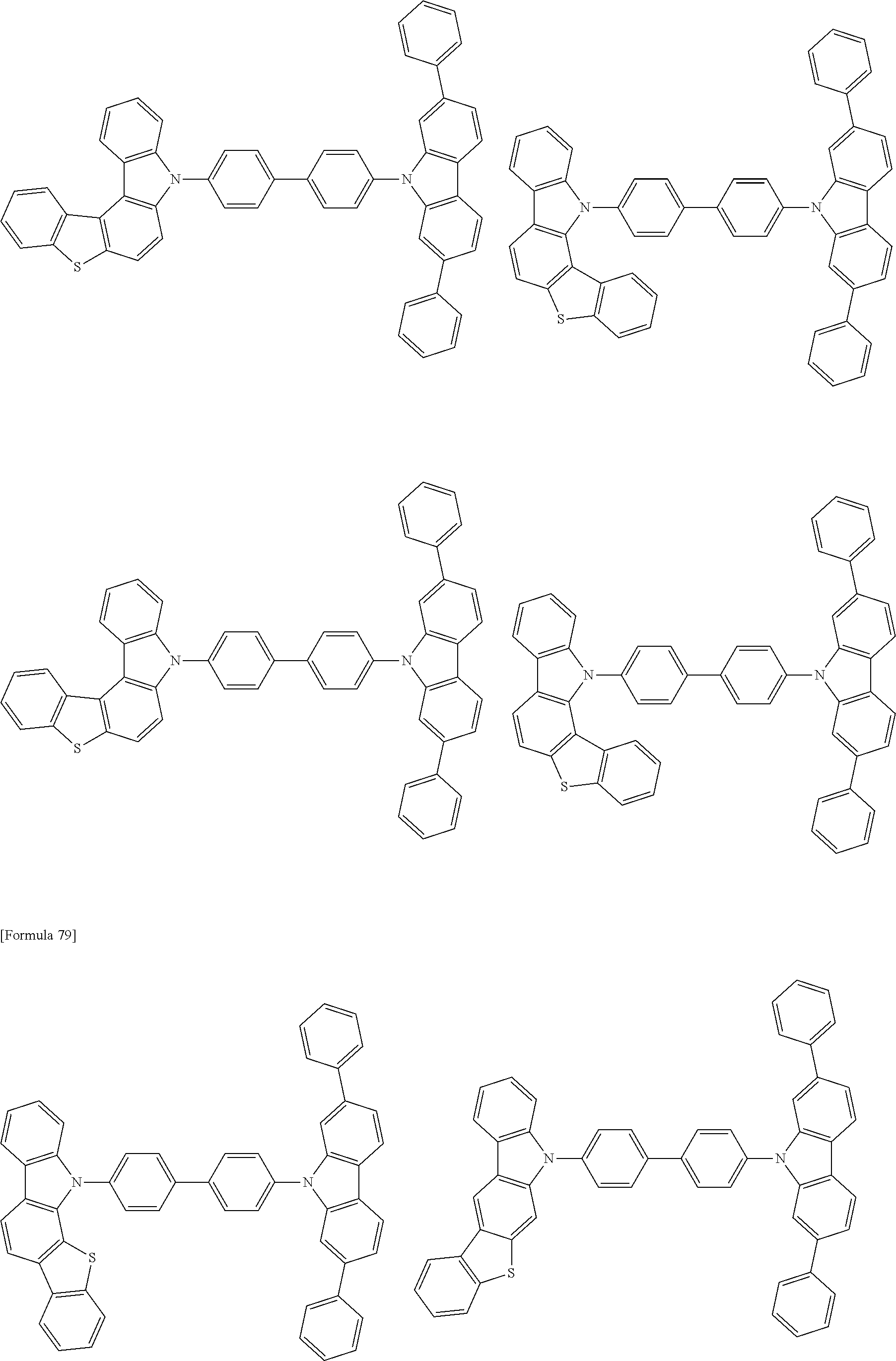

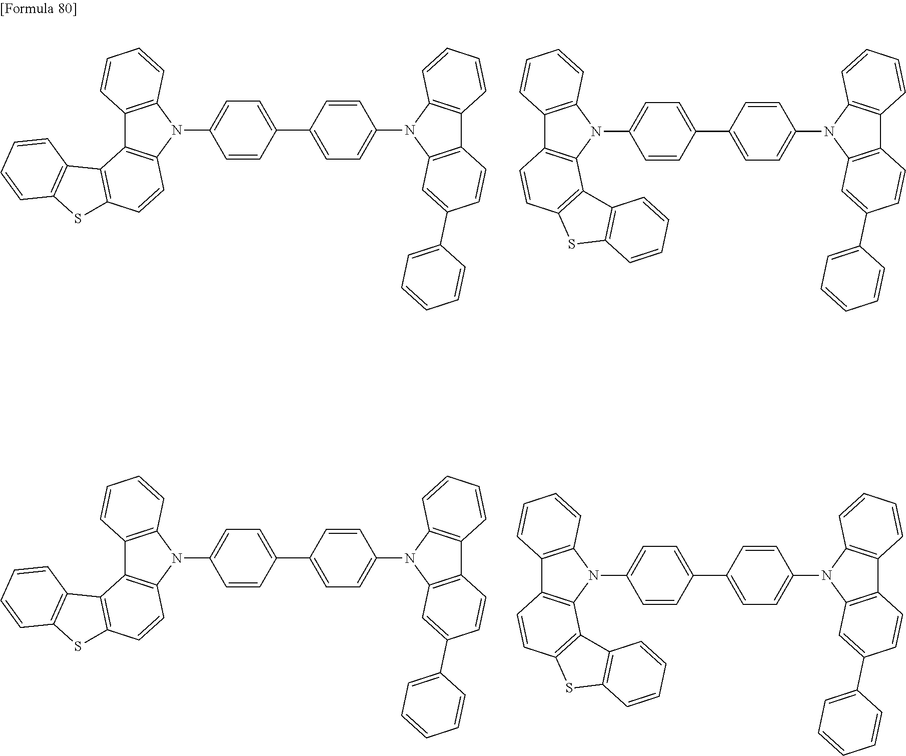

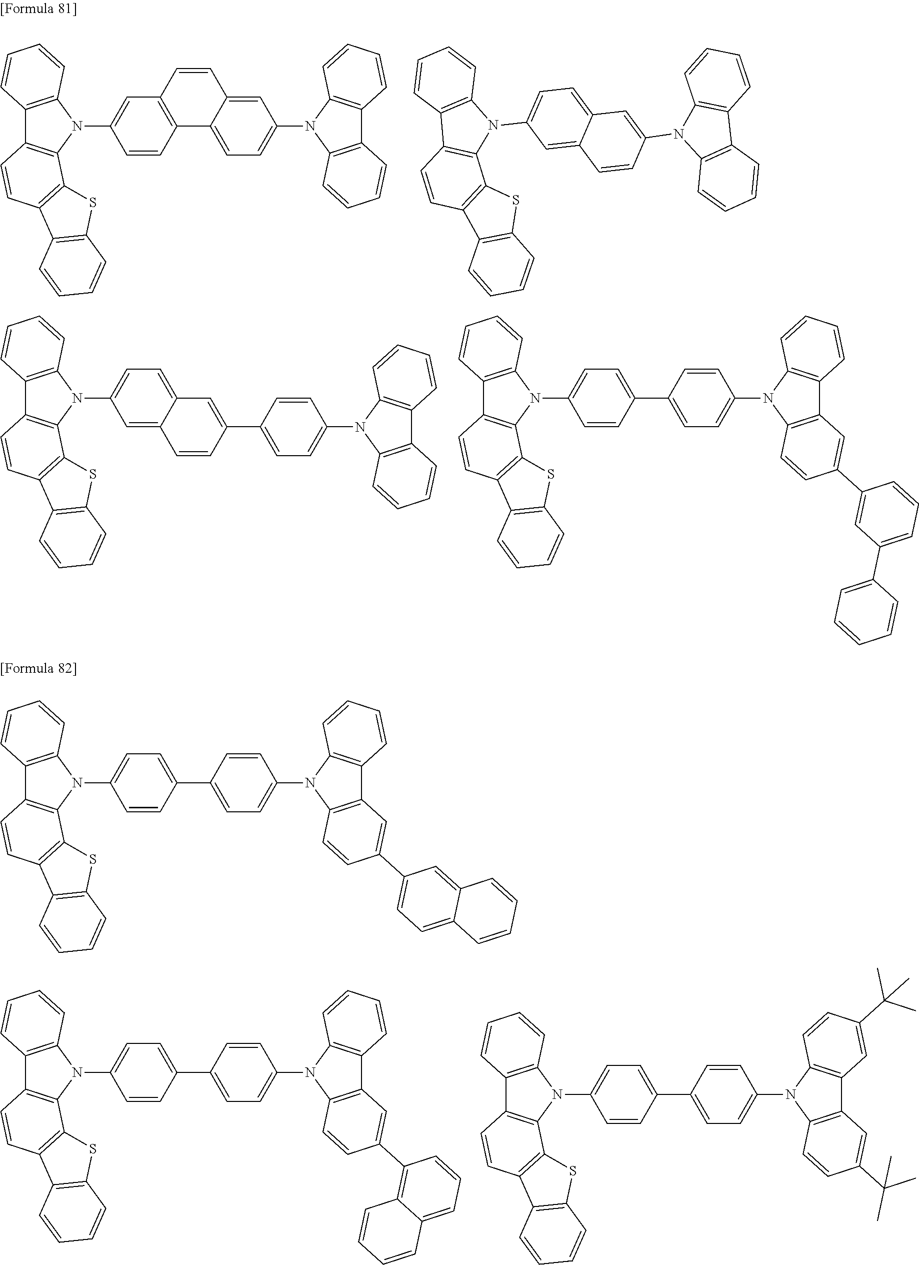

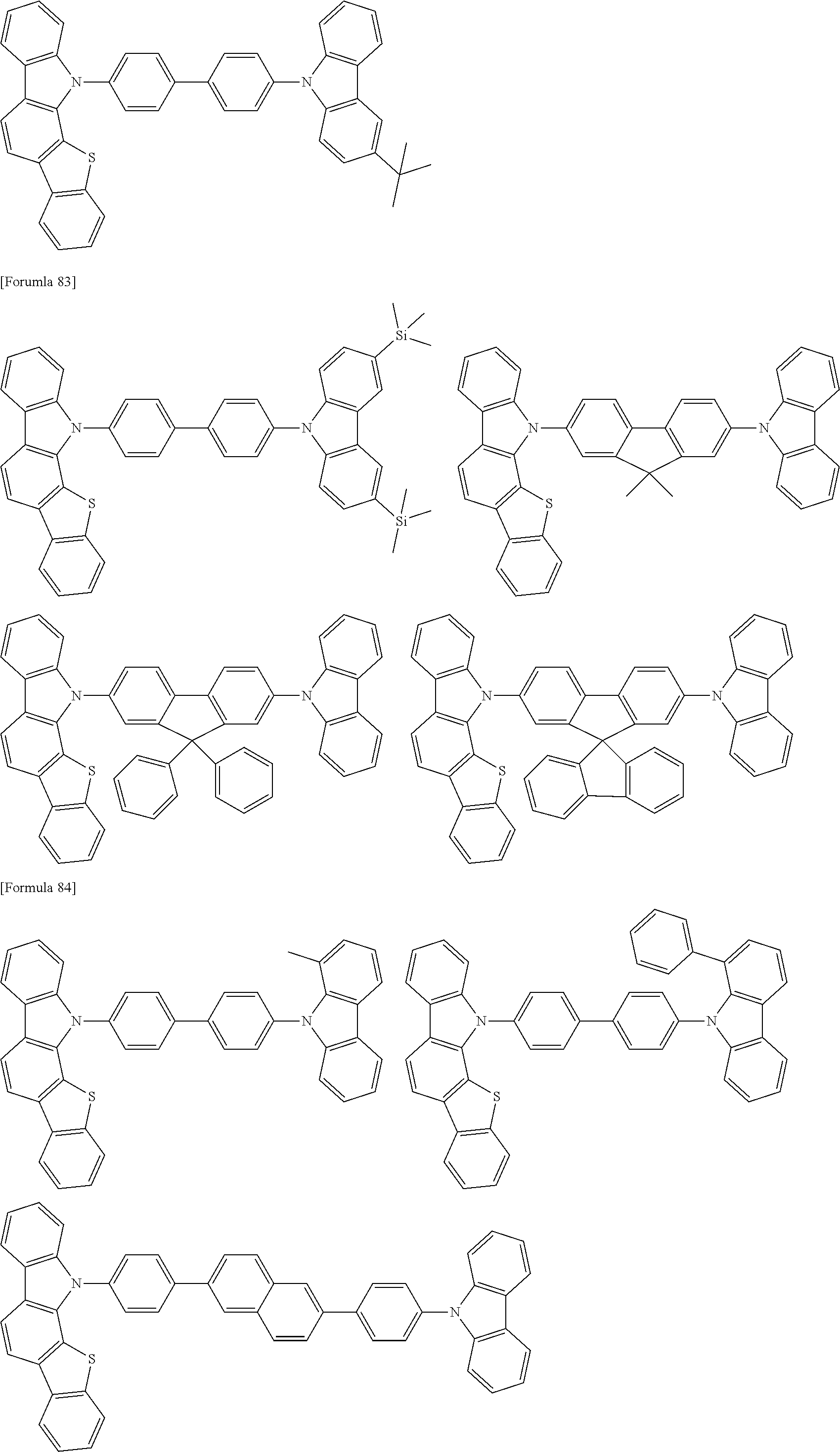

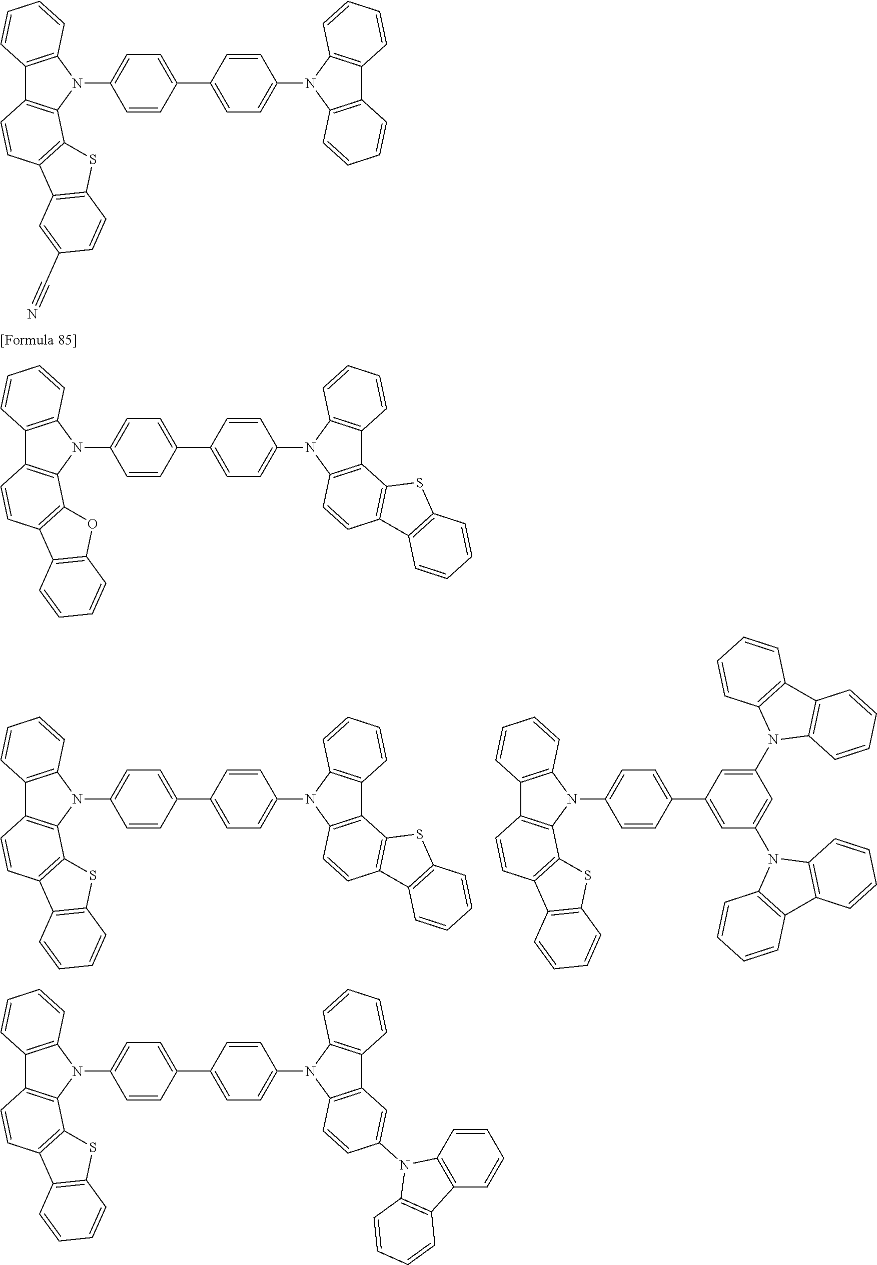

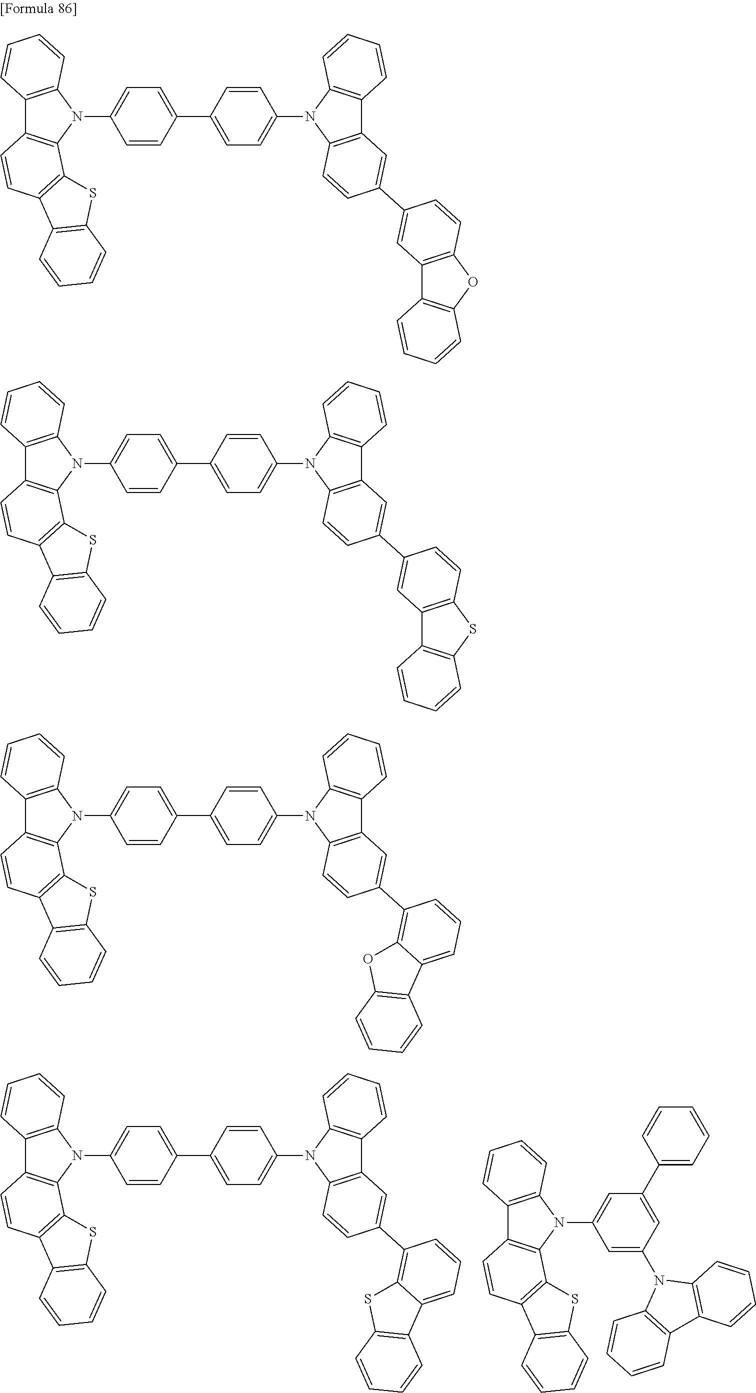

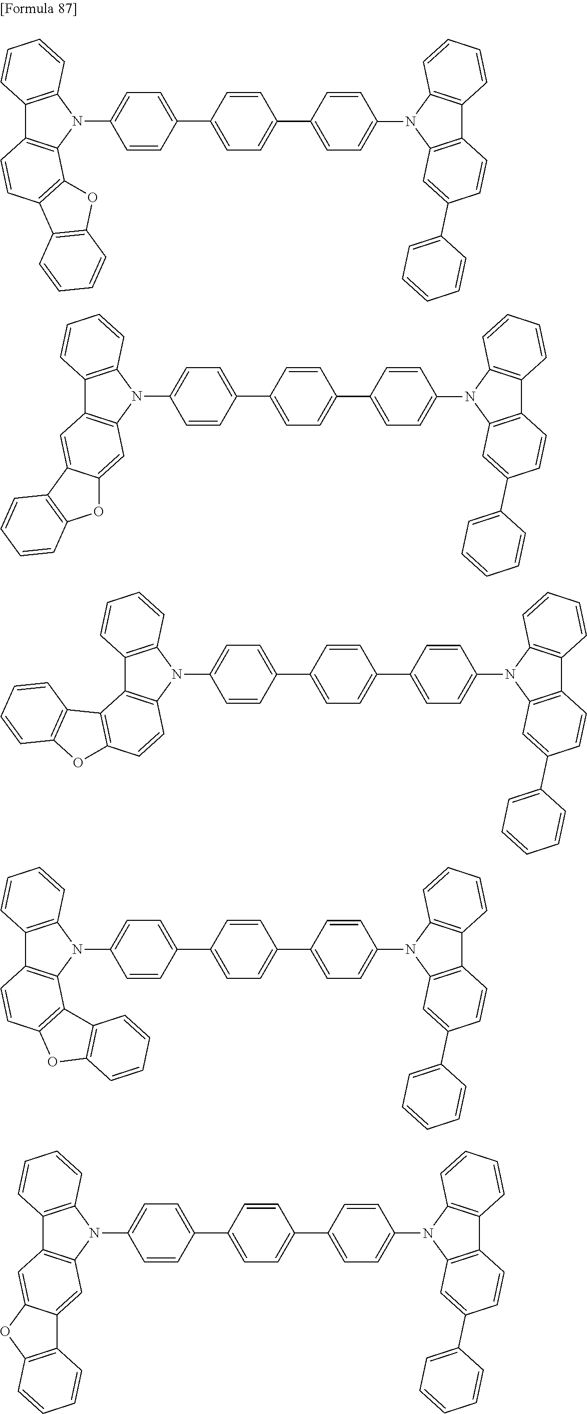

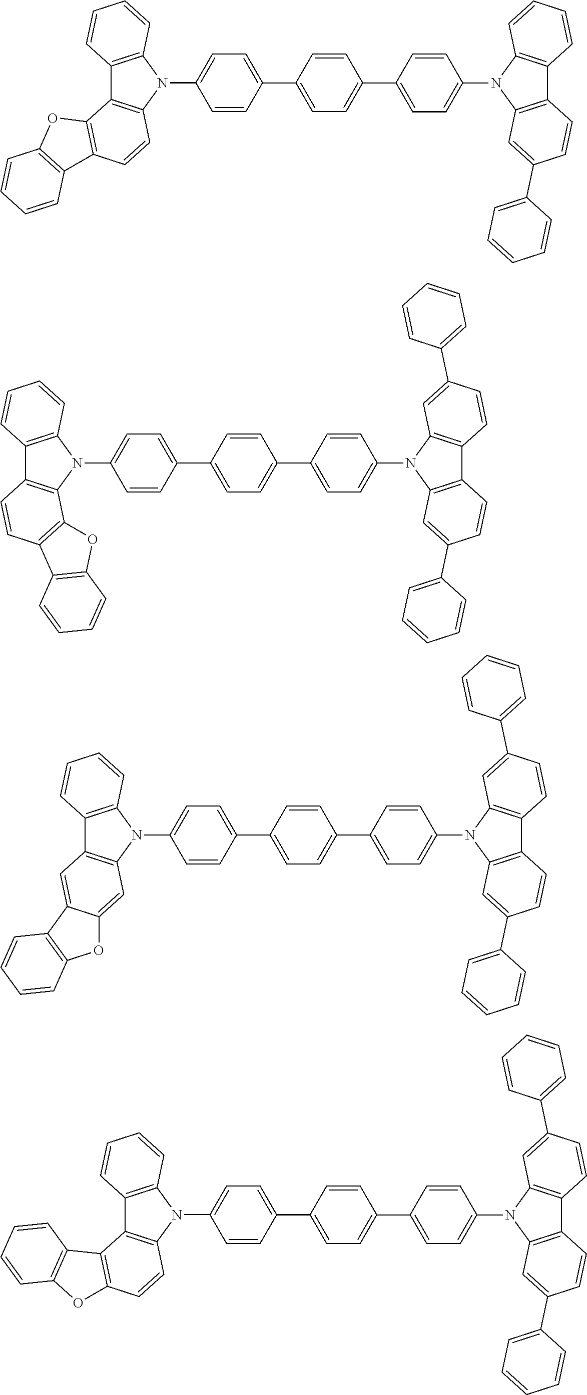

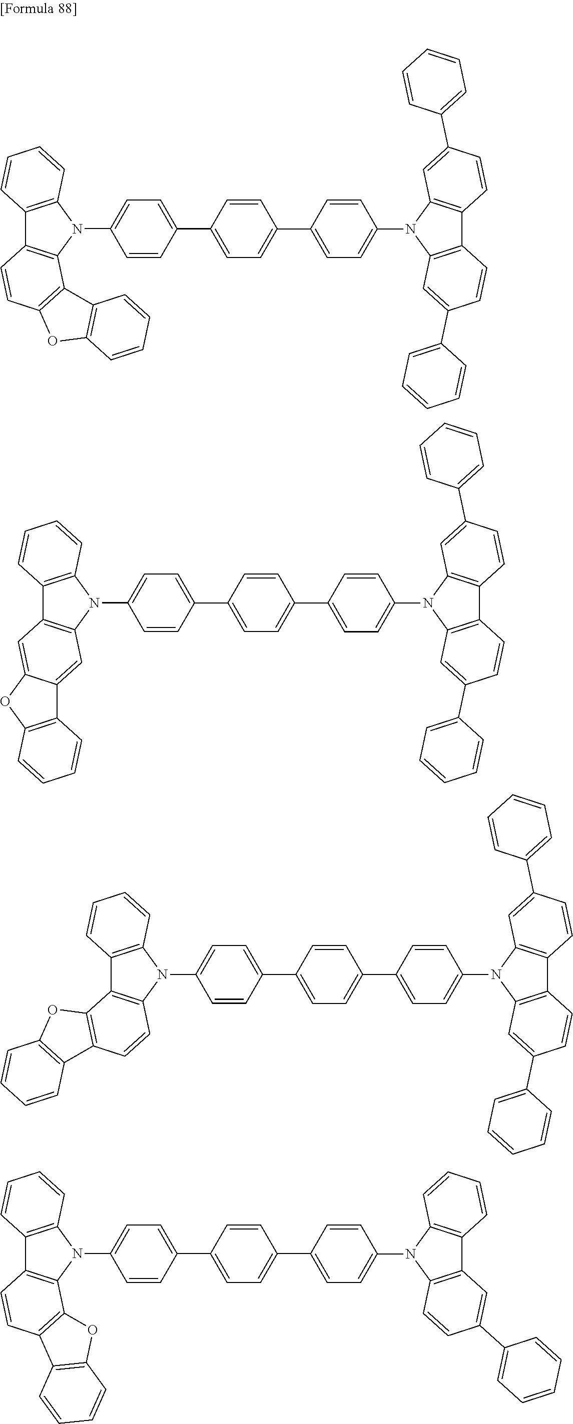

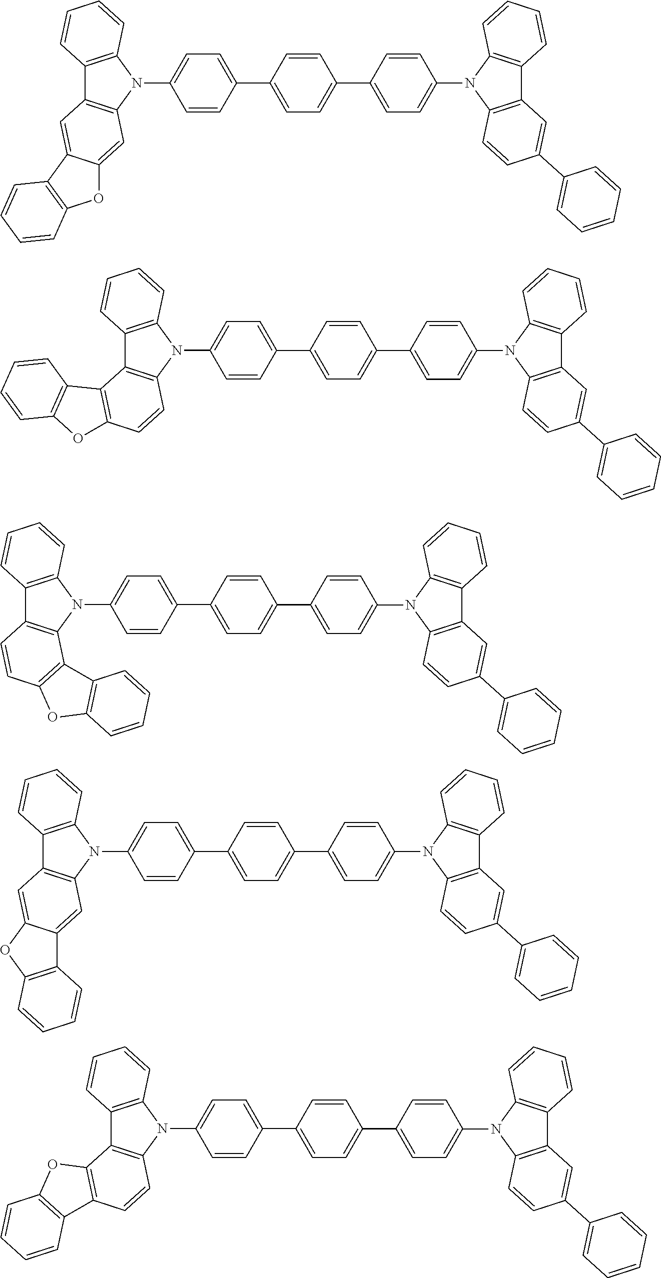

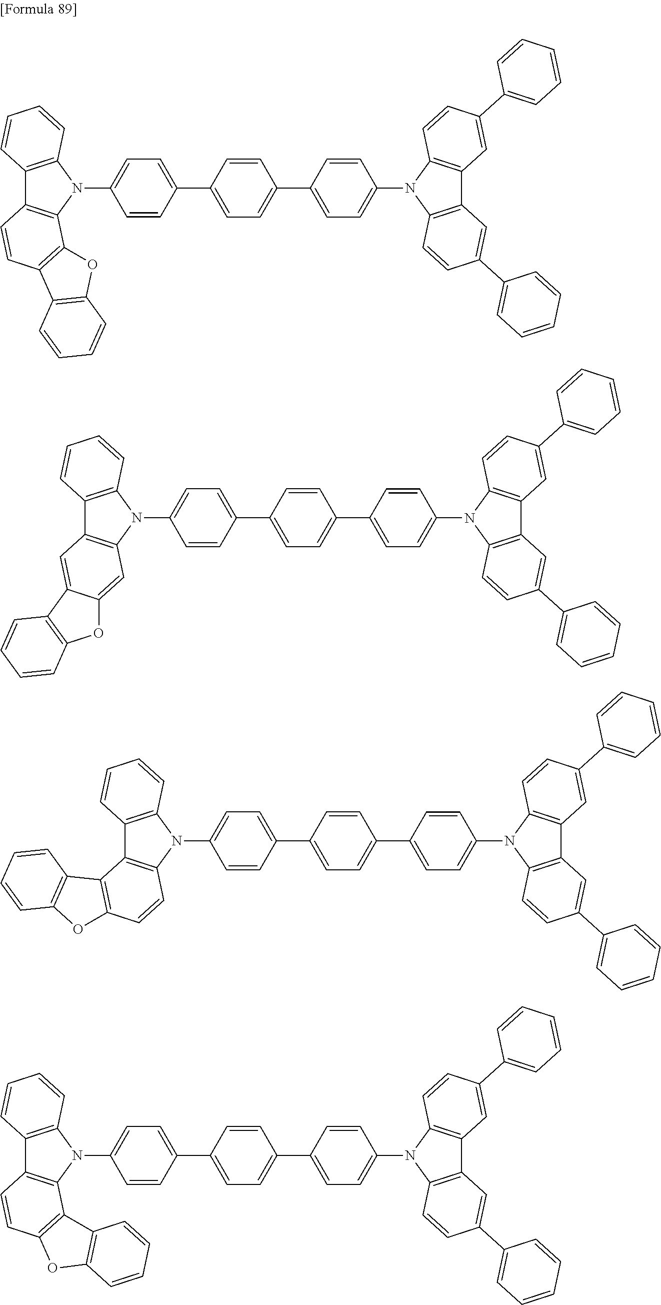

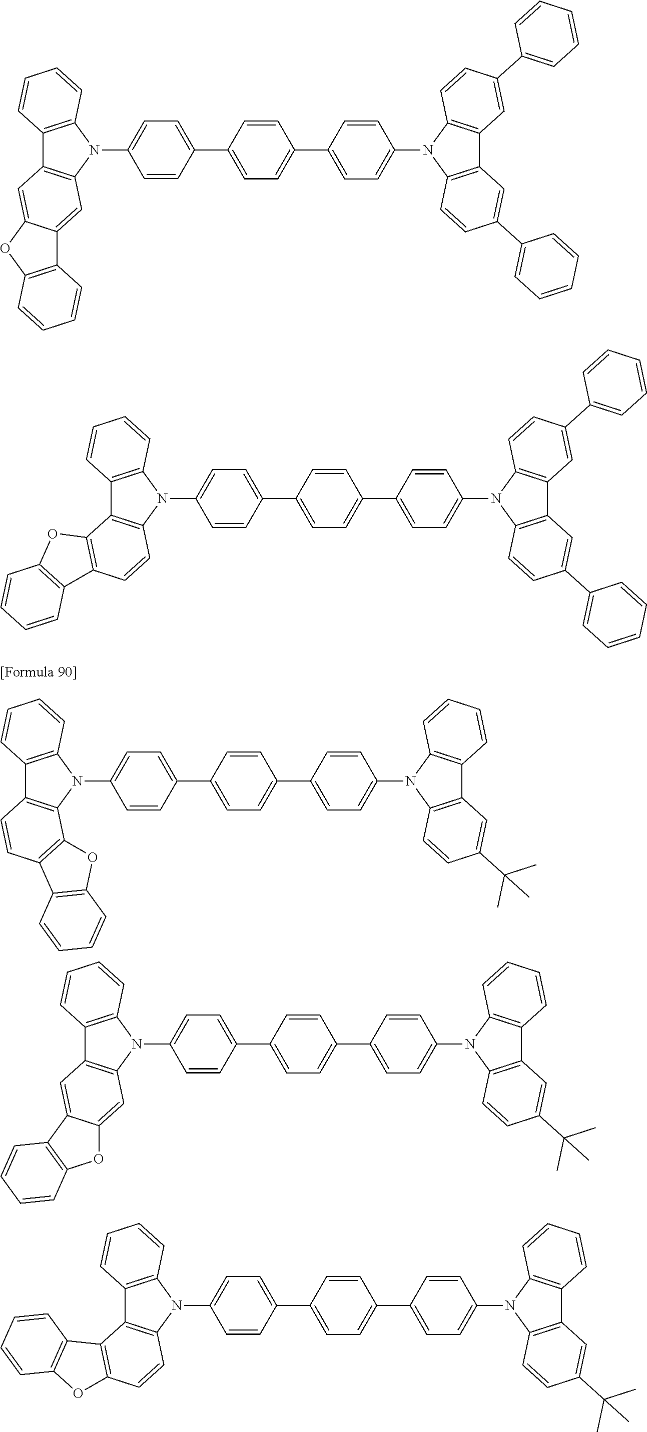

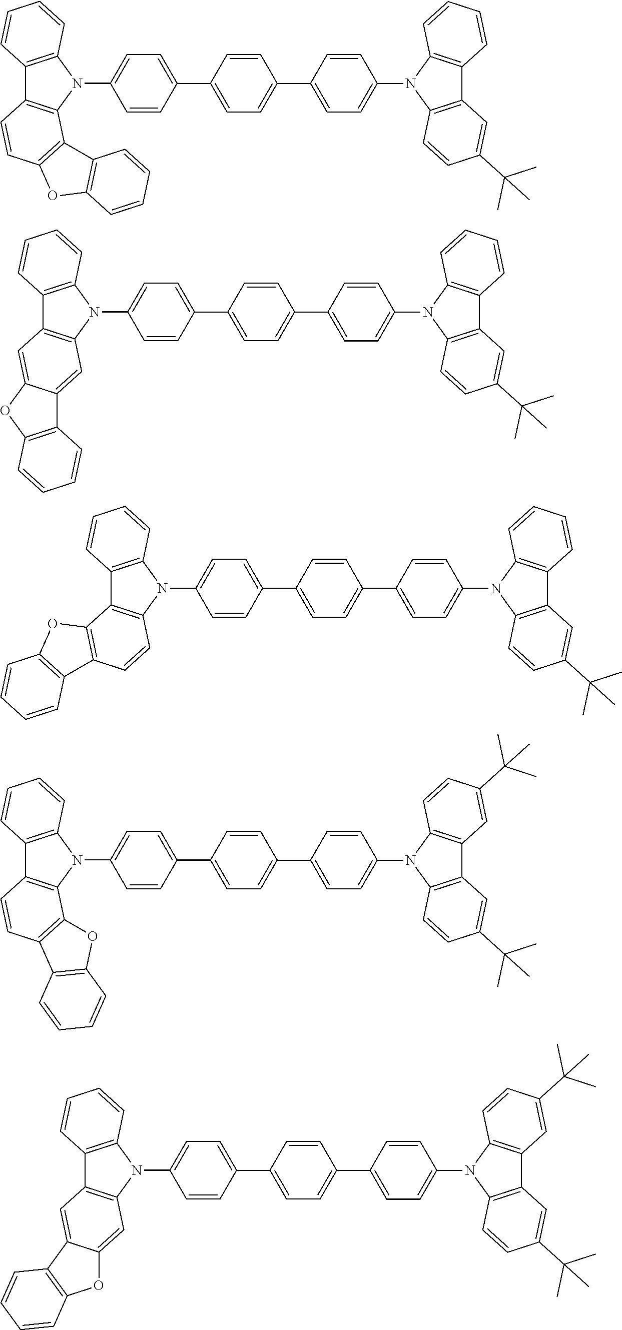

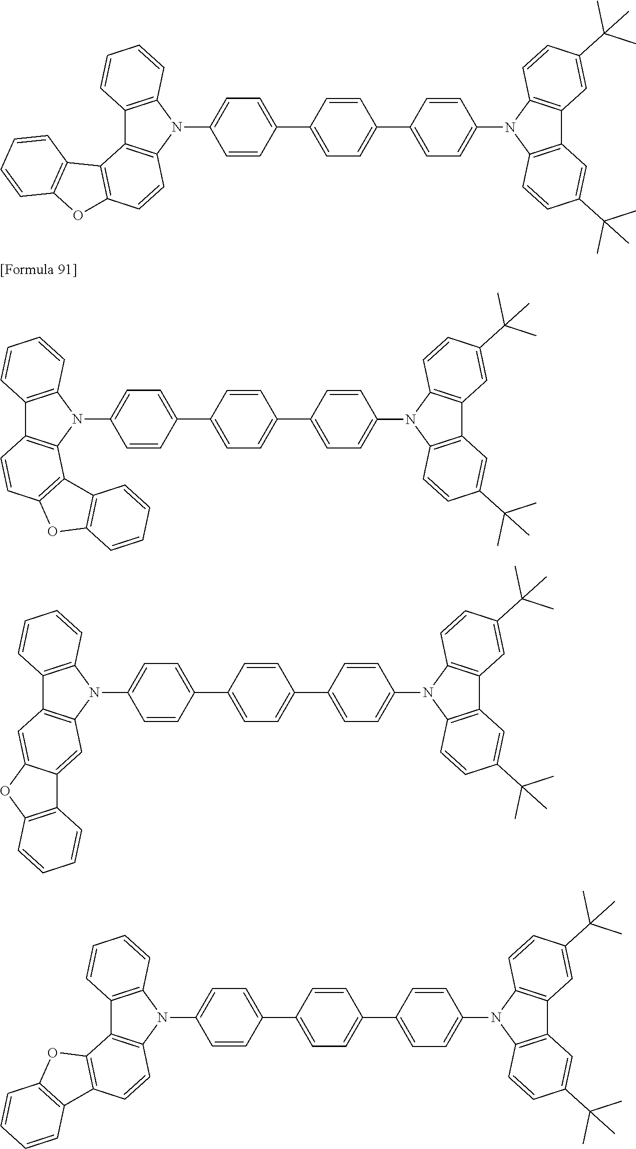

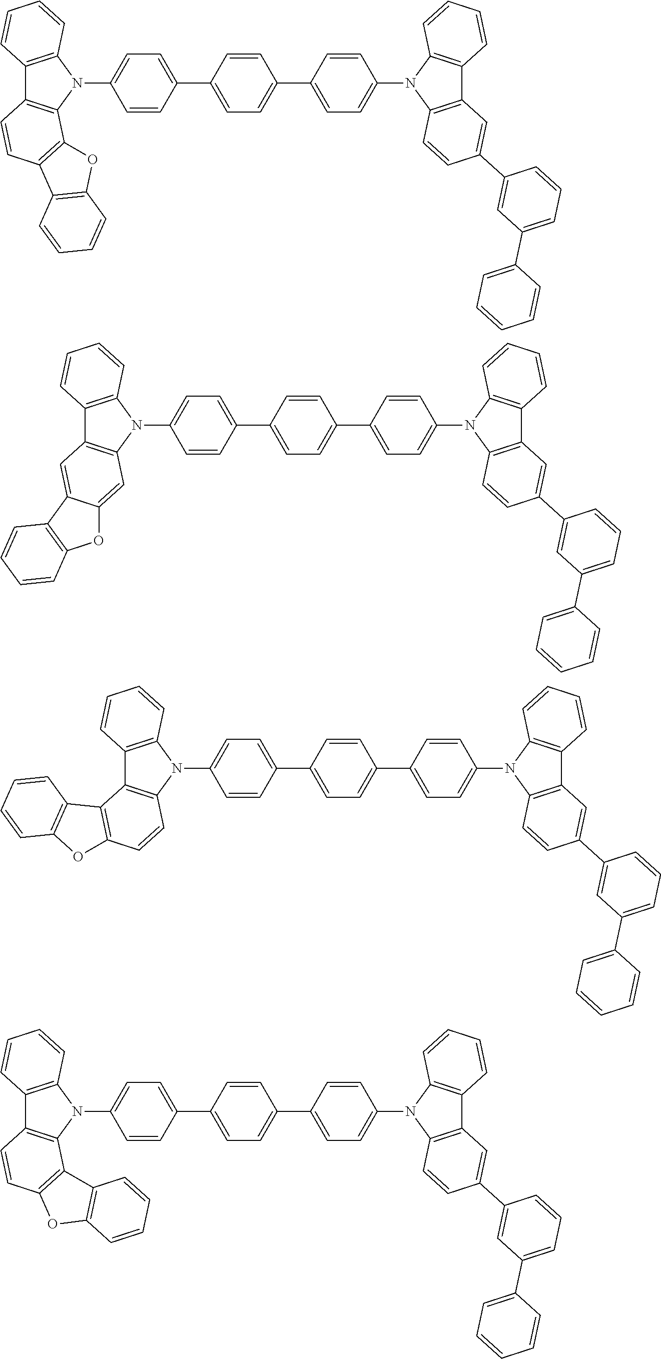

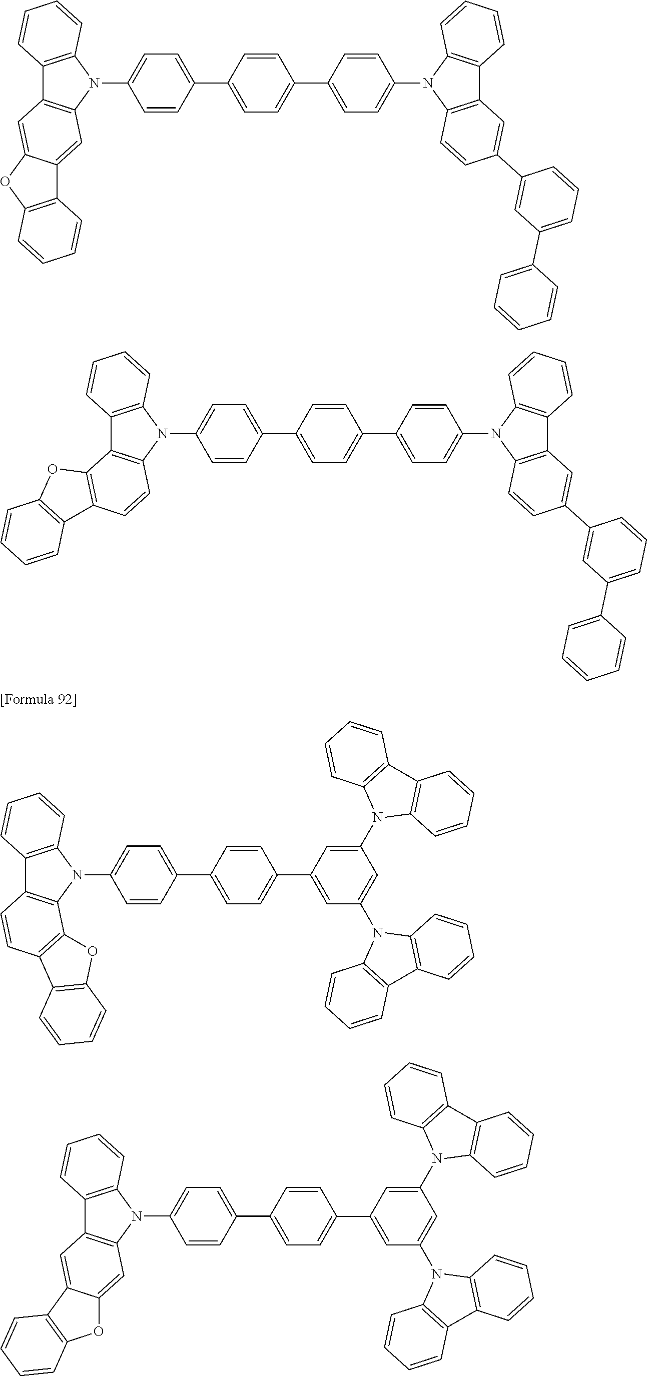

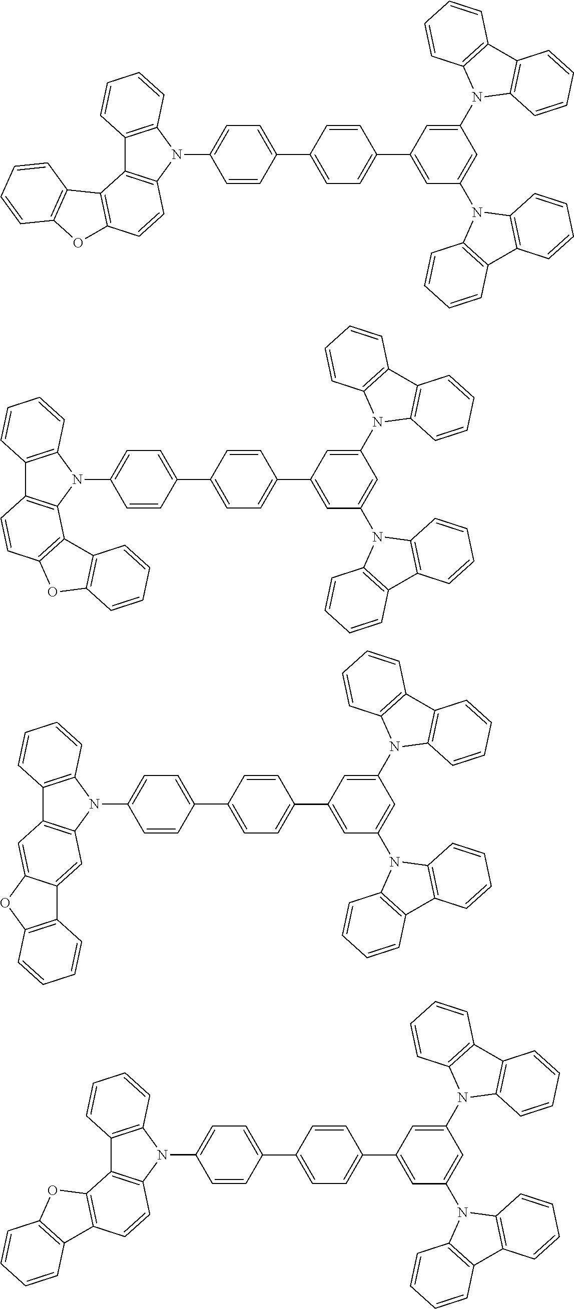

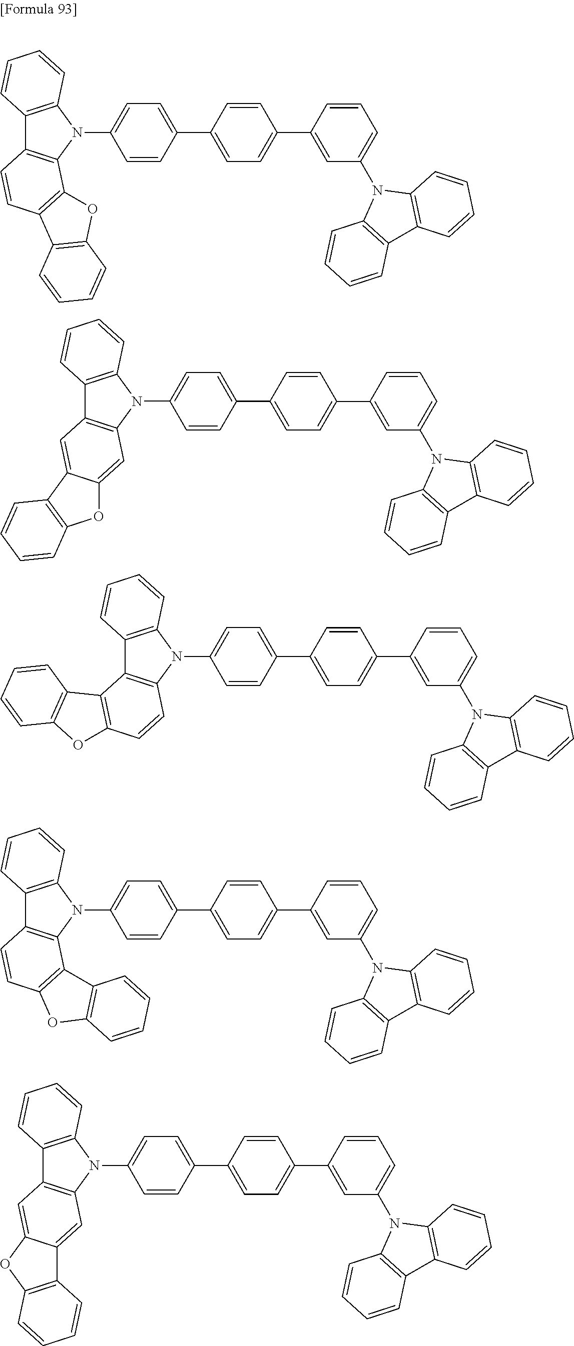

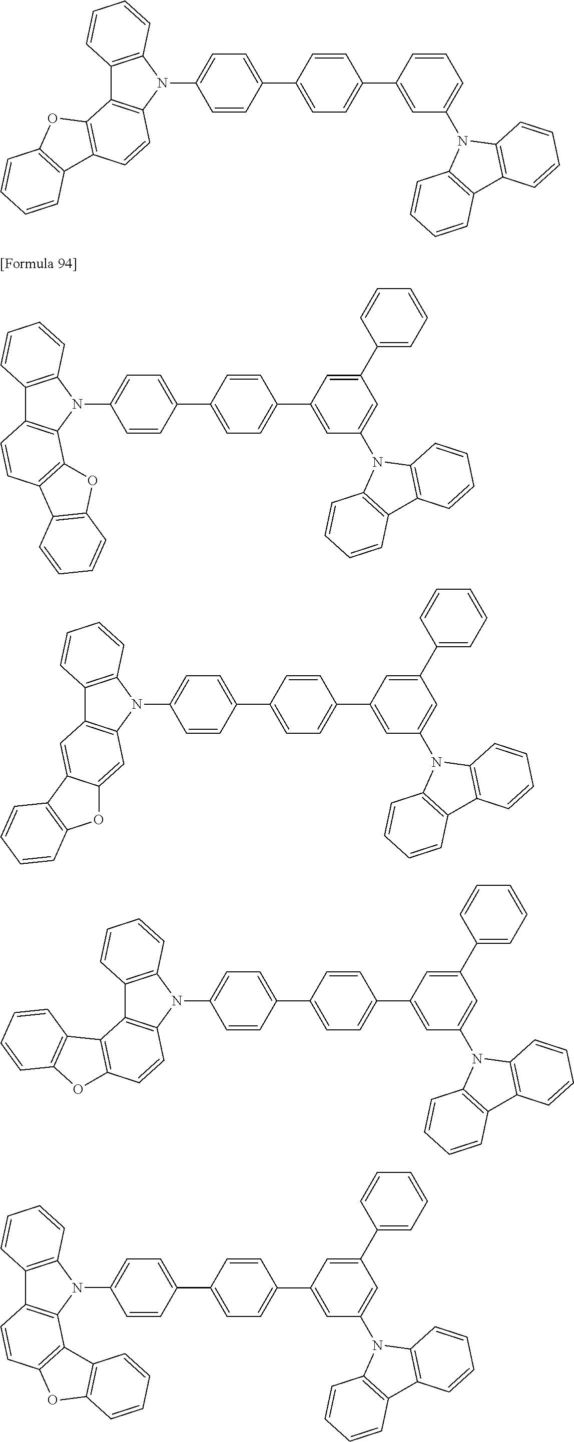

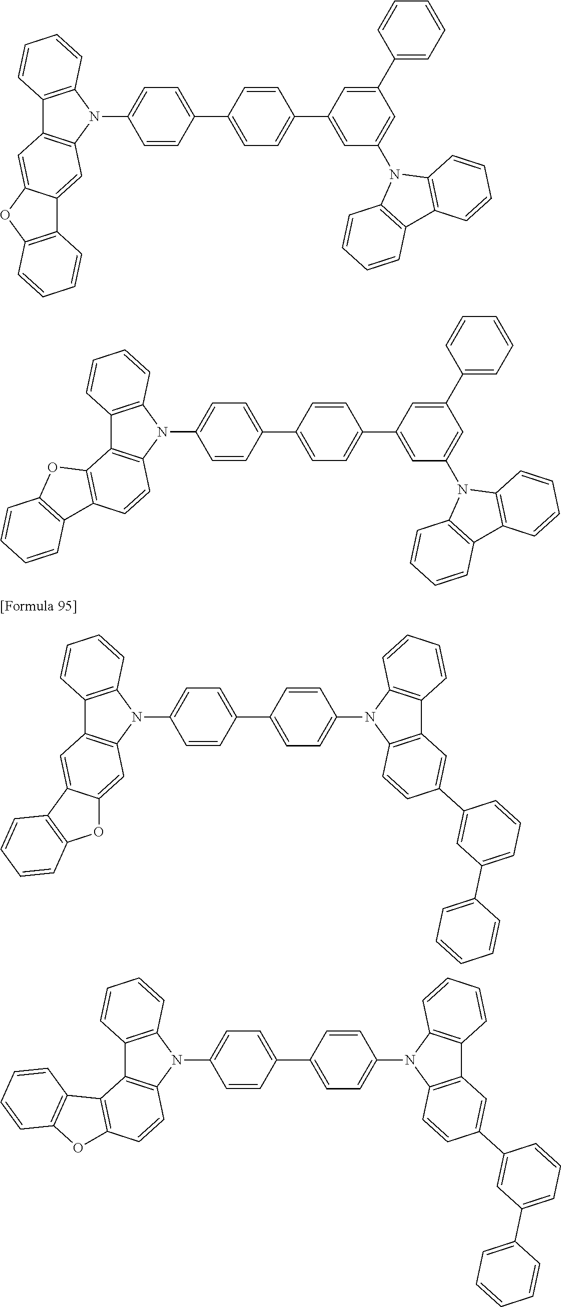

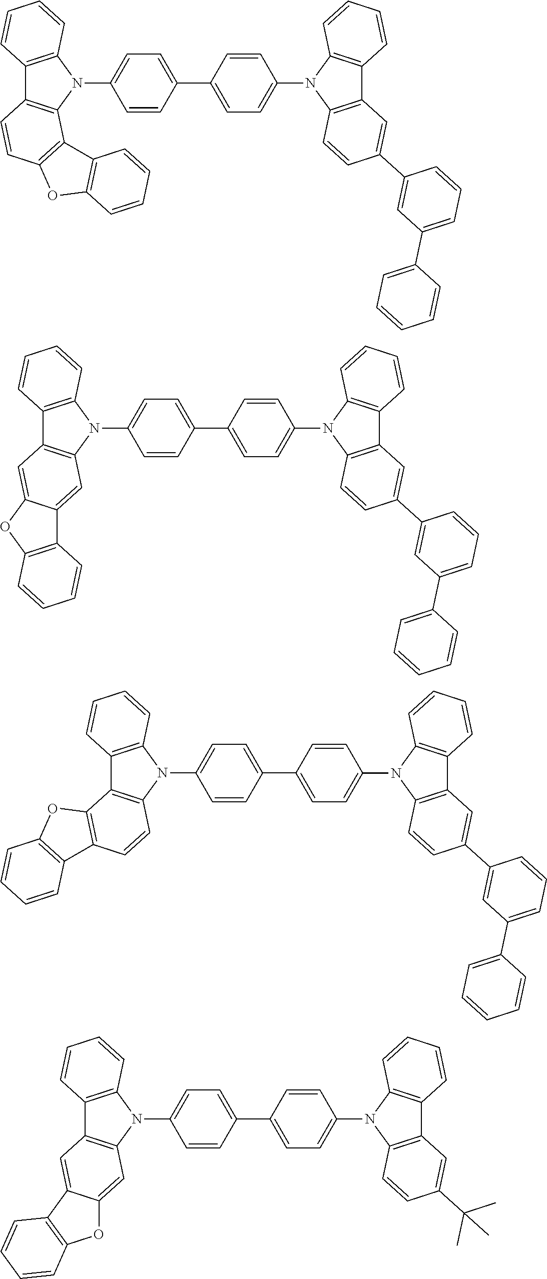

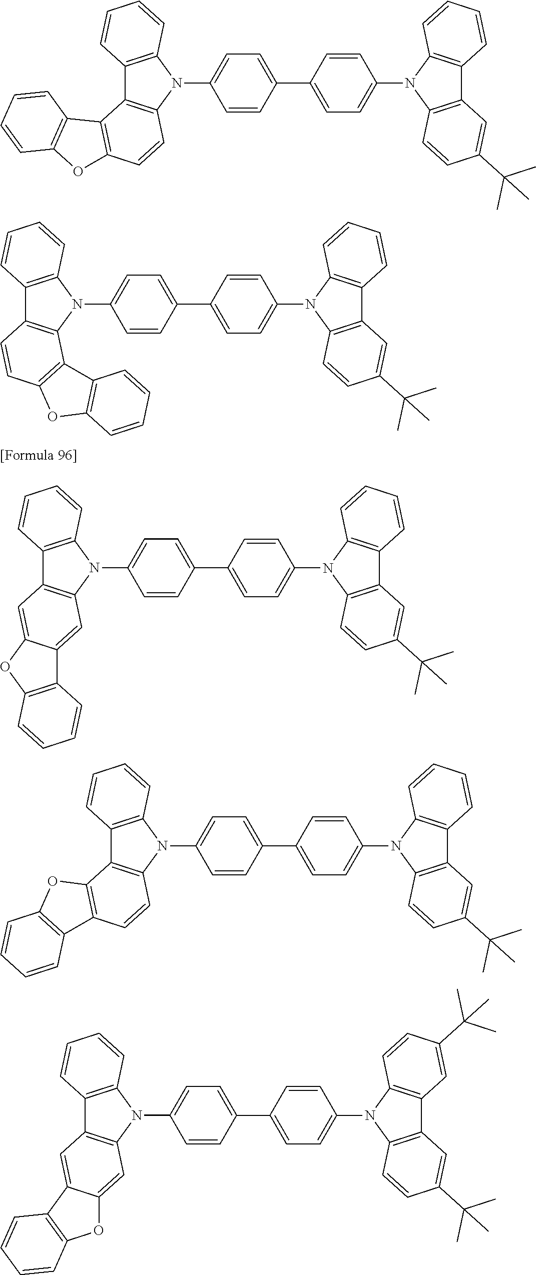

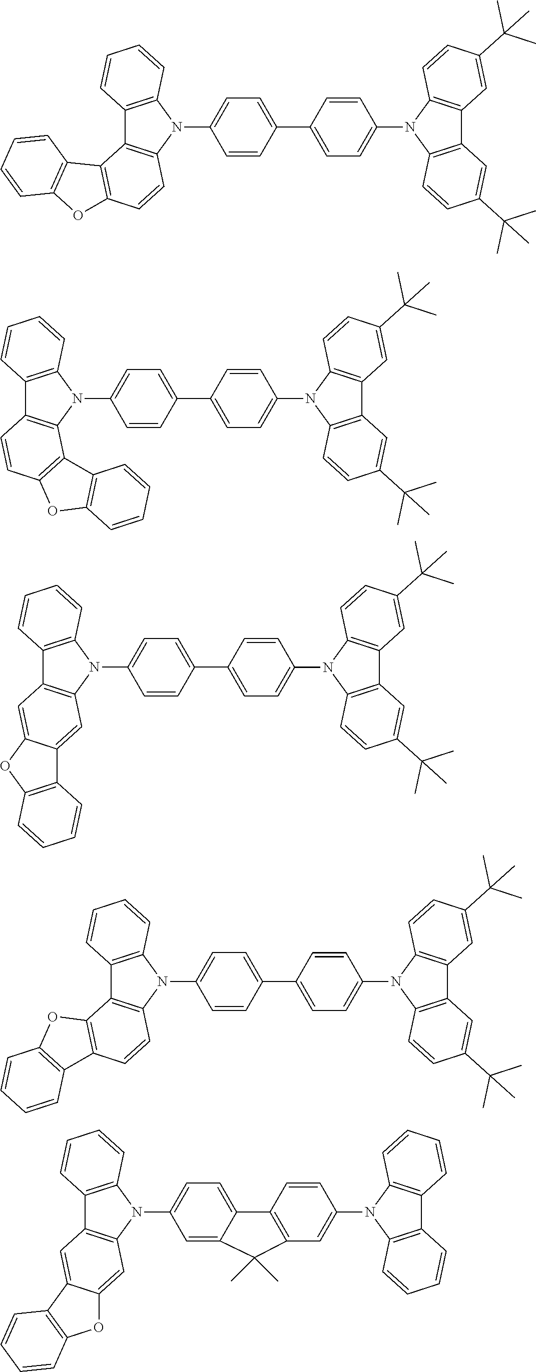

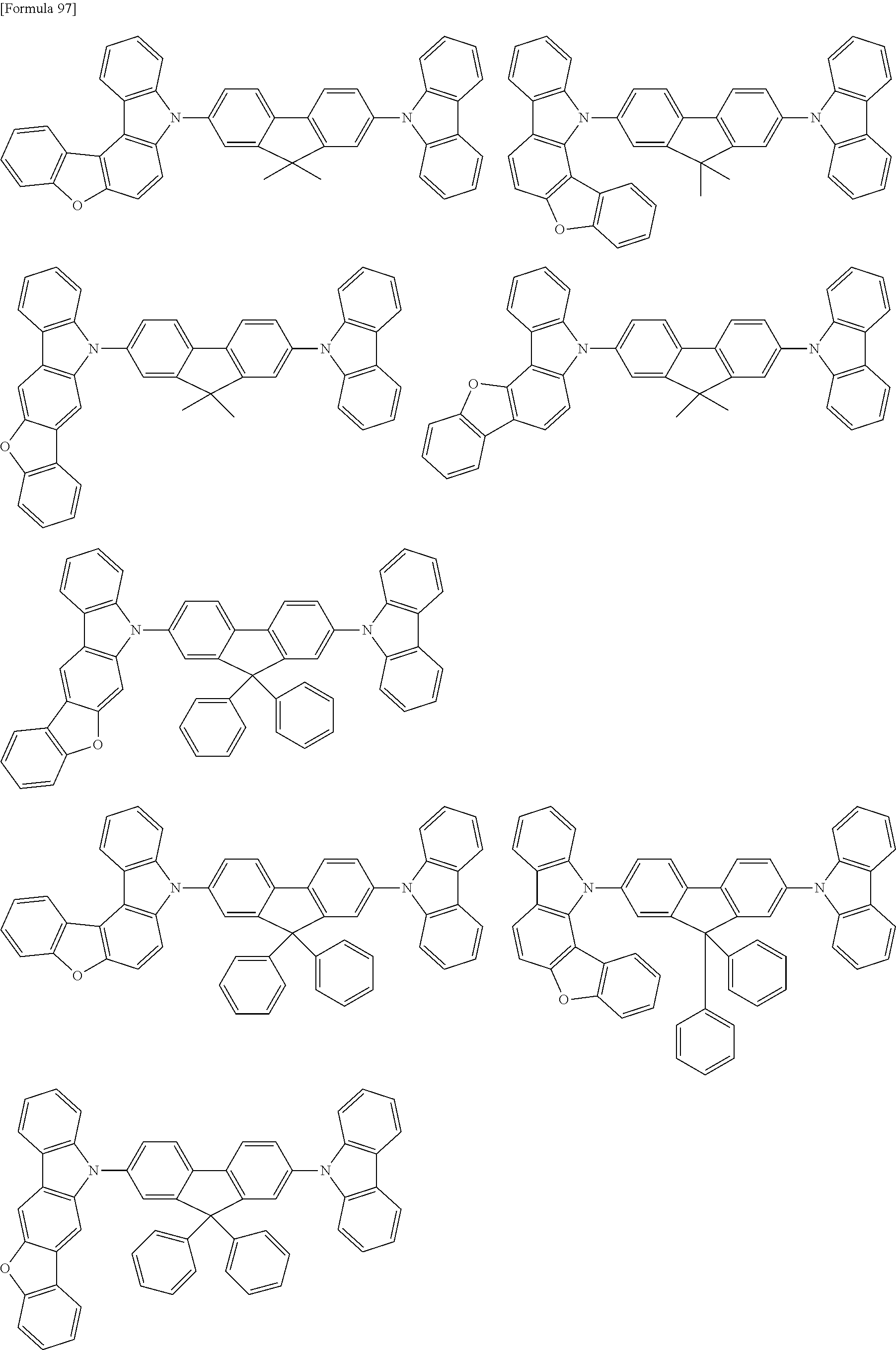

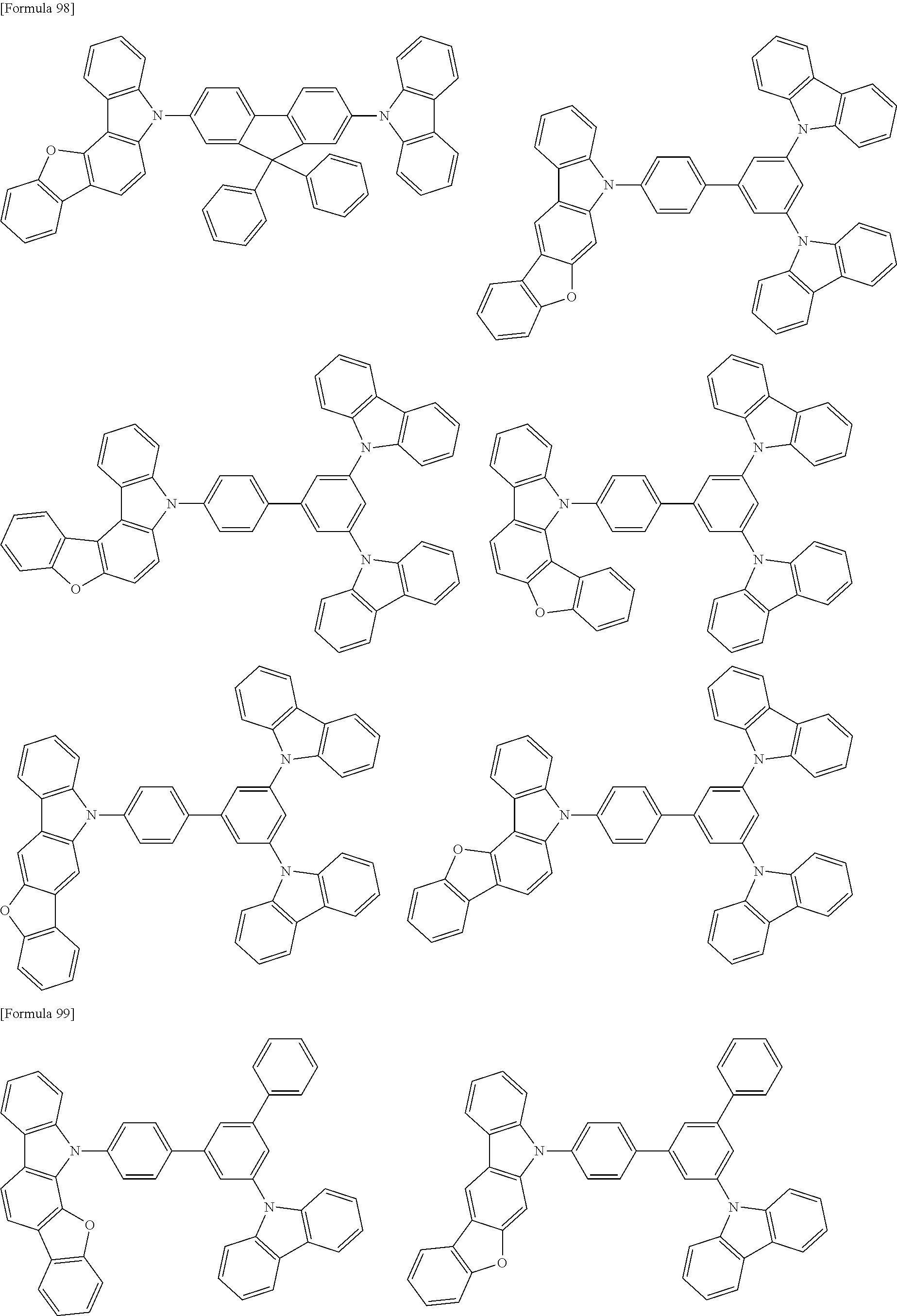

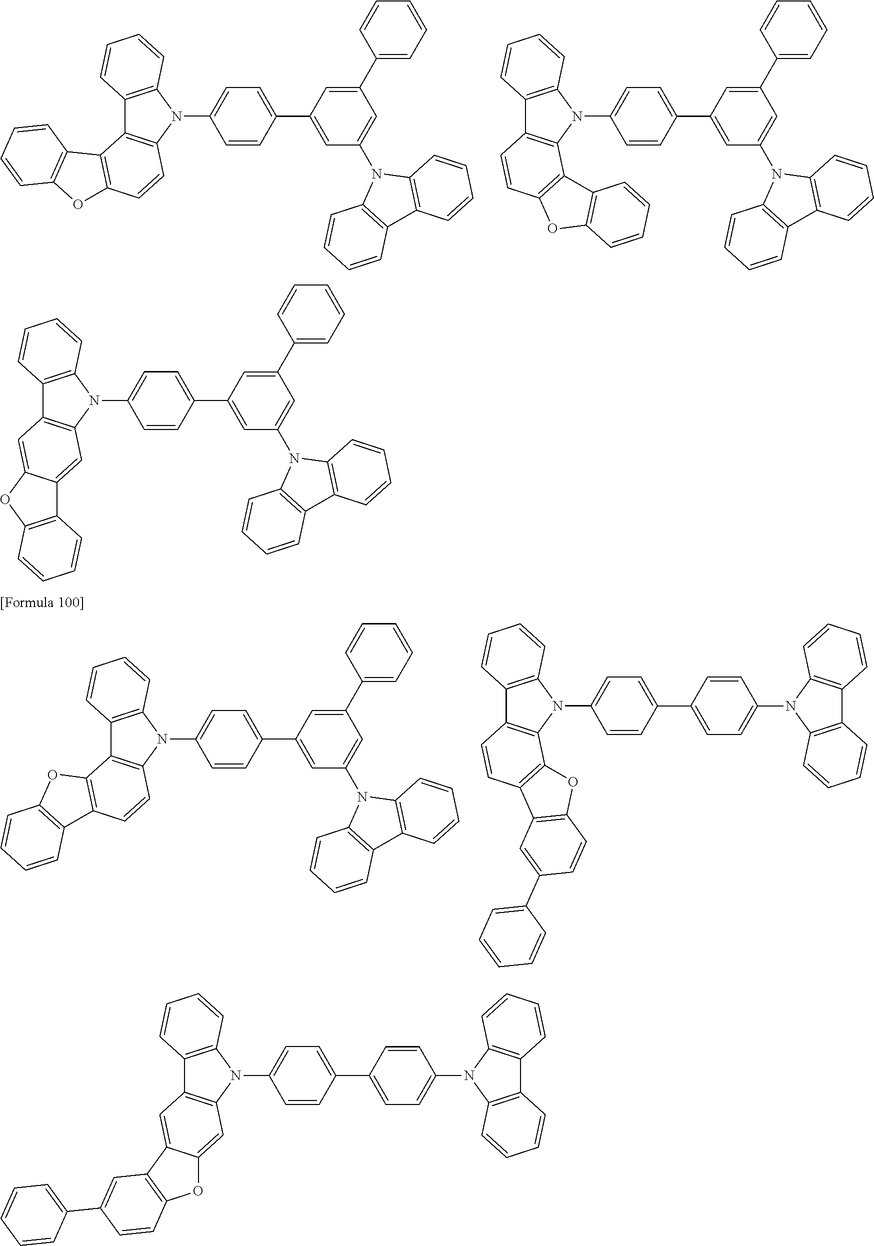

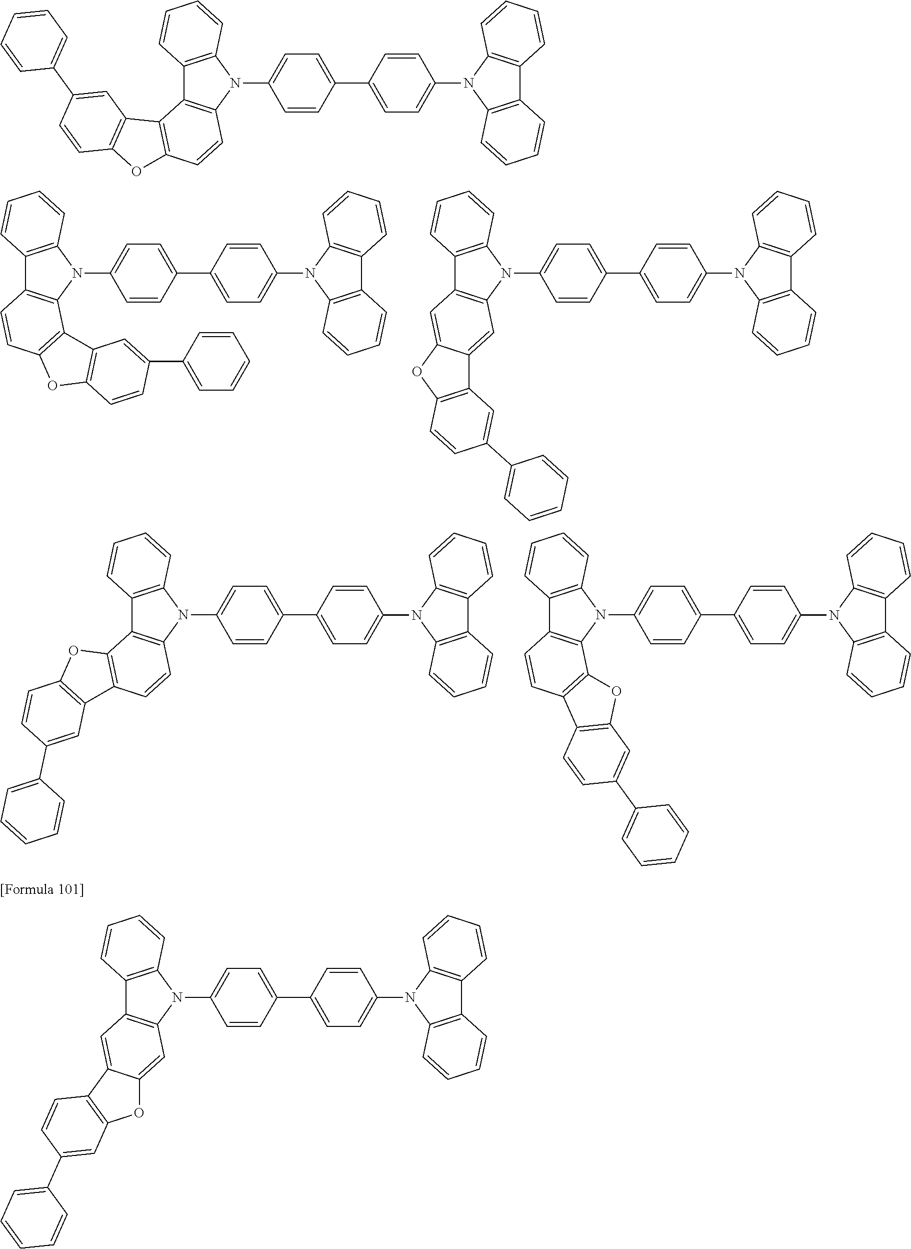

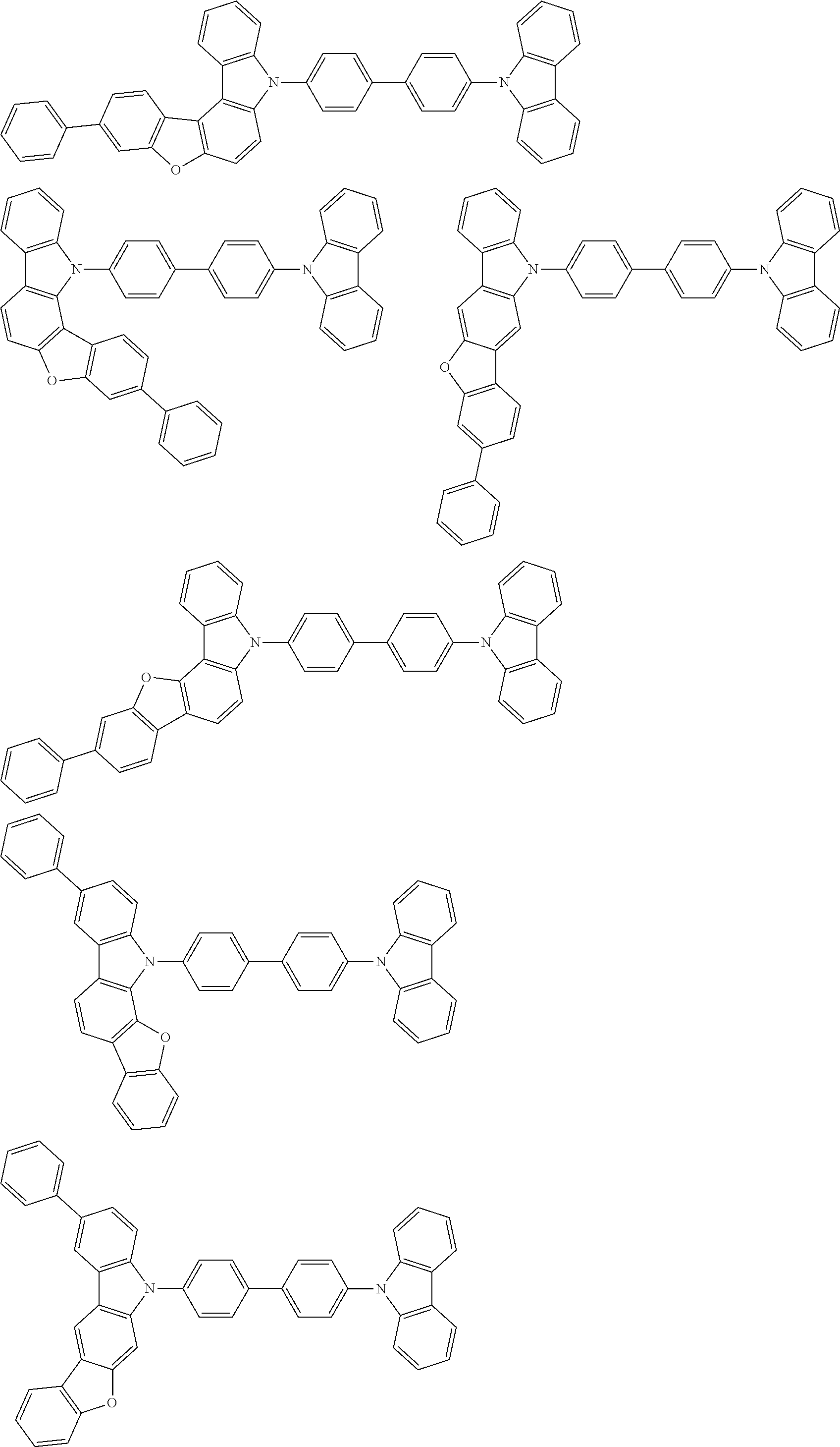

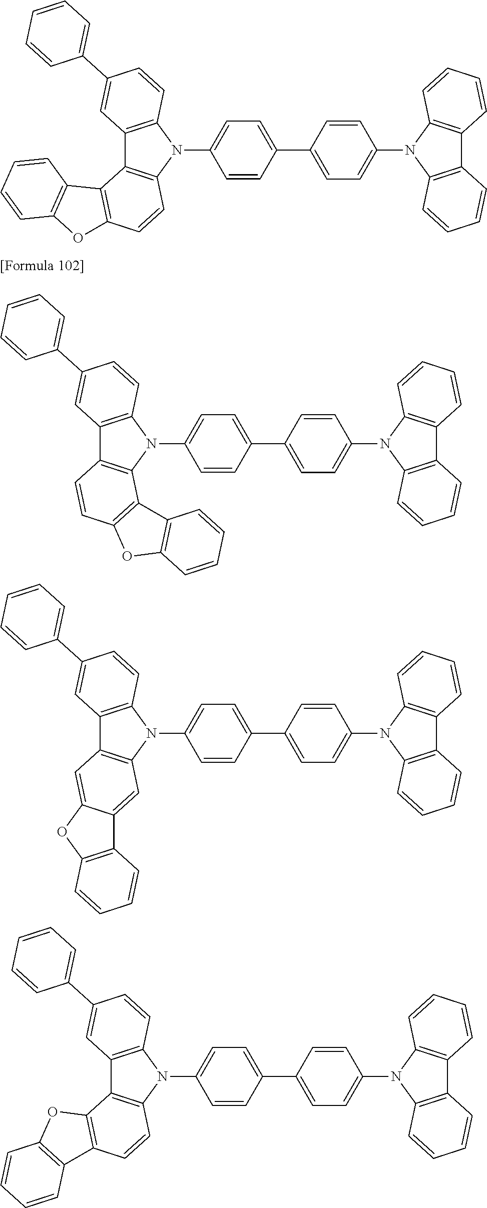

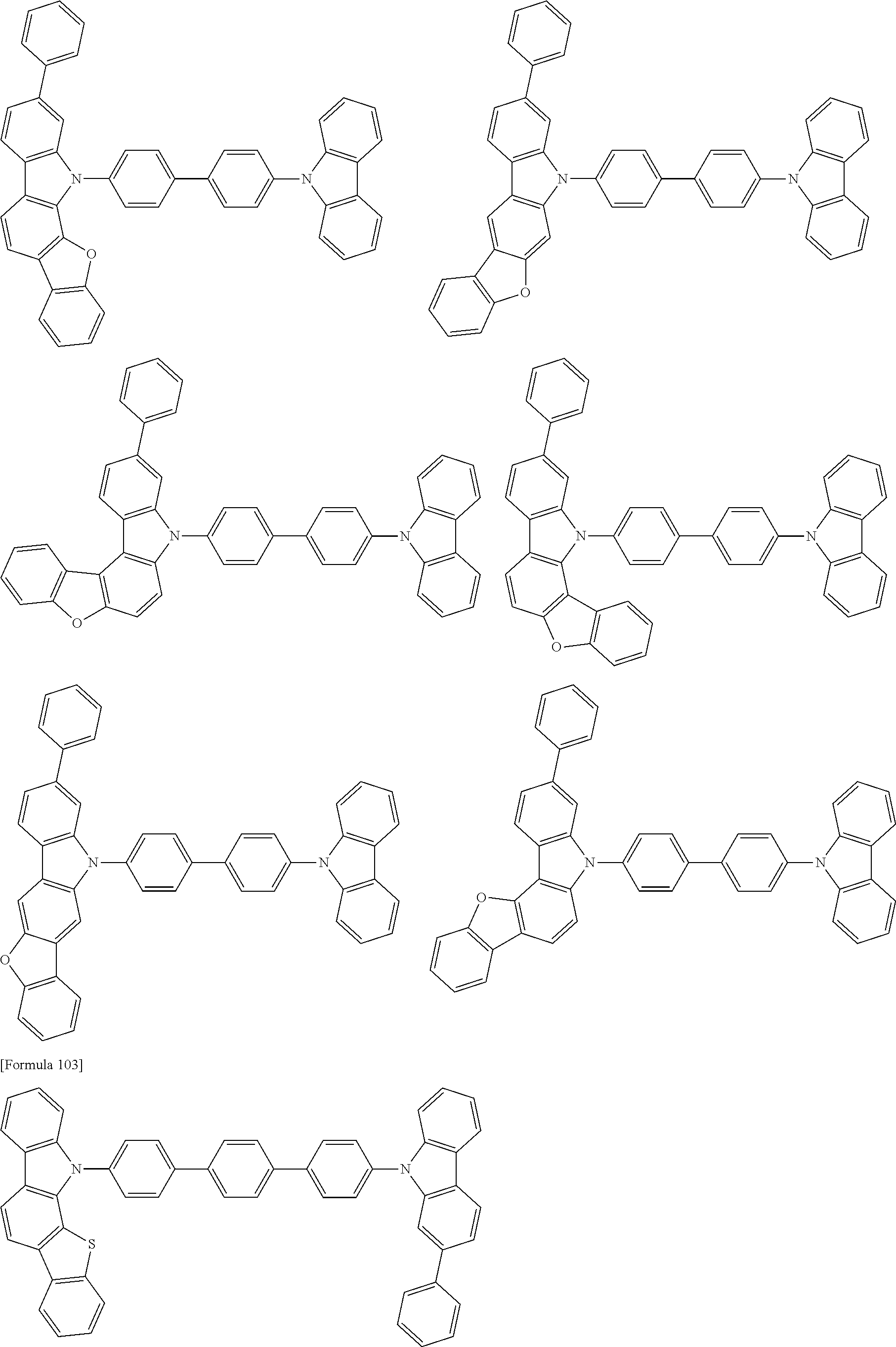

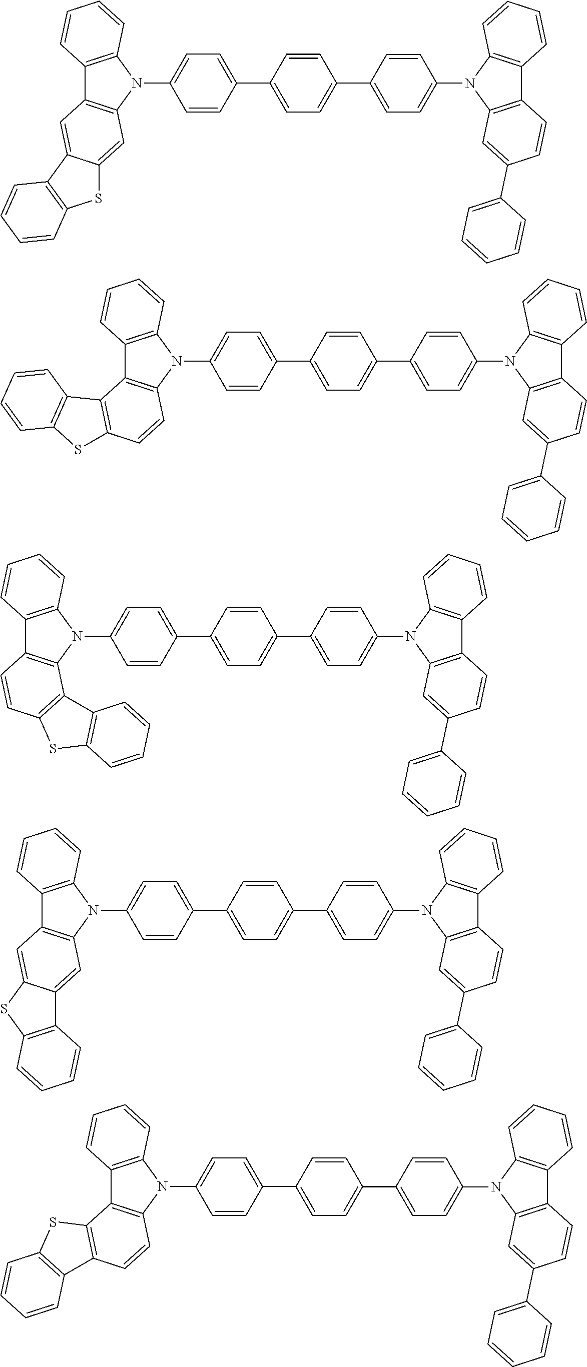

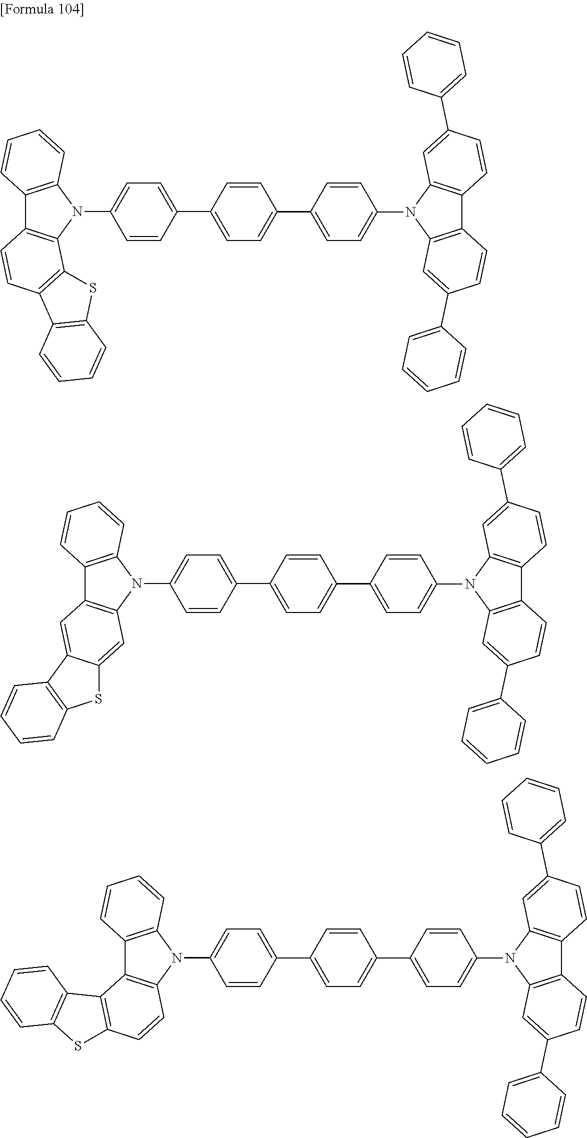

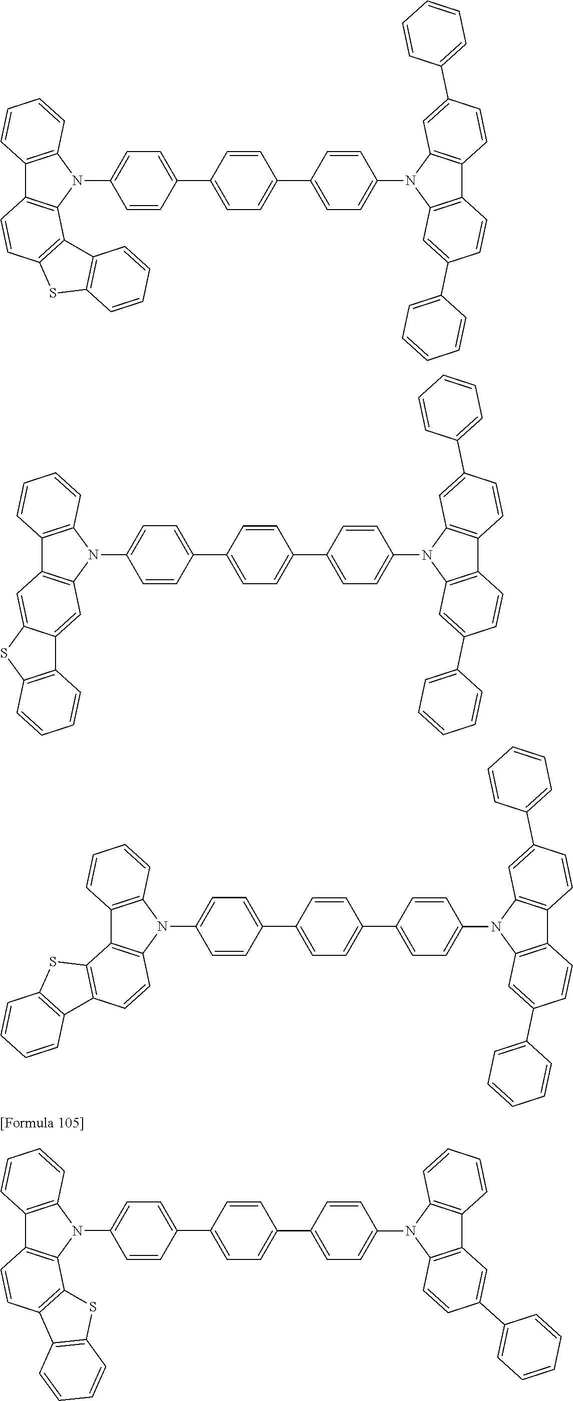

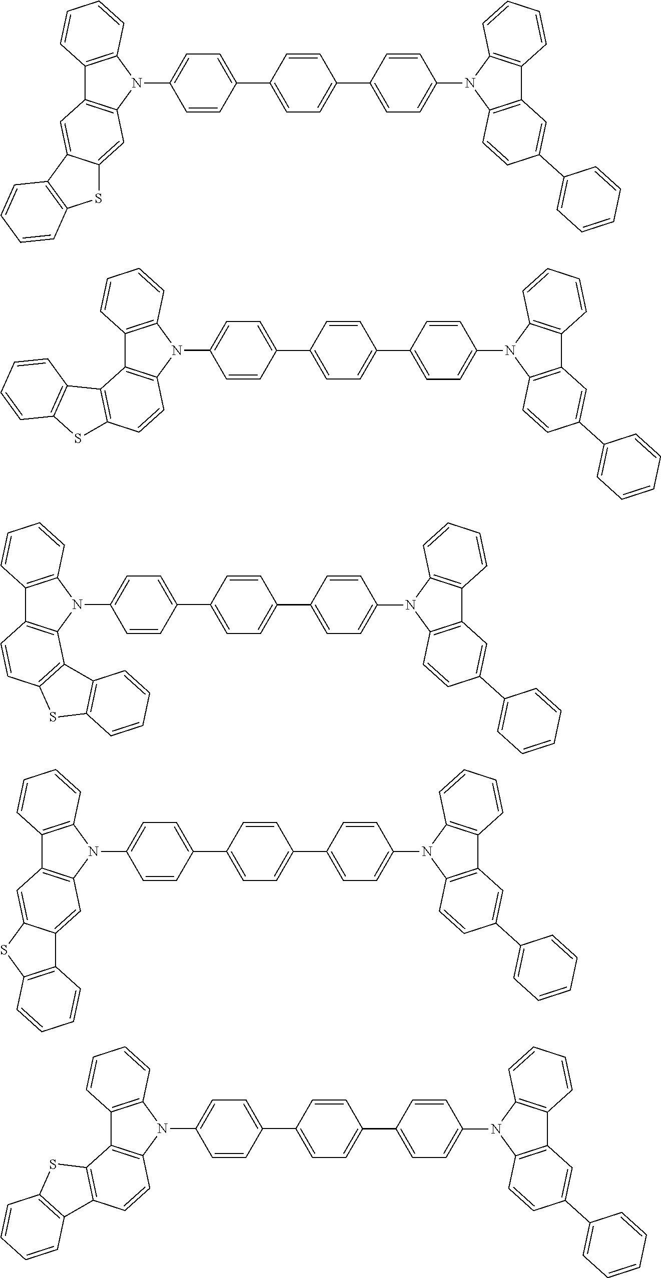

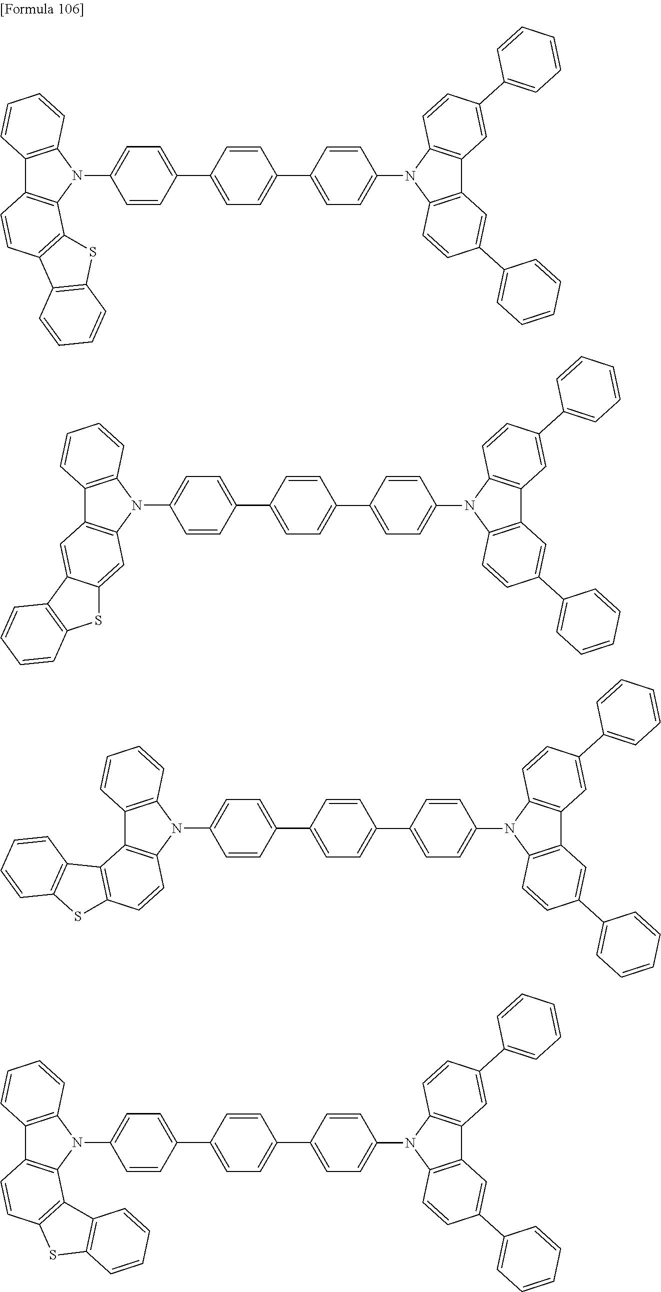

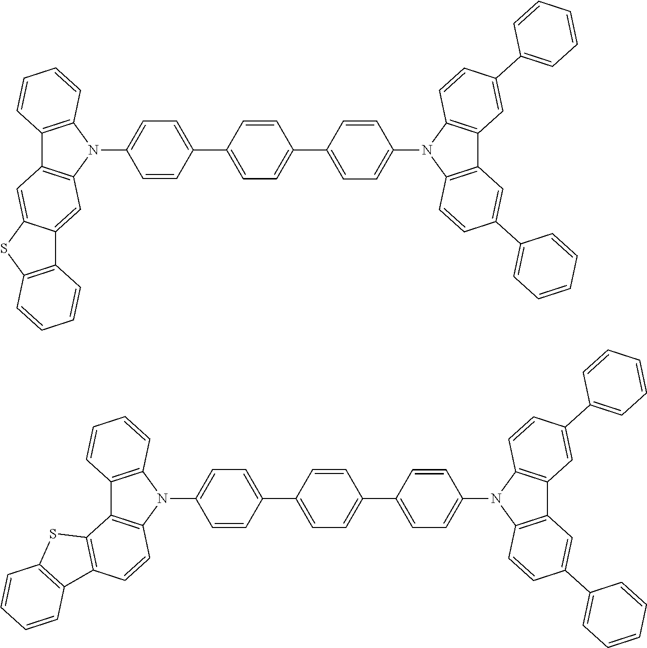 [SMILES: c1ccc(-c2ccc3c(c2)c2cc(-c4ccccc4)ccc2n3-c2ccc(-c3ccc(-c4ccc(-n5c6ccccc6c6cc7sc8ccccc8c7cc65)cc4)cc3)cc2)cc1 ; c1ccc(-c2ccc3c(c2)c2cc(-c4ccccc4)ccc2n3-c2ccc(-c3ccc(-c4ccc(-n5c6ccccc6c6c7sc8ccccc8c7ccc65)cc4)cc3)cc2)cc1]

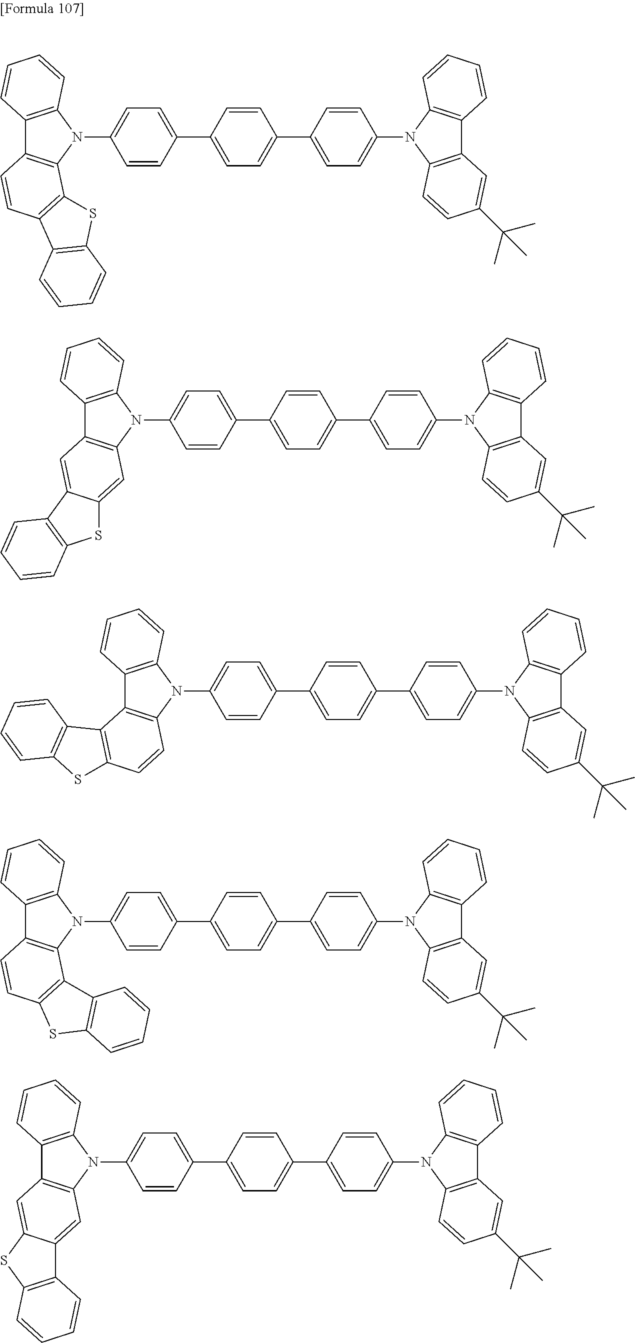

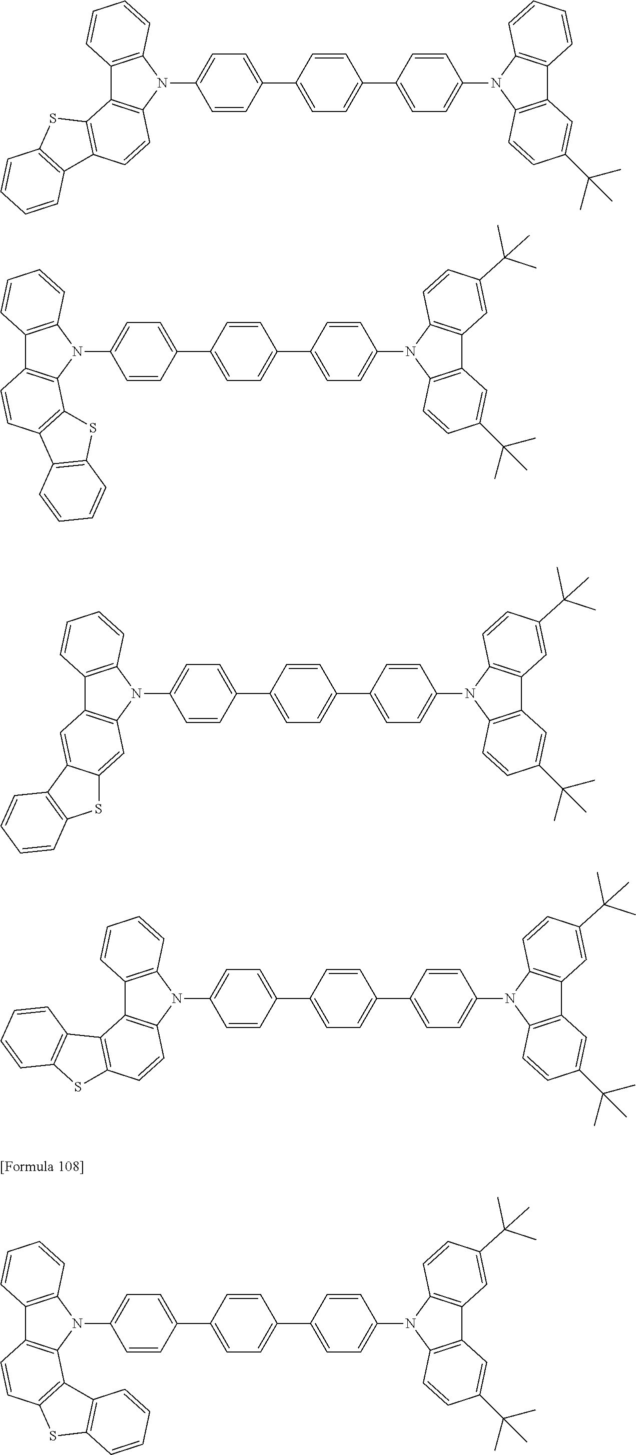

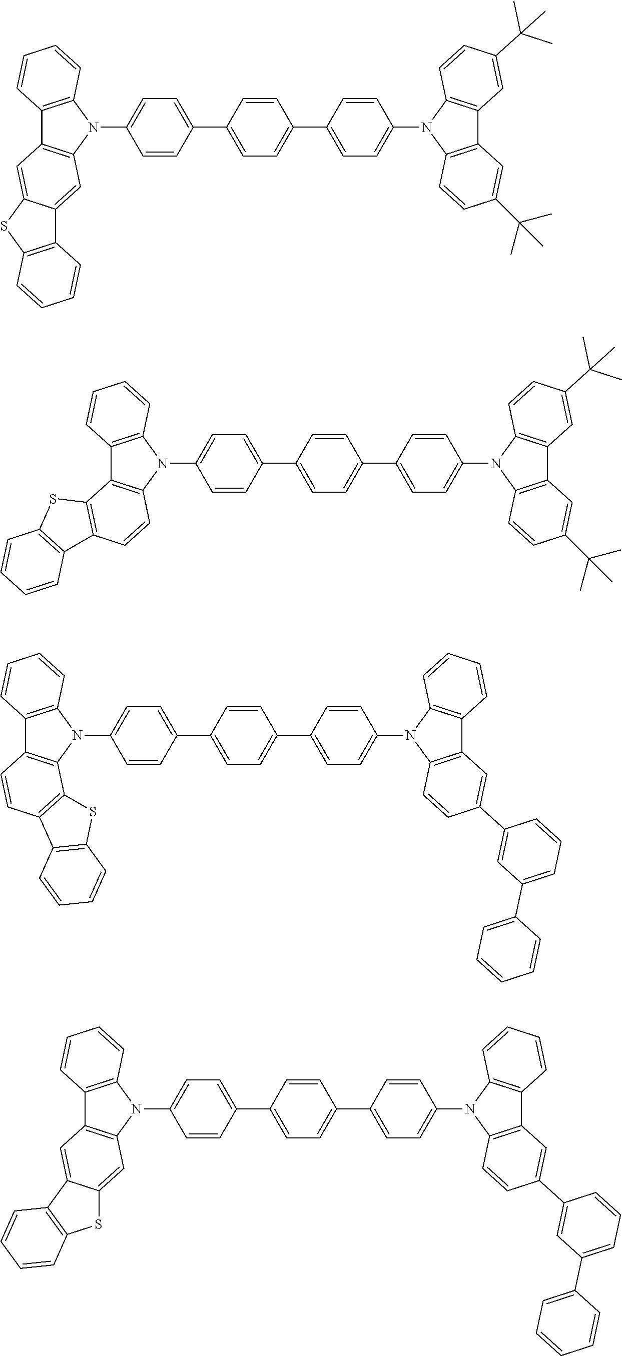

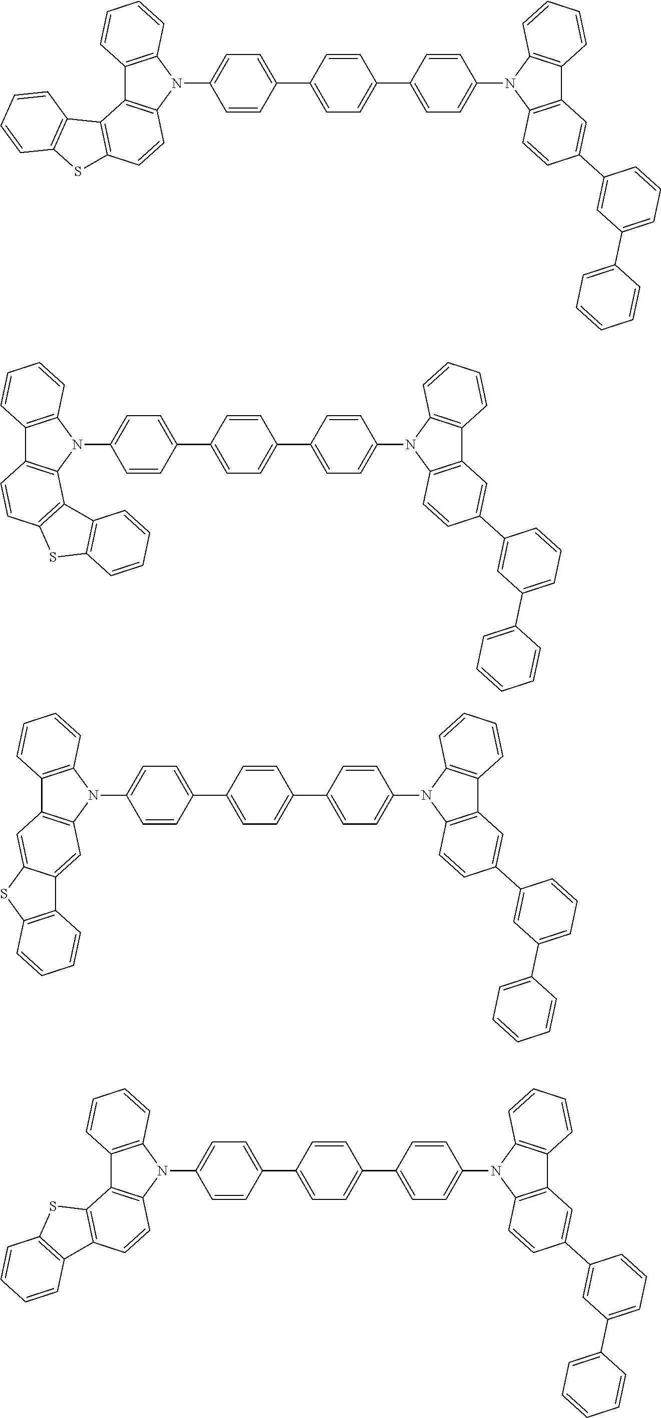

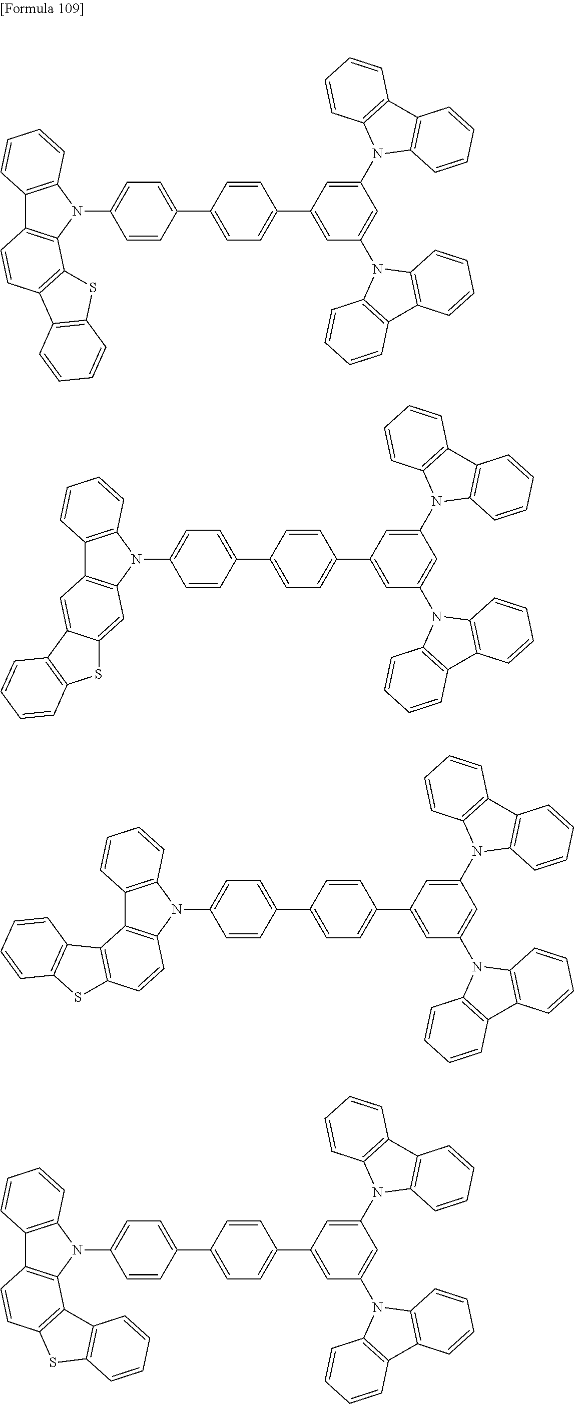 [SMILES: c1ccc2c(c1)sc1c2ccc2c3ccccc3n(-c3ccc(-c4ccc(-c5cc(-n6c7ccccc7c7ccccc76)cc(-n6c7ccccc7c7ccccc76)c5)cc4)cc3)c21 ; c1ccc2c(c1)sc1cc3c(cc12)c1ccccc1n3-c1ccc(-c2ccc(-c3cc(-n4c5ccccc5c5ccccc54)cc(-n4c5ccccc5c5ccccc54)c3)cc2)cc1 ; c1ccc2c(c1)sc1ccc3c(c4ccccc4n3-c3ccc(-c4ccc(-c5cc(-n6c7ccccc7c7ccccc76)cc(-n6c7ccccc7c7ccccc76)c5)cc4)cc3)c12 ; c1ccc2c(c1)sc1ccc3c4ccccc4n(-c4ccc(-c5ccc(-c6cc(-n7c8ccccc8c8ccccc87)cc(-n7c8ccccc8c8ccccc87)c6)cc5)cc4)c3c12]

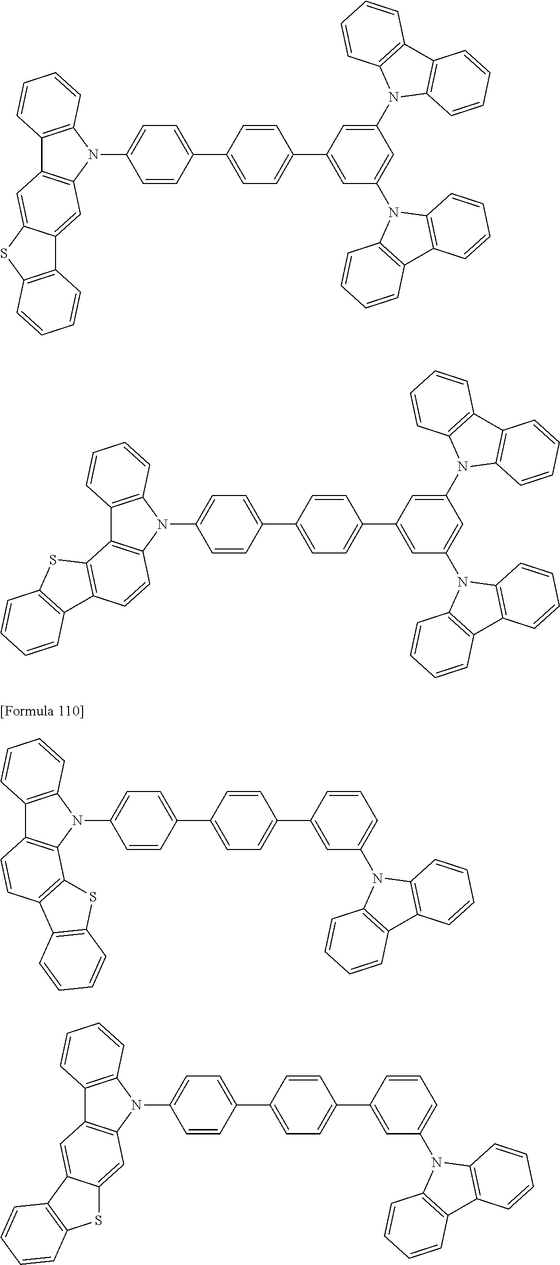

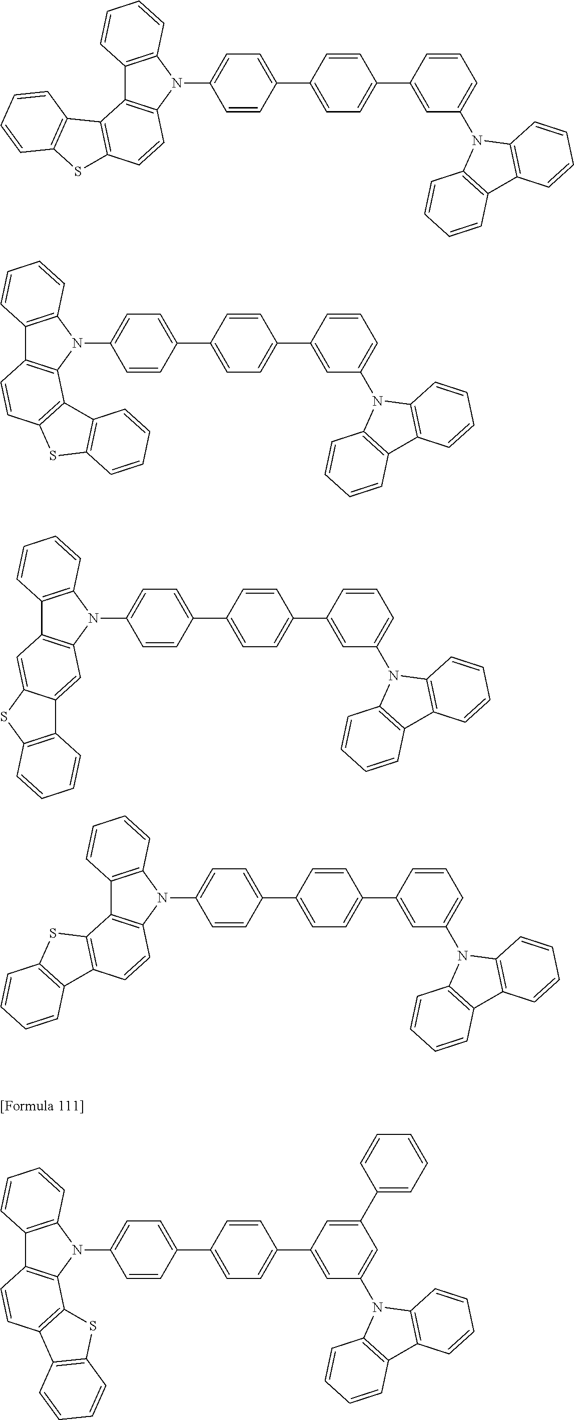

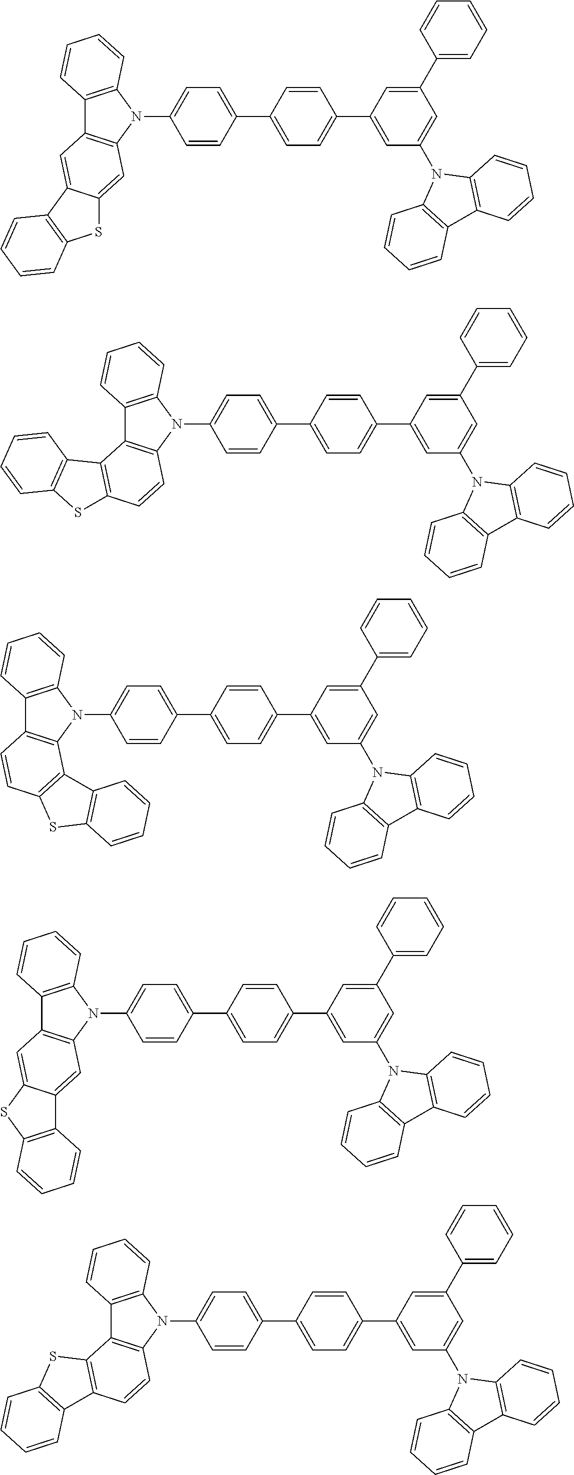

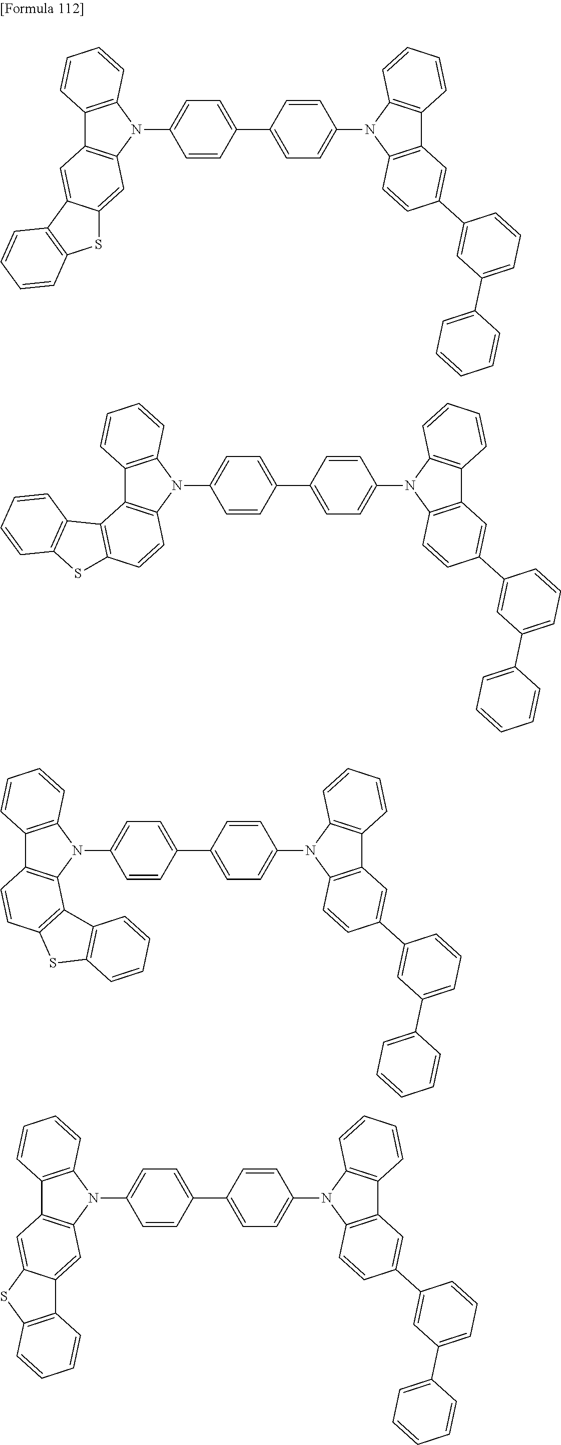

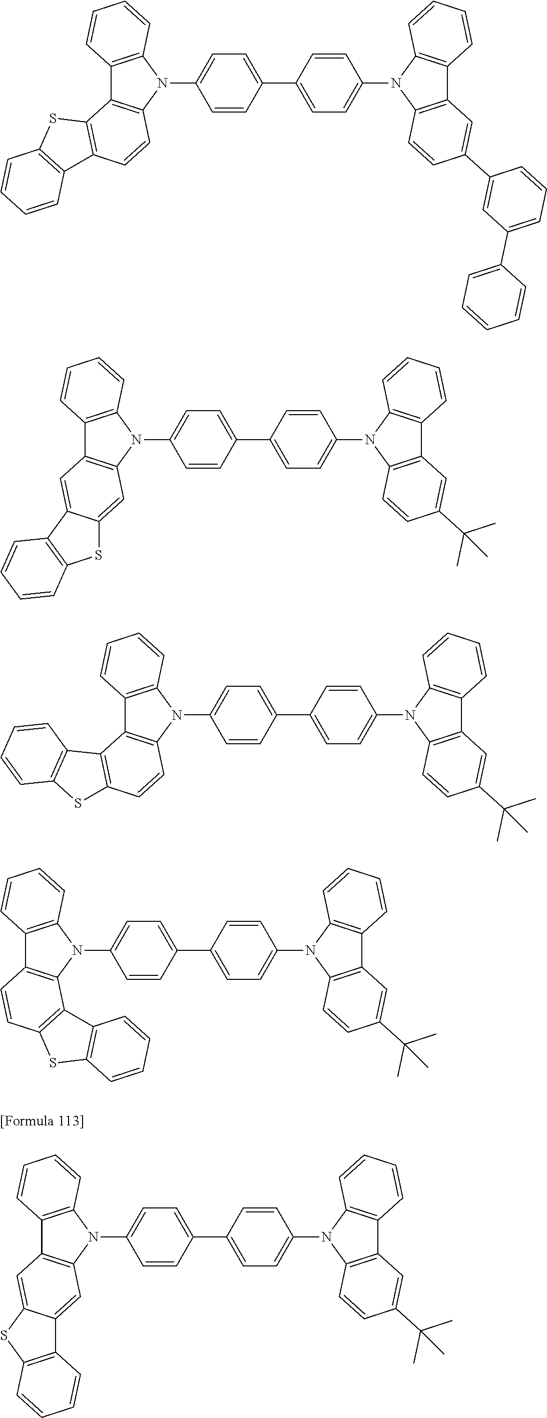 [SMILES: c1ccc(-c2cccc(-c3ccc4c(c3)c3ccccc3n4-c3ccc(-c4ccc(-n5c6ccccc6c6c7sc8ccccc8c7ccc65)cc4)cc3)c2)cc1 ; CC(C)(C)c1ccc2c(c1)c1ccccc1n2-c1ccc(-c2ccc(-n3c4ccccc4c4cc5c(cc43)sc3ccccc35)cc2)cc1 ; CC(C)(C)c1ccc2c(c1)c1ccccc1n2-c1ccc(-c2ccc(-n3c4ccccc4c4c5c(ccc43)sc3ccccc35)cc2)cc1 ; CC(C)(C)c1ccc2c(c1)c1ccccc1n2-c1ccc(-c2ccc(-n3c4ccccc4c4ccc5sc6ccccc6c5c43)cc2)cc1 ; CC(C)(C)c1ccc2c(c1)c1ccccc1n2-c1ccc(-c2ccc(-n3c4ccccc4c4cc5sc6ccccc6c5cc43)cc2)cc1]

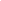

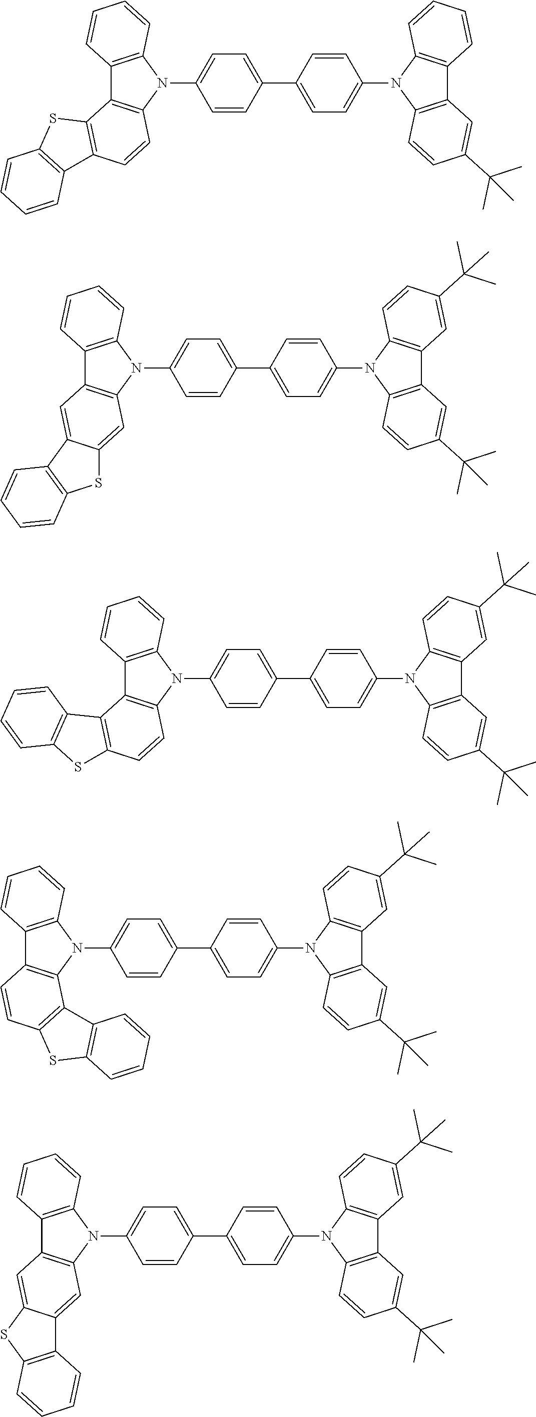

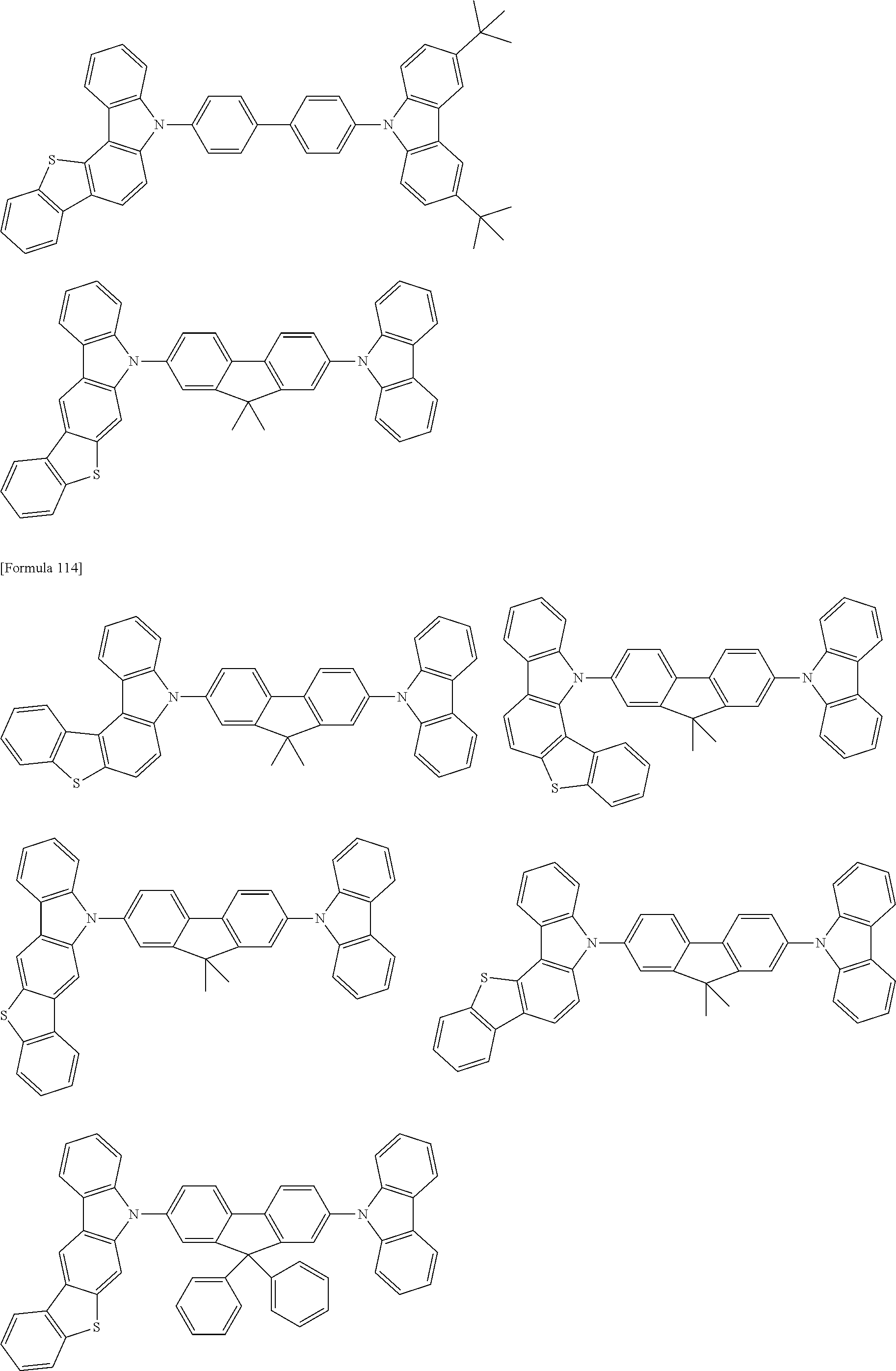

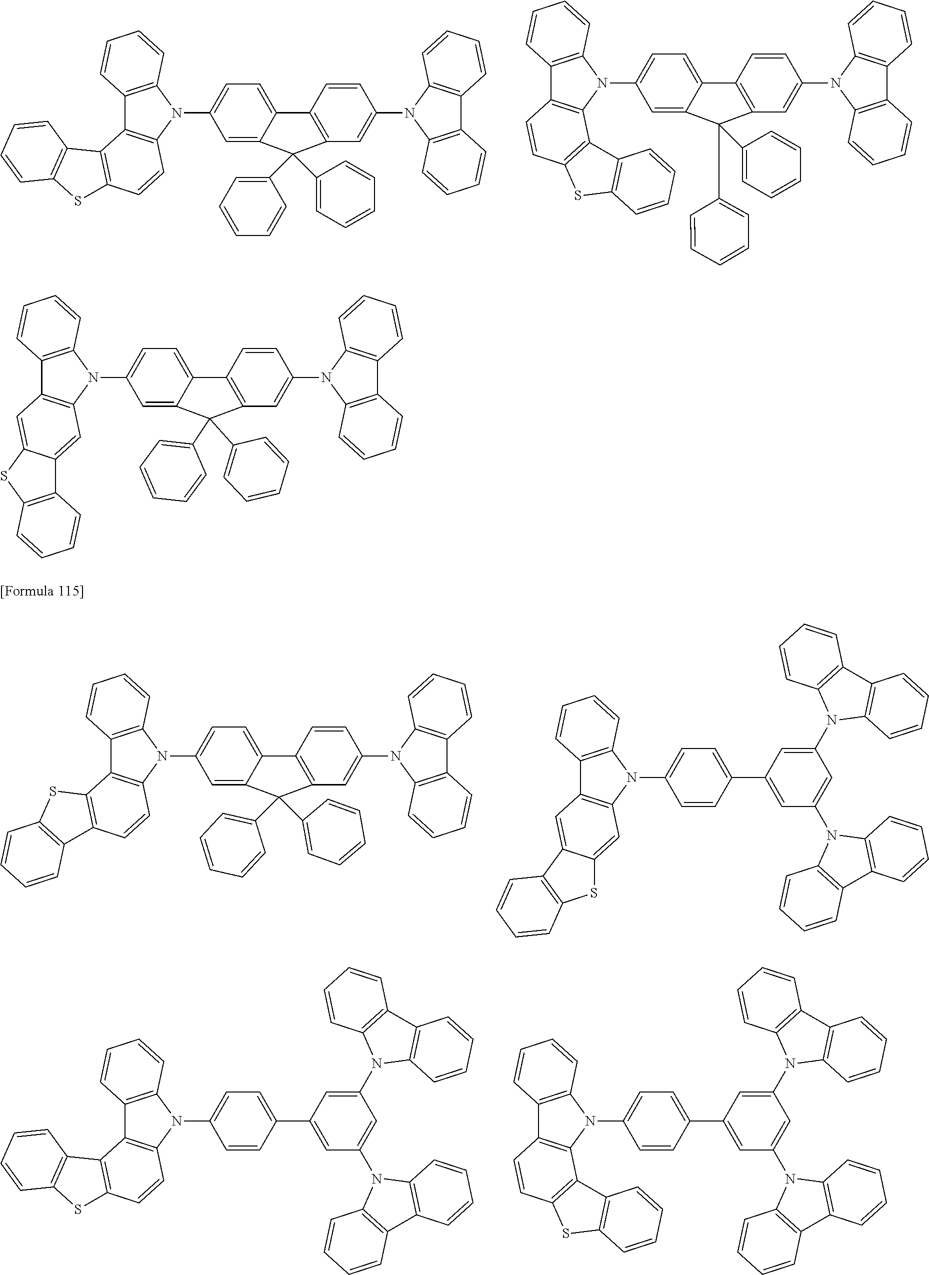

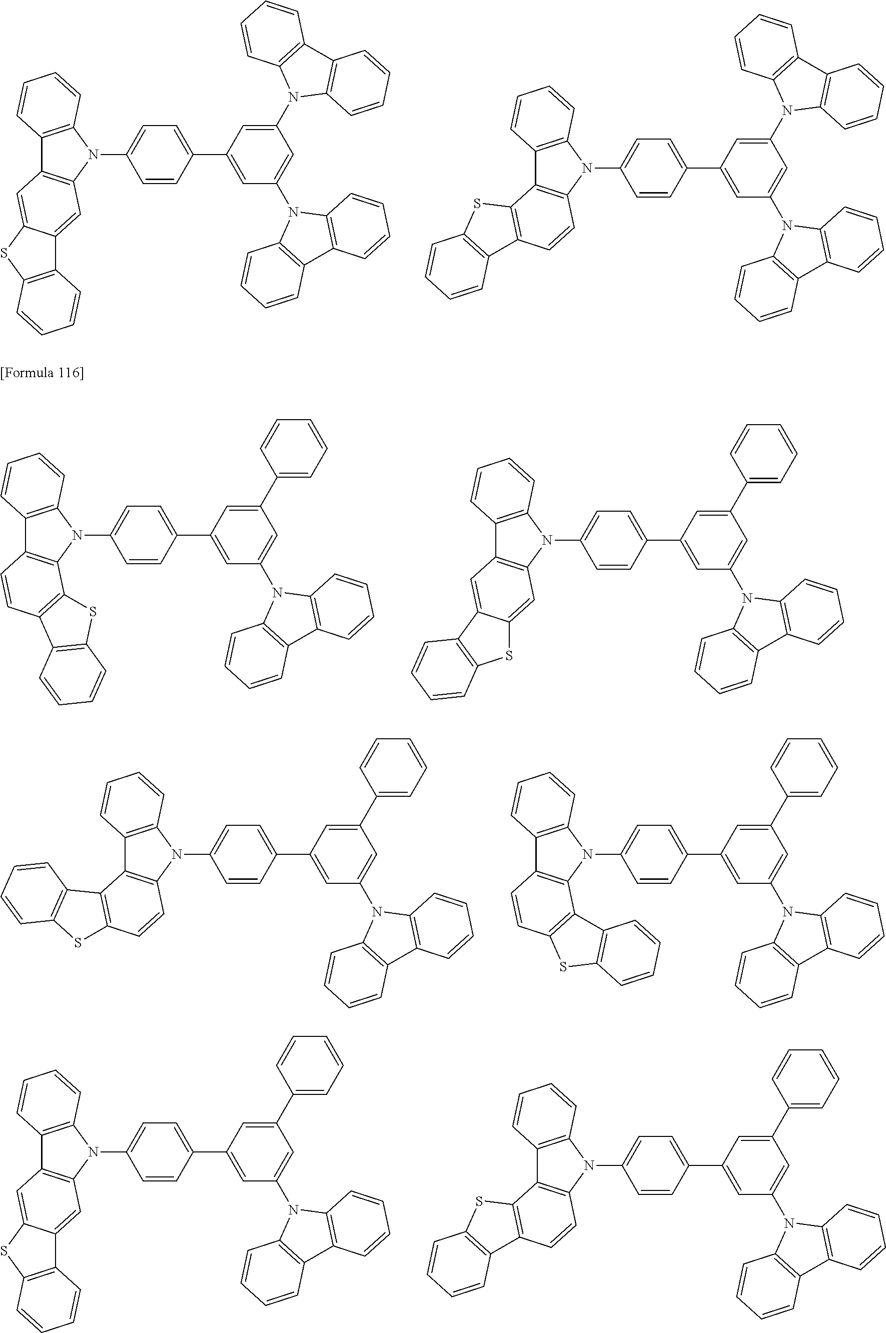

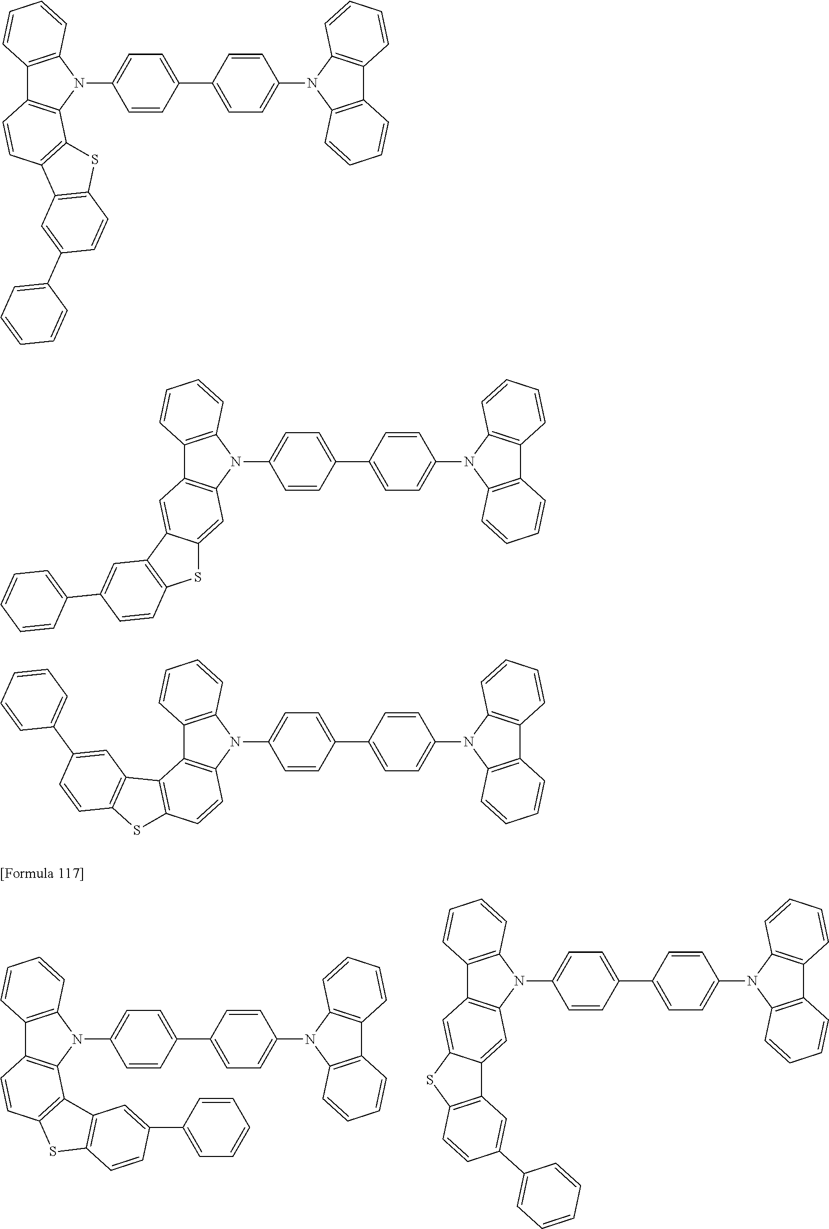

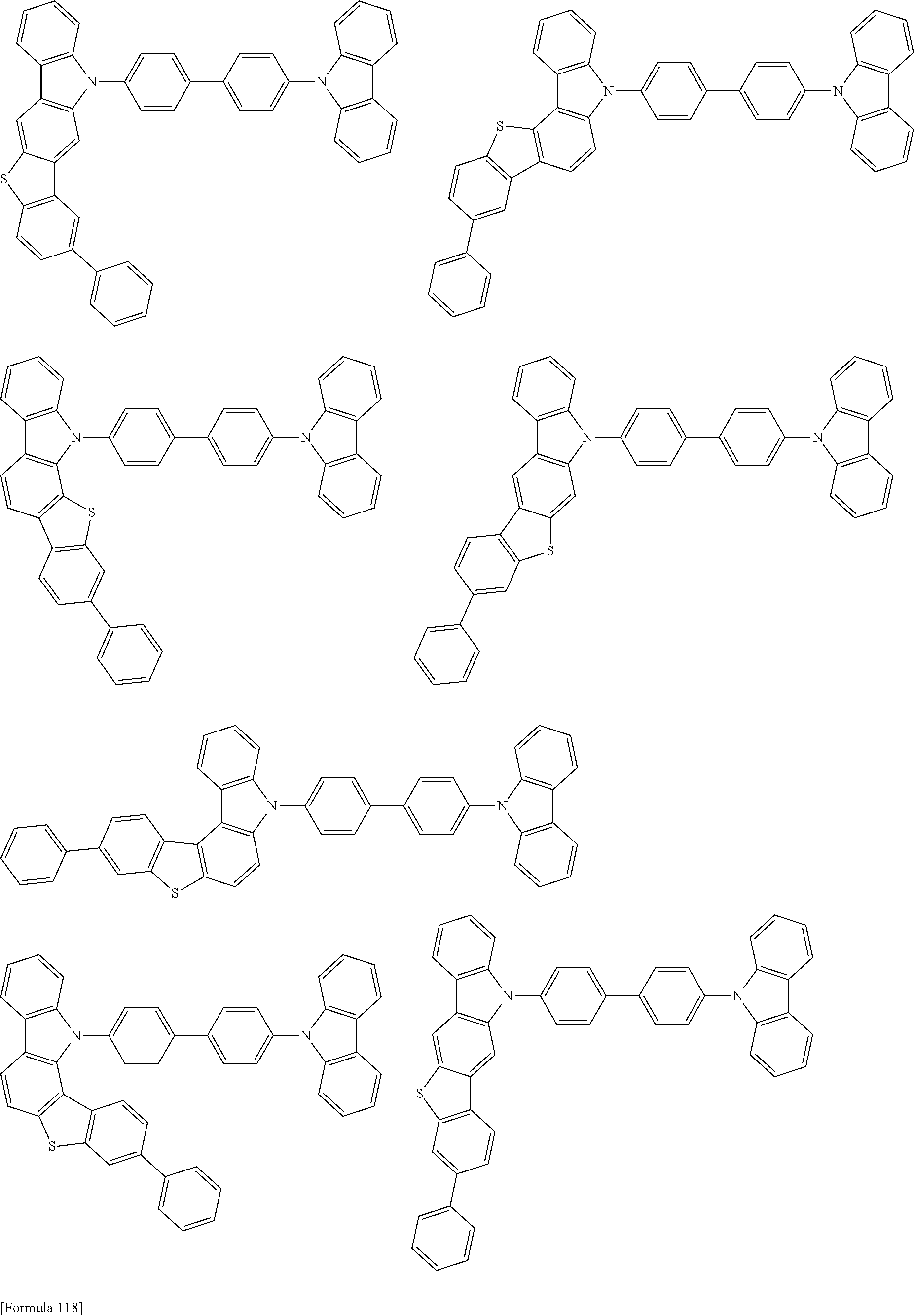

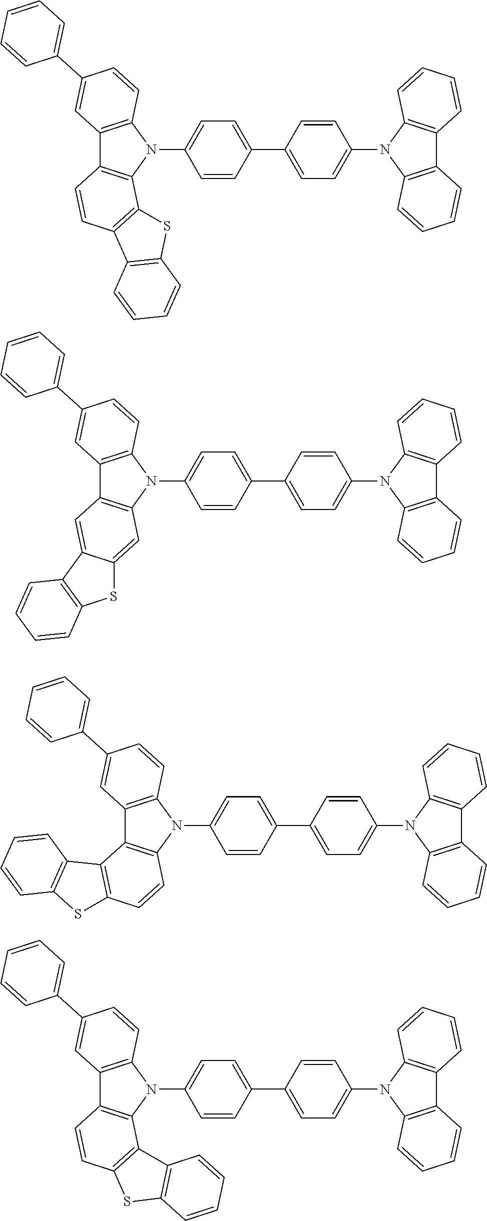

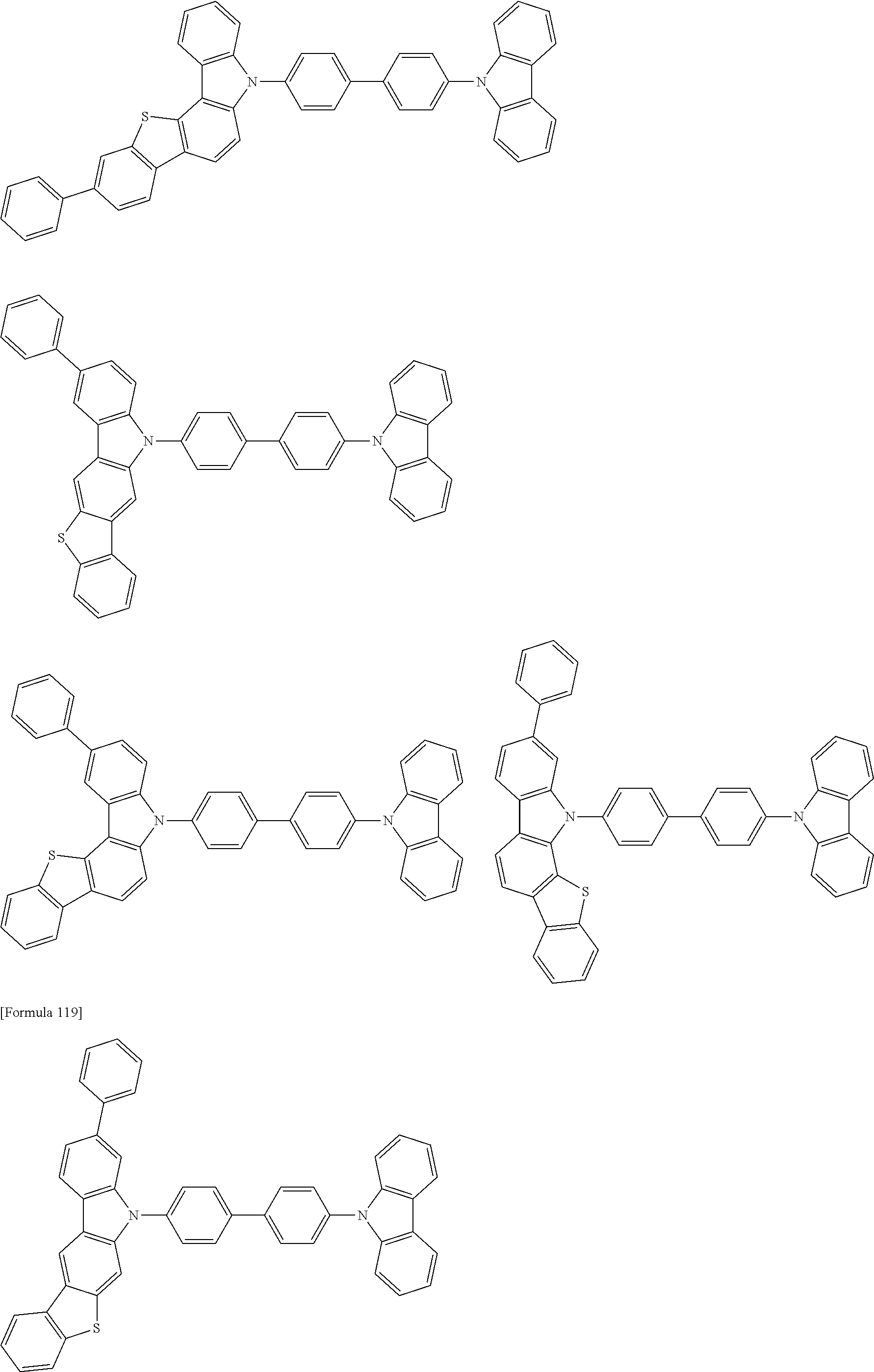

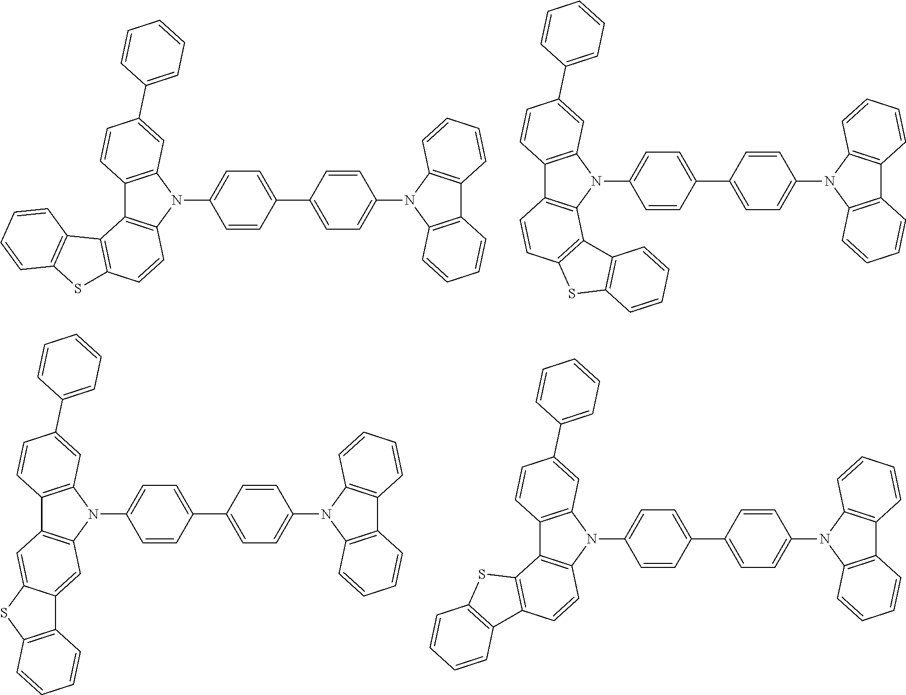

### Compound M2

The emitting layer of the exemplary embodiment includes a delayed fluorescent compound M2.

### Delayed Fluorescence

Delayed fluorescence is explained in “Yuki Hando-tai no Debaisu Bussei (Device Physics of Organic Semiconductors)” (edited by ADACHI, Chihaya, published by Kodansha, on pages 261-268). This document describes that, if an energy difference ΔE₁₃ of a fluorescent material between a singlet state and a triplet state is reducible, a reverse energy transfer from the triplet state to the singlet state, which usually occurs at a low transition probability, would occur at a high efficiency to express thermally activated delayed fluorescence (TADF). Further, a mechanism of generating delayed fluorescence is explained in Fig. 10.38 in the document. The compound M2 of the exemplary embodiment is preferably a compound exhibiting thermally activated delayed fluorescence generated by such a mechanism.

In general, emission of delayed fluorescence can be confirmed by measuring the transient PL (Photo Luminescence).

The behavior of delayed fluorescence can also be analyzed based on the decay curve obtained from the transient PL measurement. The transient PL measurement is a method of irradiating a sample with a pulse laser to excite the sample, and measuring the decay behavior (transient characteristics) of PL emission after the irradiation is stopped. PL emission in TADF materials is classified into a light emission component from a singlet exciton generated by the first PL excitation and a light emission component from a singlet exciton generated via a triplet exciton. The lifetime of the singlet exciton generated by the first PL excitation is on the order of nanoseconds and is very short. Therefore, light emission from the singlet exciton rapidly attenuates after irradiation with the pulse laser.

On the other hand, the delayed fluorescence is gradually attenuated due to light emission from a singlet exciton generated via a triplet exciton having a long lifetime. As described above, there is a large temporal difference between the light emission from the singlet exciton generated by the first PL excitation and the light emission from the singlet exciton generated via the triplet exciton. Therefore, the luminous intensity derived from delayed fluorescence can be determined.

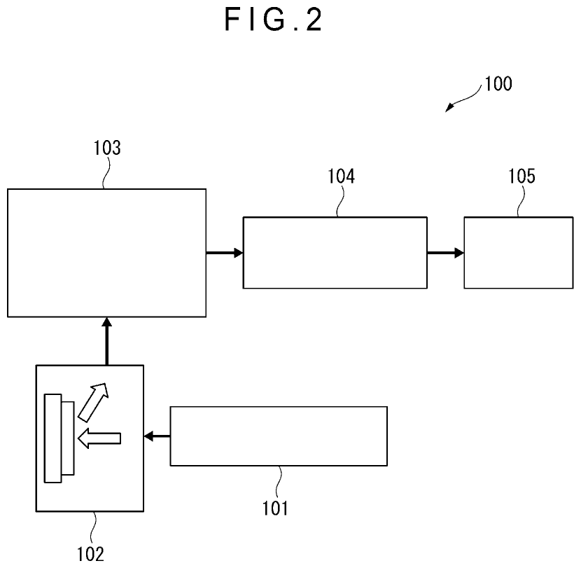

FIG. 2 shows a schematic diagram of an exemplary device for measuring the transient PL.

An example of a method of measuring a transient PL using FIG. 2 and an example of behavior analysis of delayed fluorescence will be described.

A transient PL measuring device 100 in FIG. 2 includes: a pulse laser 101 capable of radiating a light having a predetermined wavelength; a sample chamber 102 configured to house a measurement sample; a spectrometer 103 configured to divide a light radiated from the measurement sample; a streak camera 104 configured to provide a two-dimensional image; and a personal computer 105 configured to import and analyze the two-dimensional image. A device for measuring the transient PL is not limited to the device described in the exemplary embodiment.

The sample to be housed in the sample chamber 102 is obtained by doping a matrix material with a doping material at a concentration of 12 mass % and forming a thin film on a quartz substrate.

The thin film sample housed in the sample chamber 102 is irradiated with a pulse laser from the pulse laser 101 to excite the doping material. Emission is extracted in a direction of 90 degrees with respect to an irradiation direction of the excitation light. The extracted emission is divided by the spectrometer 103 to form a two-dimensional image in the streak camera 104. As a result, the two-dimensional image is obtainable in which the ordinate axis represents a time, the abscissa axis represents a wavelength, and a bright spot represents a luminous intensity. When this two-dimensional image is taken out at a predetermined time axis, an emission spectrum in which the ordinate axis represents the luminous intensity and the abscissa axis represents the wavelength is obtainable. Moreover, when this two-dimensional image is taken out at the wavelength axis, a decay curve (transient PL) in which the ordinate axis represents a logarithm of the luminous intensity and the abscissa axis represents the time is obtainable.

For instance, a thin film sample A was manufactured as described above from a reference compound H1 as the matrix material and a reference compound D1 as the doping material and was measured in terms of the transient PL.

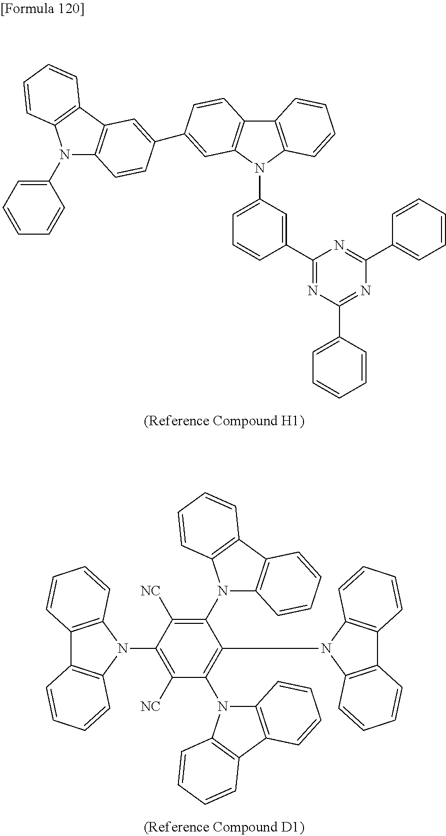 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; N#Cc1c(-n2c3ccccc3c3ccccc32)c(C#N)c(-n2c3ccccc3c3ccccc32)c(-n2c3ccccc3c3ccccc32)c1-n1c2ccccc2c2ccccc21]

Herein, the decay curve was analyzed with respect to the above thin film sample A and a thin film sample B. The thin film sample B was manufactured in the same manner as described above from a reference compound H2 as the matrix material and the reference compound D1 as the doping material.

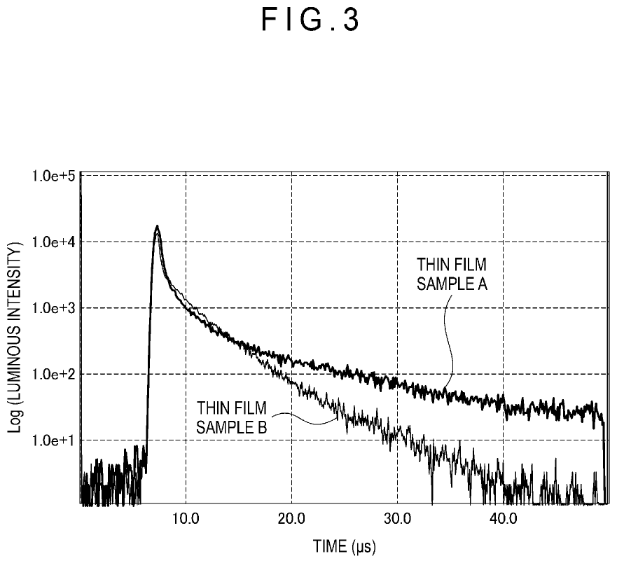

FIG. 3 shows decay curves obtained from the transient PL obtained by measuring the thin film samples A and B.

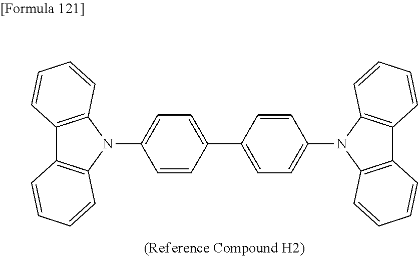 [SMILES: c1ccc2c(c1)c1ccccc1n2-c1ccc(-c2ccc(-n3c4ccccc4c4ccccc43)cc2)cc1]

As described above, an emission decay curve in which the ordinate axis represents the luminous intensity and the abscissa axis represents the time can be obtained by the transient PL measurement. Based on the emission decay curve, a fluorescence intensity ratio between fluorescence emitted from a singlet state generated by photo-excitation and delayed fluorescence emitted from a singlet state generated by inverse energy transfer via a triplet state can be estimated. In a delayed fluorescent material, a ratio of the intensity of the slowly decaying delayed fluorescence to the intensity of the promptly decaying fluorescence is relatively large.

Specifically, Prompt emission and Delay emission are present as emission from the delayed fluorescent material. Prompt emission is observed promptly when the excited state is achieved by exciting the compound of the exemplary embodiment with a pulse beam (i.e., a beam emitted from a pulse laser) having a wavelength absorbable by the delayed fluorescent material. Delay emission is observed not promptly when the excited state is achieved but after the excited state is achieved.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

In the exemplary embodiment, a sample manufactured by a method shown below is used for measuring delayed fluorescence of the compound M2. For instance, the compound M2 is dissolved in toluene to prepare a dilute solution with an absorbance of 0.05 or less at the excitation wavelength to eliminate the contribution of self-absorption. In order to prevent quenching due to oxygen, the sample solution is frozen and degassed and then sealed in a cell with a lid under an argon atmosphere to obtain an oxygen-free sample solution saturated with argon.

The fluorescence spectrum of the sample solution is measured with a spectrofluorometer FP-8600 (manufactured by JASCO Corporation), and the fluorescence spectrum of a 9,10-diphenylanthracene ethanol solution is measured under the same conditions. Using the fluorescence area intensities of both spectra, the total fluorescence quantum yield is calculated by an equation (1) in Morris et al. J. Phys. Chem. 80 (1976) 969.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

In the exemplary embodiment, provided that an amount of Prompt emission of a measurement target compound (compound M2) is denoted by X_{P} and the amount of Delay emission is denoted by X_{D}, a value of X_{D}/X_{P} is preferably 0.05 or more.

The amounts of Prompt emission and Delay emission and a ratio of the amounts thereof in compounds other than the compound M2 herein are measured in the same manner as those of the compound M2.

The compound M2 is exemplified by a compound represented by a formula (2) below.

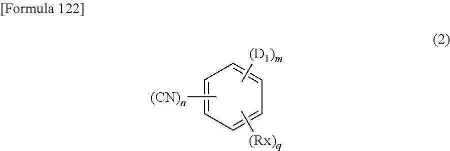

In the formula (2):

n is 1, 2, 3 or 4;

m is 1, 2, 3 or 4;

q is 0, 1, 2, 3 or 4;

m+n+q=6 is satisfied;

CN is a cyano group;

D₁ is a group represented by a formula (2a), (2b) or (2c) below, and when a plurality of D₁ are present, the plurality of D₁ are mutually the same or different;

Rx is a hydrogen atom or a substituent, or a pair of adjacent ones of Rx are mutually bonded to form a ring, and when a plurality of Rx are present, the plurality of Rx are mutually the same or different;

Rx as a substituent is each independently a halogen atom, a substituted or unsubstituted aryl group having 6 to 30 ring carbon atoms, a substituted or unsubstituted heterocyclic group having 5 to 30 ring atoms, a substituted or unsubstituted amino group, a substituted or unsubstituted carbonyl group, a substituted or unsubstituted alkyl group having 1 to 30 carbon atoms, a substituted or unsubstituted alkyl halide group having 1 to 30 carbon atoms, a substituted or unsubstituted alkylsilyl group having 3 to 30 carbon atoms, or a substituted or unsubstituted arylsilyl group having 6 to 60 ring carbon atoms; and

CN, D₁ and Rx are bonded to respective carbon atoms of a six-membered ring.

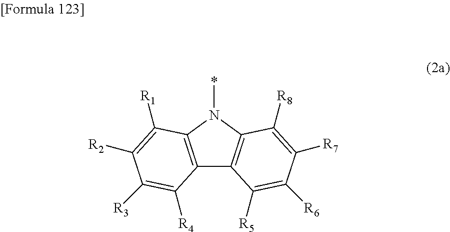

In the formula (2a):

R₁ to R₈ are each independently a hydrogen atom or a substituent, or at least one pair of a pair of R₁ and R₂, a pair of R₂ and R₃, a pair of R₃ and R₄, a pair of R₅ and R₆, a pair of R₆ and R₇, or a pair of R₇ and R₈ are mutually bonded to form a ring;

R₁ to R₈ as a substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkylsilyl group having 3 to 30 carbon atoms, a substituted or unsubstituted arylsilyl group having 6 to 60 ring carbon atoms, a hydroxy group, a substituted or unsubstituted alkoxy group having 1 to 30 carbon atoms, a substituted or unsubstituted alkoxy halide group having 1 to 30 carbon atoms, a substituted or unsubstituted aryloxy group having 6 to 30 ring carbon atoms, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

* represents a bonding position to a carbon atom of a benzene ring in the formula (2).

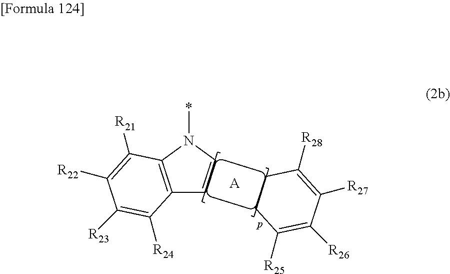

In the formula (2b):

R₂₁ to R₂₈ are each independently a hydrogen atom or a substituent, or at least one pair of a pair of R₂₁ and R₂₂, a pair of R₂₂ and R₂₃, a pair of R₂₃ and R₂₄, a pair of R₂₅ and R₂₆, a pair of R₂₆ and R₂₇, or a pair of R₂₇ and R₂₈ are mutually bonded to form a ring;

R₂₁ to R₂₈ as a substituent each independently represent the same as R₁ to R₈ in the formula (2a);

A represents a cyclic structure represented by a formula (211) or (212) below, and the cyclic structure A is fused with any position(s) of adjacent cyclic structure(s);

p is 1, 2, 3 or 4;

when p is 2, 3 or 4, a plurality of cyclic structures A are mutually the same or different; and

* represents a bonding position to a carbon atom of a benzene ring in the formula (2).

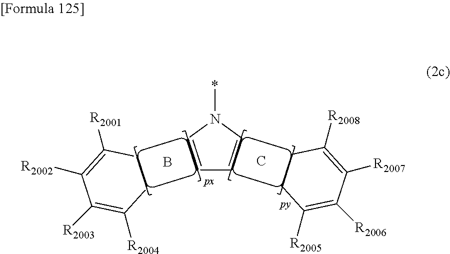

In the formula (2c):

R₂₀₀₁ to R₂₀₀₈ are each independently a hydrogen atom or a substituent, or at least one pair of a pair of R₂₀₀₁ and R₂₀₀₂, a pair of R₂₀₀₂ and R₂₀₀₃, a pair of R₂₀₀₃ and R₂₀₀₄, a pair of R₂₀₀₅ and R₂₀₀₆, a pair of R₂₀₀₆ and R₂₀₀₇, or a pair of R₂₀₀₇ and R₂₀₀₈ are mutually bonded to form a ring;

R₂₀₀₁ to R₂₀₀₈ as a substituent each independently represent the same as R₁ to R₈ as a substitutent in the formula (2a);

B represents a cyclic structure represented by the formula (211) or (212), and the cyclic structure B is fused with any position(s) of adjacent cyclic structure(s);

px is 1, 2, 3 or 4;

when px is 2, 3 or 4, a plurality of cyclic structures B are mutually the same or different;

C represents a cyclic structure represented by the formula (211) or (212), and the cyclic structure C is fused with any position(s) of adjacent cyclic structure(s);

py is 1, 2, 3 or 4;

when py is 2, 3 or 4, a plurality of cyclic structures C are mutually the same or different; and

* represents a bonding position to a carbon atom of a benzene ring in the formula (2).

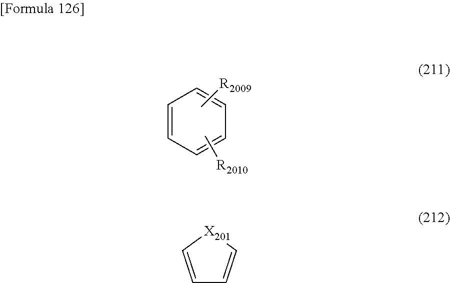

In the formula (211), R₂₀₀₉ and R₂₀₁₀ are each independently a hydrogen atom or a substituent, or bonded to a part of an adjacent cyclic structure, or a pair of R₂₀₀₉ and R₂₀₁₀ are mutually bonded to form a ring;

in the formula (212), X₂₀₁ is CR₂₀₁₁R₂₀₁₂, NR₂₀₁₃, a sulfur atom, or an oxygen atom, and R₂₀₁₁, R₂₀₁₂ and R₂₀₁₃ are each independently a hydrogen atom or a substituent, or R₂₀₁₁ and R₂₀₁₂ are mutually bonded to form a ring; and

R₂₀₀₉, R₂₀₁₀, R₂₀₁₁, R₂₀₁₂ and R₂₀₁₃ as a substituent each independently represent the same as R₁ to R₈ as a substituent in the formula (2a).

In the formula (211), R₂₀₀₉ and R₂₀₁₀ are each independently bonded to a part of an adjacent cyclic structure, which specifically means any of (I) to (IV) below.

In the formula (211), a pair of R₂₀₀₉ and R₂₀₁₀ are mutually bonded to form a ring, which specifically means (V) below.

(I) When the cyclic structures represented by the formula (211) are adjacent to each other, between the two adjacent rings, at least one pair of the following are mutually bonded to form a ring: R₂₀₀₉ of one of the rings and R₂₀₀₉ of the other of the rings; R₂₀₀₉ of one of the rings and R₂₀₁₀ of the other of the rings; or R₂₀₁₀ of one of the rings and R₂₀₁₀ of the other of the rings.

(II) When the cyclic structure represented by the formula (211) and the benzene ring having R₂₅ to R₂₈ in the formula (2b) are adjacent to each other, between the two adjacent rings, at least one pair of the following are mutually bonded to form a ring: R₂₀₀₉ of one of the rings and R₂₅ of the other of the rings; R₂₀₀₉ of one of the rings and R₂₈ of the other of the rings; R₂₀₁₀ of one of the rings and R₂₅ of the other of the rings; or R₂₀₁₀ of one of the rings and R₂₈ of the other of the rings.

(III) When the cyclic structure represented by the formula (211) and the benzene ring having R₂₀₀₁ to R₂₀₀₄ in the formula (2c) are adjacent to each other, between the two adjacent rings, at least one pair of the following are mutually bonded to form a ring: R₂₀₀₉ of one of the rings and R₂₀₀₁ of the other of the rings; R₂₀₀₉ of one of the rings and R₂₀₀₄ of the other of the rings; R₂₀₁₀ of one of the rings and R₂₀₀₁ of the other of the rings; or R₂₀₁₀ of one of the rings and R₂₀₀₄ of the other of the rings.

(IV) When the cyclic structure represented by the formula (211) and the benzene ring having R₂₀₀₅ to R₂₀₀₈ in the formula (2c) are adjacent to each other, between the two adjacent rings, at least one pair of the following are mutually bonded to form a ring: R₂₀₀₉ of one of the rings and R₂₀₀₅ of the other of the rings; R₂₀₀₉ of one of the rings and R₂₀₀₈ of the other of the rings; R₂₀₁₀ of one of the rings and R₂₀₀₅ of the other of the rings; or R₂₀₁₀ of one of the rings and R₂₀₀₈ of the other of the rings.

(V) The pair of R₂₀₀₉ and R₂₀₁₀ of the cyclic structure represented by the formula (211) are mutually bonded to form a ring. In other words, (V) means that the pair of R₂₀₀₉ and R₂₀₁₀, which are bonded to the same ring, are mutually bonded to form a ring.

In the compound M2 of the exemplary embodiment, it is preferable that: Rx is a hydrogen atom, an unsubstituted aryl group having 6 to 30 ring carbon atoms, an unsubstituted heterocyclic group having 5 to 30 ring atoms, or an unsubstituted alkyl group having 1 to 30 carbon atoms; and when Rx is an unsubstituted heterocyclic group having 5 to 30 ring atoms, Rx as the unsubstituted heterocyclic group having 5 to 30 ring atoms is a pyridyl group, pyrimidinyl group, triazinyl group, dibenzofuranyl group, or dibenzothienyl group.

Herein, the triazinyl group refers to a group obtained by excluding one hydrogen atom from 1,3,5-triazine, 1,2,4-triazine, or 1,2,3-triazine.

The triazinyl group is preferably a group obtained by excluding one hydrogen atom from 1,3,5-triazine.

In the compound M2 of the exemplary embodiment, Rx is more preferably a hydrogen atom, an unsubstituted aryl group having 6 to 30 ring carbon atoms, an unsubstituted dibenzofuranyl group, or an unsubstituted dibenzothienyl group.

In the compound M2 of the exemplary embodiment, Rx is further preferably a hydrogen atom.

In the compound M2 of the exemplary embodiment, it is preferable that R₁ to R₈, R₂₁ to R₂₈, R₂₀₀₁ to R₂₀₀₈, R₂₀₀₉ to R₂₀₁₀ and R₂₀₁₁ to R₂₀₁₃ as a substituent are each independently an unsubstituted aryl group having 6 to 30 ring carbon atoms, an unsubstituted heterocyclic group having 5 to 30 ring atoms, or an unsubstituted alkyl group having 1 to 30 carbon atoms.

In the compound M2 of the exemplary embodiment, D₁ is also preferably a group represented by a formula (D-21), (D-22), (D-23) or (D-24) below.

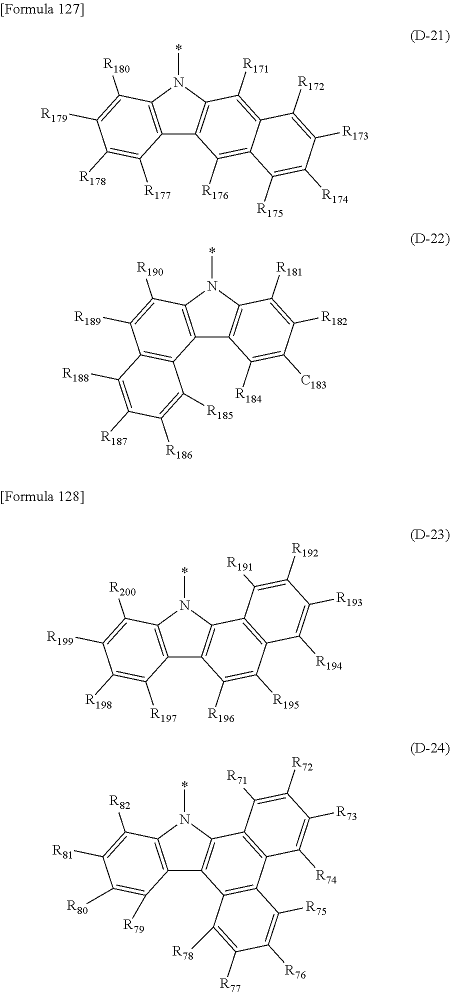

In the formulae (D-21) to (D-24): R₁₇₁ to R₂₀₀ and R₇₁ to R₈₂ are each independently a hydrogen atom or a substituent, or at least one pair of a pair of R₁₇₁ and R₁₇₂, a pair of R₁₇₂ and R₁₇₃, a pair of R₁₇₃ and R₁₇₄, a pair of R₁₇₄ and R₁₇₅, a pair of R₁₇₅ and R₁₇₆, a pair of R₁₇₇ and R₁₇₈, a pair of R₁₇₈ and R₁₇₉, a pair of R₁₇₉ and R₁₈₀, a pair of R₁₈₁ and R₁₈₂, a pair of R₁₈₂ and R₁₈₃, a pair of R₁₈₃ and R₁₈₄, a pair of R₁₈₅ and R₁₈₆, a pair of R₁₈₆ and R₁₈₇, a pair of R₁₈₇ and R₁₈₈, a pair of R₁₈₈ and R₁₈₉, a pair of R₁₈₉ and R₁₉₀, a pair of R₁₉₁ and R₁₉₂, a pair of R₁₉₂ and R₁₉₃, a pair of R₁₉₃ and R₁₉₄, a pair of R₁₉₄ and R₁₉₅, a pair of R₁₉₅ and R₁₉₆, a pair of R₁₉₇ and R₁₉₈, a pair of R₁₉₈ and R₁₉₉, a pair of R₁₉₉ and R₂₀₀, a pair of R₇₁ and R₇₂, a pair of R₇₂ and R₇₃, a pair of R₇₃ and R₇₄, a pair of R₇₅ and R₇₆, a pair of R₇₆ and R₇₇, a pair of R₇₇ and R₇₈, a pair of R₇₉ and R₈₀, a pair of R₈₀ and R₈₁, or a pair of R₈₁ and R₈₂ are mutually bonded to form a ring;

R₁₇₁ to R₂₀₀ and R₇₁ to R₈₂ as a substituent are each independently a halogen atom, a substituted or unsubstituted aryl group having 6 to 14 ring carbon atoms, a substituted or unsubstituted heterocyclic group having 5 to 14 ring atoms, a substituted or unsubstituted alkyl group having 1 to 6 carbon atoms, a substituted or unsubstituted alkyl halide group having 1 to 30 carbon atoms, a substituted or unsubstituted alkylsilyl group having 3 to 6 carbon atoms, a hydroxy group, a substituted or unsubstituted alkoxy group having 1 to 6 carbon atoms, a substituted or unsubstituted alkoxy halide group having 1 to 6 carbon atoms, a substituted or unsubstituted aryloxy group having 6 to 14 ring carbon atoms, a substituted or unsubstituted alkylamino group having 2 to 12 carbon atoms, a thiol group, a substituted or unsubstituted alkylthio group having 1 to 6 carbon atoms, or a substituted or unsubstituted arylthio group having 6 to 14 ring carbon atoms; and

* represents a bonding position to a carbon atom of a benzene ring in the formula (2).

In the compound M2 of the exemplary embodiment, D₁ is also more preferably a group represented by the formula (D-21), (D-23) or (D-24).

In the compound M2 of the exemplary embodiment, D₁ is also further preferably a group represented by the formula (D-21) or (D-23).

It is preferable that R₁₇₁ to R₂₀₀ and R₇₁ to R₈₂ as a substituent in the compound M2 of the exemplary embodiment are each independently an unsubstituted aryl group having 6 to 14 ring carbon atoms, an unsubstituted heterocyclic group having 5 to 14 ring atoms, or an unsubstituted alkyl group having 1 to 6 carbon atoms.

It is also preferable that R₁₇₁ to R₂₀₀ and R₇₁ to R₈₂ in the compound M2 of the exemplary embodiment are hydrogen atoms.

The compound M2 is exemplified by a compound represented by a formula (22) below.

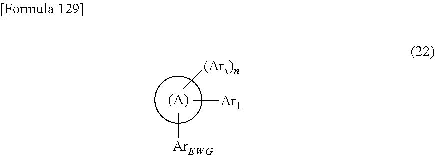

In the formula (22), Ar₁ is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by formulae (1a) to (1j) below.

In the formula (22), Ar_{EWG} is a substituted or unsubstituted heteroaryl group having 5 to 30 ring atoms that includes at least one nitrogen atom in a ring, or an aryl group having 6 to 30 ring carbon atoms and substituted by at least one cyano group.

In the formula (22), Ar_{X} is each independently a hydrogen atom or a substituent, and Ar_{X} as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by the formulae (1a) to (1j).

In the formula (22), n is 0, 1, 2, 3, 4 or 5, and when n is 2, 3, 4 or 5, a plurality of Ar_{X} are mutually the same or different.

In the formula (22), a ring (A) is a five-membered ring, a six-membered ring, or a seven-membered ring. The ring (A) may be an aromatic hydrocarbon ring or a heterocycle. Ar₁ and Ar_{X} are bonded to respective ones of elements forming the ring (A).

In the formula (22), at least one of Ar₁ or Ar_{X} is a group selected from the group consisting of groups represented by the formulae (1a) to (1j).

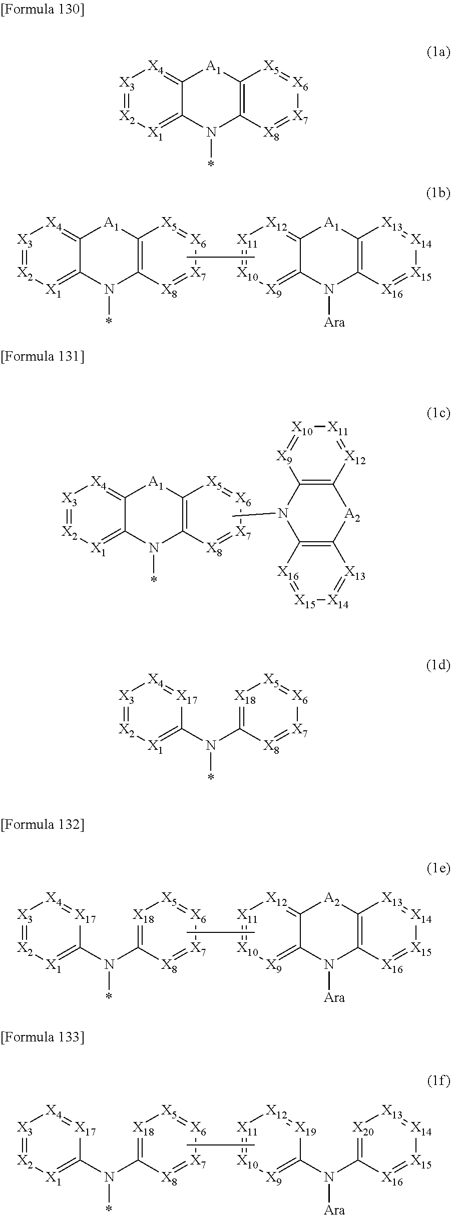

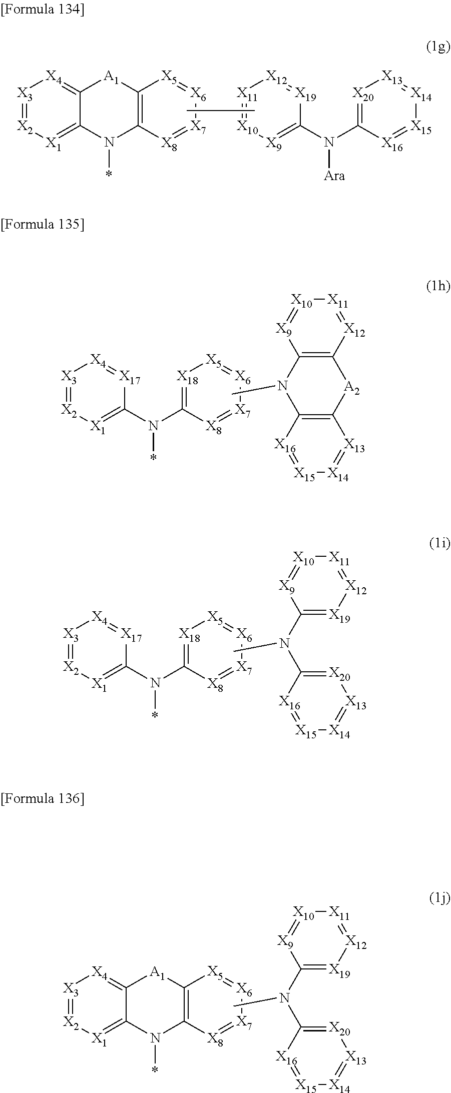

In the formulae (1a) to (1j), X₁ to X₂₀ are each independently a nitrogen atom (N) or a carbon atom bonded with R_{A1} (C—R_{A1}).

In the formula (1b), one of X₅ to X₈ is a carbon atom bonded to one of X₉ to X₁₂, and one of X₉ to X₁₂ is a carbon atom bonded to one of X₅ to X₈.

In the formula (1c), one of X₅ to X₈ is a carbon atom bonded to a nitrogen atom in a ring including A₂.

In the formula (1e), one of X₅ to X₈ and X₁₈ is a carbon atom bonded to one of X₉ to X₁₂, and one of X₉ to X₁₂ is a carbon atom bonded to one of X₅ to X₈ and X₁₈.

In the formula (1f), one of X₅ to X₈ and X₁₈ is a carbon atom bonded to one of X₉ to X₁₂ and X₁₉, and one of X₉ to X₁₂ and X₁₉ is a carbon atom bonded to one of X₅ to X₈ and X₁₈.

In the formula (1g), one of X₅ to X₈ is a carbon atom bonded to one of X₉ to X₁₂ and X₁₉, and one of X₉ to X₁₂ and X₁₉ is a carbon atom bonded to one of X₅ to X₈.

In the formula (1h), one of X₅ to X₈ and X₁₈ is a carbon atom bonded to a nitrogen atom in a ring including A₂.

In the formula (1i), one of X₅ to X₈ and X₁₈ is a carbon atom bonded to a nitrogen atom linking a ring including X₉ to X₁₂ and X₁₉ with a ring including X₁₃ to X₁₆ and X₂₀.

In the formula (1j), one of X₅ to X₈ is a carbon atom bonded to a nitrogen atom linking a ring including X₉ to X₁₂ and X₁₉ with a ring including X₁₃ to X₁₆ and X₂₀.

R_{A1} is each independently a hydrogen atom or a substituent, or at least one pair of pairs among a plurality of R_{A1} are mutually directly bonded to form a ring or bonded via a hetero atom to form a ring, and

R_{A1} as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group.

When a plurality of R_{A1} as a substituent are present, the plurality of R_{A1} are mutually the same or different.

In the formula (1a), when X₁ to X₈ are a carbon atom bonded with R_{A1} (C—R_{A1}), a plurality of R_{A1} preferably form no ring.

In the formulae (1a) to (1j), * represents a bonding position to the ring (A).

In the formulae (1a) to (1j), A₁ and A₂ are each independently a single bond, an oxygen atom (O), a sulfur atom (S), C(R₂₀₂₁)(R₂₀₂₂), Si(R₂₀₂₃)(R₂₀₂₄), C(═O), S(═O), SO₂ or N(R₂₀₂₅). R₂₀₂₁ to R₂₀₂₅ are each independently a hydrogen atom or a substituent, and R₂₀₂₁ to R₂₀₂₅ as a substituent are each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group.

In the formulae (1a) to (1j), Ara is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, and a substituted silyl group. Ara is preferably a substituted or unsubstituted aryl group having 6 to 30 ring carbon atoms or a substituted or unsubstituted heteroaryl group having 5 to 30 ring atoms.

The formula (1a) is represented by a formula (1aa) below when A₁ is a single bond, represented by a formula (1ab) below when A₁ is O, represented by a formula (1ac) below when A₁ is S, represented by a formula (1ad) below when A₁ is C(R₂₀₂₁)(R₂₀₂₂), represented by a formula (1ae) below when A₁ is Si(R₂₀₂₃)(R₂₀₂₄), represented by a formula (1af) below when A₁ is C(═O), represented by a formula (1ag) below when A₁ is S(═O), represented by a formula (1ah) below when A₁ is SO₂, and represented by a formula (1ai) below when A₁ is N(R₂₀₂₅). In the formulae (1aa) to (1ai), X₁ to X₈ and R₂₀₂₁ to R₂₀₂₅ represent the same as described above. Linkages between rings via A₁ and A₂ in the formulae (1b), (1c), (1e) and (1g) to (1j) are the same as those in the formulae (1aa) to (1ai). In the formula (1aa), when X₁ to X₈ are a carbon atom bonded with R_{A1} (C—R_{A1}), a plurality of R_{A1} as a substituent preferably form no ring.

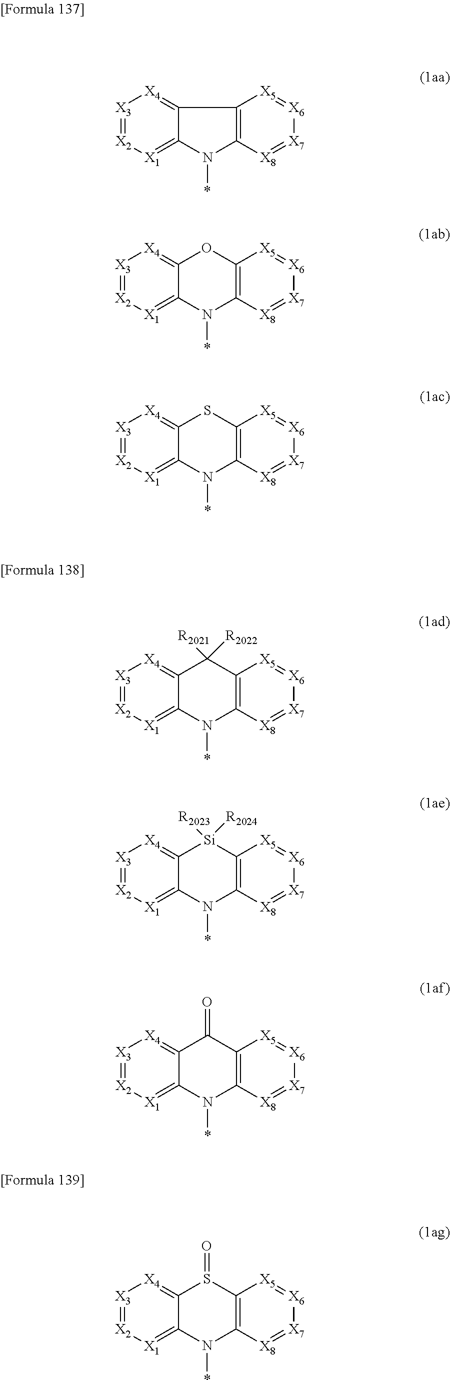

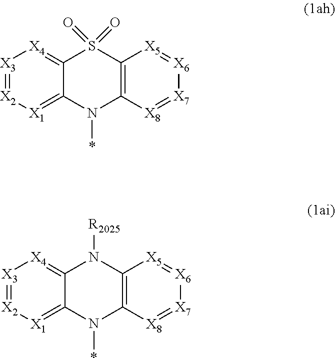

The compound M2 is preferably represented by a formula (221) below.

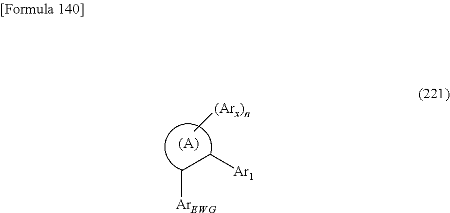

Ar₁, Ar_{EWG}, Arₓ, n and a ring (A) in the formula (221) respectively represent the same as Ar₁, Ar_{EWG}, Arₓ, n and the ring (A) in the formula (22).

The compound M2 is also preferably represented by a formula (222) below.

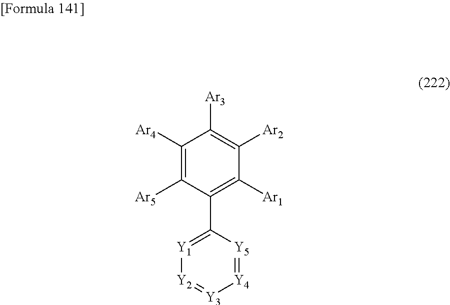

In the formula (222), Y₁ to Y₅ are each independently a nitrogen atom (N), a carbon atom bonded with a cyano group (C—CN), or a carbon atom bonded with R_{A2} (C—R_{A2}), and at least one of Y₁ to Y₅ is N or C—CN. A plurality of R_{A2} are mutually the same or different. R_{A2} is each independently a hydrogen atom or a substituent, and R_{A2} as a substituent is a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group; and a plurality of R_{A2} are mutually the same or different.

In the formula (222), Ar₁ represents the same as Ar₁ in the formula (22).

In the formula (222), Ar₂ to Ar₅ are each independently a hydrogen atom or a substituent, and Ar₂ to Ar₅ as a substituent are each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, a carboxy group, and groups represented by the formulae (1a) to (1c).

In the formula (222), at least one of Ar₁ to Ar₅ is a group selected from the group consisting of groups represented by the formulae (1a) to (1c).

The compound M2 is also preferably a compound represented by a formula (11aa), (11bb) or (11cc) below.

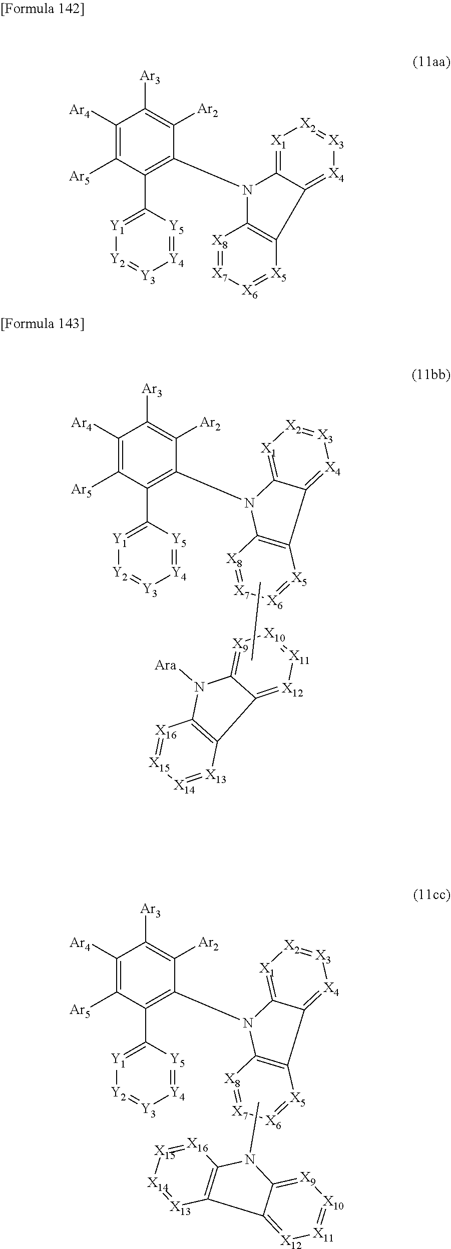

In the formulae (11aa), (11bb) and (11cc), Y₁ to Y₅, R_{A2}, Ar₂ to Ar₅, X₁ to X₁₆, R_{A1} and Ara respectively represent the same as the above-described Y₁ to Y₅, R_{A2}, Ar₂ to Ar₅, X₁ to X₁₆, R_{A1} and Ara.

The compound M2 is exemplified by a compound represented by a formula (23) below.

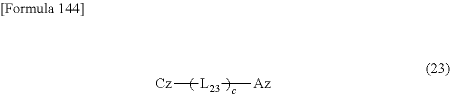

In the formula (23):

Az is a cyclic structure selected from the group consisting of a substituted or unsubstituted pyridine ring, a substituted or unsubstituted pyrimidine ring, a substituted or unsubstituted triazine ring, and a substituted or unsubstituted pyrazine ring;

c is 0, 1, 2, 3, 4 or 5;

when c is 0, Cz and Az are bonded by a single bond;

when c is 1, 2, 3, 4 or 5, L₂₃ is a linking group selected from the group consisting of a substituted or unsubstituted arylene group having 6 to 30 ring carbon atoms, and a substituted or unsubstituted heteroarylene group having 5 to 30 ring atoms;

when c is 2, 3, 4 or 5, a plurality of L₂₃ are mutually the same or different;

the plurality of L₂₃ are mutually bonded to form a ring or not bonded to form no ring; and

Cz is represented by a formula (23a) below.

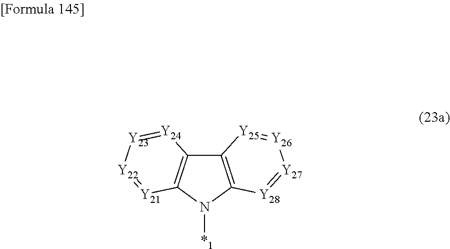

In the formula (23a):

Y₂₁ to Y₂₈ are each independently a nitrogen atom or CR_{A3};

R_{A3} is each independently a hydrogen atom or a substituent, or at least one pair of pairs among a plurality of R_{A3} are mutually bonded to form a ring;

R_{A3} as a substituent is each independently a group selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group;

a plurality of R_{A3} are mutually the same or different;

*1 represents a bonding position to a carbon atom in a structure of a linking group represented by L₂₃, or a bonding position to a carbon atom in a cyclic structure represented by Az.

Y₂₁ to Y₂₈ are also preferably CR_{A3}.

c in the formula (23) is preferably 0 or 1.

Cz is also preferably represented by a formula (23b), (23c) or (23d) below.

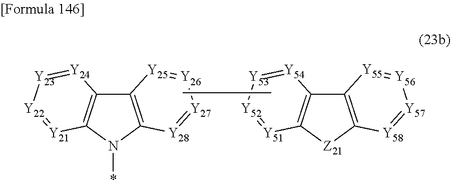

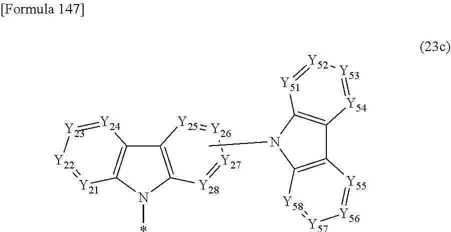

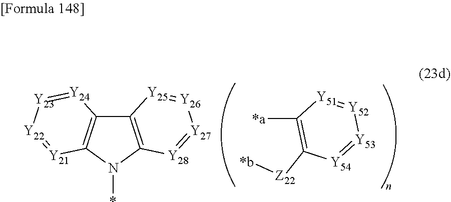

In the formulae (23b), (23c) and (23d), Y₂₁ to Y₂₈ and Y₅₁ to Y₅₈ are each independently a nitrogen atom or CR_{A4};

in the formula (23b), at least one of Y₂₅ to Y₂₈ is a carbon atom bonded to one of Y₅₁ to Y₅₄, and at least one of Y₅₁ to Y₅₄ is a carbon atom bonded to one of Y₂₅ to Y₂₈;

in the formula (23c), at least one of Y₂₅ to Y₂₈ is a carbon atom bonded to a nitrogen atom in a five-membered ring of a nitrogen-containing fused ring including Y₅₁ to Y₅₈;

in the formula (23d), *a and *b each represent a bonding position to one of Y₂₁ to Y₂₈, at least one of Y₂₅ to Y₂₈ is the bonding position represented by *a, and at least one of Y₂₅ to Y₂₈ is the bonding position represented by *b;

n is 1, 2, 3 or 4;

R_{A4} is each independently a hydrogen atom or a substituent, or at least one pair of pairs of a plurality of R_{A4} are mutually bonded to form a ring;

R_{A4} as a substituent is each independently a substituent selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group, a plurality of R_{A4} are mutually the same or different;

Z₂₁ and Z₂₁ are each independently one selected from the group consisting of an oxygen atom, a sulfur atom, NR₄₅ and CR₄₆R₄₇;

R₄₅ is a hydrogen atom or a substituent;

R₄₆ and R₄₇ are each independently a hydrogen atom or a substituent, or a pair of R₄₆ and R₄₇ are mutually bonded to form a ring;

R₄₅, R₄₆ and R₄₇ as a substituent are each independently a substituent selected from the group consisting of a substituted or unsubstituted aryl group having 6 to 30 ring carbon atoms, a substituted or unsubstituted heteroaryl group having 5 to 30 ring atoms, a substituted or unsubstituted alkyl group having 1 to 30 carbon atoms, a substituted or unsubstituted fluoroalkyl group having 1 to 30 carbon atoms, a substituted or unsubstituted cycloalkyl group having 3 to 30 ring carbon atoms, a substituted or unsubstituted aralkyl group having 7 to 30 carbon atoms, a substituted phosphoryl group, a substituted silyl group, a cyano group, a nitro group, and a carboxy group; a plurality of R₄₅ are mutually the same or different;

a plurality of R₄₆ are mutually the same or different;

a plurality of R₄₇ are mutually the same or different; and

* represents a bonding position to a carbon atom in a cyclic structure represented by Az.

Z₂₁ is preferably NR₄₅.

When Z₂₁ is NR₄₅, R₄₅ is preferably a substituted or unsubstituted aryl group having 6 to 30 ring carbon atoms.

Z₂₂ is preferably NR₄₅.

When Z₂₂ is NR₄₅, R₄₅ is preferably a substituted or unsubstituted aryl group having 6 to 30 ring carbon atoms.

Y₅₁ to Y₅₈ are preferably CR_{A4}, provided that at least one of Y₅₁ to Y₅₈ is a carbon atom bonded to a cyclic structure represented by the formula (23a).

It is also preferable that Cz is represented by the formula (23d) and n is 1.

Az is preferably a cyclic structure selected from the group consisting of a substituted or unsubstituted pyrimidine group and a substituted or unsubstituted triazine group.

Az is a cyclic structure selected from the group consisting of a substituted pyrimidine ring and a substituted triazine ring, in which a substituent of each of the substituted pyrimidine ring and the substituted triazine ring is more preferably a group selected from the group consisting of a substituted or unsubstituted aryl group having 6 to 30 ring carbon atoms and a substituted or unsubstituted heteroaryl group having 5 to 30 ring atoms, further preferably a substituted or unsubstituted aryl group having 6 to 30 ring carbon atoms.

When the pyrimidine ring and the triazine ring as Az have a substituted or unsubstituted aryl group as a substituent, the aryl group preferably has 6 to 20 ring carbon atoms, more preferably 6 to 14 ring carbon atoms, further preferably 6 to 12 ring carbon atoms.

When Az has a substituted or unsubstituted aryl group as a substituent, the substituent is preferably a group selected from the group consisting of a substituted or unsubstituted phenyl group, a substituted or unsubstituted biphenyl group, a substituted or unsubstituted naphthyl group, a substituted or unsubstituted phenanthryl group, a substituted or unsubstituted terphenyl group, and a substituted or unsubstituted fluorenyl group, more preferably a group selected from the group consisting of a substituted or unsubstituted phenyl group, a substituted or unsubstituted biphenyl group, and a substituted or unsubstituted naphthyl group.

When Az has a substituted or unsubstituted heteroaryl group as a substituent, the substituent is preferably a substituent selected from the group consisting of a substituted or unsubstituted carbazolyl group, a substituted or unsubstituted dibenzofuranyl group, and a substituted or unsubstituted dibenzothiophenyl group.

R_{A4} is each independently a hydrogen atom or a substituent. R_{A4} as a substituent is preferably a substituent selected from the group consisting of a substituted or unsubstituted aryl group having 6 to 30 ring carbon atoms and a substituted or unsubstituted heteroaryl group having 5 to 30 ring atoms.

When R_{A4} as a substituent is a substituted or unsubstituted aryl group having 6 to 30 ring carbon atoms, R_{A4} as a substituent is preferably a substituent selected from the group consisting of a substituted or unsubstituted phenyl group, a substituted or unsubstituted biphenyl group, a substituted or unsubstituted naphthyl group, a substituted or unsubstituted phenanthryl group, a substituted or unsubstituted terphenyl group, and a substituted or unsubstituted fluorenyl group, more preferably a substituent selected from the group consisting of a substituted or unsubstituted phenyl group, a substituted or unsubstituted biphenyl group, and a substituted or unsubstituted naphthyl group.

When R_{A4} as a substituent is a substituted or unsubstituted heteroaryl group having 5 to 30 ring atoms, R_{A4} as a substituent is preferably a substituent selected from the group consisting of a substituted or unsubstituted carbazolyl group, a substituted or unsubstituted dibenzofuranyl group, and a substituted or unsubstituted dibenzothiophenyl group.

R₄₅, R₄₆ and R₄₇ as a substituent are preferably each independently a substituent selected from the group consisting of a substituted or unsubstituted aryl group having 6 to 30 ring carbon atoms, a substituted or unsubstituted heteroaryl group having 5 to 30 ring atoms, and a substituted or unsubstituted alkyl group having 1 to 30 carbon atoms.

### Manufacturing Method of Compound M2 According to Exemplary Embodiment

The compound M2 can be manufactured by a known method.

### Specific Examples of Compound M2

Specific examples of the compound M2 of the exemplary embodiment include compounds below. It should however be noted that the invention is not limited to the specific examples of the compound.

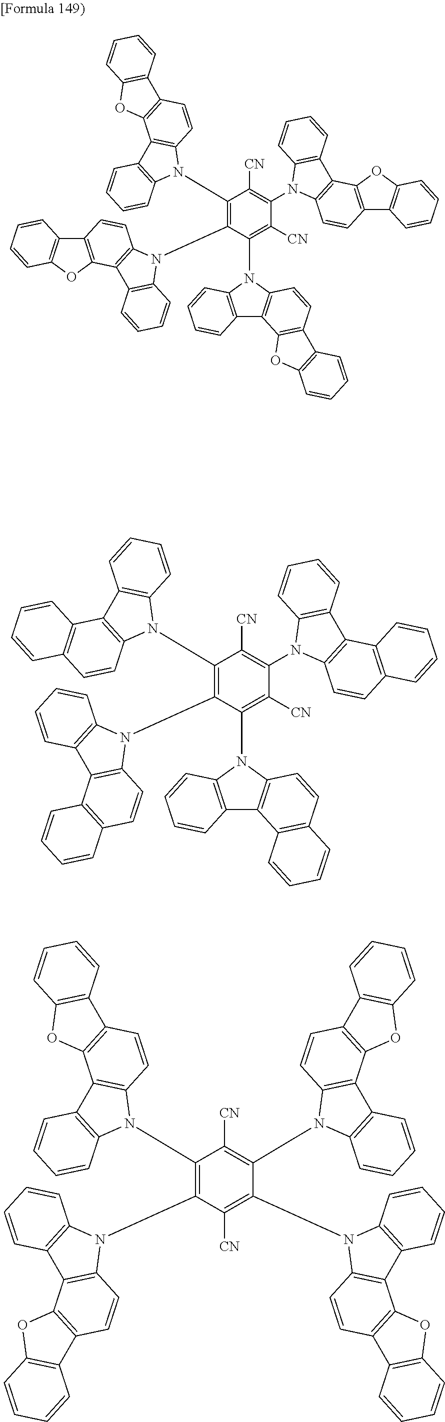 [SMILES: N#Cc1c(-n2c3ccccc3c3c4oc5ccccc5c4ccc32)c(C#N)c(-n2c3ccccc3c3c4oc5ccccc5c4ccc32)c(-n2c3ccccc3c3c4oc5ccccc5c4ccc32)c1-n1c2ccccc2c2c3oc4ccccc4c3ccc21 ; N#Cc1c(-n2c3ccccc3c3c4ccccc4ccc32)c(C#N)c(-n2c3ccccc3c3c4ccccc4ccc32)c(-n2c3ccccc3c3c4ccccc4ccc32)c1-n1c2ccccc2c2c3ccccc3ccc21 ; N#Cc1c(-n2c3ccccc3c3c4oc5ccccc5c4ccc32)c(-n2c3ccccc3c3c4oc5ccccc5c4ccc32)c(C#N)c(-n2c3ccccc3c3c4oc5ccccc5c4ccc32)c1-n1c2ccccc2c2c3oc4ccccc4c3ccc21]

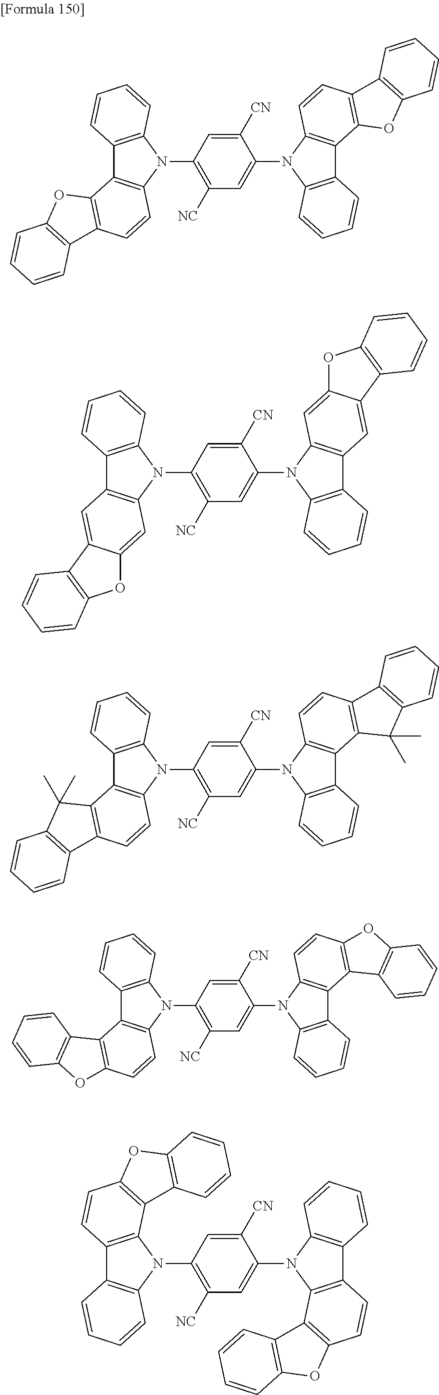 [SMILES: N#Cc1cc(-n2c3ccccc3c3c4oc5ccccc5c4ccc32)c(C#N)cc1-n1c2ccccc2c2c3oc4ccccc4c3ccc21 ; N#Cc1cc(-n2c3ccccc3c3cc4c(cc32)oc2ccccc24)c(C#N)cc1-n1c2ccccc2c2cc3c(cc21)oc1ccccc13 ; CC1(C)c2ccccc2-c2ccc3c(c21)c1ccccc1n3-c1cc(C#N)c(-n2c3ccccc3c3c4c(ccc32)-c2ccccc2C4(C)C)cc1C#N ; N#Cc1cc(-n2c3ccccc3c3c4c(ccc32)oc2ccccc24)c(C#N)cc1-n1c2ccccc2c2c3c(ccc21)oc1ccccc13 ; N#Cc1cc(-n2c3ccccc3c3ccc4oc5ccccc5c4c32)c(C#N)cc1-n1c2ccccc2c2ccc3oc4ccccc4c3c21]

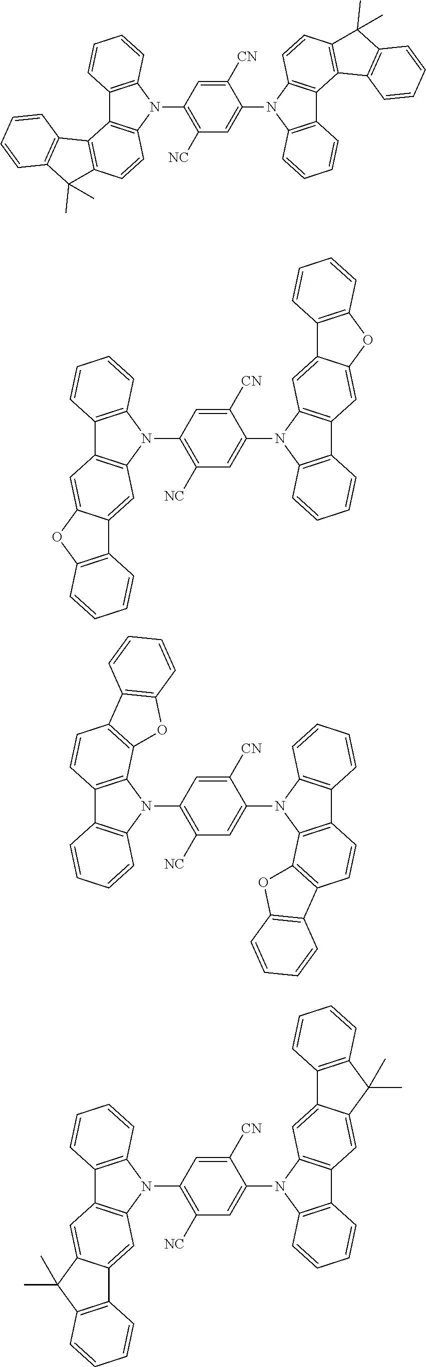

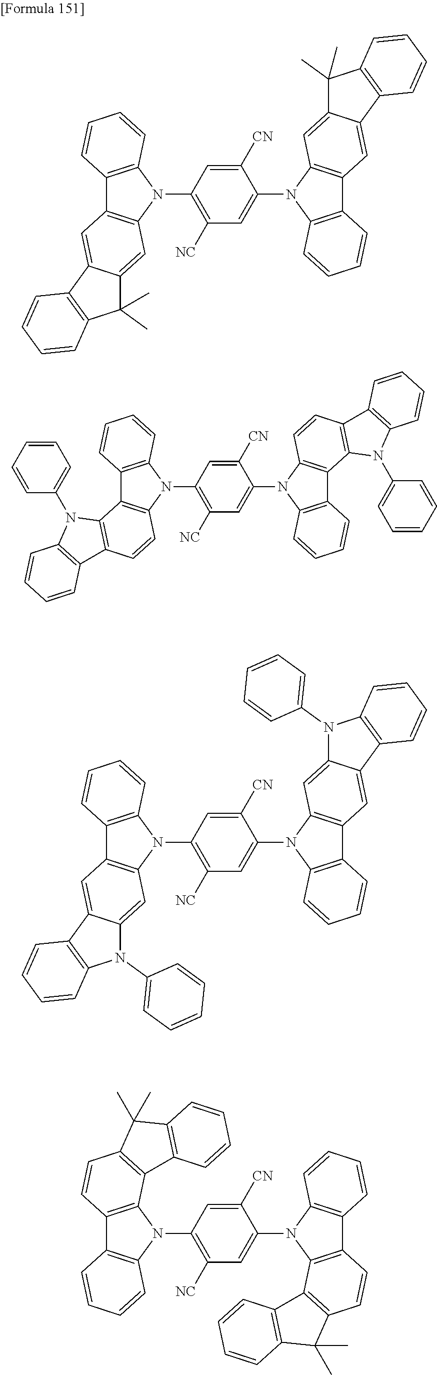 [SMILES: CC1(C)c2ccccc2-c2cc3c4ccccc4n(-c4cc(C#N)c(-n5c6ccccc6c6cc7c(cc65)C(C)(C)c5ccccc5-7)cc4C#N)c3cc21 ; N#Cc1cc(-n2c3ccccc3c3c2ccc2c4ccccc4n(-c4ccccc4)c23)c(C#N)cc1-n1c2ccccc2c2c1ccc1c3ccccc3n(-c3ccccc3)c12 ; N#Cc1cc(-n2c3ccccc3c3cc4c5ccccc5n(-c5ccccc5)c4cc32)c(C#N)cc1-n1c2ccccc2c2cc3c4ccccc4n(-c4ccccc4)c3cc21 ; CC1(C)c2ccccc2-c2c1ccc1c3ccccc3n(-c3cc(C#N)c(-n4c5ccccc5c5ccc6c(c54)-c4ccccc4C6(C)C)cc3C#N)c21]

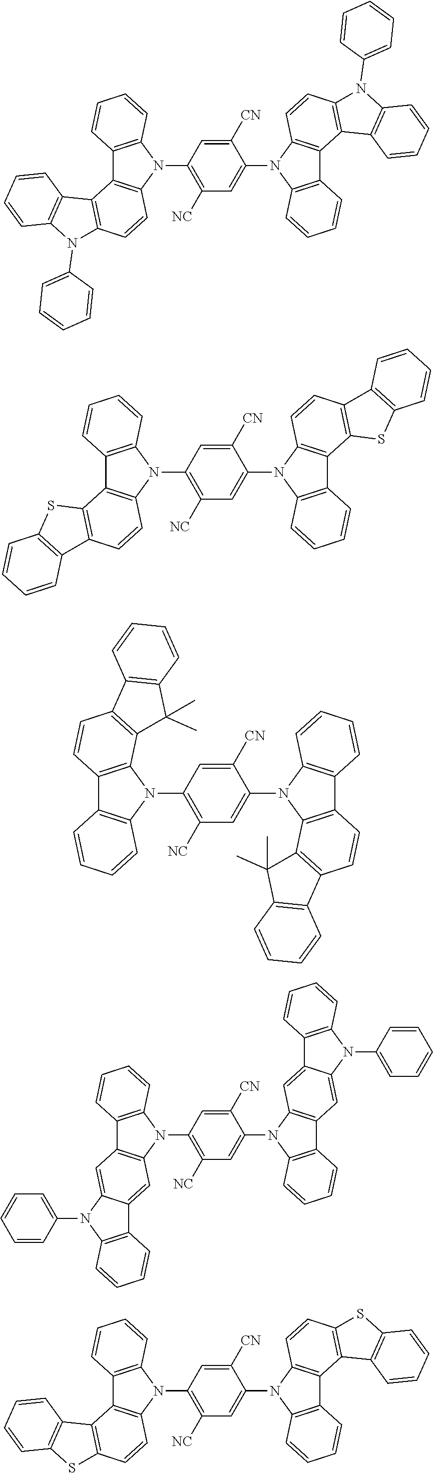

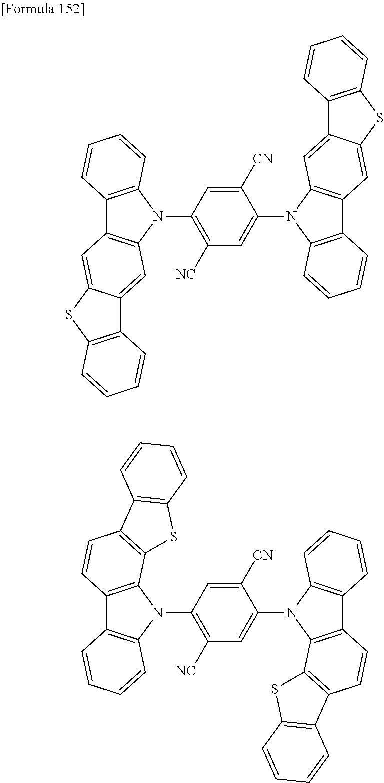 [SMILES: N#Cc1cc(-n2c3ccccc3c3cc4sc5ccccc5c4cc32)c(C#N)cc1-n1c2ccccc2c2cc3sc4ccccc4c3cc21 ; N#Cc1cc(-n2c3ccccc3c3ccc4c5ccccc5sc4c32)c(C#N)cc1-n1c2ccccc2c2ccc3c4ccccc4sc3c21]

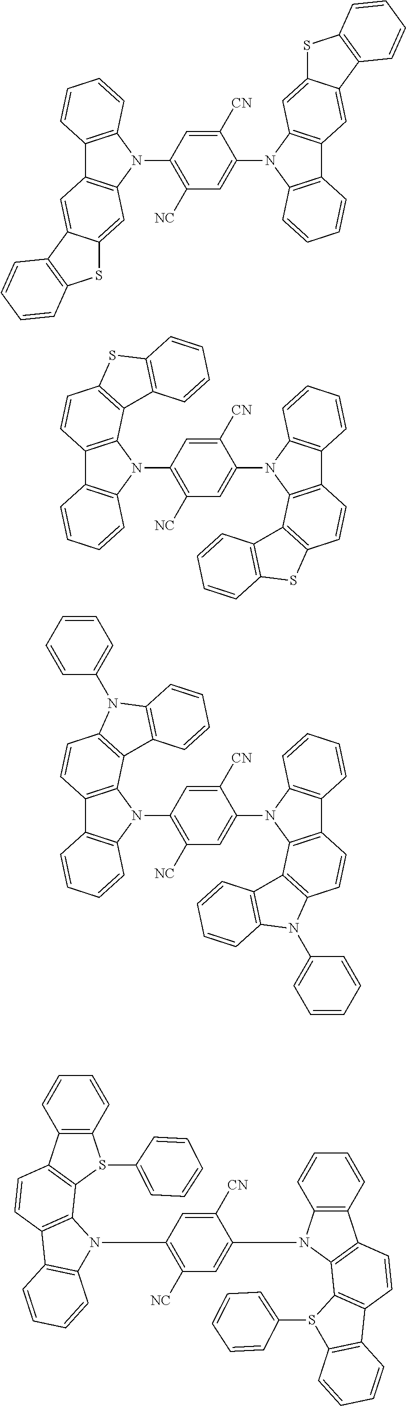

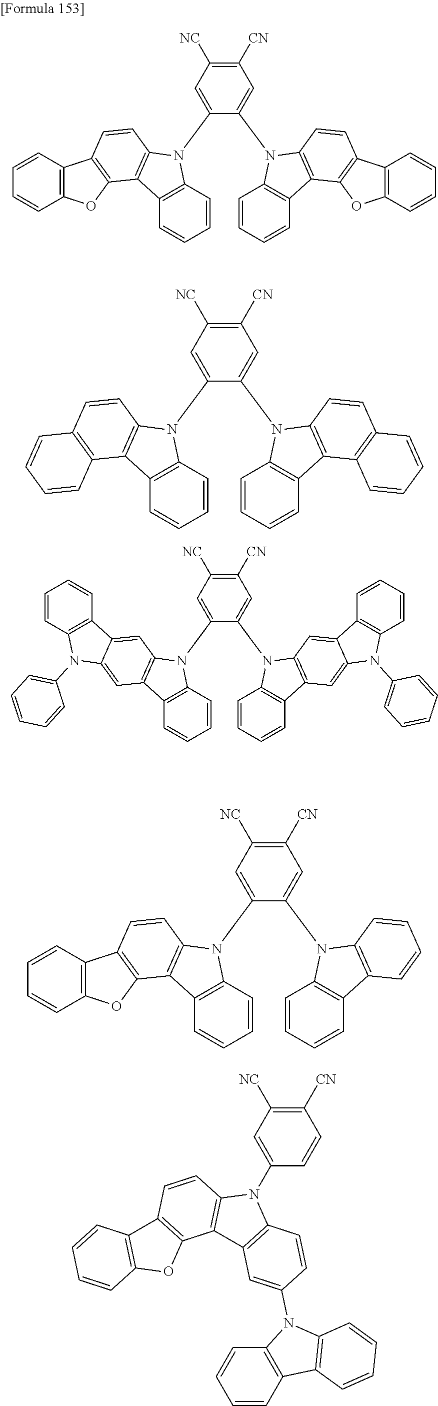

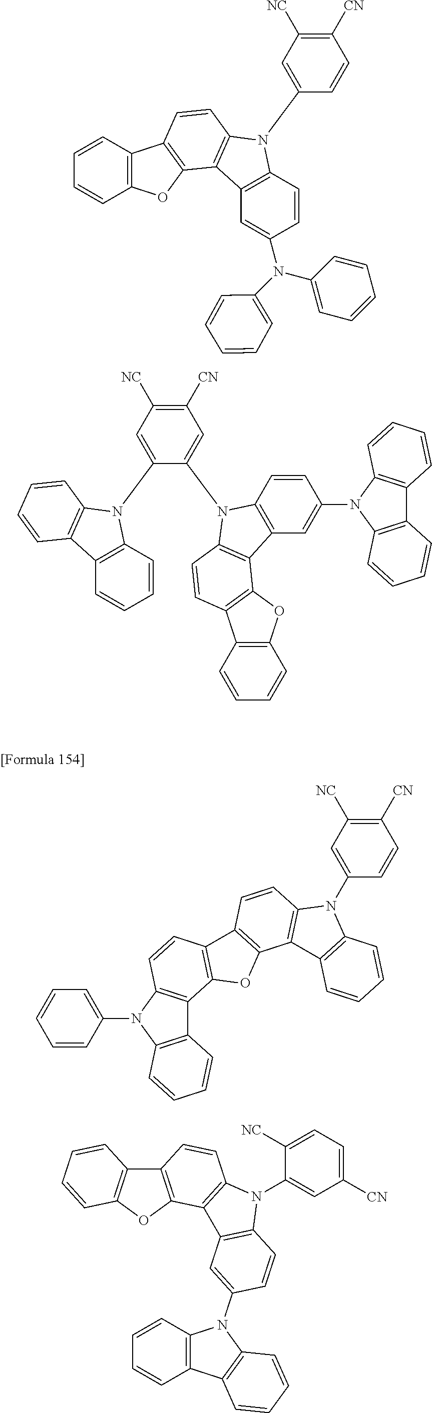

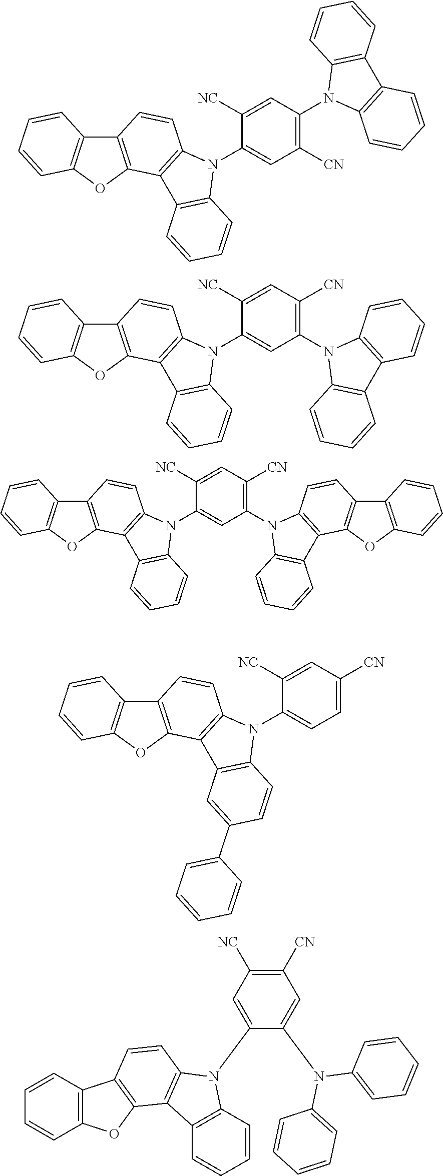

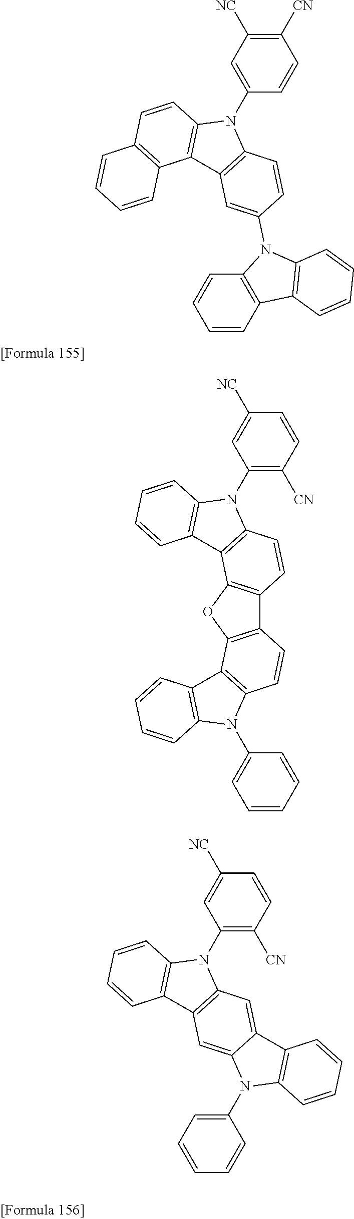 [SMILES: N#Cc1ccc(-n2c3ccc(-n4c5ccccc5c5ccccc54)cc3c3c4ccccc4ccc32)cc1C#N ; N#Cc1ccc(C#N)c(-n2c3ccccc3c3c4oc5c(ccc6c5c5ccccc5n6-c5ccccc5)c4ccc32)c1 ; N#Cc1ccc(C#N)c(-n2c3ccccc3c3cc4c(cc32)c2ccccc2n4-c2ccccc2)c1]

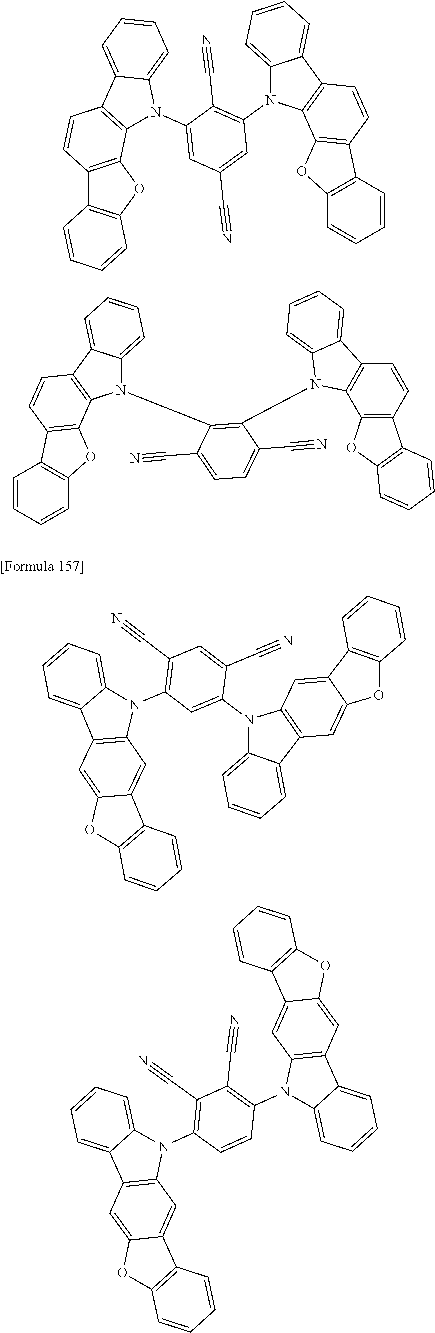

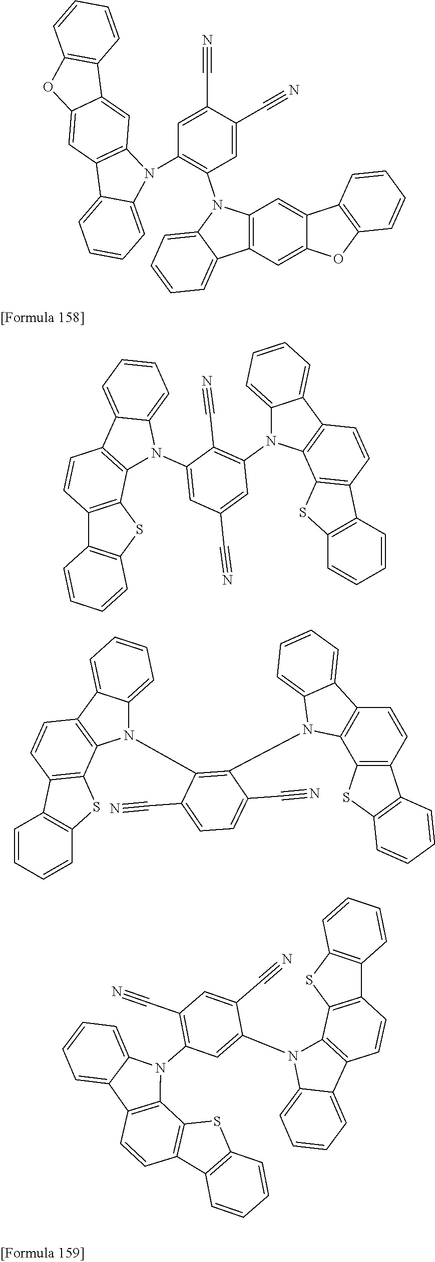

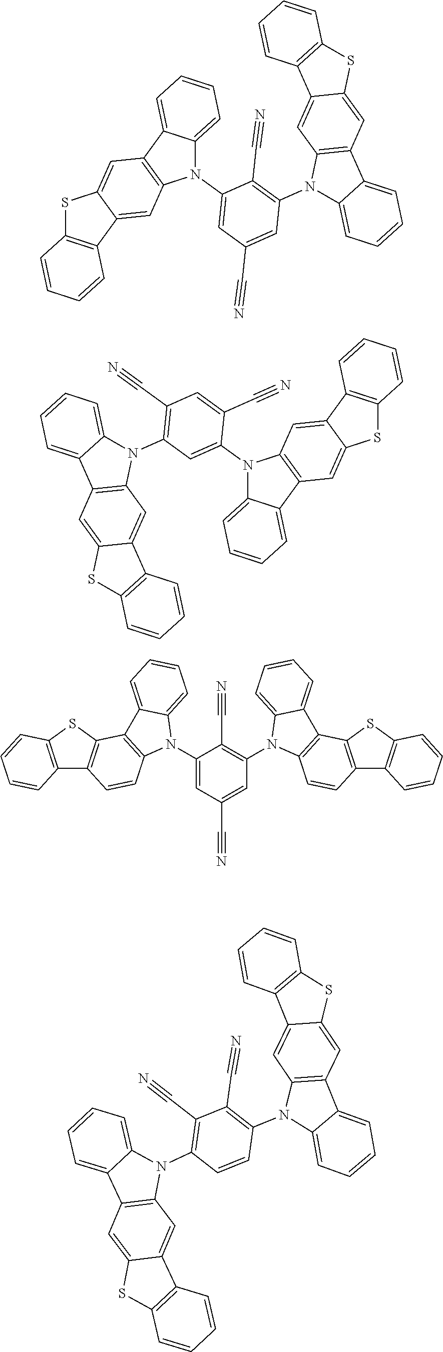

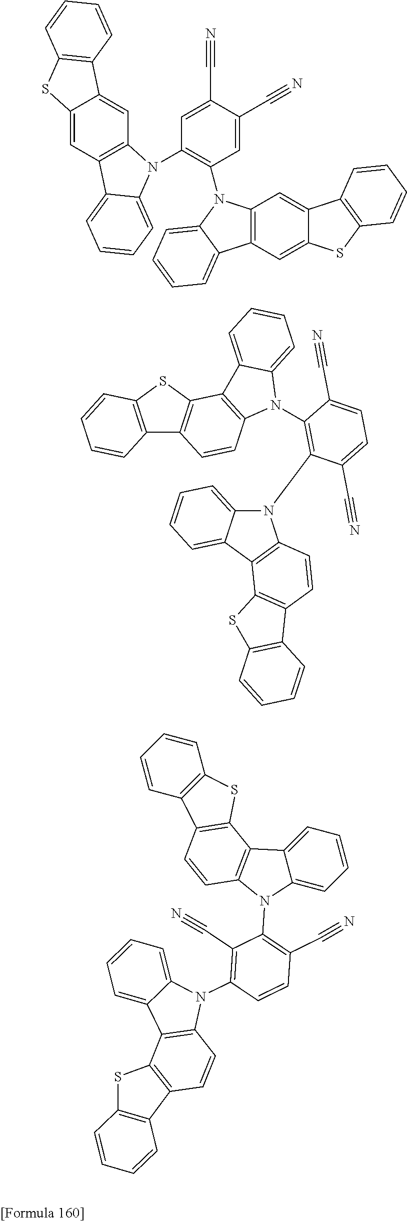 [SMILES: N#Cc1cc(-n2c3ccccc3c3cc4sc5ccccc5c4cc32)c(-n2c3ccccc3c3cc4sc5ccccc5c4cc32)cc1C#N ; N#Cc1ccc(C#N)c(-n2c3ccccc3c3c4sc5ccccc5c4ccc32)c1-n1c2ccccc2c2c3sc4ccccc4c3ccc21 ; N#Cc1ccc(-n2c3ccccc3c3c4sc5ccccc5c4ccc32)c(C#N)c1-n1c2ccccc2c2c3sc4ccccc4c3ccc21]

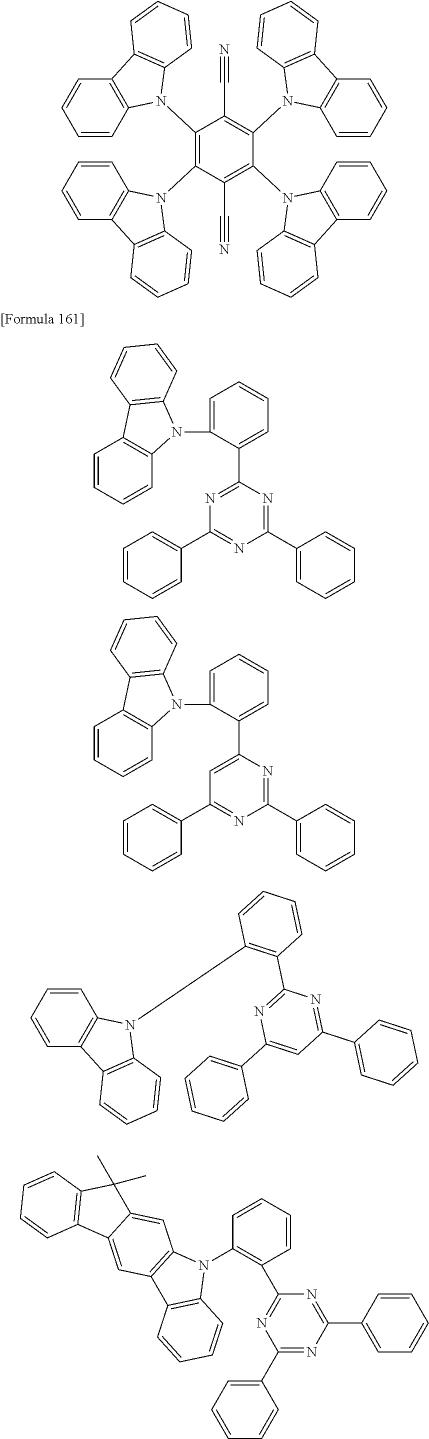

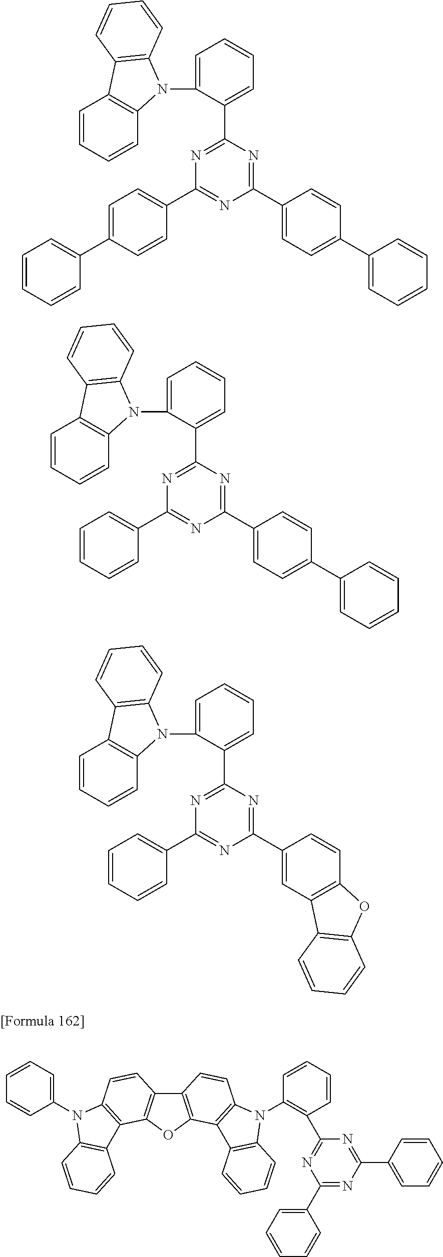

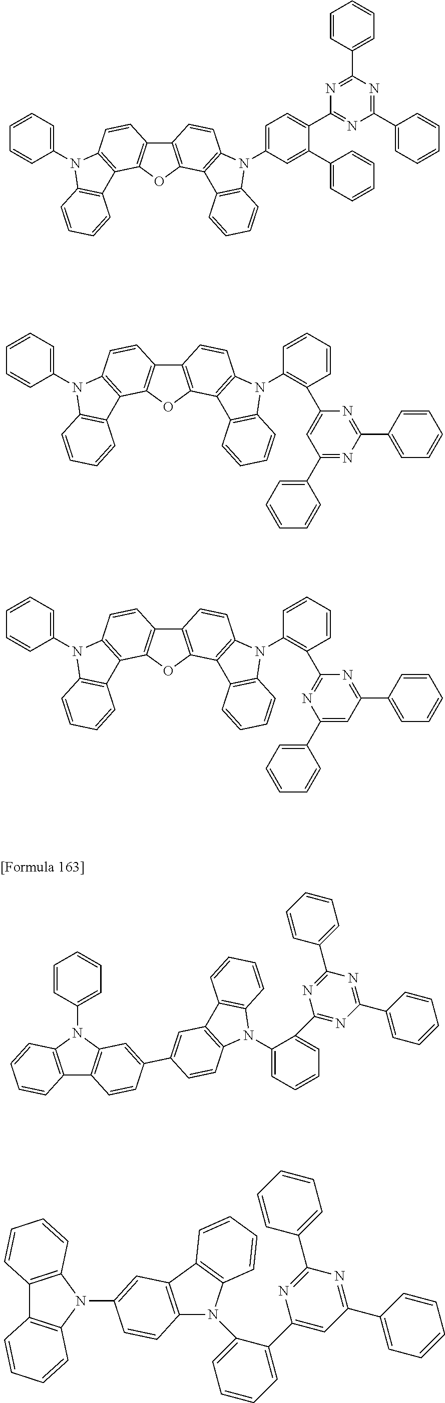 [SMILES: c1ccc(-c2nc(-c3ccccc3)nc(-c3ccc(-n4c5ccccc5c5c6oc7c(ccc8c7c7ccccc7n8-c7ccccc7)c6ccc54)cc3-c3ccccc3)n2)cc1 ; c1ccc(-c2cc(-c3ccccc3-n3c4ccccc4c4c5oc6c(ccc7c6c6ccccc6n7-c6ccccc6)c5ccc43)nc(-c3ccccc3)n2)cc1 ; c1ccc(-c2cc(-c3ccccc3)nc(-c3ccccc3-n3c4ccccc4c4c5oc6c(ccc7c6c6ccccc6n7-c6ccccc6)c5ccc43)n2)cc1 ; c1ccc(-c2nc(-c3ccccc3)nc(-c3ccccc3-n3c4ccccc4c4cc(-c5ccc6c7ccccc7n(-c7ccccc7)c6c5)ccc43)n2)cc1 ; c1ccc(-c2cc(-c3ccccc3-n3c4ccccc4c4cc(-n5c6ccccc6c6ccccc65)ccc43)nc(-c3ccccc3)n2)cc1]

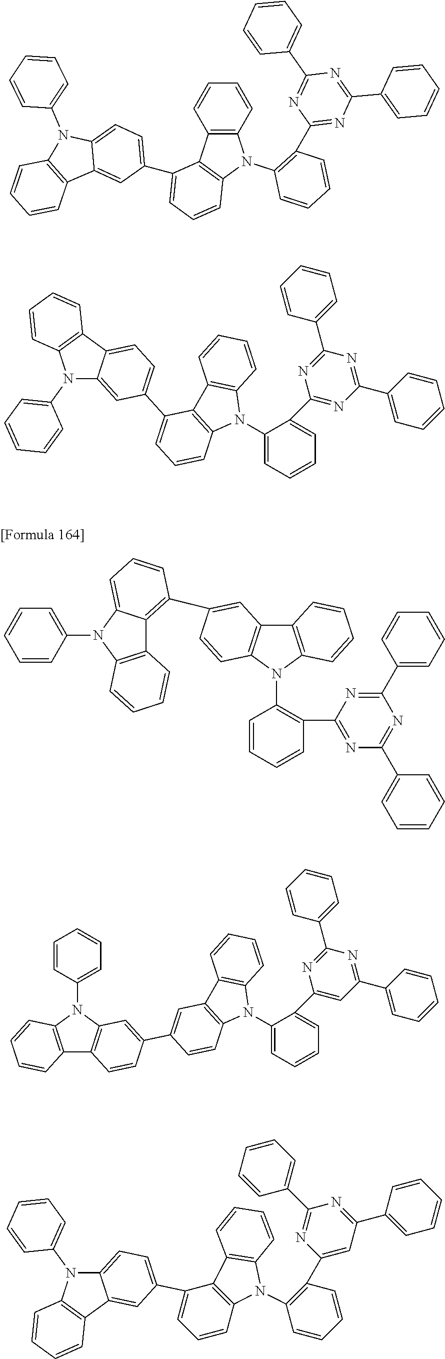 [SMILES: c1ccc(-c2nc(-c3ccccc3)nc(-c3ccccc3-n3c4ccccc4c4c(-c5ccc6c(c5)c5ccccc5n6-c5ccccc5)cccc43)n2)cc1 ; c1ccc(-c2nc(-c3ccccc3)nc(-c3ccccc3-n3c4ccccc4c4c(-c5ccc6c7ccccc7n(-c7ccccc7)c6c5)cccc43)n2)cc1 ; c1ccc(-c2nc(-c3ccccc3)nc(-c3ccccc3-n3c4ccccc4c4cc(-c5cccc6c5c5ccccc5n6-c5ccccc5)ccc43)n2)cc1 ; c1ccc(-c2cc(-c3ccccc3-n3c4ccccc4c4cc(-c5ccc6c7ccccc7n(-c7ccccc7)c6c5)ccc43)nc(-c3ccccc3)n2)cc1 ; c1ccc(-c2cc(-c3ccccc3-n3c4ccccc4c4c(-c5ccc6c(c5)c5ccccc5n6-c5ccccc5)cccc43)nc(-c3ccccc3)n2)cc1]

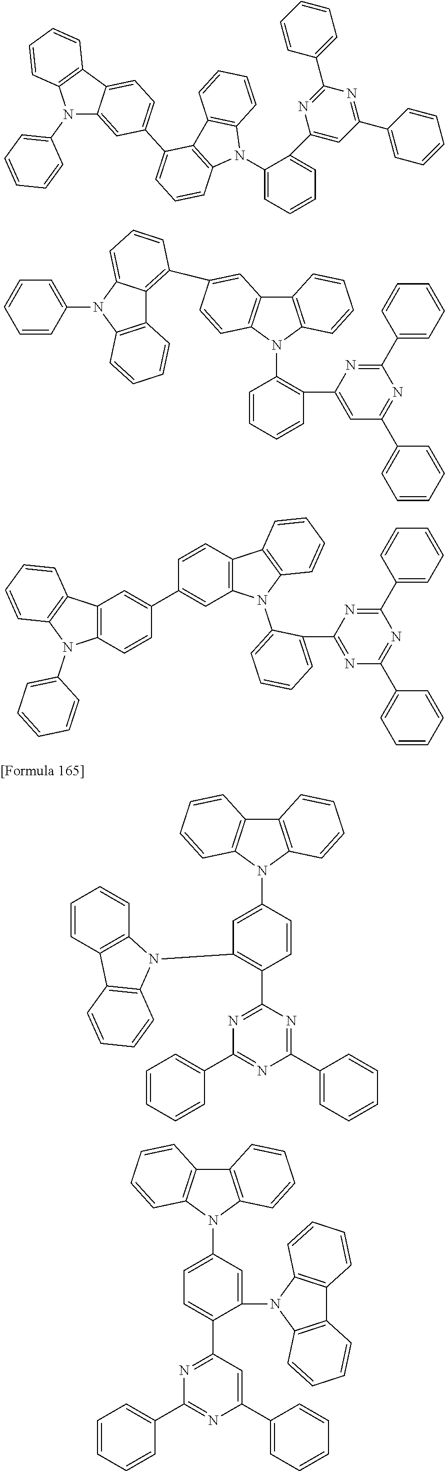

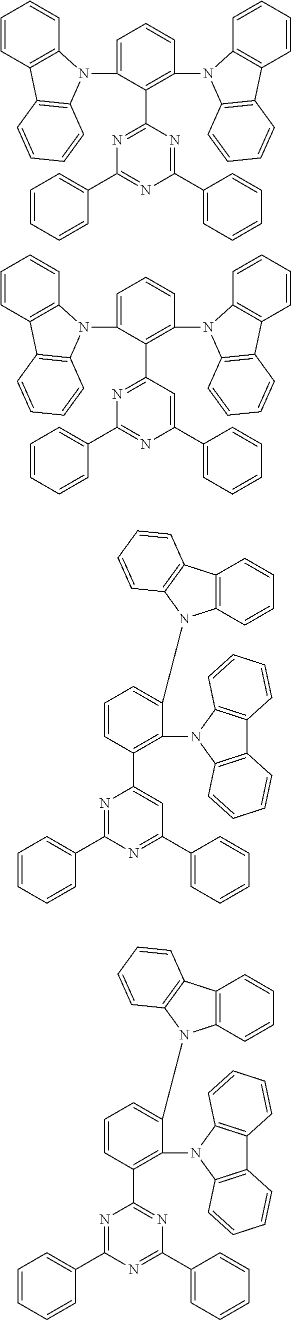

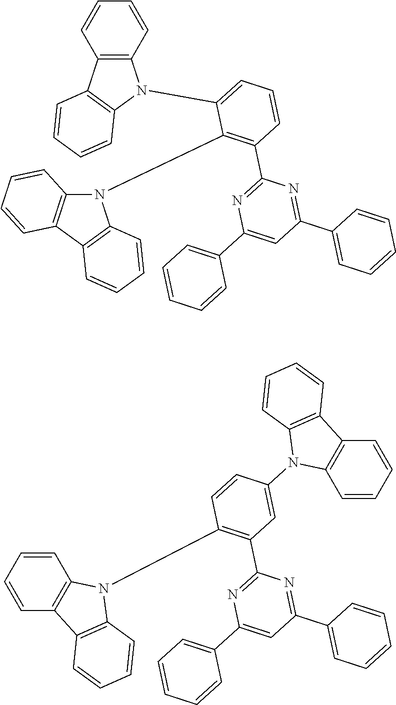 [SMILES: c1ccc(-c2cc(-c3ccccc3)nc(-c3cccc(-n4c5ccccc5c5ccccc54)c3-n3c4ccccc4c4ccccc43)n2)cc1 ; c1ccc(-c2cc(-c3ccccc3)nc(-c3cc(-n4c5ccccc5c5ccccc54)ccc3-n3c4ccccc4c4ccccc43)n2)cc1]

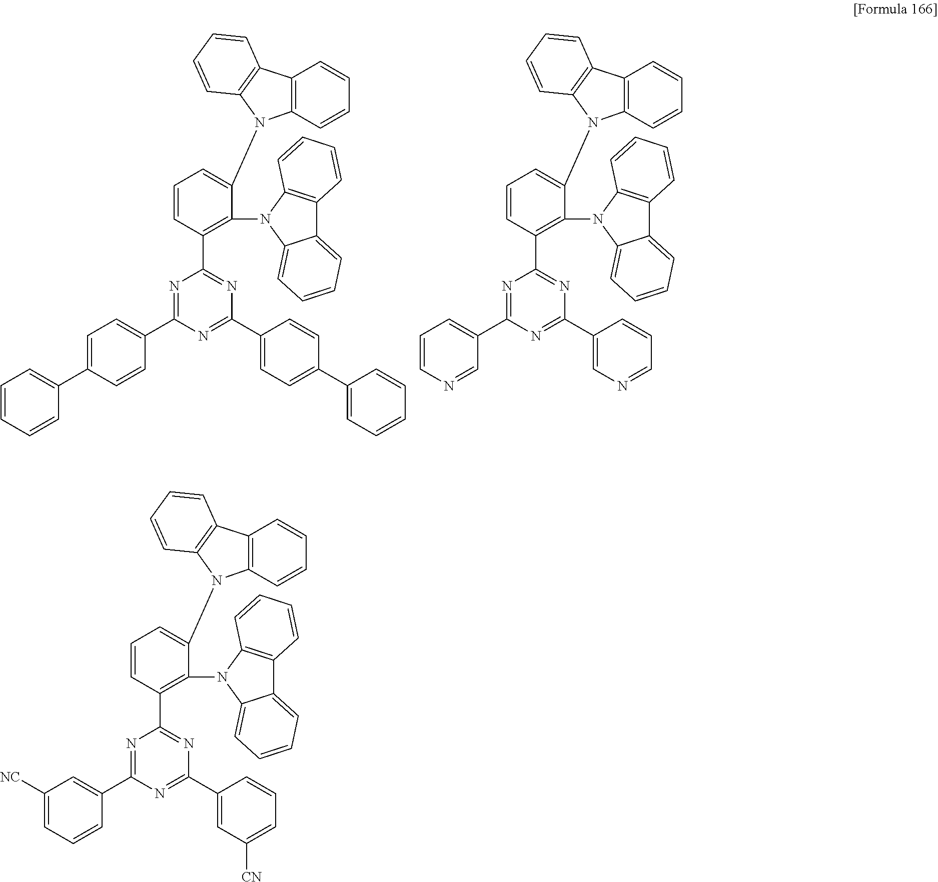

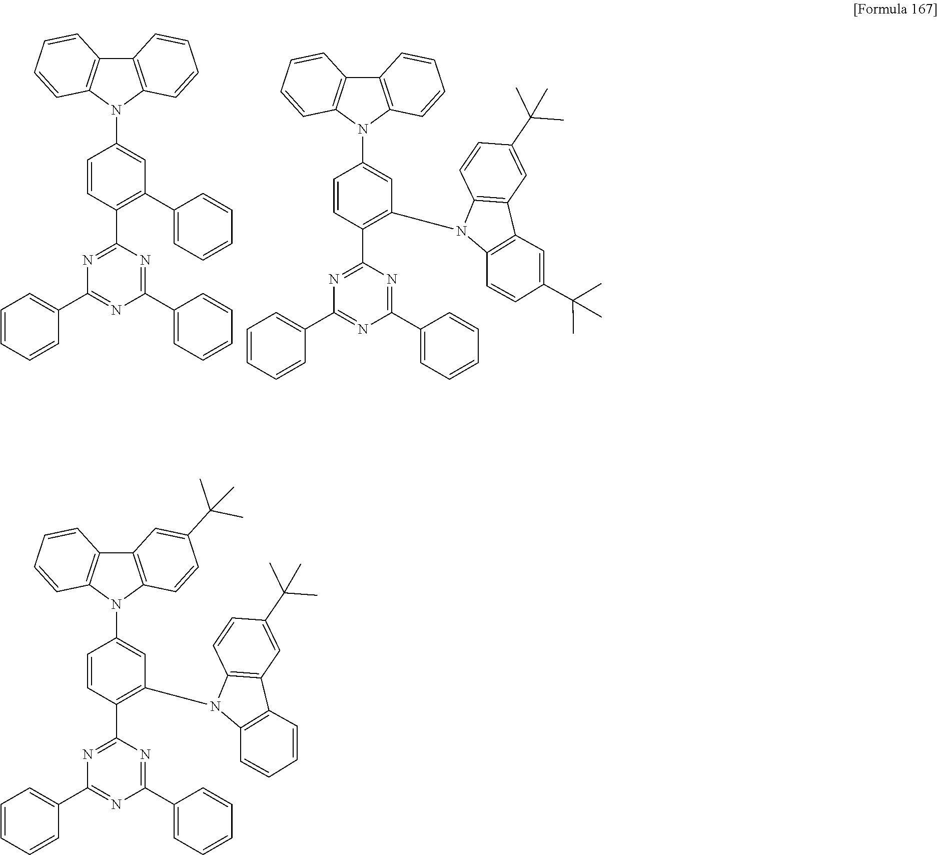

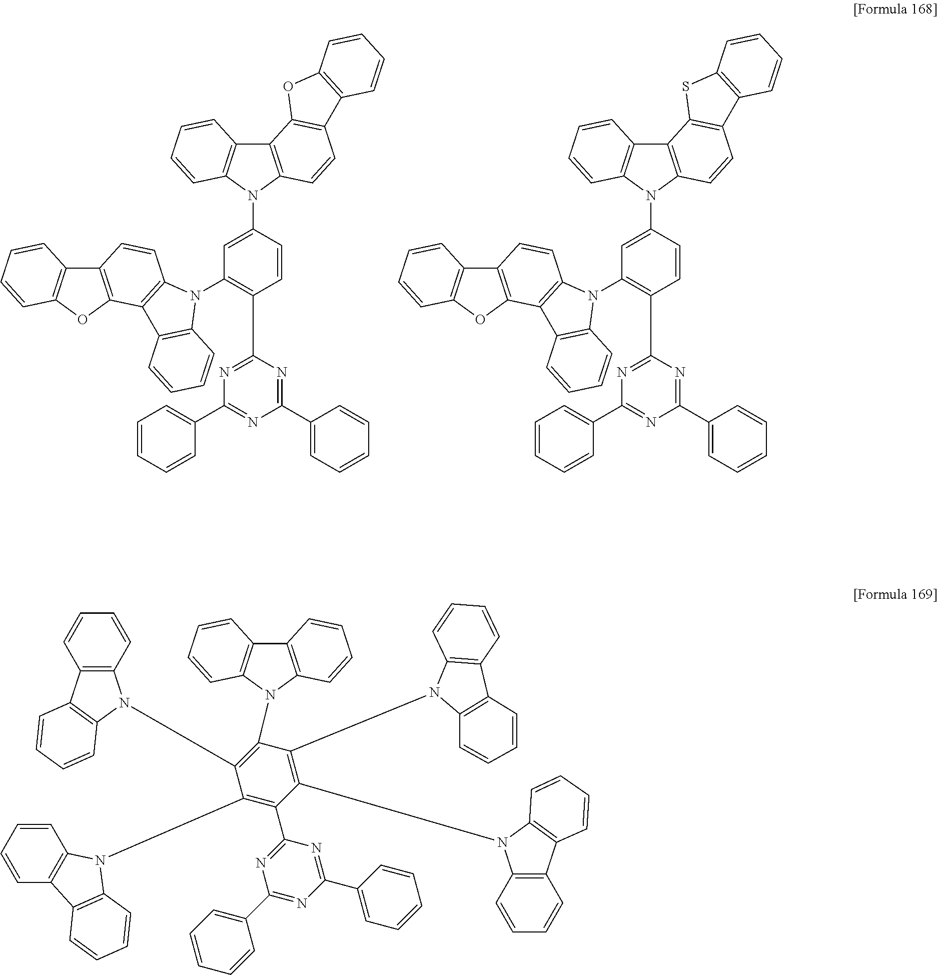 [SMILES: c1ccc(-c2nc(-c3ccccc3)nc(-c3ccc(-n4c5ccccc5c5c6oc7ccccc7c6ccc54)cc3-n3c4ccccc4c4c5oc6ccccc6c5ccc43)n2)cc1 ; c1ccc(-c2nc(-c3ccccc3)nc(-c3ccc(-n4c5ccccc5c5c6sc7ccccc7c6ccc54)cc3-n3c4ccccc4c4c5oc6ccccc6c5ccc43)n2)cc1 ; c1ccc(-c2nc(-c3ccccc3)nc(-c3c(-n4c5ccccc5c5ccccc54)c(-n4c5ccccc5c5ccccc54)c(-n4c5ccccc5c5ccccc54)c(-n4c5ccccc5c5ccccc54)c3-n3c4ccccc4c4ccccc43)n2)cc1]

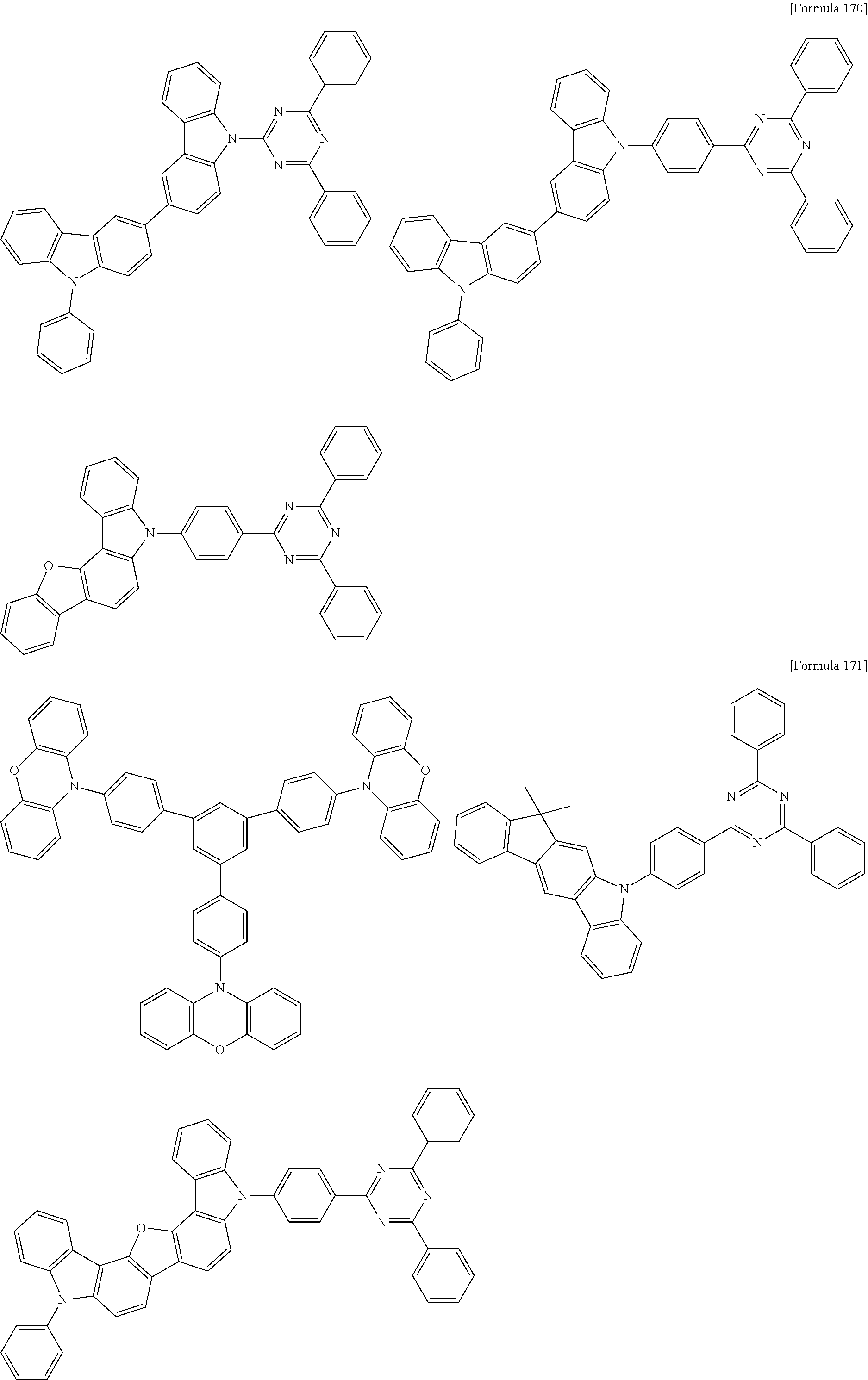

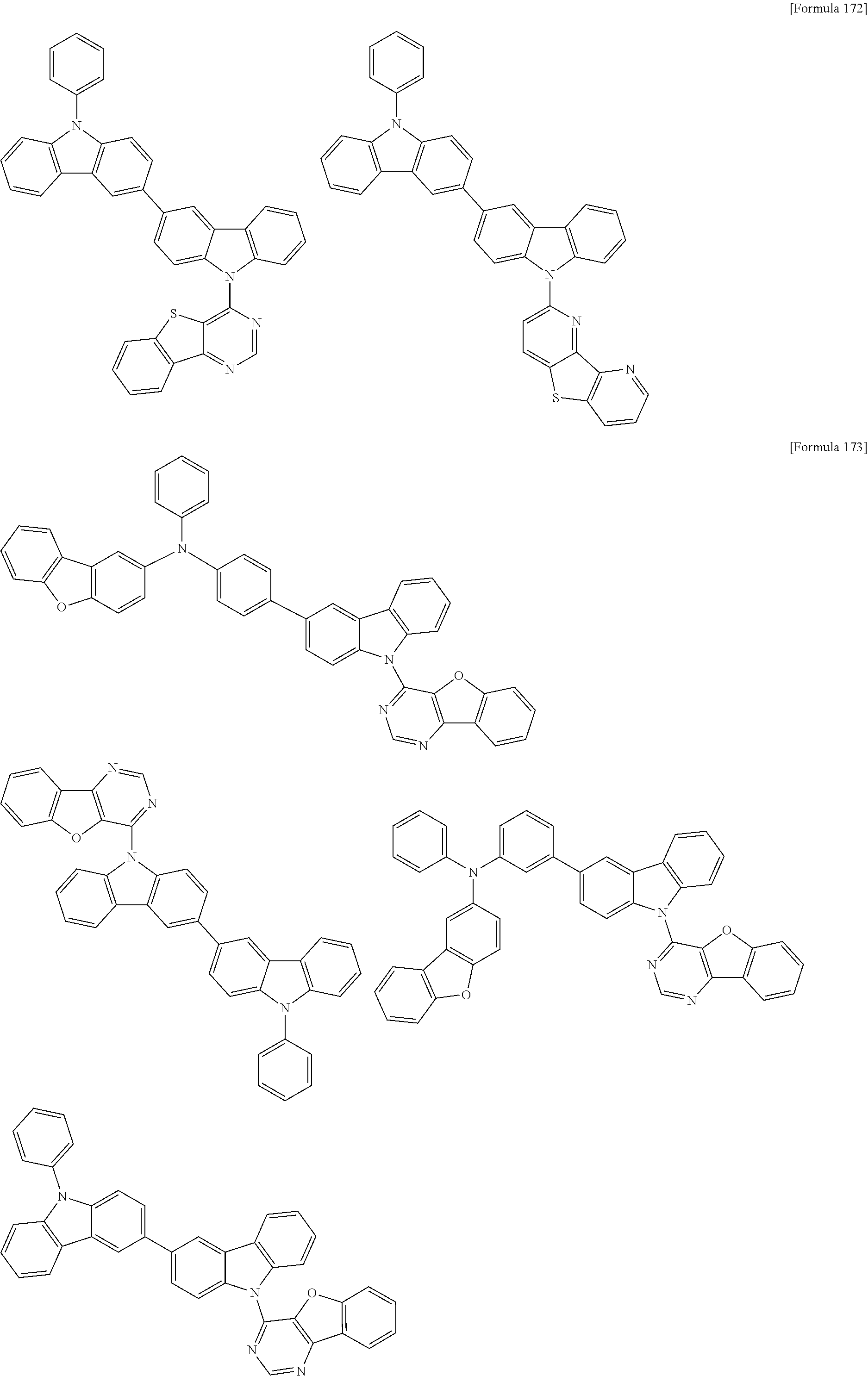

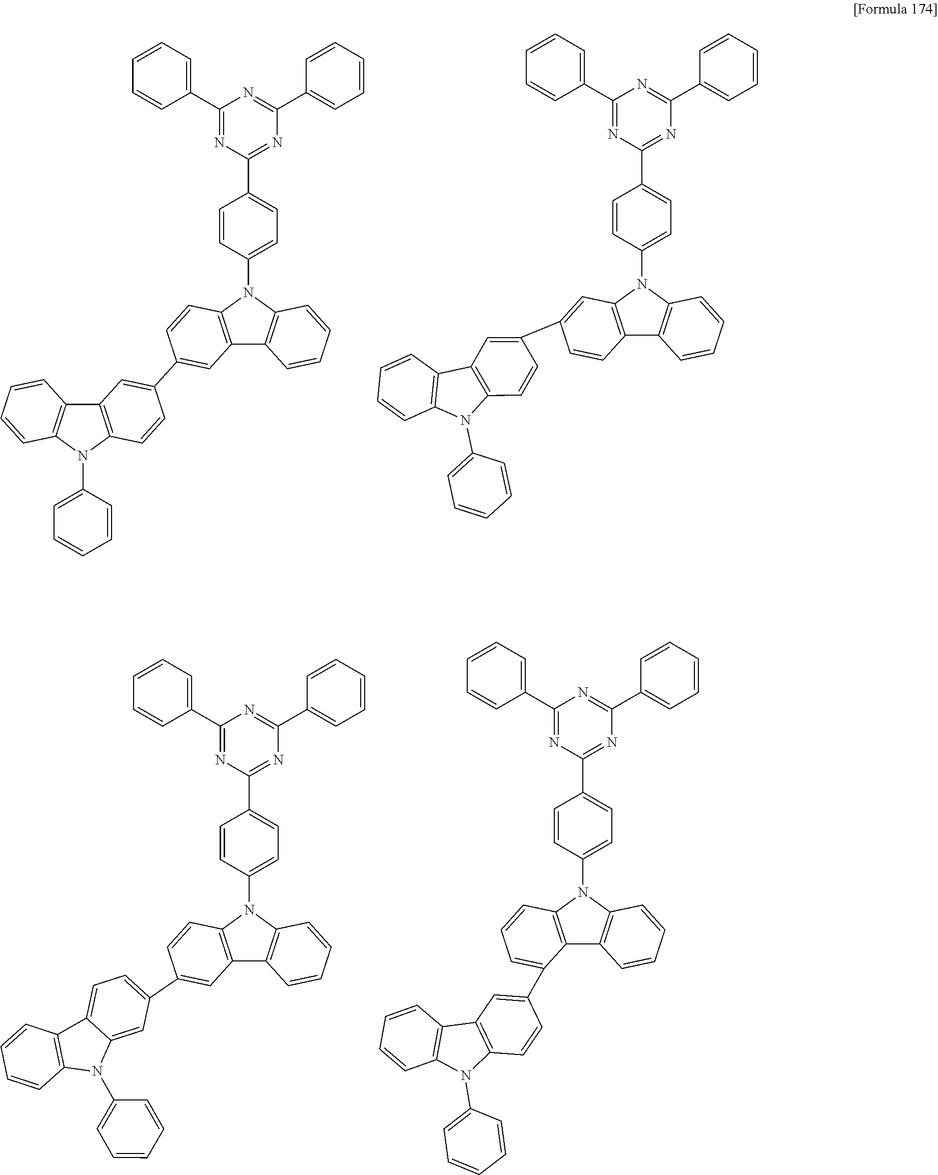 [SMILES: c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-c3ccc(-n4c5ccccc5c5ccc(-c6ccc7c(c6)c6ccccc6n7-c6ccccc6)cc54)cc3)n2)cc1 ; c1ccc(-c2nc(-c3ccccc3)nc(-c3ccc(-n4c5ccccc5c5cc(-c6ccc7c8ccccc8n(-c8ccccc8)c7c6)ccc54)cc3)n2)cc1 ; c1ccc(-c2nc(-c3ccccc3)nc(-c3ccc(-n4c5ccccc5c5c(-c6ccc7c(c6)c6ccccc6n7-c6ccccc6)cccc54)cc3)n2)cc1]

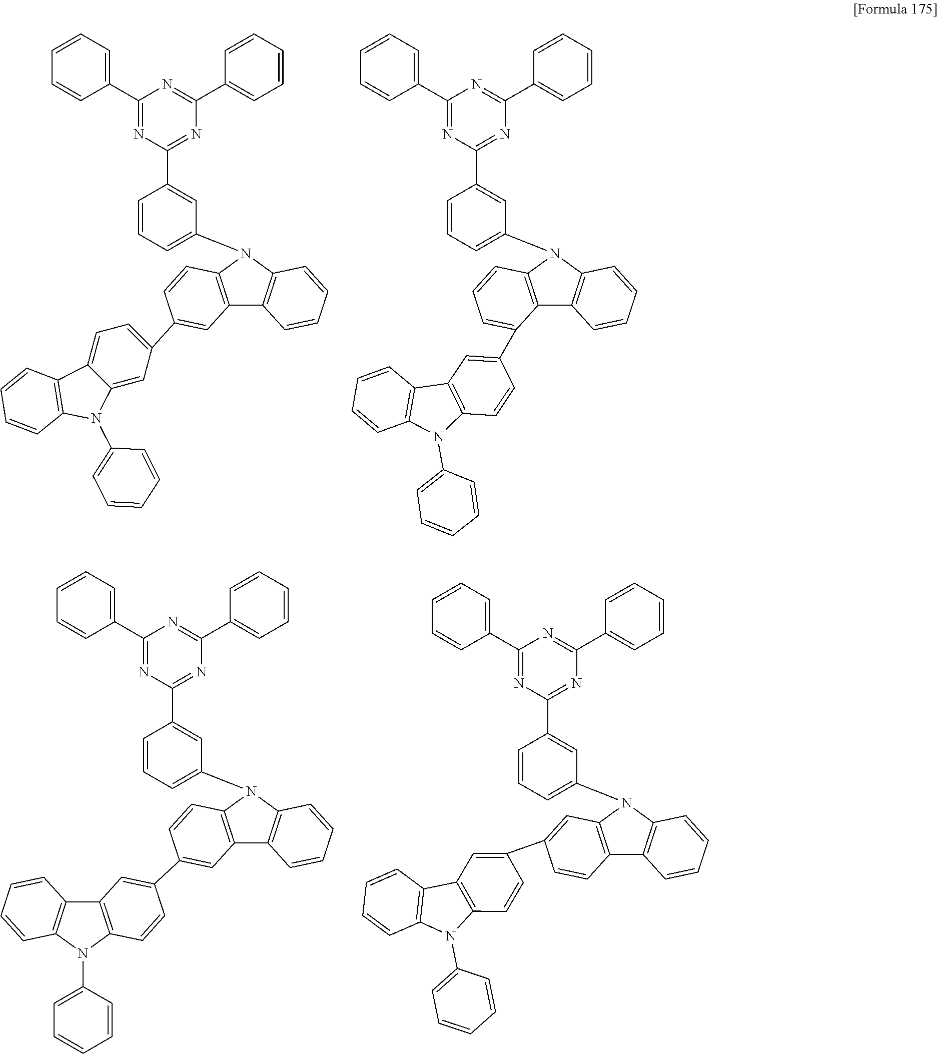 [SMILES: c1ccc(-c2nc(-c3ccccc3)nc(-c3cccc(-n4c5ccccc5c5cc(-c6ccc7c8ccccc8n(-c8ccccc8)c7c6)ccc54)c3)n2)cc1 ; c1ccc(-c2nc(-c3ccccc3)nc(-c3cccc(-n4c5ccccc5c5c(-c6ccc7c(c6)c6ccccc6n7-c6ccccc6)cccc54)c3)n2)cc1 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1]

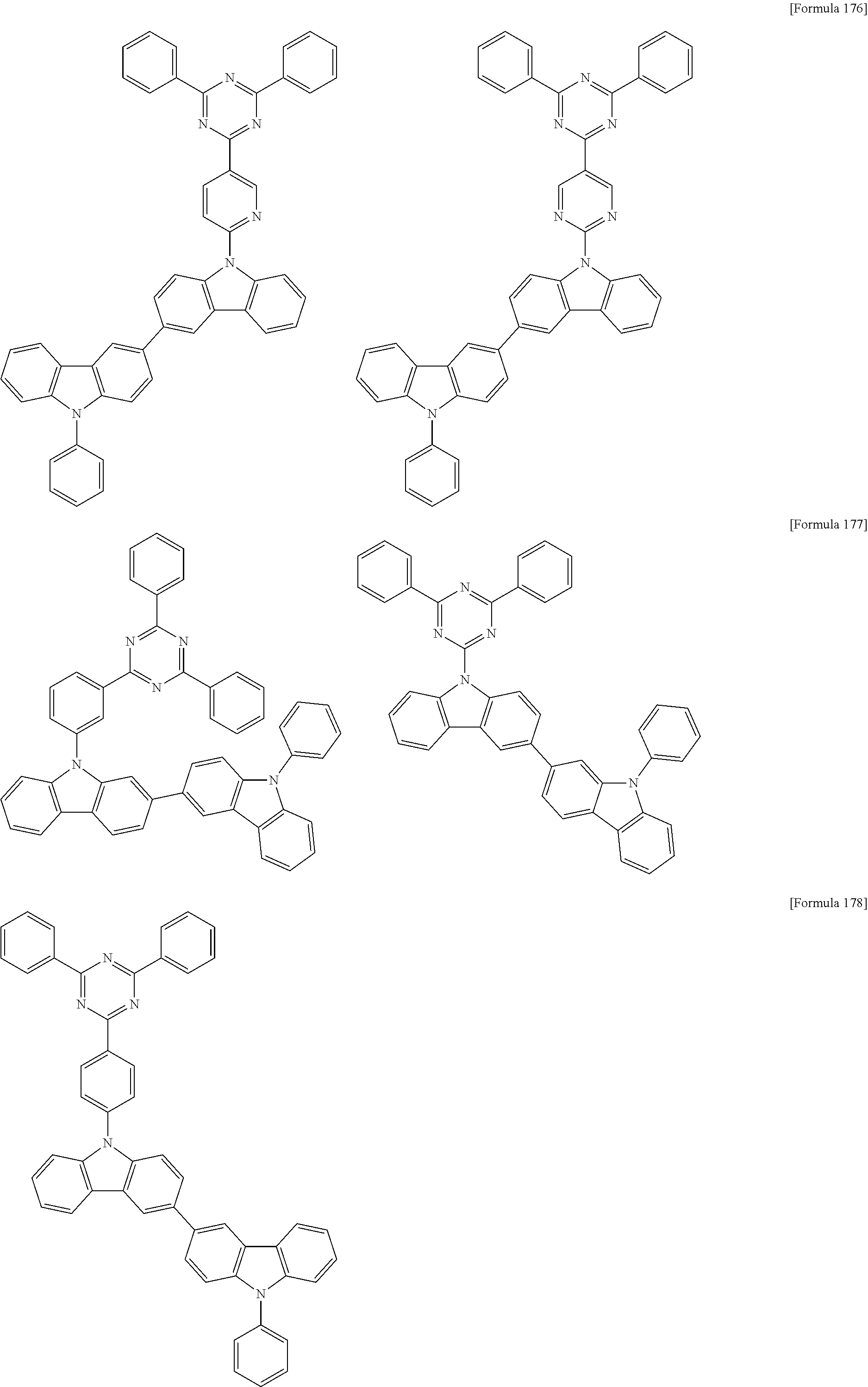 [SMILES: c1ccc(-c2nc(-c3ccccc3)nc(-c3ccc(-n4c5ccccc5c5cc(-c6ccc7c(c6)c6ccccc6n7-c6ccccc6)ccc54)nc3)n2)cc1 ; c1ccc(-c2nc(-c3ccccc3)nc(-c3cnc(-n4c5ccccc5c5cc(-c6ccc7c(c6)c6ccccc6n7-c6ccccc6)ccc54)nc3)n2)cc1 ; c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; c1ccc(-c2nc(-c3ccccc3)nc(-n3c4ccccc4c4cc(-c5ccc6c7ccccc7n(-c7ccccc7)c6c5)ccc43)n2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

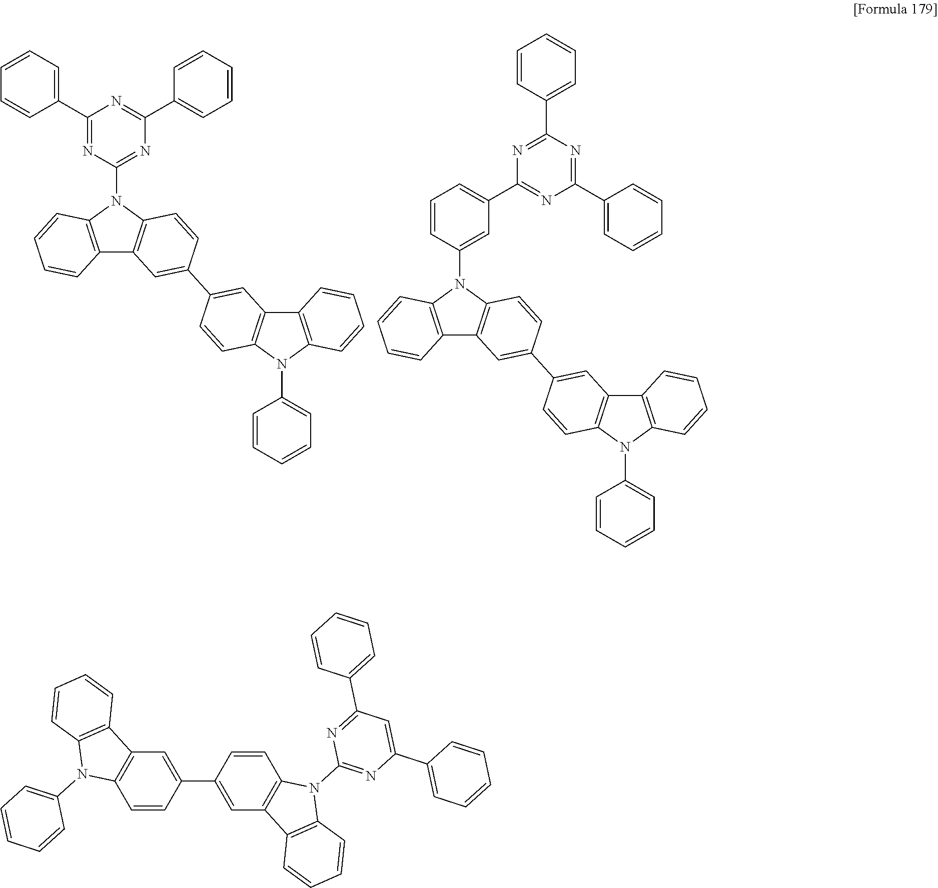

### Relationship Between Compound M3 and Compound M2 in Emitting Layer

In the organic EL device according to the exemplary embodiment, the singlet energy S₁(M2) of the compound M2 and a singlet energy S₁(M3) of the compound M3 satisfy a relationship of a numerical formula (Numerical Formula 1) below.

$${S}_{1} ( M 3 ) > {S}_{1} ( M 2 ) \qquad ( Numerical Formula 1 )$$

An energy gap T_{77K}(M3) at 77K of the compound M3 is preferably larger than an energy gap T_{77K}(M2) at 77K of the compound M2. In other words, a relationship of the following numerical formula (Numerical Formula 11) is preferably satisfied.

$${T}_{77 K} ( M 3 ) > {T}_{77 K} ( M 2 ) \qquad ( Numerical Formula 11 )$$

When the organic EL device according to the exemplary embodiment emits light, it is preferable that the compound M3 does not mainly emit light in the emitting layer.

### Relationship Between Triplet Energy and Energy Gap at 77K

Here, a relationship between a triplet energy and an energy gap at 77K will be described. In the exemplary embodiment, the energy gap at 77K is different from a typical triplet energy in some aspects.

The triplet energy is measured as follows. Firstly, a solution in which a compound (measurement target) is dissolved in an appropriate solvent is encapsulated in a quartz glass tube to prepare a sample. A phosphorescence spectrum (ordinate axis: phosphorescent luminous intensity, abscissa axis: wavelength) of the sample is measured at a low temperature (77K). A tangent is drawn to the rise of the phosphorescence spectrum close to the short-wavelength region. The triplet energy is calculated by a predetermined equation on a basis of a wavelength value at an intersection of the tangent and the abscissa axis.

Here, the thermally activated delayed fluorescent compound among the compounds of the exemplary embodiment is preferably a compound having a small ΔST. When ΔST is small, intersystem crossing and inverse intersystem crossing are likely to occur even at a low temperature (77K), so that the singlet state and the triplet state coexist. As a result, the spectrum to be measured in the same manner as the above includes emission from both the singlet state and the triplet state. Although it is difficult to distinguish the emission from the singlet state from the emission from the triplet state, the value of the triplet energy is basically considered dominant.

Accordingly, in the exemplary embodiment, the triplet energy is measured by the same method as a typical triplet energy T, but a value measured in the following manner is referred to as an energy gap T_{77K} in order to differentiate the measured energy from the typical triplet energy in a strict meaning. The measurement target compound is dissolved in EPA (diethylether:isopentane:ethanol=5:5:2 in volume ratio) at a concentration of 10 μmol/L, and the obtained solution is encapsulated in a quartz cell to provide a measurement sample. A phosphorescence spectrum (ordinate axis: phosphorescent luminous intensity, abscissa axis: wavelength) of the measurement sample is measured at a low temperature (77K). A tangent is drawn to the rise of the phosphorescence spectrum close to the short-wavelength region. An energy amount is calculated by a conversion equation (F1) below on a basis of a wavelength value λ_{edge} [nm] at an intersection of the tangent and the abscissa axis. The calculated energy amount is defined as an energy gap T_{77K} at 77K.

$${T}_{77 K} [ eV ] = 1239.85 / {λ}_{edge} \qquad Conversion Equation ( F1 )$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength region of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (manufactured by Hitachi High-Technologies Corporation) is usable. Any device for phosphorescence measurement is usable. A combination of a cooling unit, a low temperature container, an excitation light source and a light-receiving unit may be used for phosphorescence measurement.

### Singlet Energy S1

A method of measuring a singlet energy S₁ with use of a solution (occasionally referred to as a solution method) is exemplified by a method below.

A toluene solution of a measurement target compound at a concentration of 10 μmol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum close to the long-wavelength region, and a wavelength value λedge (nm) at an intersection of the tangent and the abscissa axis is assigned to a conversion equation (F2) below to calculate singlet energy.

$${S}_{1} [ eV ] = 1239.85 / {λ}_{edge} \qquad Conversion Equation ( F2 )$$

Any device for measuring absorption spectrum is usable. For instance, a spectrophotometer (U3310 manufactured by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

In the exemplary embodiment, a difference (S₁−T_{77K}) between the singlet energy S₁ and the energy gap T_{77K} at 77K is defined as ΔST.

In the exemplary embodiment, a difference ΔST(M2) between the singlet energy S₁(M2) of the compound M2 and the energy gap T_{77K}(M2) at 77K of the compound M2 is preferably less than 0.3 eV, more preferably less than 0.2 eV, further preferably less than 0.1 eV, more further preferably less than 0.01 eV. In other words, ΔST(M2) preferably satisfies a relationship of one of numerical formulae (Numerical Formula 1A to Numerical Formula 1D).

$$\begin{matrix}Δ ST ( M 2 ) = {S}_{1} ( M 2 ) - {T}_{77 K} ( M 2 ) < 0.3 eV & ( Numerical Formula 1 A ) \\ Δ ST ( M 2 ) = {S}_{1} ( M 2 ) - {T}_{77 K} ( M 2 ) < 0.2 eV & ( Numerical Formula 1 B ) \\ Δ ST ( M 2 ) = {S}_{1} ( M 2 ) - {T}_{77 K} ( M 2 ) < 0.1 eV & ( Numerical Formula 1 C ) \\ Δ ST ( M 2 ) = {S}_{1} ( M 2 ) - {T}_{77 K} ( M 2 ) < 0.01 eV & ( Numerical Formula 1 D )\end{matrix}$$

### Film Thickness of Emitting Layer

A film thickness of the emitting layer of the organic EL device in the exemplary embodiment is preferably in a range from 5 nm to 50 nm, more preferably in a range from 7 nm to 50 nm, most preferably in a range from 10 nm to 50 nm. When the film thickness of the emitting layer is 5 nm or more, the formation of the emitting layer and the adjustment of the chromaticity are easy. When the film thickness of the emitting layer is 50 nm or less, an increase in the drive voltage is likely to be reducible.

### Content Ratios of Compounds in Emitting Layer

Content ratios of the compounds M2 and M3 in the emitting layer preferably fall, for instance, within a range below.

The content ratio of the compound M2 is preferably in a range from 10 mass % to 80 mass %, more preferably in a range from 10 mass % to 60 mass %, further preferably in a range from 20 mass % to 60 mass %.

The content ratio of the compound M3 is preferably in a range from 20 mass % to 90 mass %, more preferably in a range from 40 mass % to 90 mass %, further preferably in a range from 40 mass % to 80 mass %.

It should be noted that the emitting layer of the exemplary embodiment may further contain material(s) other than the compounds M2 and M3.

The emitting layer may include a single type of the compound M2 or may include two or more types of the compound M2. The emitting layer may include a single type of the compound M3 or may include two or more types of the compound M3.

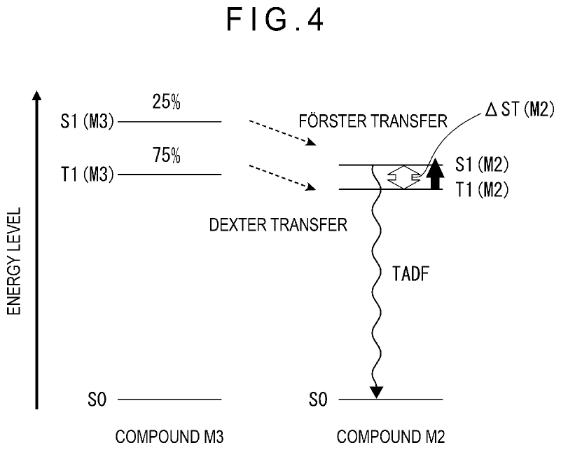

FIG. 4 shows a relationship in energy level and energy transfer between the compound M3 and the compound M2 in the emitting layer.

In FIG. 4, S0 represents a ground state. S1(M2) represents the lowest singlet state of the compound M2. T1(M2) represents the lowest triplet state of the compound M2. S1(M3) represents the lowest singlet state of the compound M3. T1(M3) represents the lowest triplet state of the compound M3.

Dashed arrows in FIG. 4 show energy transfer between the excited states. An energy transfer occurs by Förster transfer from the lowest singlet state S1 of the compound M3 to the lowest singlet state S1 of the compound M2 or an energy transfer occurs by Dexter transfer from the lowest triplet state T1 of the compound M3 to the lowest triplet state T1 of the compound M2.

Further, when a material having a small ΔST(M2) is used as the compound M2, inverse intersystem crossing can be caused by a heat energy from the lowest triplet state T1 to the lowest singlet state S1 in the compound M2. Consequently, fluorescence from the lowest singlet state S1 of the compound M2 can be observed. It is inferred that the internal quantum efficiency can be theoretically raised up to 100% also by using delayed fluorescence by the TADF mechanism.

The organic EL device according to the exemplary embodiment contains the delayed fluorescent compound M2 and the compound M3 having the singlet energy larger than that of the compound M2 in the emitting layer.

According to the exemplary embodiment, a high-performance organic EL device such as an organic EL device emitting light with a high efficiency is achievable.

The organic EL device according to the exemplary embodiment is applicable to an electronic device such as a display device and a light-emitting device.

An arrangement of an organic EL device will be further described below.

### Substrate

The substrate is used as a support for the organic EL device. For instance, glass, quartz, plastics and the like are usable for the substrate. A flexible substrate is also usable. The flexible substrate is a bendable substrate, which is exemplified by a plastic substrate. Examples of the material for the plastic substrate include polycarbonate, polyarylate, polyethersulfone, polypropylene, polyester, polyvinyl fluoride, polyvinyl chloride, polyimide, and polyethylene naphthalate. Moreover, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate. Specific examples of the material include ITO (Indium Tin Oxide), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the EL layers formed on the anode, since the hole injecting layer adjacent to the anode is formed of a composite material into which holes are easily injectable irrespective of the work function of the anode, a material usable as an electrode material (e.g., metal, an alloy, an electrically conductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), and alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode. Examples of the material for the cathode include elements belonging to Groups 1 and 2 in the periodic table of the elements, specifically, the alkali metal such as lithium (Li) and cesium (Cs), the alkaline earth metal such as magnesium (Mg), calcium (Ca) and strontium (Sr), alloys (e.g., MgAg and AlLi) including the alkali metal or the alkaline earth metal, the rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method and the like.

### Hole Injecting Layer

The hole injecting layer is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the highly hole-injectable substance further include: an aromatic amine compound, which is a low-molecule organic compound, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and 3-[N-(1-naphthyl)-N-(9-phenylcarbazole-3-yl)amino]-9-phenylcarbazole (abbreviation: PCzPCN1); and dipyrazino[2,3-f:20,30-h]quinoxaline-2,3,6,7,10,11-hexacarbonitrile (HAT-CN).

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer is a layer containing a highly hole-transporting substance. An aromatic amine compound, carbazole derivative, anthracene derivative and the like are usable for the hole transporting layer. Specific examples of a material for the hole transporting layer include an aromatic amine compound such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

When the hole transporting layer includes two or more layers, one of the layers with a larger energy gap is preferably provided closer to the emitting layer. Examples of the material with a larger energy gap include compounds EBL and EBL-2 used in later-described Examples.

### Electron Transporting Layer

The electron transporting layer is a layer containing a highly electron-transporting substance. For the electron transporting layer, 1) a metal complex such as an aluminum complex, beryllium complex, and zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinato)aluminum (abbreviation: Almq₃), bis(10-hydroxybenzo[h]quinolinato)beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is preferably usable. The above-described substances mostly have an electron mobility of 10⁻⁶ cm²/Vs or more. It should be noted that any substance other than the above substance may be used for the electron transporting layer as long as the substance exhibits a higher electron transportability than the hole transportability. The electron transporting layer may be provided in the form of a single layer or a laminate of two or more layers of the above substance(s).

Further, a high polymer compound is usable for the electron transporting layer. For instance, poly[(9,9-dihexylfluorene-2,7-diyl)-co-(pyridine-3,5-diyl)](abbreviation: PF-Py), poly[(9,9-dioctylfluorene-2,7-diyl)-co-(2,2′-bipyridine-6,6′-diyl)](abbreviation: PF-BPy) and the like are usable.

### Electron Injecting Layer

The electron injecting layer is a layer containing a highly electron-injectable substance. Examples of a material for the electron injecting layer include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method for forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

A thickness of each of the organic layers in the organic EL device according to the exemplary embodiment is not limited except for the above particular description. In general, the thickness preferably ranges from several nanometers to 1 μm because excessively small film thickness is likely to cause defects (e.g. pin holes) and excessively large thickness leads to the necessity of applying high voltage and consequent reduction in efficiency.

The inventors have found that a high-performance organic EL device is achievable by using the compound M3 represented by the formula (3) with the delayed fluorescent compound M2.

Since the compound M3 has both carbazole being at least one of a hole-injectable moiety or a hole transporting moiety and A₃₀ (preferably benzofurocarbazole) being higher in at least one of hole injectability or hole transportability than carbazole, the compound M3 is suitably higher in at least one of the hole injectability or hole transportability than a compound having only carbazole and no A₃₀ (e.g., CBP).

In the exemplary embodiment, it is considered that by containing, in the emitting layer, the compound M3 having at least one of hole injectability or hole transportability that is appropriately enhanced, an appropriate amount of holes can be supplied to the emitting layer to bring the amount of holes in line with an amount of electrons. As a result, it is considered that the device is highly improved in efficiency. In general, a moiety having a large absolute value of an energy level of LUMO (lowest unoccupied molecular orbital) is often introduced into a molecule of a delayed fluorescent compound to reduce ΔST, which would also often increase an absolute value of an energy level of HOMO (highest occupied molecular orbital) of the entire molecule thereof. However, having a large absolute value of an energy level of HOMO may hinder hole injection into the emitting layer, failing to supply an appropriate amount of holes to the emitting layer.

In the exemplary embodiment, it is considered that since the emitting layer contains the compound M3 having at least one of hole injectability or hole transportability that is appropriately enhanced and the delayed fluorescent compound M2, hole injection into the emitting layer is prevented from being hindered to supply an appropriate amount of holes to the emitting layer, thus highly improving the device in efficiency.

Accordingly, according to the exemplary embodiment, a high-performance organic EL device is achievable.

“High performance” in the exemplary embodiment means at least one of light emission with a long lifetime, an improvement in a luminous efficiency, or a decrease in a drive voltage.

### Second Exemplary Embodiment

An arrangement of an organic EL device according to a second exemplary embodiment will be described below. In the description of the second exemplary embodiment, the same components as those in the first exemplary embodiment are denoted by the same reference signs and names to simplify or omit an explanation of the components. In the second exemplary embodiment, any materials and compounds that are not specified may be the same as those in the first exemplary embodiment.

The organic EL device according to the second exemplary embodiment is different from the organic EL device according to the first exemplary embodiment in that the emitting layer further includes a fluorescent compound M1. The second exemplary embodiment is the same as the first exemplary embodiment in other respects.

In other words, in the second exemplary embodiment, the emitting layer contains the compound M3 represented by the formula (3), the delayed fluorescent compound M2, and the fluorescent compound M1.

In this arrangement, the compound M1 is preferably a dopant material, the compound M2 is preferably a host material, and the compound M3 is preferably not a dopant material.

### Compound M1

The emitting layer of the exemplary embodiment includes the fluorescent compound M1.

The compound M1 of the exemplary embodiment is not a phosphorescent metal complex. The compound M1 of the exemplary embodiment is preferably not a heavy-metal complex. The compound M1 of the exemplary embodiment is preferably not a metal complex.

A fluorescent material is usable as the compound M1 of the exemplary embodiment. Specific examples of the fluorescent material include a bisarylaminonaphthalene derivative, aryl-substituted naphthalene derivative, bisarylaminoanthracene derivative, aryl-substituted anthracene derivative, bisarylaminopyrene derivative, aryl-substituted pyrene derivative, bisarylamino chrysene derivative, aryl-substituted chrysene derivative, bisarylaminofluoranthene derivative, aryl-substituted fluoranthene derivative, indenoperylene derivative, acenaphthofluoranthene derivative, compound including a boron atom, pyromethene boron complex compound, compound having a pyromethene skeleton, metal complex of the compound having a pyrromethene skeleton, diketopyrrolopyrrole derivative, perylene derivative, and naphthacene derivative.

The compound M1 of the exemplary embodiment is preferably a compound represented by a formula (10) below.

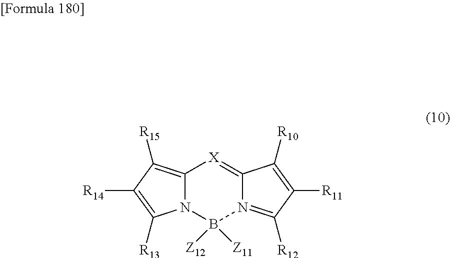

In the formula (10):

X is a nitrogen atom, or a carbon atom bonded to Y;

Y is a hydrogen atom or a substituent;

R₁₀ to R₁₅ are each independently a hydrogen atom or a substituent, or at least one pair of a pair of R₁₀ and R₁₁, a pair of R₁₁ and R₁₂, a pair of R₁₃ and R₁₄, or a pair of R₁₄ and R₁₅ are mutually bonded to form a ring;

Y and R₁₀ to R₁₅ as a substituent are each independently selected from the group consisting of a substituted or unsubstituted alkyl group having 1 to 30 carbon atoms, a substituted or unsubstituted alkyl halide group having 1 to 30 carbon atoms, a substituted or unsubstituted cycloalkyl group having 3 to 30 ring carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, a substituted or unsubstituted alkylthio group having 1 to 30 carbon atoms, a substituted or unsubstituted aryloxy group having 6 to 30 ring carbon atoms, a substituted or unsubstituted arylthio group having 6 to 30 ring carbon atoms, a substituted or unsubstituted alkenyl group having 2 to 30 carbon atoms, a substituted or unsubstituted aralkyl group having 7 to 30 carbon atoms, a substituted or unsubstituted heteroaryl group having 5 to 30 ring atoms, a halogen atom, a carboxy group, a substituted or unsubstituted ester group, a substituted or unsubstituted carbamoyl group, a substituted or unsubstituted amino group, a nitro group, a cyano group, a substituted or unsubstituted silyl group, and a substituted or unsubstituted siloxanyl group;

Z₁₁ and Z₁₂ are each independently a substituent, or Z₁₁ and Z₁₂ are mutually bonded to form a ring; and

Z₁₁ and Z₁₂ as a substituent are each independently selected from the group consisting of a halogen atom, a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

In the formula (10), for instance, when a pair of R₁₄ and R₁₅ are mutually bonded to form a ring, the compound M1 is represented by a formula (11) below.

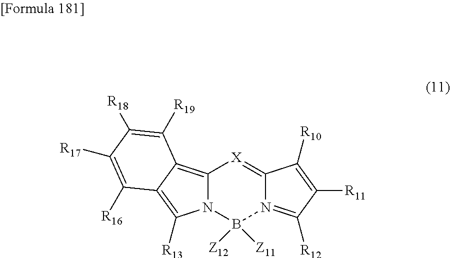

In the formula (11): X, Y, R₁₀ to R₁₃, Z₁₁ and Z₁₂ respectively represent the same as X, Y, R₁₀ to R₁₃, Z₁₁ and Z₁₂ in the formula (10); R₁₆ to R₁₉ are each independently a hydrogen atom or a substituent; and R₁₆ to R₁₉ as a substituent each independently represent the same as R₁₀ to R₁₃ as a substituent.

In the formula (10), when Z₁₁ and Z₁₂ are mutually bonded to form a ring, the compound M1 is represented by, for instance, a formula (10A) or (10B) below. However, a structure of the compound M1 is not limited to structures below.

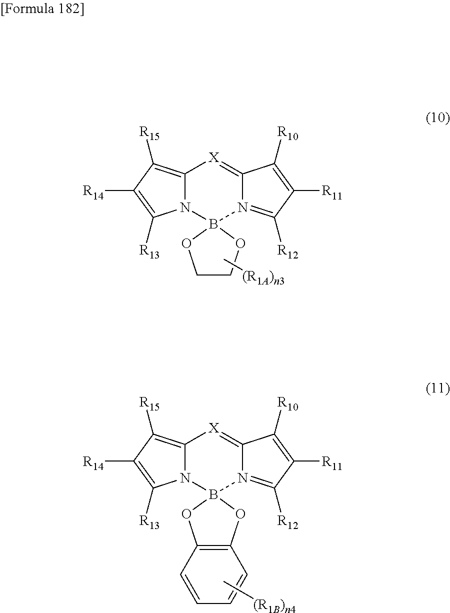

In the formula (10A): X, Y and R₁₀ to R₁₅ respectively represent the same as X, Y and R₁₀ to R₁₅ in the formula (10); R_{1A} is each independently a hydrogen atom or a substituent; R_{1A} as a substituent represents the same as R₁₀ to R₁₅ as a substituent; and n3 is 4.

In the formula (10B): X, Y and R₁₀ to R₁₅ respectively represent the same as X, Y and R₁₀ to R₁₅ in the formula (10); R_{1B} is each independently a hydrogen atom or a substituent; R_{1B} as a substituent represents the same as R₁₀ to R₁₅ as a substituent; and n4 is 4.

It is preferable that at least one of Z₁₁ or Z₁₂ (preferably both of Z₁₁ and Z₁₂) is a group selected from the group consisting of a substituted or unsubstituted alkyl group having 1 to 30 carbon atoms, a substituted or unsubstituted alkyl halide group having 1 to 30 carbon atoms, a substituted or unsubstituted aryl group having 6 to 30 ring carbon atoms, a substituted or unsubstituted alkoxy group having 1 to 30 carbon atoms, a substituted or unsubstituted alkoxy halide group having 1 to 30 carbon atoms, and a substituted or unsubstituted aryloxy group having 6 to 30 ring carbon atoms.

It is more preferable that at least one of Z₁₁ or Z₁₂ is a group selected from the group consisting of a fluorine-substituted alkoxy group having 1 to 30 carbon atoms, a fluorine-substituted aryloxy group having 6 to 30 ring carbon atoms, and an aryloxy group having 6 to 30 ring carbon atoms and substituted with a fluoroalkyl group having 1 to 30 carbon atoms.

Further preferably, at least one of Z₁₁ or Z₁₂ is a fluorine-substituted alkoxy group having 1 to 30 carbon atoms. Further more preferably, both of Z₁₁ and Z₁₂ are a fluorine-substituted alkoxy group having 1 to 30 carbon atoms.

It is also preferable that both of Z₁₁ and Z₁₂ are the same.

Meanwhile, it is also preferable that at least one of Z₁₁ or Z₁₂ is a fluorine atom. It is also more preferable that both of Z₁₁ and Z₁₂ are fluorine atoms.

It is also preferable that at least one of Z₁₁ or Z₁₂ is a group represented by a formula (10a) below.

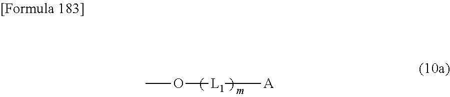

In the formula (10a): A is a substituted or unsubstituted alkyl group having 1 to 6 carbon atoms, a substituted or unsubstituted alkyl halide group having 1 to 6 carbon atoms, or a substituted or unsubstituted aryl group having 6 to 12 ring carbon atoms; L₁ is a substituted or unsubstituted alkylene group having 1 to 6 carbon atoms, or a substituted or unsubstituted arylene group having 6 to 12 ring carbon atoms; m is 0, 1, 2, 3, 4, 5, 6 or 7; and when m is 2, 3, 4, 5, 6 or 7, a plurality of L₁ are mutually the same or different. m is preferably 0, 1 or 2. When m is 0, A is directly bonded to O (oxygen atom).

When Z₁₁ and Z₁₂ in the formula (10) are each the group represented by the formula (10a), the compound M1 is a compound represented by a formula (12) below.

The compound M1 is also preferably the compound represented by the formula (12) below.

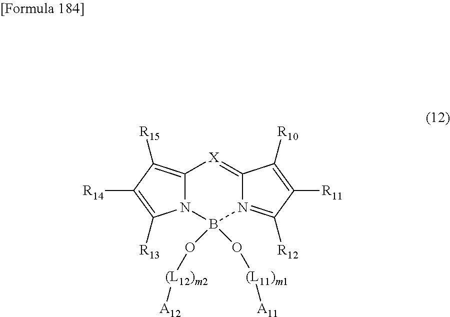

In the formula (12), X, Y bonded with a carbon atom as X, and R₁₀ to R₁₅ respectively represent the same as X, Y and R₁₀ to R₁₅ in the formula (10). A₁₁ and A₁₂ represent the same as A in the formula (10a) and may be mutually the same or different. L₁₁ and L₁₂ represent the same as L₁ in the formula (10a) and may be mutually the same or different. m1 and m2 are each independently 0, 1, 2, 3, 4, 5, 6 or 7, preferably 0, 1 or 2. When m1 is 2, 3, 4, 5, 6 or 7, a plurality of L₁₁ are mutually the same or different. When m2 is 2, 3, 4, 5, 6 or 7, a plurality of L₁₂ are mutually the same or different. When m1 is 0, A₁₁ is directly bonded to O (oxygen atom). When m2 is 0, A₁₂ is directly bonded to O (oxygen atom).

At least one of A or L₁ in the formula (10a) is preferably substituted with a halogen atom, more preferably substituted with a fluorine atom.

A in the formula (10a) is more preferably a perfluoroalkyl group having 1 to 6 carbon atoms or a perfluoroaryl group having 6 to 12 ring carbon atoms, further preferably a perfluoroalkyl group having 1 to 6 carbon atoms.

L₁ in the formula (10a) is more preferably a perfluoroalkylene group having 1 to 6 carbon atoms or a perfluoroarylene group having 6 to 12 ring carbon atoms, further preferably a perfluoroalkylene group having 1 to 6 carbon atoms.

In other words, the compound M1 is also preferably a compound represented by a formula (12a) below.

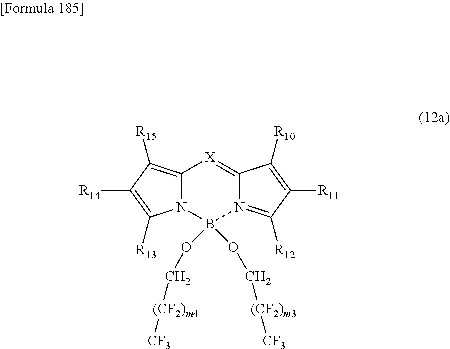

In the formula (12a):

X represents the same as X in the formula (10);

Y bonded with a carbon atom as X represents the same as Y in the formula (10);

R₁₀ to R₁₅ each independently represent the same as R₁₀ to R₁₅ in the formula (10);

m3 is 0, 1, 2, 3 or 4;

m4 is 0, 1, 2, 3 or 4; and

m3 and m4 are mutually the same or different.

In the formulae (10), (11), (12) and (12a):

X is a carbon atom bonded to Y; and Y is a hydrogen atom or a substituent.

Y as a substituent is preferably a substituent selected from the group consisting of a substituted or unsubstituted alkyl group having 1 to 30 carbon atoms, a substituted or unsubstituted alkyl halide group having 1 to 30 carbon atoms and a substituted or unsubstituted aryl group having 6 to 30 ring carbon atoms, more preferably a substituted or unsubstituted aryl group having 6 to 30 ring carbon atoms.

In the formulae (10), (11), (12) and (12a), it is more preferable that: X is a carbon atom bonded to Y; Y is a hydrogen atom or a substituent; Y as a substituent is a substituted or unsubstituted aryl group having 6 to 30 ring carbon atoms; and when Y as a substituent is an aryl group having 6 to 30 ring carbon atoms having a substituent, the substituent is a substituted or unsubstituted alkyl group having 1 to 30 carbon atoms, a substituted or unsubstituted alkyl halide group having 1 to 30 carbon atoms, a substituted or unsubstituted alkoxy group having 1 to 30 carbon atoms, a substituted or unsubstituted alkoxy halide group having 1 to 30 carbon atoms, or an aryl group having 6 to 30 ring carbon atoms and substituted by an alkyl group having 1 to 30 carbon atoms.

In the compound M1, Z₁₁ and Z₁₂ may be mutually bonded to form a ring. However, it is preferable that Z₁₁ and Z₁₂ are not mutually bonded to form no ring.

In the formulae (10), (12) and (12a), at least one of R₁₀, R₁₂, R₁₃ or R₁₅ is preferably a substituted or unsubstituted alkyl group having 1 to 30 carbon atoms or a substituted or unsubstituted alkyl halide group having 1 to 30 carbon atoms.

In the formulae (10), (12) and (12a), R₁₀, R₁₂, R₁₃ and R₁₅ are more preferably a substituted or unsubstituted alkyl group having 1 to 30 carbon atoms or a substituted or unsubstituted alkyl halide group having 1 to 30 carbon atoms. In this case, R₁₁ and R₁₄ are preferably hydrogen atoms.

In the formulae (10), (12) and (12a), at least one of R₁₀, R₁₂, R₁₃ or R₁₅ is preferably a substituted or unsubstituted aryl group having 6 to 30 ring carbon atoms.

In the formulae (10), (12) and (12a), R₁₀, R₁₂, R₁₃ and R₁₅ are more preferably a substituted or unsubstituted aryl group having 6 to 30 ring carbon atoms. In this case, R₁₁ and R₁₄ are preferably hydrogen atoms.

In the formulae (10), (12) and (12a), it is more preferable that: R₁₀, R₁₂, R₁₃ and R₁₅ are each independently a substituted or unsubstituted alkyl group having 1 to 30 carbon atoms (preferably 1 to 6 carbon atoms), a substituted or unsubstituted alkyl halide group having 1 to 30 carbon atoms (preferably 1 to 6 carbon atoms), or an aryl group having 6 to 30 ring carbon atoms (preferably 6 to 12 ring carbon atoms) and substituted with an alkyl group having 1 to 30 carbon atoms, and R₁₁ and R₁₄ are hydrogen atoms.

In the formula (11), at least one of R₁₀, R₁₂ or R₁₃ is preferably a substituted or unsubstituted alkyl group having 1 to 30 carbon atoms or a substituted or unsubstituted alkyl halide group having 1 to 30 carbon atoms.

In the formula (11), R₁₀, R₁₂ and R₁₃ are more preferably a substituted or unsubstituted alkyl group having 1 to 30 carbon atoms or a substituted or unsubstituted alkyl halide group having 1 to 30 carbon atoms. In this case, R₁₁ is preferably a hydrogen atom.

In the formula (11), at least one of R₁₀, R₁₂ or R₁₃ is preferably a substituted or unsubstituted aryl group having 6 to 30 ring carbon atoms.

In the formula (11), R₁₀, R₁₂ and R₁₃ are more preferably a substituted or unsubstituted aryl group having 6 to 30 ring carbon atoms. In this case, R₁₁ is preferably a hydrogen atom.

In the formula (11), it is more preferable that: R₁₀, R₁₂ and R₁₃ are each independently a substituted or unsubstituted alkyl group having 1 to 30 carbon atoms (preferably 1 to 6 carbon atoms), a substituted or unsubstituted alkyl halide group having 1 to 30 carbon atoms (preferably 1 to 6 carbon atoms), or an aryl group having 6 to 30 ring carbon atoms (preferably 6 to 12 ring carbon atoms) and substituted with an alkyl group having 1 to 30 carbon atoms, and R₁₁ is a hydrogen atom.

In the compound M1, examples of the fluorine-substituted alkoxy group include 2,2,2-trifluoroethoxy group, 2,2-difluoroethoxy group, 2,2,3,3,3-pentafluoro-1-propoxy group, 2,2,3,3-tetrafluoro-1-propoxy group, 1,1,1,3,3,3-hexafluoro-2-propoxy group, 2,2,3,3,4,4,4-heptafluoro-1-butyloxy group, 2,2,3,3,4,4-hexafluoro-1-butyloxy group, nonafluoro-tertiary-butyloxy group, 2,2,3,3,4,4,5,5,5-nonafluoropentanoxy group, 2,2,3,3,4,4,5,5,6,6,6-undecafluorohexanoxy group, 2,3-bis(trifluoromethyl)-2,3-butanedioxy group, 1,1,2,2-tetra(trifluoromethyl)ethylene glycoxy group, 4,4,5,5,6,6,6-heptafluorohexane-1,2-dioxy group, and 4,4,5,5,6,6,7,7,8,8,9,9,9-tridecafluorononane-1,2-dioxy group.

In the compound M1, examples of the fluorine-substituted aryloxy group or the aryloxy group substituted with a fluoroalkyl group include a pentafluorophenoxy group, 3,4,5-trifluorophenoxy group, 4-trifluoromethylphenoxy group, 3,5-bistrifluoromethylphenoxy group, 3-fluoro-4-trifluoromethylphenoxy group, 2,3,5,6-tetrafluoro-4-trifluoromethylphenoxy group, 4-fluorocatecholato group, 4-trifluoromethylcatecholato group, and 3,5-bistrifluoromethylcatecholato group.

When the compound M1 is a fluorescent compound, the compound M1 preferably emits light having a main peak wavelength in a range from 400 nm to 700 nm.

Herein, the main peak wavelength means a peak wavelength of an emission spectrum exhibiting a maximum luminous intensity among fluorescence spectra measured in a toluene solution in which a measurement target compound is dissolved at a concentration ranging from 10⁻⁶ mol/l to 10⁻⁵ mol/l. A spectrophotofluorometer (F-7000 manufactured by Hitachi High-Tech Science Corporation) is used as a measurement device.

The compound M1 preferably exhibits red or green light emission.

Herein, the red light emission refers to a light emission in which a main peak wavelength of fluorescence spectrum is in a range from 600 nm to 660 nm.

When the compound M1 is a red fluorescent compound, the main peak wavelength of the compound M1 is preferably in a range from 600 nm to 660 nm, more preferably in a range from 600 nm to 640 nm, further preferably in a range from 610 nm to 630 nm.

Herein, the green light emission refers to a light emission in which a main peak wavelength of fluorescence spectrum is in a range from 500 nm to 560 nm.

When the compound M1 is a green fluorescent compound, the main peak wavelength of the compound M1 is preferably in a range from 500 nm to 560 nm, more preferably in a range from 500 nm to 540 nm, further preferably in a range from 510 nm to 540 nm.

Herein, the blue light emission refers to a light emission in which a main peak wavelength of fluorescence spectrum is in a range from 430 nm to 480 nm.

When the compound M1 is a blue fluorescent compound, the main peak wavelength of the compound M1 is preferably in a range from 430 nm to 480 nm, more preferably in a range from 440 nm to 480 nm.

A main peak wavelength of the light emitted from the organic EL device is measured as follows.

Voltage is applied on the organic EL devices such that a current density becomes 10 mA/cm², where spectral radiance spectrum is measured by a spectroradiometer CS-2000 (manufactured by Konica Minolta, Inc.).

A peak wavelength of an emission spectrum, at which the luminous intensity of the resultant spectral radiance spectrum is at the maximum, is measured and defined as the main peak wavelength (unit: nm).

### Manufacturing Method of Compound M1 According to Exemplary Embodiment

The compound M1 can be manufactured by a known method.

### Specific Examples of Compound M1

Specific examples of the compound M1 according to the exemplary embodiment are shown below. It should however be noted that the invention is not limited to the specific examples of the compound.

A coordinate bond between a boron atom and a nitrogen atom in a pyrromethene skeleton is shown by various means such as a solid line, a broken line, an arrow, and omission. Herein, the coordinate bond is shown by a solid line or a broken line, or the description of the coordinate bond is omitted.

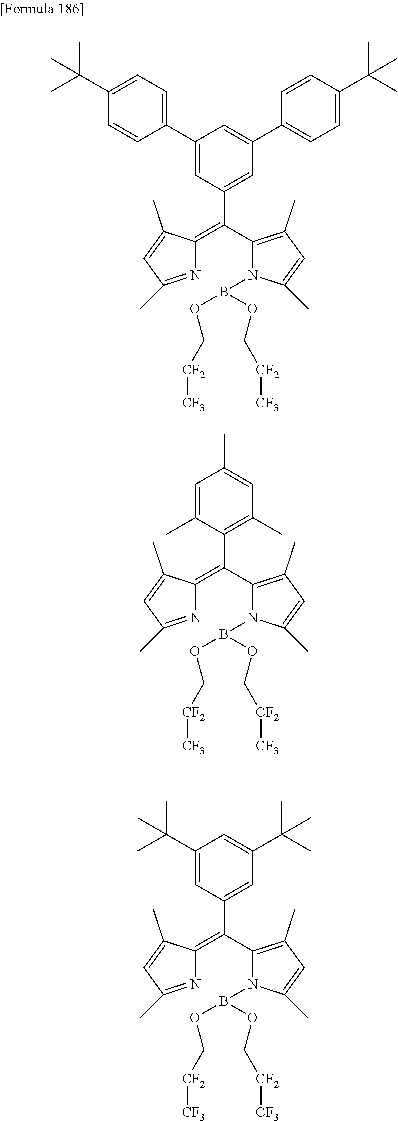

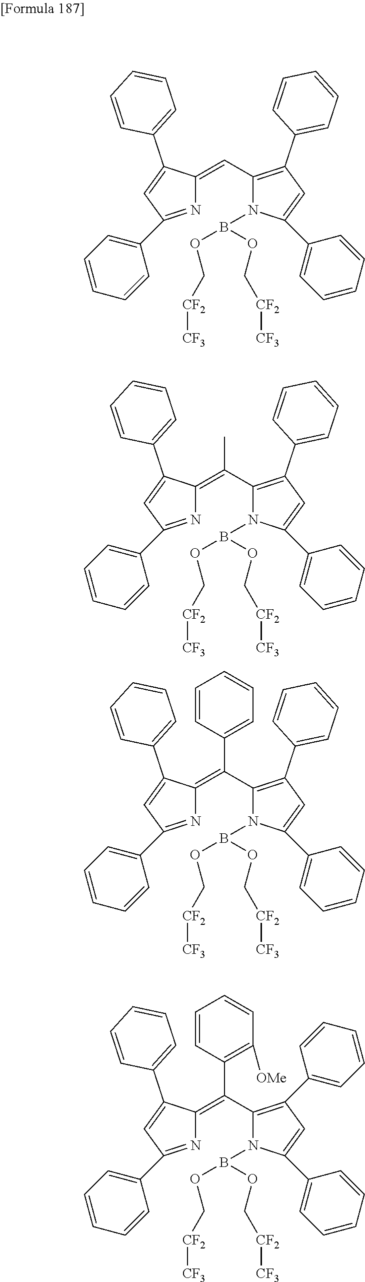

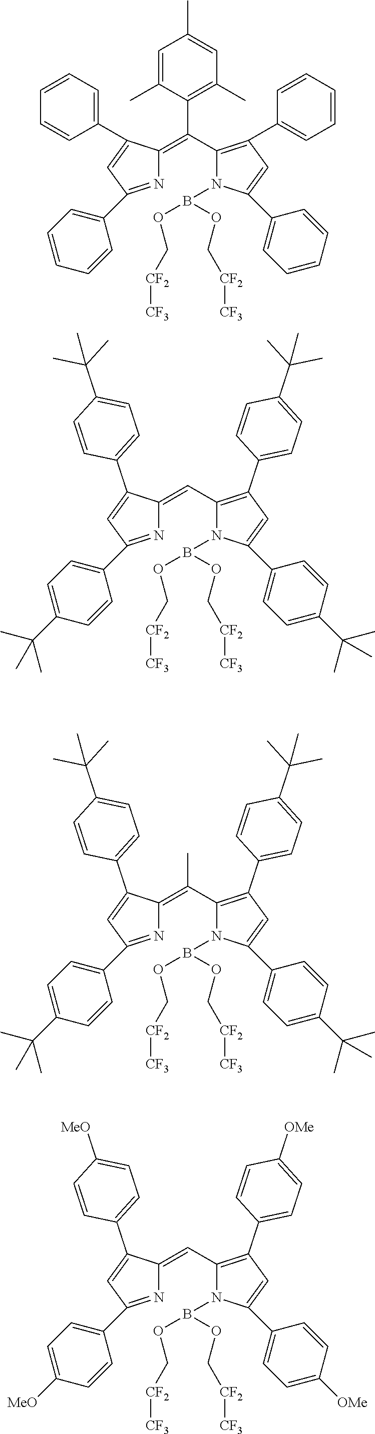

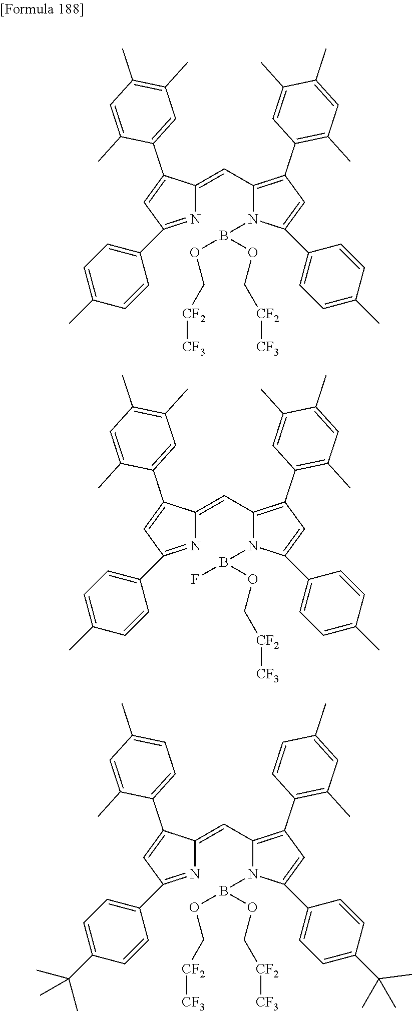

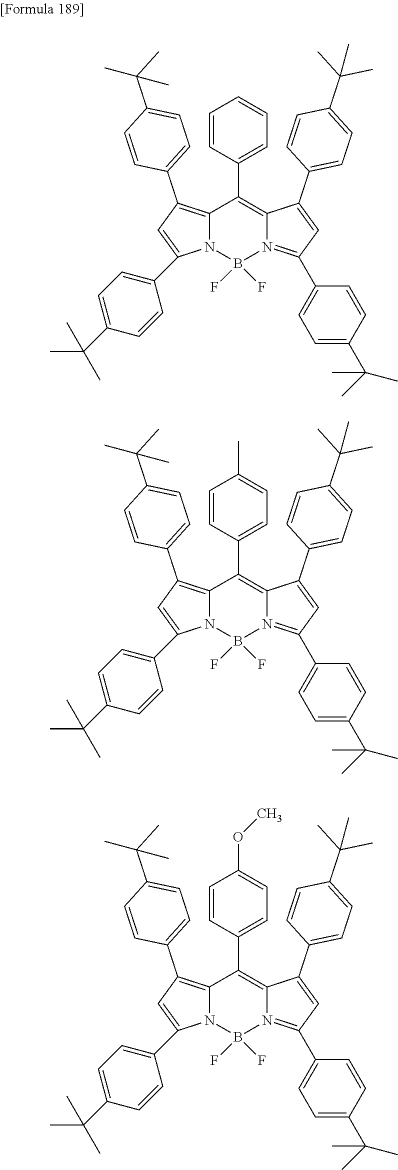

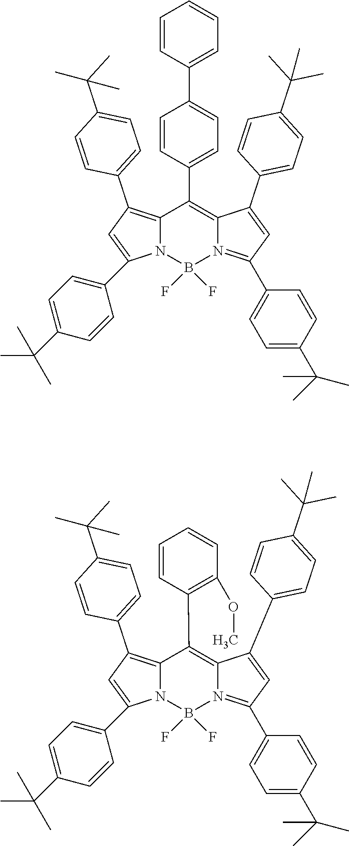

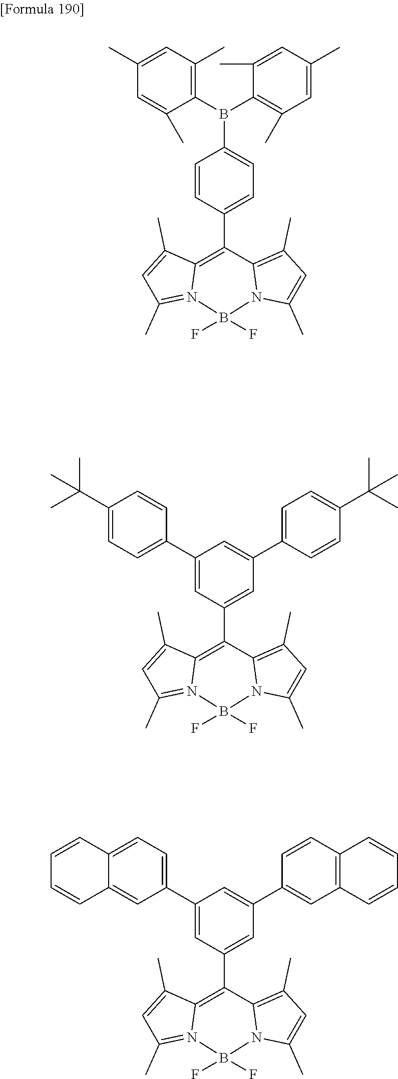

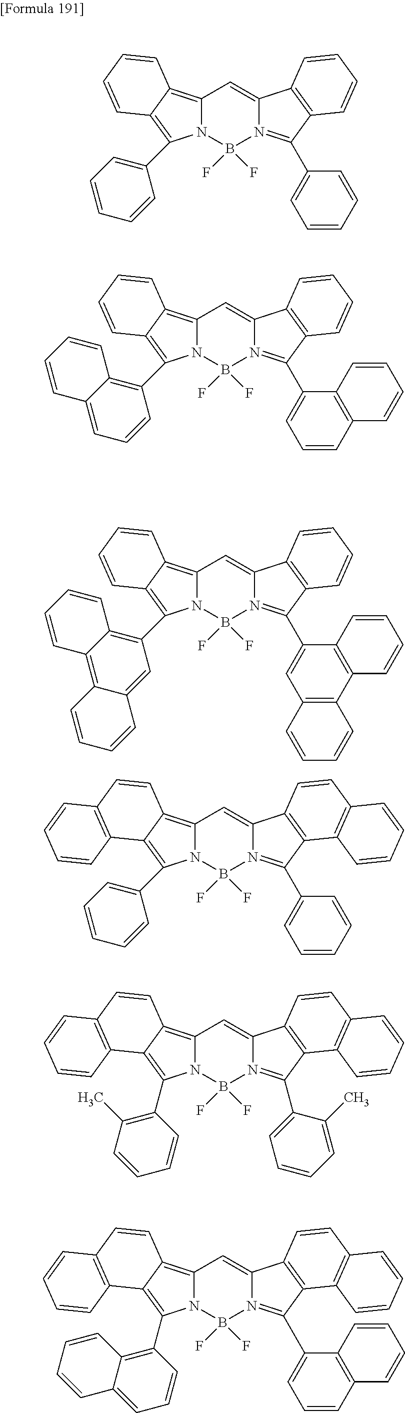

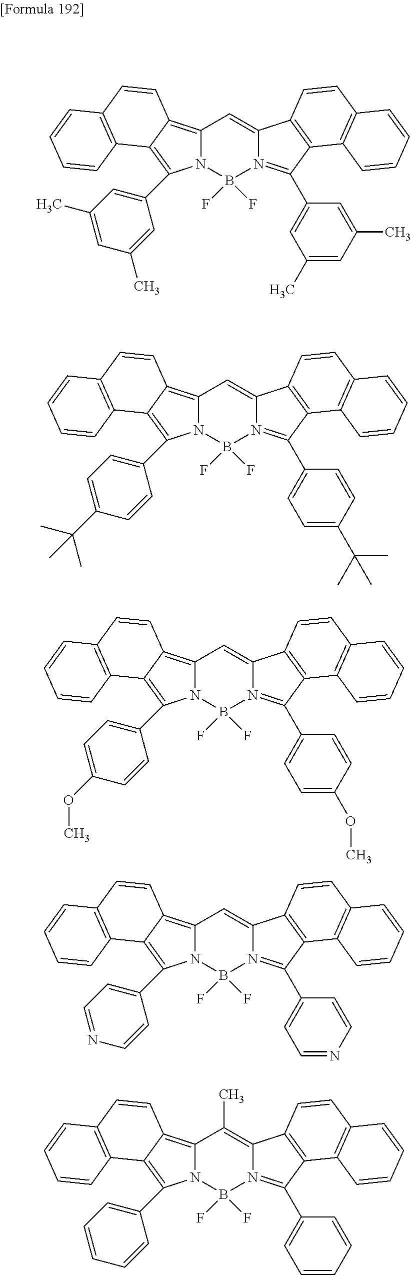

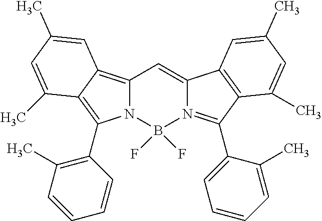

### Relationship Between Compound M3, Compound M2 and Compound M1 in Emitting Layer

In the organic EL device according to the exemplary embodiment, the singlet energy S₁(M2) of the compound M2 and a singlet energy S₁(M1) of the compound M1 preferably satisfy a relationship of a numerical formula (Numerical Formula 2) below.

$${S}_{1} ( M 2 ) > {S}_{1} ( M 1 ) \qquad ( Numerical Formula 2 )$$

The singlet energy S₁(M3) of the compound M3 is preferably larger than the singlet energy S₁(M1) of the compound M1.

$${S}_{1} ( M 3 ) > {S}_{1} ( M 1 ) \qquad ( Numerical Formula 2 A )$$

The singlet energy S₁(M3) of the compound M3, the singlet energy S₁(M2) of the compound M2, and the singlet energy S₁(M1) of the compound M1 preferably satisfy a relationship of a numerical formula (Numerical Formula 2B) below.

$${S}_{1} ( M 3 ) > {S}_{1} ( M 2 ) > {S}_{1} ( M 1 ) \qquad ( Numerical Formula 2 B )$$

When the organic EL device according to the exemplary embodiment emits light, it is preferable that the fluorescent compound M1 in the emitting layer mainly emits light.

The organic EL device according to the exemplary embodiment preferably emits red light or green light.

### Content Ratios of Compounds in Emitting Layer

Content ratios of the compounds M3, M2 and M1 in the emitting layer are preferably fall within, for instance, the following range.

The content ratio of the compound M3 is preferably in a range from 10 mass % to 80 mass %.

The content ratio of the compound M2 is preferably in a range from 10 mass % to 80 mass %, more preferably in a range from 10 mass % to 60 mass %, further preferably in a range from 20 mass % to 60 mass %.

The content ratio of the compound M1 is preferably in a range from 0.01 mass % to 10 mass %, more preferably in a range from 0.01 mass % to 5 mass %, further preferably in a range from 0.01 mass % to 1 mass %.

An upper limit of the total of the respective content ratios of the compounds M3, M2 and M1 in the emitting layer is 100 mass %. It should be noted that the emitting layer of the exemplary embodiment may further contain material(s) other than the compounds M3, M2 and M1.

The emitting layer may include a single type of the compound M3 or may include two or more types of the compound M3. The emitting layer may include a single type of the compound M2 or may include two or more types of the compound M2. The emitting layer may include a single type of the compound M1 or may include two or more types of the compound M1.

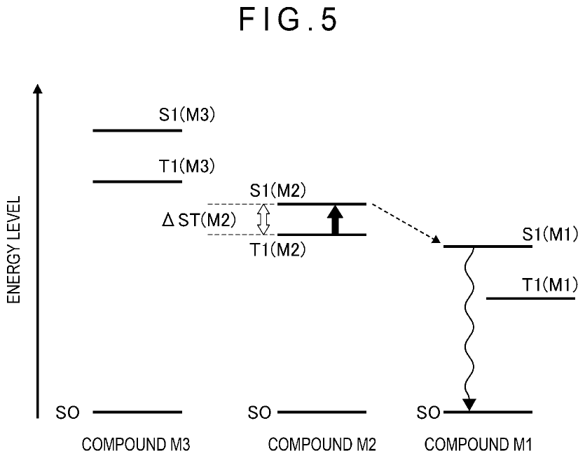

FIG. 5 shows an example of a relationship between energy levels of the compounds M3, M2 and M1 in the emitting layer. In FIG. 5, S0 represents a ground state. S1(M1) represents the lowest singlet state of the compound M1. T1(M1) represents the lowest triplet state of the compound M1. S1(M2) represents the lowest singlet state of the compound M2. T1(M2) represents the lowest triplet state of the compound M1. S1(M3) represents the lowest singlet state of the compound M3. T1(M3) represents the lowest triplet state of the compound M3. A dashed arrow directed from S1(M2) to S1(M1) in FIG. 5 represents Förster energy transfer from the lowest singlet state of the compound M2 to the lowest singlet state of the compound M1.

As shown in FIG. 5, when a compound having a small ΔST(M2) is used as the compound M2, inverse intersystem crossing from the lowest triplet state T1(M2) to the lowest singlet state S1(M2) can be caused by a heat energy. Subsequently, Förster energy transfer from the lowest singlet state S1(M2) of the compound M2 to the compound M1 occurs to generate the lowest singlet state S1(M1). Consequently, fluorescence from the lowest singlet state S1(M1) of the compound M1 can be observed. It is inferred that the internal quantum efficiency can be theoretically raised up to 100% also by using delayed fluorescence by the TADF mechanism.

The organic EL device according to the second exemplary embodiment contains the delayed fluorescent compound M2, the compound M3 having the singlet energy larger than that of the compound M2, and the compound M1 having the singlet energy smaller than that of the delayed fluorescent compound M2 in the emitting layer.

According to the second exemplary embodiment, a high-performance organic EL device such as an organic EL device emitting light with a high efficiency can be achieved.

The organic EL device according to the second exemplary embodiment is applicable to an electronic device such as a display device and a light-emitting device.

### Third Exemplary Embodiment

### Compound

A compound of a third exemplary embodiment is a compound represented by a formula (301) below. A compound falling under the formula (301) among the specific examples of M3 of the first embodiment is also used as a specific example of the compound of the third exemplary embodiment.

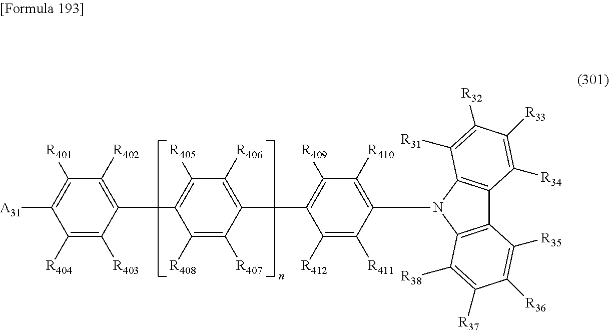

In the formula (301), A₃₁ is a group represented by a formula (31a), (31b), (31c), (31d), (31e) or (31f) below.

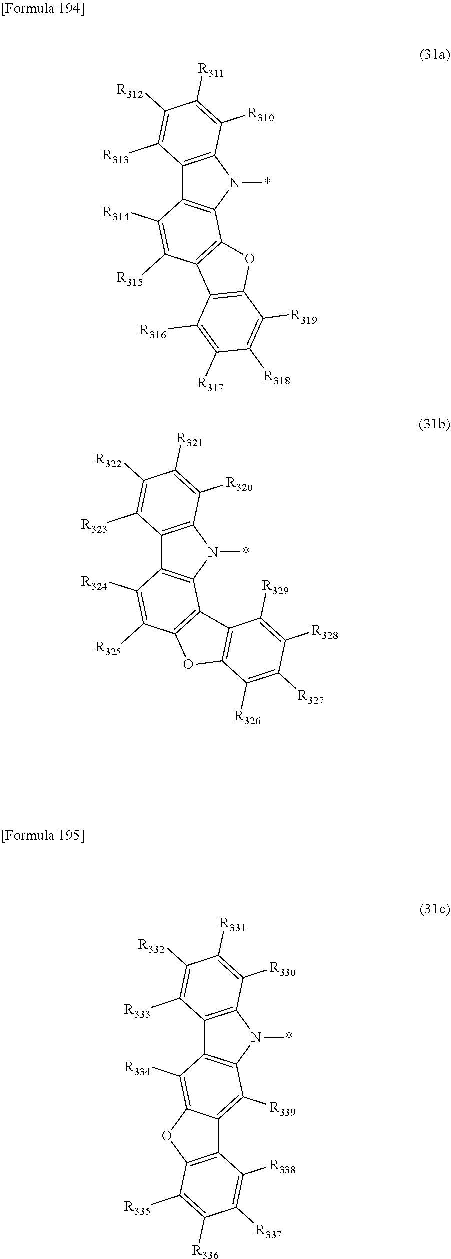

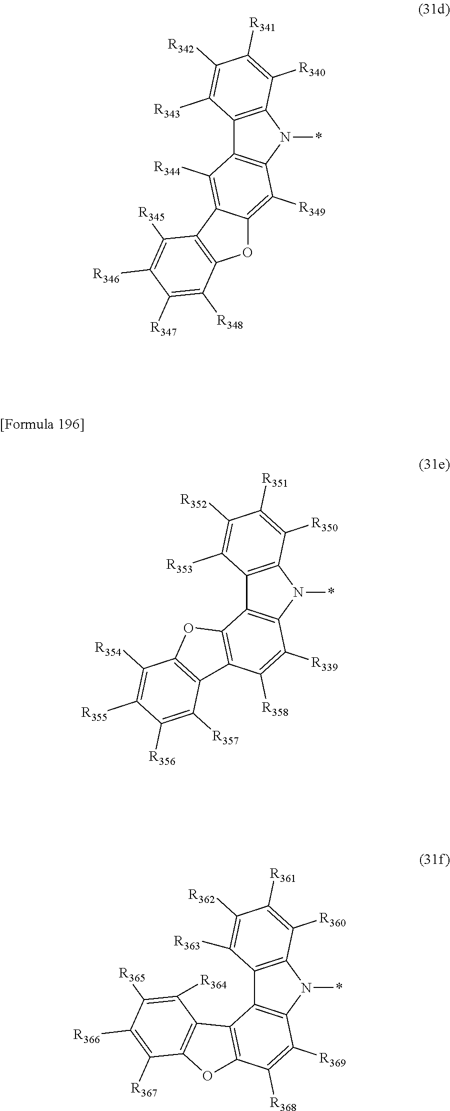

In the formulae (31a), (31b), (31c), (31d), (31e) and (31f):

R₃₁₀ to R₃₁₉ are each independently a hydrogen atom or a substituent;

R₃₂₀ to R₃₂₉ are each independently a hydrogen atom or a substituent;

R₃₃₀ to R₃₃₉ are each independently a hydrogen atom or a substituent;

R₃₄₀ to R₃₄₉ are each independently a hydrogen atom or a substituent;

R₃₅₀ to R₃₅₉ are each independently a hydrogen atom or a substituent;

R₃₆₀ to R₃₆₉ are each independently a hydrogen atom or a substituent;

R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ as a substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms; and

* represents a bonding position to a benzene ring bonded with R₄₀₁ to R₄₀₄.

In the formula (301):

R₃₁ to R₃₈ are each independently a hydrogen atom or a substituent;

R₄₀₁ to R₄₁₂ are each independently a hydrogen atom or a substituent;

R₃₁ to R₃₈ as a substituent and R₄₀₁ to R₄₁₂ as a substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms;

n is 0, 1, 2 or 3;

when a plurality of R₄₀₅ are present, the plurality of R₄₀₅ are mutually the same or different;

when a plurality of R₄₀₆ are present, the plurality of R₄₀₆ are mutually the same or different;

when a plurality of R₄₀₇ are present, the plurality of R₄₀₇ are mutually the same or different; and

when a plurality of R₄₀₈ are present, the plurality of R₄₀₈ are mutually the same or different.

In the formula (31a), a pair of R₃₁₀ and R₃₁₁, a pair of R₃₁₁ and R₃₁₂, a pair of R₃₁₂ and R₃₁₃, a pair of R₃₁₄ and R₃₁₅, a pair of R₃₁₆ and R₃₁₇, a pair of R₃₁₇ and R₃₁₈, and a pair of R₃₁₈ and R₃₁₉ are not mutually bonded.

In the formula (31b), a pair of R₃₂₀ and R₃₂₁, a pair of R₃₂₁ and R₃₂₂, a pair of R₃₂₂ and R₃₂₃, a pair of R₃₂₄ and R₃₂₅, a pair of R₃₂₆ and R₃₂₇, a pair of R₃₂₇ and R₃₂₈, and a pair of R₃₂₈ and R₃₂₉ are not mutually bonded.

In the formula (31c), a pair of R₃₃₀ and R₃₃₁, a pair of R₃₃₁ and R₃₃₂, a pair of R₃₃₂ and R₃₃₃, a pair of R₃₃₅ and R₃₃₆, a pair of R₃₃₆ and R₃₃₇, and a pair of R₃₃₇ and R₃₃₈ are not mutually bonded.

In the formula (31d), a pair of R₃₄₀ and R₃₄₁, a pair of R₃₄₁ and R₃₄₂, a pair of R₃₄₂ and R₃₄₃, a pair of R₃₄₅ and R₃₄₆, a pair of R₃₄₆ and R₃₄₇, and a pair of R₃₄₇ and R₃₄₈ are not mutually bonded.

In the formula (31e), a pair of R₃₅₀ and R₃₅₁, a pair of R₃₅₁ and R₃₅₂, a pair of R₃₅₂ and R₃₅₃, a pair of R₃₅₄ and R₃₅₅, a pair of R₃₅₅ and R₃₅₆, a pair of R₃₅₆ and R₃₅₇, and a pair of R₃₅₈ and R₃₅₉ are not mutually bonded.

In the formula (31f), a pair of R₃₆₀ and R₃₆₁, a pair of R₃₆₁ and R₃₆₂, a pair of R₃₆₂ and R₃₆₃, a pair of R₃₆₄ and R₃₆₅, a pair of R₃₆₅ and R₃₆₆, a pair of R₃₆₆ and R₃₆₇, and a pair of R₃₆₈ and R₃₆₉ are not mutually bonded.

In the compound of the third exemplary embodiment, n is preferably 0 or 1.

The compound of the third exemplary embodiment is preferably represented by a formula (302) below.

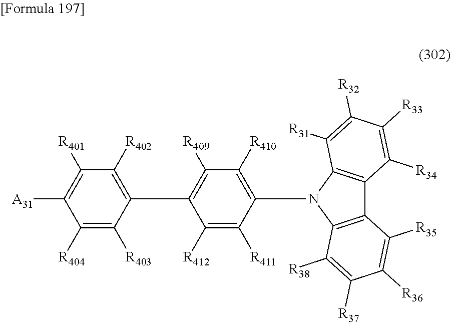

A₃₁, R₄₀₁ to R₄₀₄, R₄₀₉ to R₄₁₂ and R₃₁ to R₃₈ in the formula (302) respectively represent the same as A₃₁, R₄₀₁ to R₄₀₄, R₄₀₉ to R₄₁₂ and R₃₁ to R₃₈ in the formula (301).

In the compound of the third exemplary embodiment, A₃₀ is preferably a group represented by the formula (31a) or (31d).

The compound of the third exemplary embodiment is preferably represented by a formula (303) below.

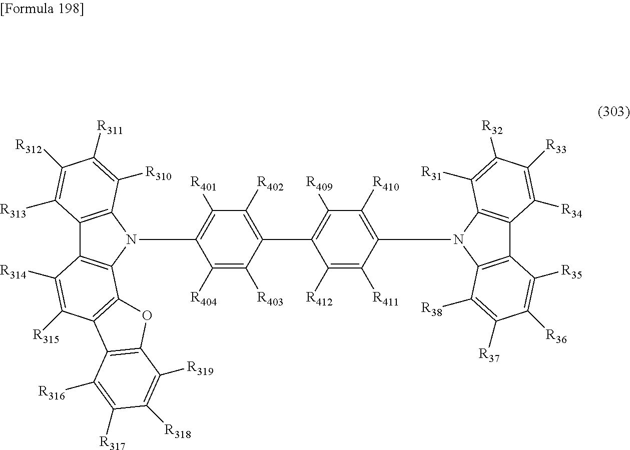

R₄₀₁ to R₄₀₄, R₄₀₉ to R₄₁₂ and R₃₁ to R₃₈ in the formula (303) respectively represent the same as R₄₀₁ to R₄₀₄, R₄₀₉ to R₄₁₂ and R₃₁ to R₃₈ in the formula (301).

In the compound of the third exemplary embodiment, R₃₁ to R₃₈ are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound of the third exemplary embodiment, R₃₁ to R₃₈ are more preferably each independently a hydrogen atom, a substituted or unsubstituted aryl group having 6 to 30 ring carbon atoms, or a substituted or unsubstituted heterocyclic group having 5 to 30 ring atoms.

In the compound of the third exemplary embodiment, R₃₁ to R₃₈ are further preferably each independently a hydrogen atom, or a substituted or unsubstituted aryl group having 6 to 30 ring carbon atoms.

In the compound of the third exemplary embodiment, R₃₁ to R₃₈ are further more preferably a hydrogen atom.

In the compound of the third exemplary embodiment, R₄₀₁ to R₄₁₂ are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound of the third exemplary embodiment, R₄₀₁ to R₄₁₂ are more preferably each independently a hydrogen atom, a substituted or unsubstituted aryl group having 6 to 30 ring carbon atoms, or a substituted or unsubstituted heterocyclic group having 5 to 30 ring atoms.

In the compound of the third exemplary embodiment, R₄₀₁ to R₄₁₂ are further preferably each independently a hydrogen atom, or a substituted or unsubstituted aryl group having 6 to 30 ring carbon atoms.

In the compound of the third exemplary embodiment, R₄₀₁ to R₄₁₂ are further more preferably a hydrogen atom.

In the compound of the third exemplary embodiment, it is preferable that R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ are each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound of the third exemplary embodiment, R₄₀₁ to R₄₁₂ and R₃₁ to R₃₈ are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound of the third exemplary embodiment, R₄₀₁ to R₄₁₂ and R₃₁ to R₃₈ are more preferably each independently a hydrogen atom, a substituted or unsubstituted aryl group having 6 to 30 ring carbon atoms, or a substituted or unsubstituted heterocyclic group having 5 to 30 ring atoms.

In the compound of the third exemplary embodiment, R₄₀₁ to R₄₁₂ and R₃₁ to R₃₈ are further preferably each independently a hydrogen atom or a substituted or unsubstituted aryl group having 6 to 30 ring carbon atoms.

In the compound of the third exemplary embodiment, R₄₀₁ to R₄₁₂ and R₃₁ to R₃₈ are further more preferably a hydrogen atom.

In the compound of the third exemplary embodiment, it is more preferable that R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ are each independently a hydrogen atom, a substituted or unsubstituted aryl group having 6 to 30 ring carbon atoms, or a substituted or unsubstituted heterocyclic group having 5 to 30 ring atoms.

In the compound of the third exemplary embodiment, it is further preferable that R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ are each independently a hydrogen atom or a substituted or unsubstituted aryl group having 6 to 30 ring carbon atoms.

In the compound of the third exemplary embodiment, it is further more preferable that R₃₁₀ to R₃₁₉, R₃₂₀ to R₃₂₉, R₃₃₀ to R₃₃₉, R₃₄₀ to R₃₄₉, R₃₅₀ to R₃₅₉ and R₃₆₀ to R₃₆₉ are a hydrogen atom.

It should be noted that the compound of the third exemplary embodiment also corresponds to a compound with an exemplary arrangement of the compound M3 described in the first exemplary embodiment. Accordingly, specific examples of the compound of the third exemplary embodiment are also shown in the specific examples of the compound M3 described in the first exemplary embodiment.

### Organic EL Device

An organic EL device in an exemplary arrangement according to the third exemplary embodiment is an organic EL device in which the compound M3 in the organic EL device according to the first exemplary embodiment is replaced by the compound of the third exemplary embodiment.

The compound of the third exemplary embodiment allows an organic EL device to have higher performance such as improved luminous efficiency.

Accordingly, an organic EL device in an exemplary arrangement of the third exemplary embodiment also has high-performance such as high luminous efficiency.

### Fourth Exemplary Embodiment

### Compound

A compound of a fourth exemplary embodiment is a compound represented by a formula (310) below. A compound falling under the formula (310) among the specific examples of M3 of the first embodiment is also used as a specific example of the compound of the fourth exemplary embodiment.

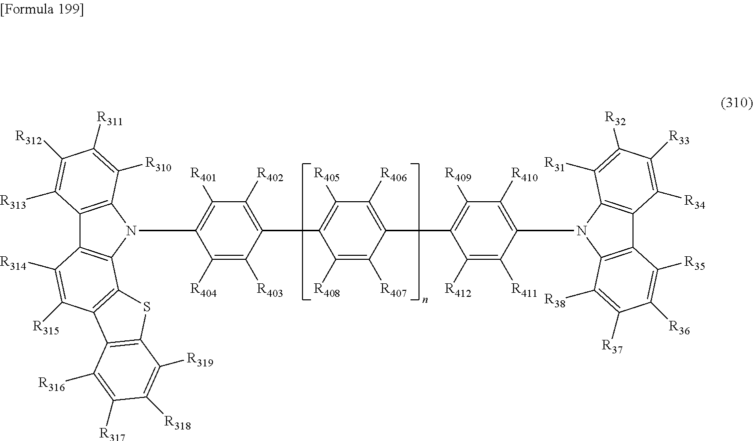

In the formula (310):

R₃₁₀ to R₃₁₉ are each independently a hydrogen atom or a substituent;

R₃₁ to R₃₈ are each independently a hydrogen atom or a substituent;

R₄₀₁ to R₄₁₂ are each independently a hydrogen atom or a substituent;

R₃₁₀ to R₃₁₉ as a substituent, R₃₁ to R₃₈ as a substituent and R₄₀₁ to R₄₁₂ as a substituent are each independently a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, or a substituted or unsubstituted arylthio group having 6 to 30 ring carbon atoms;

n is 0, 1, 2 or 3;

when a plurality of R₄₀₅ are present, the plurality of R₄₀₅ are mutually the same or different;

when a plurality of R₄₀₆ are present, the plurality of R₄₀₆ are mutually the same or different;

when a plurality of R₄₀₇ are present, the plurality of R₄₀₇ are mutually the same or different; and

when a plurality of R₄₀₈ are present, the plurality of R₄₀₈ are mutually the same or different.

In the compound of the fourth exemplary embodiment, a pair of R₃₁₀ and R₃₁₁, a pair of R₃₁₁ and R₃₁₂, a pair of R₃₁₂ and R₃₁₃, a pair of R₃₁₃ and R₃₁₄, a pair of R₃₁₄ and R₃₁₅, a pair of R₃₁₅ and R₃₁₆, a pair of R₃₁₆ and R₃₁₇, a pair of R₃₁₇ and R₃₁₈, and a pair of R₃₁₈ and R₃₁₉ are not mutually bonded.

In the compound of the fourth exemplary embodiment, n is preferably 0 or 1.

The compound of the fourth exemplary embodiment is preferably represented by a formula (311) below.

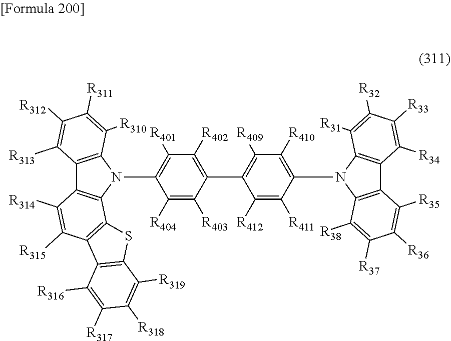

R₃₁₀ to R₃₁₉, R₄₀₁ to R₄₀₄, R₄₀₉ to R₄₁₂ and R₃₁ to R₃₈ in the formula (311) respectively represent the same as R₃₁₀ to R₃₁₉, R₄₀₁ to R₄₀₄, R₄₀₉ to R₄₁₂ and R₃₁ to R₃₈ in the formula (310).

In the compound of the fourth exemplary embodiment, R₃₁ to R₃₈ are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound of the fourth exemplary embodiment, R₃₁ to R₃₈ are more preferably each independently a hydrogen atom, a substituted or unsubstituted aryl group having 6 to 30 ring carbon atoms, or a substituted or unsubstituted heterocyclic group having 5 to 30 ring atoms.

In the compound of the fourth exemplary embodiment, R₃₁ to R₃₈ are further preferably each independently a hydrogen atom or a substituted or unsubstituted aryl group having 6 to 30 ring carbon atoms.

In the compound of the fourth exemplary embodiment, R₃₁ to R₃₈ are further more preferably a hydrogen atom.

In the compound of the fourth exemplary embodiment, R₄₀₁ to R₄₁₂ are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound of the fourth exemplary embodiment, R₄₀₁ to R₄₁₂ are more preferably each independently a hydrogen atom, a substituted or unsubstituted aryl group having 6 to 30 ring carbon atoms, or a substituted or unsubstituted heterocyclic group having 5 to 30 ring atoms.

In the compound of the fourth exemplary embodiment, R₄₀₁ to R₄₁₂ are further preferably each independently a hydrogen atom or a substituted or unsubstituted aryl group having 6 to 30 ring carbon atoms.

In the compound of the fourth exemplary embodiment, R₄₀₁ to R₄₁₂ are further more preferably a hydrogen atom.

In the compound of the fourth exemplary embodiment, R₄₀₁ to R₄₁₂ and R₃₁ to R₃₈ are preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound of the fourth exemplary embodiment, R₄₀₁ to R₄₁₂ and R₃₁ to R₃₈ are more preferably each independently a hydrogen atom, a substituted or unsubstituted aryl group having 6 to 30 ring carbon atoms, or a substituted or unsubstituted heterocyclic group having 5 to 30 ring atoms.

In the compound of the fourth exemplary embodiment, R₄₀₁ to R₄₁₂ and R₃₁ to R₃₈ are further preferably each independently a hydrogen atom or a substituted or unsubstituted aryl group having 6 to 30 ring carbon atoms.

In the compound of the fourth exemplary embodiment, R₄₀₁ to R₄₁₂ and R₃₁ to R₃₈ are further more preferably a hydrogen atom.

In the compound of the fourth exemplary embodiment, R₃₁₀ to R₃₁₉ are further preferably each independently a hydrogen atom, a substituted or unsubstituted aryl group having 6 to 30 ring carbon atoms, a substituted or unsubstituted heterocyclic group having 5 to 30 ring atoms, or a substituted or unsubstituted alkyl group having 1 to 30 carbon atoms.

In the compound of the fourth exemplary embodiment, R₃₁₀ to R₃₁₉ are more preferably each independently a hydrogen atom, a substituted or unsubstituted aryl group having 6 to 30 ring carbon atoms, or a substituted or unsubstituted heterocyclic group having 5 to 30 ring atoms.

In the compound of the fourth exemplary embodiment, R₃₁₀ to R₃₁₉ are further preferably each independently a hydrogen atom or a substituted or unsubstituted aryl group having 6 to 30 ring carbon atoms.

In the compound of the fourth exemplary embodiment, R₃₁₀ to R₃₁₉ are further more preferably a hydrogen atom.

It should be noted that the compound of the fourth exemplary embodiment also corresponds to a compound with an exemplary arrangement of the compound M3 described in the first exemplary embodiment. Accordingly, specific examples of the compound of the fourth exemplary embodiment are also shown in the specific examples of the compound M3 described in the first exemplary embodiment.

### Organic EL Device

An organic EL device in an exemplary arrangement according to the fourth exemplary embodiment is an organic EL device in which the compound M3 in the organic EL device according to the first exemplary embodiment is replaced by the compound of the fourth exemplary embodiment.

The compound of the fourth exemplary embodiment allows an organic EL device to have higher performance such as improved luminous efficiency.

Accordingly, an organic EL device in an exemplary arrangement of the fourth exemplary embodiment also has high-performance such as high luminous efficiency.

### Fifth Exemplary Embodiment

### Electronic Device

An electronic device according to a fifth exemplary embodiment is installed with any one of the organic EL devices according to the above exemplary embodiments. Examples of the electronic device include a display device and a light-emitting device. Examples of the display device include a display component (e.g., an organic EL panel module), TV, mobile phone, tablet and personal computer. Examples of the light-emitting device include an illuminator and a vehicle light.

### Sixth Exemplary Embodiment

### Organic-EL-Device Material

An organic-EL-device material of a sixth exemplary embodiment contains at least one of the compound of the third exemplary embodiment or the compound of the fourth exemplary embodiment.

The organic-EL-device material of the sixth exemplary embodiment allows an organic EL device to have higher performance such as improved luminous efficiency.

The organic-EL-device material according to the sixth exemplary embodiment may further contain an additional compound. When the organic-EL-device material according to the sixth exemplary embodiment further contains the additional compound, the additional compound may be solid or liquid.

### Modification of Embodiment(s)

The scope of the invention is not limited by the above-described exemplary embodiments but includes any modification and improvement as long as such modification and improvement are compatible with the invention.

For instance, the emitting layer is not limited to a single layer, but may be provided by laminating a plurality of emitting layers. When the organic EL device has a plurality of emitting layers, it is only required that at least one of the emitting layers satisfies the conditions described in the above exemplary embodiments. For instance, the rest of the emitting layers may be a fluorescent emitting layer or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device, in which a plurality of emitting units are laminated via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least any of holes, electrons, and excitons.

For instance, when the blocking layer is provided in contact with the side of the emitting layer close to the cathode, the blocking layer permits transport of electrons and blocks holes from reaching a layer provided closer to the cathode (e.g., the electron transporting layer) than the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer to the anode (e.g., the hole transporting layer) than the blocking layer. When the organic EL device includes the hole transporting layer, the blocking layer is preferably interposed between the emitting layer and the hole transporting layer.

Alternatively, the blocking layer may be provided adjacent to the emitting layer so that excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) than the blocking layer.

The emitting layer is preferably bonded with the blocking layer.

Specific structure, shape and the like of the components in the invention may be designed in any manner as long as an object of the invention can be achieved.

### Other Explanations

Herein, numerical ranges represented by “x to y” represents a range whose lower limit is the value (x) recited before “to” and whose upper limit is the value (y) recited after “to.”

Rx and Ry are mutually bonded to form a ring, which means herein, for instance, that Rx and Ry contain a carbon atom, a nitrogen atom, an oxygen atom, a sulfur atom or a silicon atom, the atom (a carbon atom, a nitrogen atom, an oxygen atom, a sulfur atom or a silicon atom) contained in Rx and the atom (a carbon atom, a nitrogen atom, an oxygen atom, a sulfur atom or a silicon atom) contained in Ry are mutually bonded via a single bond, a double bond, a triple bond or a divalent linking group to form a ring having 5 or more ring atoms (specifically, a heterocyclic ring or an aromatic hydrocarbon ring). x represents a number, a character or a combination of a number and a character. y represents a number, a character or a combination of a number and a character.

The divalent linking group is not particularly limited and is exemplified by —O—, —CO—, —CO₂—, —S—, —SO—, —SO₂—, —NH—, —NRa—, and a group obtained by combining two or more linking groups of these linking group.

Specific examples of the heterocyclic ring include a cyclic structure (heterocyclic ring) obtained by removing a bond from a “heteroaryl group Sub₂” exemplarily shown in the later-described “Description of Each Substituent in Formula.” The heterocyclic ring may have a substituent.

Specific examples of the aromatic hydrocarbon ring include cyclic structures (aromatic hydrocarbon rings) obtained by removing a bond from an “aryl group Sub₁” exemplarily shown in the later-described “Description of Each Substituent in Formula.” The aromatic hydrocarbon ring may have a substituent.

Examples of Ra include a substituted or unsubstituted alkyl group Sub₃ having 1 to 30 carbon atoms, a substituted or unsubstituted aryl group Sub₁ having 6 to 30 ring carbon atoms, and a substituted or unsubstituted heteroaryl group Sub₂ having 5 to 30 ring atoms, which are exemplarily shown in the later-described “Description of Each Substituent in Formula.”

Rx and Ry are mutually bonded to form a ring, which means, for instance, that: an atom contained in Rx₁ and an atom contained in Ry₁ in a molecular structure represented by a formula (E1) below form a ring (cyclic structure) E represented by a formula (E2); an atom contained in Rx₁ and an atom contained in Ry₁ in a molecular structure represented by a formula (F1) below form a ring (cyclic structure) F represented by a formula (F2); an atom contained in Rx₁ and an atom contained in Ry₁ in a molecular structure represented by a formula (G1) below form a ring (cyclic structure) G represented by a formula (G2); an atom contained in Rx₁ and an atom contained in Ry₁ in a molecular structure represented by a formula (H1) below form a ring (cyclic structure) H represented by a formula (H2); and an atom contained in Rx₁ and an atom contained in Ry₁ in a molecular structure represented by a formula (I1) below form a ring (cyclic structure) I represented by a formula (I2).

In the formulae (E1) to (I1), * each independently represents a bonding position to another atom in a molecule. Two * in the formula (E1) correspond one-to-one to two * in the formula (E2). Two * in the formula (F1) correspond one-to-one to two * in the formula (F2). Two * in the formula (G1) correspond one-to-one to two * in the formula (G2). Two * in the formula (H1) correspond one-to-one to two * in the formula (H2). Two * in the formula (I1) correspond one-to-one to two * in the formula (I2).

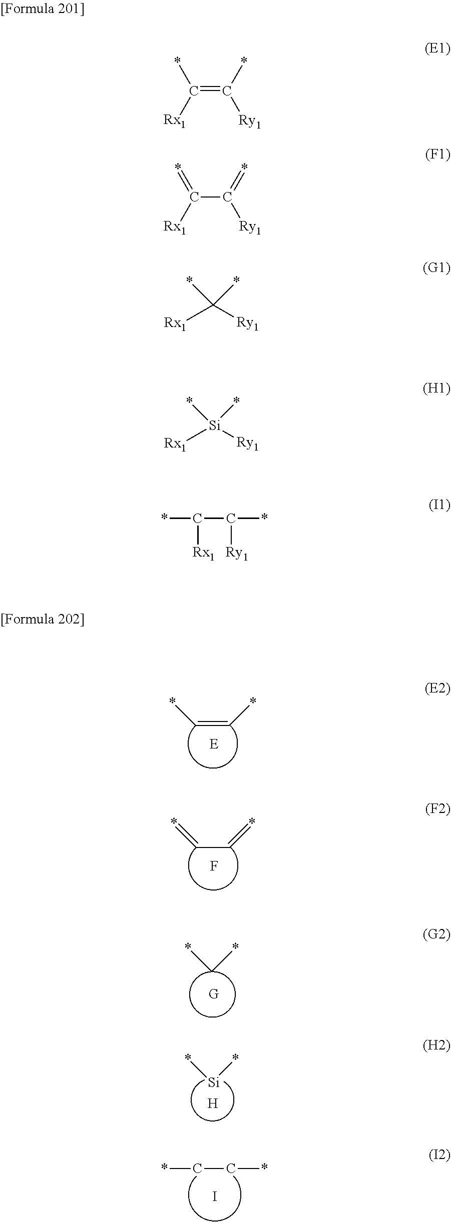

In the molecular structures represented by the respective formulae (E2) to (I2), E to I each represent a cyclic structure (the ring having 5 or more ring atoms). In the formulae (E2) to (I2), * each independently represents a bonding position to another atom in a molecule. Two * in the formula (E2) correspond one-to-one to two * in the formula (E1). Similarly, two * in each of the formulae (F2) to (I2) correspond one-to-one to two * in in each of the formulae (F1) to (I1).

For instance, when in the formula (E1), Rx₁ and Ry₁ are mutually bonded to form the ring E in the formula (E2) and the ring E is an unsubstituted benzene ring, the molecular structure represented by the formula (E1) is a molecular structure represented by a formula (E3) below. Herein, two * in the formula (E3) correspond one-to-one to two * in each of the formulae (E2) and (E1).

For instance, when in the formula (E1), Rx₁ and Ry₁ are mutually bonded to form the ring E in the formula (E2) and the ring E is an unsubstituted pyrrole ring, the molecular structure represented by the formula (E1) is a molecular structure represented by a formula (E4) below. Herein, two * in the formula (E4) correspond one-to-one to two * in each of the formulae (E2) and (E1). In the formulae (E3) and (E4), * each independently represents a bonding position to another atom in a molecule.

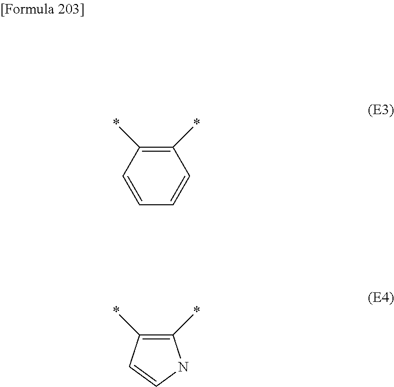 [SMILES: *c1ccccc1* ; *c1cc[nH]c1*]

Herein, the ring carbon atoms refer to the number of carbon atoms among atoms forming a ring of a compound (e.g., a monocyclic compound, fused-ring compound, crosslinking compound, carbon ring compound, and heterocyclic compound) in which the atoms are bonded to each other to form the ring. When the ring is substituted by a substituent(s), carbon atom(s) contained in the substituent(s) is not counted in the ring carbon atoms. Unless specifically described, the same applies to the “ring carbon atoms” described later. For instance, a benzene ring has 6 ring carbon atoms, a naphthalene ring has 10 ring carbon atoms, a pyridinyl group has 5 ring carbon atoms, and a furanyl group has 4 ring carbon atoms. When a benzene ring and/or a naphthalene ring is substituted by a substituent (e.g., an alkyl group), the number of carbon atoms of the alkyl group is not counted in the number of the ring carbon atoms. When a fluorene ring is substituted by a substituent (e.g., a fluorene ring) (i.e., a spirofluorene ring is included), the number of carbon atoms of the fluorene ring as the substituent is not counted in the number of the ring carbon atoms of the fluorene ring.

Herein, the ring atoms refer to the number of atoms forming a ring of a compound (e.g., a monocyclic compound, fused-ring compound, crosslinking compound, carbon ring compound, and heterocyclic compound) in which the atoms are bonded to each other to form the ring (e.g., monocyclic ring, fused ring, ring assembly). Atom(s) not forming a ring and atom(s) included in a substituent when the ring is substituted by the substituent are not counted in the number of the ring atoms. Unless specifically described, the same applies to the “ring atoms” described later. For instance, a pyridine ring has six ring atoms, a quinazoline ring has ten ring atoms, and a furan ring has five ring atoms. A hydrogen atom(s) and/or an atom(s) of a substituent which are bonded to carbon atoms of a pyridine ring and/or quinazoline ring are not counted in the ring atoms. When a fluorene ring is substituted by a substituent (e.g., a fluorene ring) (i.e., a spirofluorene ring is included), the number of atoms of the fluorene ring as the substituent is not counted in the number of the ring atoms of the fluorene ring.

### Description of Each Substituent in Formula Herein

The aryl group (occasionally referred to as an aromatic hydrocarbon group) herein is exemplified by an aryl group Sub₁. The aryl group Sub₁ is at least one group selected from the group consisting of a phenyl group, biphenyl group, terphenyl group, naphthyl group, anthryl group, phenanthryl group, fluorenyl group, pyrenyl group, chrysenyl group, fluoranthenyl group, benz[a]anthryl group, benzo[c]phenanthryl group, triphenylenyl group, benzo[k]fluoranthenyl group, benzo[g]chrysenyl group, benzo[b]triphenylenyl group, picenyl group, and perylenyl group.

Herein, the aryl group Sub₁ preferably has 6 to 30 ring carbon atoms, more preferably 6 to 22 ring carbon atoms, further preferably 6 to 20 ring carbon atoms, further more preferably 6 to 14 ring carbon atoms, still further preferably 6 to 12 ring carbon atoms. Among the aryl group Sub₁, a phenyl group, biphenyl group, naphthyl group, phenanthryl group, terphenyl group and fluorenyl group are preferable. A carbon atom in a position 9 of each of 1-fluorenyl group, 2-fluorenyl group, 3-fluorenyl group and 4-fluorenyl group is preferably substituted by a substituted or unsubstituted alkyl group Sub₃ or a substituted or unsubstituted aryl group Sub₁ described later herein.

The heteroaryl group (occasionally referred to as a heterocyclic group, heteroaromatic ring group or aromatic heterocyclic group) herein is exemplified by a heterocyclic group Sub₂. The heterocyclic group Sub₂ is a group containing, as a hetero atom(s), at least one atom selected from the group consisting of nitrogen, sulfur, oxygen, silicon, selenium atom and germanium atom. The heterocyclic group Sub₂ preferably contains, as a hetero atom(s), at least one atom selected from the group consisting of nitrogen, sulfur and oxygen.

The heterocyclic group Sub₂ herein are, for instance, at least one group selected from the group consisting of a pyridyl group, pyrimidinyl group, pyrazinyl group, pyridazinyl group, triazinyl group, quinolyl group, isoquinolinyl group, naphthyridinyl group, phthalazinyl group, quinoxalinyl group, quinazolinyl group, phenanthridinyl group, acridinyl group, phenanthrolinyl group, pyrrolyl group, imidazolyl group, pyrazolyl group, triazolyl group, tetrazolyl group, indolyl group, benzimidazolyl group, indazolyl group, imidazopyridinyl group, benzotriazolyl group, carbazolyl group, furyl group, thienyl group, oxazolyl group, thiazolyl group, isoxazolyl group, isothiazolyl group, oxadiazolyl group, thiadiazolyl group, benzofuranyl group, benzothienyl group, benzoxazolyl group, benzothiazolyl group, benzisoxazolyl group, benzisothiazolyl group, benzoxadiazolyl group, benzothiadiazolyl group, dibenzofuranyl group, dibenzothienyl group, piperidinyl group, pyrrolidinyl group, piperazinyl group, morpholyl group, phenazinyl group, phenothiazinyl group, and phenoxazinyl group.

Herein, the heterocyclic group Sub₂ preferably has 5 to 30 ring atoms, more preferably 5 to 20 ring atoms, further preferably 5 to 14 ring atoms. Among the above heterocyclic group Sub₂, a 1-dibenzofuranyl group, 2-dibenzofuranyl group, 3-dibenzofuranyl group, 4-dibenzofuranyl group, 1-dibenzothienyl group, 2-dibenzothienyl group, 3-dibenzothienyl group, 4-dibenzothienyl group, 1-carbazolyl group, 2-carbazolyl group, 3-carbazolyl group, 4-carbazolyl group, and 9-carbazolyl group are further more preferable. A nitrogen atom in position 9 of 1-carbazolyl group, 2-carbazolyl group, 3-carbazolyl group and 4-carbazolyl group is preferably substituted by the substituted or unsubstituted aryl group Sub₁ or the substituted or unsubstituted heterocyclic group Sub₂ described herein.

Herein, the heterocyclic group Sub₂ may be a group derived from any one of partial structures represented by formulae (XY-1) to (XY-18) below.

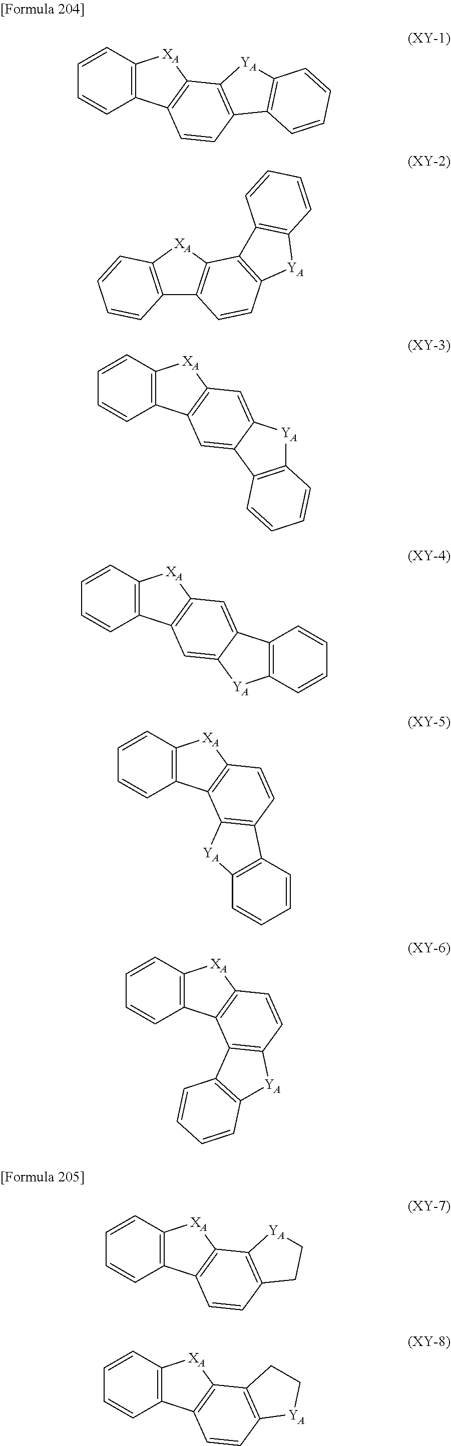

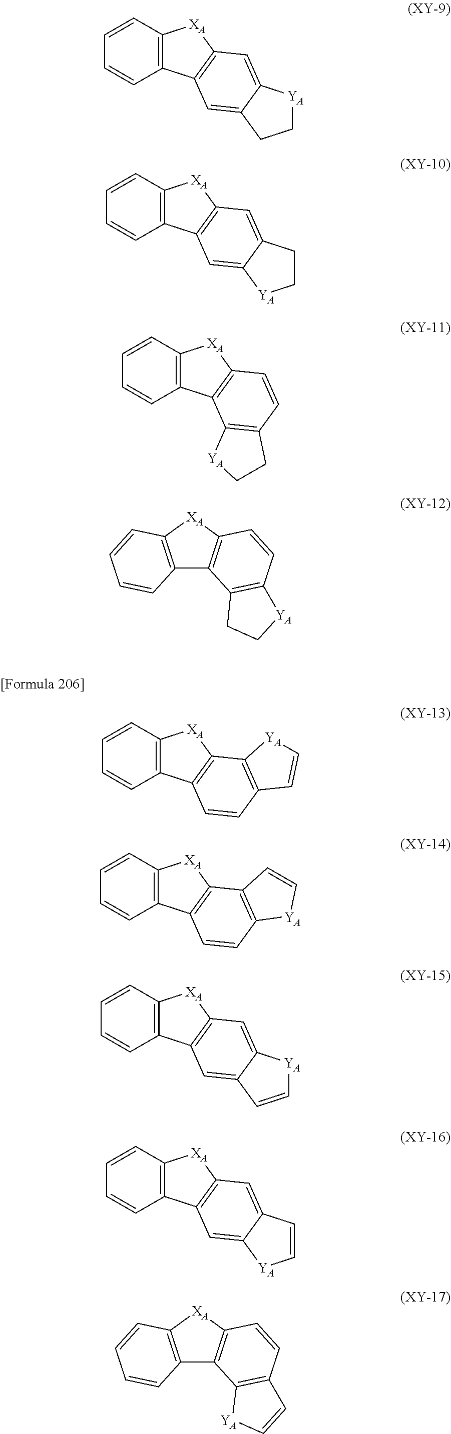

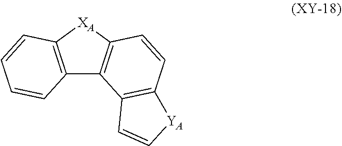

In the formulae (XY-1) to (XY-18), X_{A} and Y_{A} each independently represent a hetero atom, and preferably represent an oxygen atom, sulfur atom, selenium atom, silicon atom or germanium atom. Each of the partial structures represented by the respective formulae (XY-1) to (XY-18) has a bond at any position to provide a heterocyclic group. The heterocyclic group may be substituted.

Herein, the heterocyclic group Sub₂ may be a group represented by one of formulae (XY-19) to (XY-22) below. Moreover, the position of the bond may be changed as needed

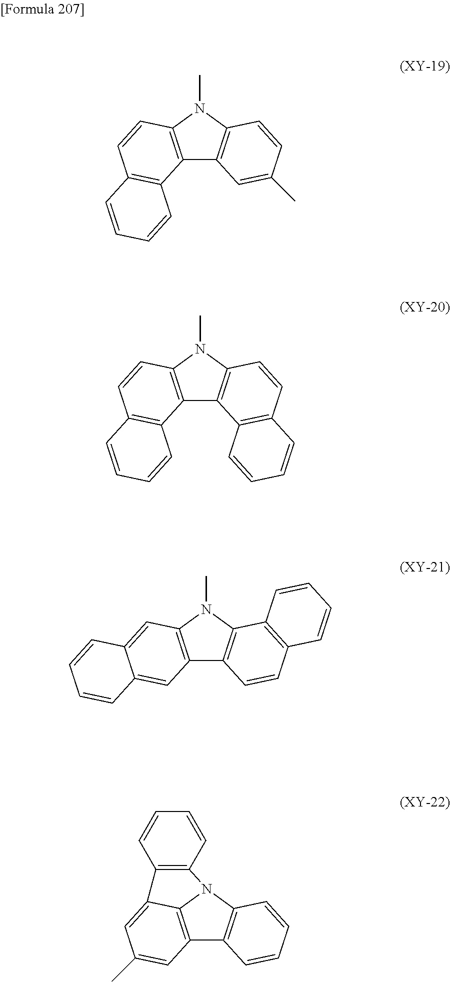

The alkyl group herein may be any one of a linear alkyl group, branched alkyl group and cyclic alkyl group.

The alkyl group herein is exemplified by an alkyl group Sub₃.

The linear alkyl group herein is exemplified by a linear alkyl group Sub₃₁.

The branched alkyl group herein is exemplified by a branched alkyl group Sub₃₂.

The cyclic alkyl group herein is exemplified by a cyclic alkyl group Sub₃₃.

For instance, the alkyl group Sub₃ is at least one group selected from the group consisting of the linear alkyl group Sub₃₁, branched alkyl group Sub₃₂, and cyclic alkyl group Sub₃₃.

The linear alkyl group Sub₃₁ or branched alkyl group Sub₃₂ is exemplified by at least one group selected from the group consisting of a methyl group, ethyl group, n-propyl group, isopropyl group, n-butyl group, s-butyl group, isobutyl group, t-butyl group, n-pentyl group, n-hexyl group, n-heptyl group, n-octyl group, n-nonyl group, n-decyl group, n-undecyl group, n-dodecyl group, n-tridecyl group, n-tetradecyl group, n-pentadecyl group, n-hexadecyl group, n-heptadecyl group, n-octadecyl group, neopentyl group, amyl group, isoamyl group, 1-methylpentyl group, 2-methylpentyl group, 1-pentylhexyl group, 1-butylpentyl group, 1-heptyloctyl group, and 3-methylpentyl group.

Herein, the linear alkyl group Sub₃₁ or branched alkyl group Sub₃₂ preferably has 1 to 30 carbon atoms, more preferably 1 to 20 carbon atoms, further preferably 1 to 10 carbon atoms, further more preferably 1 to 6 carbon atoms. The linear alkyl group Sub₃₁ or branched alkyl group Sub₃₂ is further more preferably a methyl group, ethyl group, propyl group, isopropyl group, n-butyl group, s-butyl group, isobutyl group, t-butyl group, n-pentyl group, n-hexyl group, amyl group, isoamyl group and neopentyl group.

Herein, the cyclic alkyl group Sub₃₃ is exemplified by a cycloalkyl group Sub₃₃₁.

The cycloalkyl group Sub₃₃₁ herein is exemplified by at least one group selected from the group consisting of a cyclopropyl group, cyclobutyl group, cyclopentyl group, cyclohexyl group, 4-methylcyclohexyl group, adamantyl group and norbornyl group. The cycloalkyl group Sub₃₃₁ preferably has 3 to 30 ring carbon atoms, more preferably 3 to 20 ring carbon atoms, further preferably 3 to 10 ring carbon atoms, further more preferably 5 to 8 ring carbon atoms. Among the cycloalkyl group Sub₃₃₁, a cyclopentyl group and a cyclohexyl group are further more preferable.

Herein, an alkyl halide group is exemplified by an alkyl halide group Sub₄. The alkyl halide group Sub₄ is provided by substituting the alkyl group Sub₃ with at least one halogen atom, preferably at least one fluorine atom.

Herein, the alkyl halide group Sub₄ is exemplified by at least one group selected from the group consisting of a fluoromethyl group, difluoromethyl group, trifluoromethyl group, fluoroethyl group, trifluoromethylmethyl group, trifluoroethyl group, and pentafluoroethyl group.

Herein, a substituted silyl group is exemplified by a substituted silyl group Sub₅. The substituted silyl group Sub₅ is exemplified by at least one group selected from the group consisting of an alkylsilyl group Sub₅₁ and an arylsilyl group Sub₅₂.

Herein, the alkylsilyl group Sub₅₁ is exemplified by a trialkylsilyl group Sub₅₁₁ having the above-described alkyl group Sub₃.

The trialkylsilyl group Sub₅₁₁ is exemplified by at least one group selected from the group consisting of a trimethylsilyl group, triethylsilyl group, tri-n-butylsilyl group, tri-n-octylsilyl group, triisobutylsilyl group, dimethylethylsilyl group, dimethylisopropylsilyl group, dimethyl-n-propylsilyl group, dimethyl-n-butylsilyl group, dimethyl-t-butylsilyl group, diethylisopropylsilyl group, vinyl dimethylsilyl group, propyldimethylsilyl group, and triisopropylsilyl group. Three alkyl groups Sub₃ in the trialkylsilyl group Sub₅₁₁ may be mutually the same or different.

Herein, the arylsilyl group Sub₅₂ is exemplified by at least one group selected from the group consisting of a dialkylarylsilyl group Sub₅₂₁, alkyldiarylsilyl group Sub₅₂₂ and triarylsilyl group Sub₅₂₃.

The dialkylarylsilyl group Sub₅₂₁ is exemplified by a dialkylarylsilyl group including two alkyl groups Sub₃ and one aryl group Sub₁. The dialkylarylsilyl group Sub₅₂₁ preferably has 8 to 30 carbon atoms.

The alkyldiarylsilyl group Sub₅₂₂ is exemplified by an alkyldiarylsilyl group including one alkyl group Sub₃ and two aryl groups Sub₁. The alkyldiarylsilyl group Sub₅₂₂ preferably has 13 to 30 carbon atoms.

The triarylsilyl group Sub₅₂₃ is exemplified by a triarylsilyl group including three aryl groups Sub₁. The triarylsilyl group Sub₅₂₃ preferably has 18 to 30 carbon atoms.

Herein, a substituted or unsubstituted alkyl sulfonyl group is exemplified by an alkyl sulfonyl group Sub₆. The alkyl sulfonyl group Sub₆ is represented by —SO₂R_{w}. R_{w} in —SO₂R_{w} represents a substituted or unsubstituted alkyl group Sub₃ described above.

Herein, an aralkyl group (occasionally referred to as an arylalkyl group) is exemplified by an aralkyl group Sub₇. An aryl group in the aralkyl group Sub₇ includes, for instance, at least one of the above-described aryl group Sub₁ or the above-described heteroaryl group Sub₂.

The aralkyl group Sub₇ herein is preferably a group having the aryl group Sub₁ and is represented by —Z₃—Z₄. Z₃ is exemplified by an alkylene group corresponding to the above alkyl group Sub₃. Z₄ is exemplified by the above aryl group Sub₁. In this aralkyl group Sub₇, an aryl moiety has 6 to 30 carbon atoms (preferably 6 to 20 carbon atoms, more preferably 6 to 12 carbon atoms) and an alkyl moiety has 1 to 30 carbon atoms (preferably 1 to 20 carbon atoms, more preferably 1 to 10 carbon atoms, further preferably 1 to 6 carbon atoms). The aralkyl group Sub₇ is exemplified by at least one group selected from the group consisting of a benzyl group, 2-phenylpropane-2-yl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

The alkoxy group herein is exemplified by an alkoxy group Sub₈. The alkoxy group Sub₈ is represented by —OZ₁. Z₁ is exemplified by the above alkyl group Sub₃. The alkoxy group Sub₈ is exemplified by at least one group selected from the group consisting of a methoxy group, ethoxy group, propoxy group, butoxy group, pentyloxy group and hexyloxy group. The alkoxy group Sub₈ preferably has 1 to 30 carbon atoms, more preferably 1 to 20 carbon atoms.

Herein, an alkoxy halide group is exemplified by an alkoxy halide group Sub₉. The alkoxy halide group Sub₉ is provided by substituting the alkoxy group Sub₈ with at least one halogen atom, preferably at least one fluorine atom.

Herein, an aryloxy group (occasionally referred to as an arylalkoxy group) is exemplified by an arylalkoxy group Sub₁₀. An aryl group in the arylalkoxy group Sub₁₀ includes at least one of the aryl group Sub₁ or the heteroaryl group Sub₂.

The arylalkoxy group Sub₁₀ herein is represented by —OZ₂. Z₂ is exemplified by the aryl group Sub₁ or the heteroaryl group Sub₂. The arylalkoxy group Sub₁₀ preferably has 6 to 30 ring carbon atoms, more preferably 6 to 20 ring carbon atoms. The arylalkoxy group Sub₁₀ is exemplified by a phenoxy group.

Herein, a substituted amino group is exemplified by a substituted amino group Sub₁₁. The substituted amino group Sub₁₁ is exemplified by at least one group selected from the group consisting of an arylamino group Sub₁₁₁ and an alkylamino group Sub₁₁₂.

The arylamino group Sub₁₁₁ is represented by —NHR_{V1} or —N(R_{V1})₂. R_{V1} is exemplified by the aryl group Sub₁. Two R_{V1} in —N(R_{V1})₂ are mutually the same or different.

The alkylamino group Sub₁₁₂ is represented by —NHR_{V2} or —N(R_{V2})₂. R_{V2} is exemplified by the alkyl group Sub₃. Two R_{V2} in —N(R_{V2})₂ are mutually the same or different.

Herein, the alkenyl group is exemplified by an alkenyl group Sub₁₂. The alkenyl group Sub₁₂, which is linear or branched, is exemplified by at least one group selected from the group consisting of a vinyl group, propenyl group, butenyl group, oleyl group, eicosapentaenyl group, docosahexaenyl group, styryl group, 2,2-diphenylvinyl group, 1,2,2-triphenylvinyl group, and 2-phenyl-2-propenyl group.

The alkynyl group herein is exemplified by an alkynyl group Sub₁₃. The alkynyl group Sub₁₃ may be linear or branched, and is exemplified by at least one group selected from the group consisting of an ethynyl group, a propynyl group and a 2-phenylethynyl group.

The alkylthio group herein is exemplified by an alkylthio group Sub₁₄.

The alkylthio group Sub₁₄ is represented by —SR_{V3}. R_{V3} is exemplified by the alkyl group Sub₃. The alkylthio group Sub₁₄ preferably has 1 to 30 carbon atoms, more preferably 1 to 20 carbon atoms.

The arylthio group herein is exemplified by an arylthio group Sub₁₅.

The arylthio group Sub₁₅ is represented by —SR_{V4}. R_{V4} is exemplified by the aryl group Sub₁. The arylthio group Sub₁₅ preferably has 6 to 30 ring carbon atoms, more preferably 6 to 20 ring carbon atoms.

Herein, examples of the halogen atom include a fluorine atom, a chlorine atom, a bromine atom and an iodine atom, among which a fluorine atom is preferable.

A substituted phosphino group herein is exemplified by a substituted phosphino group Sub₁₆. The substituted phosphino group Sub₁₆ is exemplified by a phenyl phosphanyl group.

An arylcarbonyl group herein is exemplified by an arylcarbonyl group Sub₁₇. The arylcarbonyl group Sub₁₇ is represented by —COY′. Y′ is exemplified by the aryl group Sub₁. Herein, the arylcarbonyl group Sub₁₇ is exemplified by at least one group selected from the group consisting of a phenyl carbonyl group, diphenyl carbonyl group, naphthyl carbonyl group, and triphenyl carbonyl group.

An acyl group herein is exemplified by an acyl group Sub₁₈. The acyl group Sub₁₈ is represented by —COR′. R′ is exemplified by the alkyl group Sub₃. The acyl group Sub₁₈ herein is exemplified by at least one group selected from the group consisting of an acetyl group and a propionyl group.

A substituted phosphoryl group herein is exemplified by a substituted phosphoryl group Sub₁₉. The substituted phosphoryl group Sub₁₉ is represented by a formula (P) below.

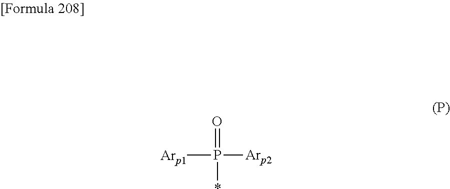

In the formula (P), Ar_{P1} and Ar_{P2} are any one substituent selected from the group consisting of the above alkyl group Sub₃ and the above aryl group Sub₁.

An ester group herein is exemplified by an ester group Sub₂₀. The ester group Sub₂₀ is exemplified by at least one group selected from the group consisting of an alkyl ester group and an aryl ester group.

An alkyl ester group herein is exemplified by an alkyl ester group Sub₂₀₁. The alkyl ester group Sub₂₀₁ is represented by —C(═O)OR^{E}. R^{E} is exemplified by a substituted or unsubstituted alkyl group Sub₃ described above (preferably having 1 to 10 carbon atoms).

An aryl ester group herein is exemplified by an aryl ester group Sub₂₀₂. The aryl ester group Sub₂₀₂ is represented by —C(═O)OR^{Ar}. R^{Ar} is exemplified by a substituted or unsubstituted aryl group Sub₁ described above.

A siloxanyl group herein is exemplified by a siloxanyl group Sub₂₁. The siloxanyl group Sub₂₁ is a silicon compound group through an ether bond. The siloxanyl group Sub₂₁ is exemplified by a trimethylsiloxanyl group.

A carbamoyl group herein is represented by —CONH₂.

A substituted carbamoyl group herein is exemplified by a carbamoyl group Sub₂₂. The carbamoyl group Sub₂₂ is represented by —CONH—Ar^{C} or —CONH—R^{C}. Ar^{C} is exemplified by at least one group selected from the group consisting of a substituted or unsubstituted aryl group Sub₁ (preferably 6 to 10 ring carbon atoms) and a substituted or unsubstituted heteroaryl group Sub₂ (preferably 5 to 14 ring atoms). Ar^{C} may be a group formed by bonding the aryl group Sub₁ and the heteroaryl group Sub₂.

R^{C} is exemplified by a substituted or unsubstituted alkyl group Sub₃ described above (preferably having 1 to 6 carbon atoms).

Herein, “carbon atoms forming a ring (ring carbon atoms)” mean carbon atoms forming a saturated ring, unsaturated ring, or aromatic ring. “Atoms forming a ring (ring atoms)” mean carbon atoms and hetero atoms forming a ring including a saturated ring, unsaturated ring, or aromatic ring.

Herein, a hydrogen atom includes isotope having different numbers of neutrons, specifically, protium, deuterium and tritium.

Hereinafter, an alkyl group Sub₃ means at least one group of a linear alkyl group Sub₃₁, a branched alkyl group Sub₃₂, or a cyclic alkyl group Sub₃₃ described in “Description of Each Substituent.”

Similarly, a substituted silyl group Sub₅ means at least one group of an alkylsilyl group Sub₅₁ or an arylsilyl group Sub₅₂.

Similarly, a substituted amino group Sub₁₁ means at least one group of an arylamino group Sub₁₁₁ or an alkylamino group Sub₁₁₂.

Herein, a substituent for a “substituted or unsubstituted” group is exemplified by a substituent R_{F1}. The substituent R_{F1} is at least one group selected from the group consisting of an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, carbamoyl group Sub₂₂, unsubstituted amino group, unsubstituted silyl group, halogen atom, cyano group, hydroxy group, nitro group, and carboxy group.

Herein, the substituent R_{F1} for a “substituted or unsubstituted” group may be a diaryl boron group (Ar_{B1}Ar_{B2}B—). Ar_{B1} and Ar_{B2} are exemplified by the above-described aryl group Sub₁. Ar_{B1} and Ar_{B2} in Ar_{B1}Ar_{B2}B— are the same or different.

Specific examples and preferable examples of the substituent R_{F1} are the same as those of the substituents described in “Description of Each Substituent” (e.g., an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, and carbamoyl group Sub₂₂).

The substituent R_{F1} for a “substituted or unsubstituted” group may be further substituted by at least one group (hereinafter, also referred to as a substitutent R_{F2}) selected from the group consisting of an aryl group Sub₁, heteroaryl group Sub₂, alkyl group Sub₃, alkyl halide group Sub₄, substituted silyl group Sub₅, alkylsulfonyl group Sub₆, aralkyl group Sub₇, alkoxy group Sub₈, alkoxy halide group Sub₉, arylalkoxy group Sub₁₀, substituted amino group Sub₁₁, alkenyl group Sub₁₂, alkynyl group Sub₁₃, alkylthio group Sub₁₄, arylthio group Sub₁₅, substituted phosphino group Sub₁₆, arylcarbonyl group Sub₁₇, acyl group Sub₁₈, substituted phosphoryl group Sub₁₉, ester group Sub₂₀, siloxanyl group Sub₂₁, carbamoyl group Sub₂₂, unsubstituted amino group, unsubstituted silyl group, halogen atom, cyano group, hydroxy group, nitro group, and carboxy group. Moreover, a plurality of substituents R_{F2} may be bonded to each other to form a ring.

“Unsubstituted” for a “substituted or unsubstituted” group means that a group is not substituted by the above-described substituent R_{F1} but bonded with a hydrogen atom.

Herein, “XX to YY carbon atoms” in the description of “substituted or unsubstituted ZZ group having XX to YY carbon atoms” represent carbon atoms of an unsubstituted ZZ group and do not include carbon atoms of the substituent R_{F1} of the substituted ZZ group.

Herein, “XX to YY atoms” in the description of “substituted or unsubstituted ZZ group having XX to YY atoms” represent atoms of an unsubstituted ZZ group and do not include atoms of the substituent R_{F1} of the substituted ZZ group.

The same description as the above applies to “substituted or unsubstituted” in compounds or partial structures thereof described herein.

Herein, when the substituents are bonded to each other to form a ring, the ring is structured to be a saturated ring, an unsaturated ring, an aromatic hydrocarbon ring or a hetero ring.

Herein, examples of the aromatic hydrocarbon group in the linking group include a divalent or multivalent group obtained by eliminating one or more atoms from the above monovalent aryl group Sub₁.

Herein, examples of the heterocyclic group in the linking group include a divalent or multivalent group obtained by eliminating one or more atoms from the above monovalent heteroaryl group Sub₂.

### EXAMPLES

### Compounds

Structures of the compound represented by the formula (3) and used for manufacturing organic EL devices in Examples 1 to 4 are shown below.

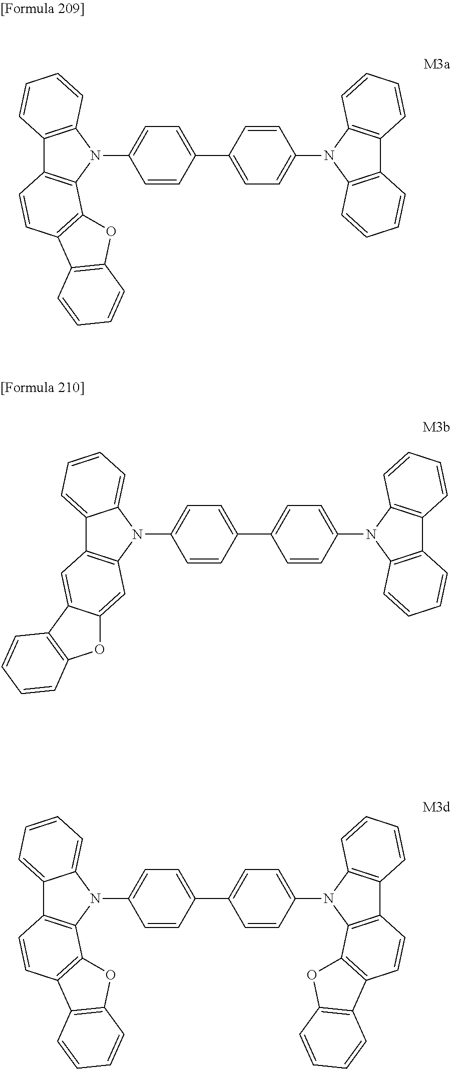 [SMILES: c1ccc2c(c1)oc1c2ccc2c3ccccc3n(-c3ccc(-c4ccc(-n5c6ccccc6c6ccccc65)cc4)cc3)c21 ; c1ccc2c(c1)oc1cc3c(cc12)c1ccccc1n3-c1ccc(-c2ccc(-n3c4ccccc4c4ccccc43)cc2)cc1 ; c1ccc2c(c1)oc1c2ccc2c3ccccc3n(-c3ccc(-c4ccc(-n5c6ccccc6c6ccc7c8ccccc8oc7c65)cc4)cc3)c21]

Structures of comparative compounds used for manufacturing organic EL devices in Comparatives 1 to 3 are shown below.

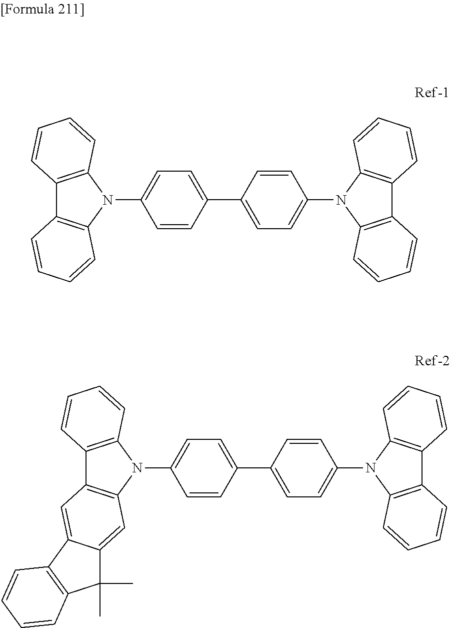 [SMILES: c1ccc2c(c1)c1ccccc1n2-c1ccc(-c2ccc(-n3c4ccccc4c4ccccc43)cc2)cc1 ; CC1(C)c2ccccc2-c2cc3c4ccccc4n(-c4ccc(-c5ccc(-n6c7ccccc7c7ccccc76)cc5)cc4)c3cc21]

Structures of other compounds used for manufacturing the organic EL devices in Examples 1 to 4 and Comparatives 1 to 3 are shown below.

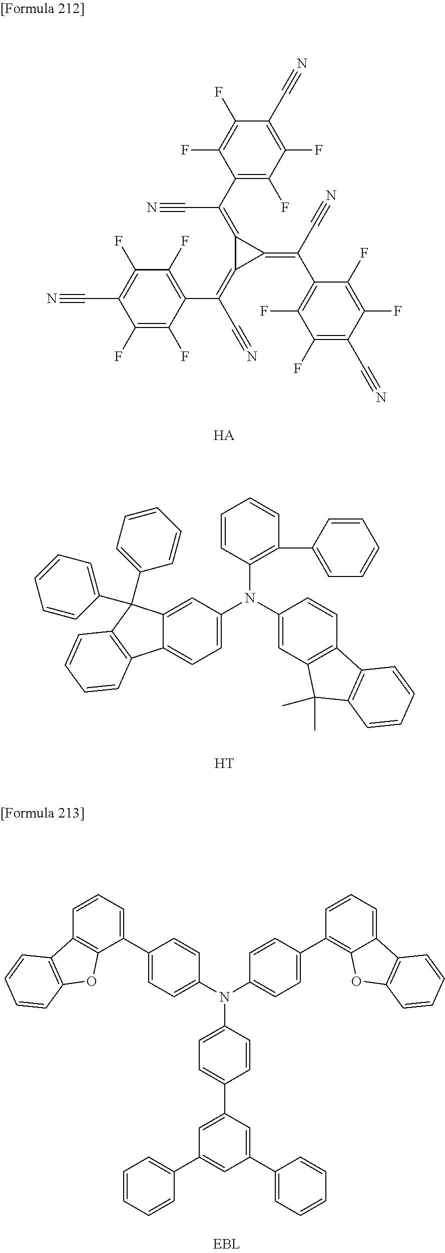

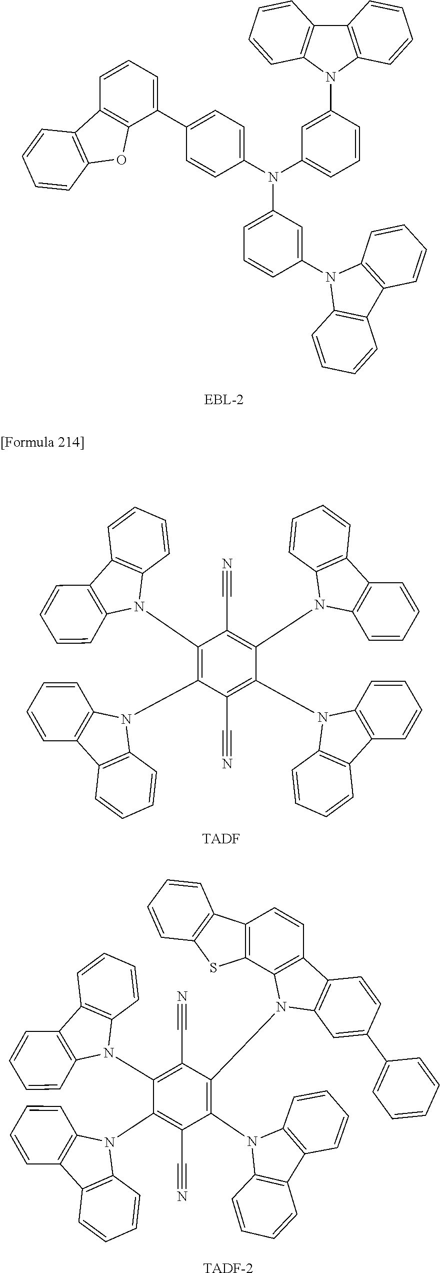

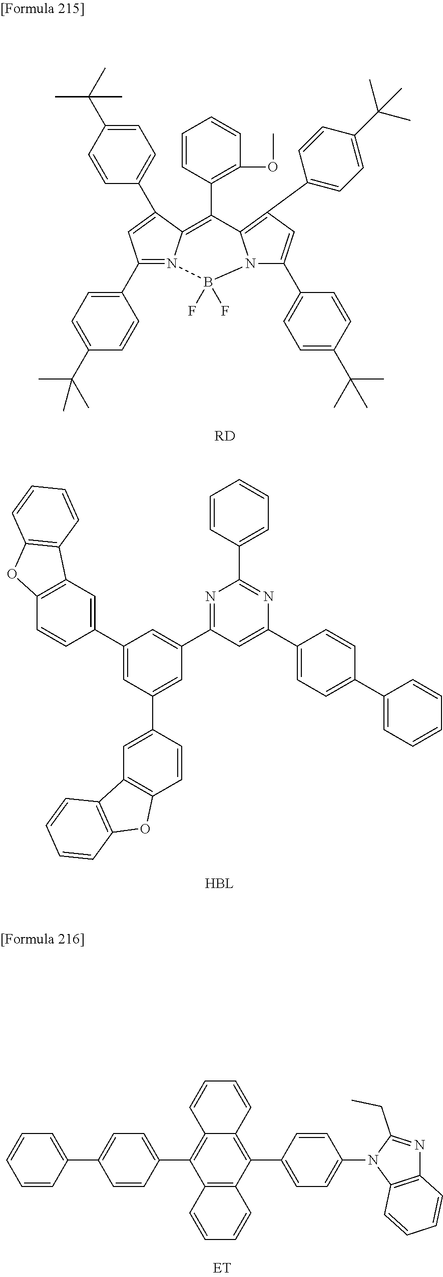

### Manufacture of Organic EL Devices

Organic EL devices were manufactured and evaluated as follows.

### Example 1

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV/ozone-cleaned for one minute. A film of ITO was 130 nm thick.

After the glass substrate having the transparent electrode line was cleaned, the glass substrate was mounted on a substrate holder of a vacuum evaporation apparatus. Firstly, a compound HT and a compound HA were co-deposited on a surface of the glass substrate where the transparent electrode line was provided in a manner to cover the transparent electrode, thereby forming a 10-nm-thick hole injecting layer. The concentrations of the compound HT and the compound HA in the hole injecting layer were 97 mass % and 3 mass %, respectively.

Next, the compound HT was vapor-deposited on the hole injecting layer to form a 200-nm-thick hole transporting layer.

Next, the compound EBL was vapor-deposited on the hole transporting layer to form a 10-nm-thick electron blocking layer.

Next, a compound M3a (the compound M3), a compound TADF (the compound M2) and a compound RD (the compound M1) were co-deposited on the electron blocking layer to form a 25-nm-thick emitting layer. The concentrations of the compound M3a, the compound TADF, and the compound RD in the emitting layer were 74 mass %, 25 mass %, and 1 mass %, respectively.

Next, a compound HBL was vapor-deposited on the emitting layer to form a 10-nm-thick hole blocking layer.

Next, a compound ET was vapor-deposited on the hole blocking layer to form a 30-nm-thick electron transporting layer.

Lithium fluoride (LiF) was vapor-deposited on the electron transporting layer to form a 1-nm-thick electron injectable electrode (cathode).

Subsequently, metal aluminum (Al) was vapor-deposited on the electron injectable electrode to form an 80-nm-thick metal Al cathode.

A device arrangement of the organic EL device of Example 1 is roughly shown as follows. ITO(130)/HT:HA(10,97%:3%)/HT(200)/EBL(10)/M3a:TADF:RD(25,74%:25%:1%)/HBL(10)/ET(30)/LiF(1)/Al(80)

Numerals in parentheses represent a film thickness (unit: nm).

The numerals (97%:3%) represented by percentage in the same parentheses indicate a ratio (mass %) between the compound HT and the compound HA in the hole injecting layer, and the numerals (74%:25%:1%) represented by percentage in the same parentheses indicate a ratio (mass %) between the compound M3a, the compound TADF, and the compound RD in the emitting layer. Similar notations apply to the description below.

### Comparative 1

The organic EL device in Comparative 1 was manufactured in the same manner as in Example 1 except that a compound shown in Table 1 was used in place of the compound M3a used in the emitting layer of the organic EL device of Example 1.

### Evaluation of Organic EL Devices

The organic EL devices manufactured in Examples 1 to 4 and Comparatives 1 to 3 were evaluated as follows. Evaluation results are shown in Table 1 or 2. It should be noted that a comparative compound Ref-1 used in Comparatives 1 and 2 and a comparative compound Ref-2 used in Comparative 3 are shown in a column of the compound M3 for convenience of explanation.

### Main Peak Wavelength (λp)

Voltage was applied on the organic EL devices such that a current density of the organic EL device was 10 mA/cm², where spectral radiance spectrum was measured by a spectroradiometer CS-2000 (manufactured by Konica Minolta, Inc.). The main peak wavelength λₚ (unit: nm) was calculated based on the obtained spectral-radiance spectra.

### Drive Voltage

A voltage (unit: V) was measured when current was applied between the anode and the cathode such that a current density was 10 mA/cm².

### External Quantum Efficiency EQE

Voltage was applied on the organic EL devices so that a current density was 10 mA/cm², where spectral radiance spectrum was measured by a spectroradiometer CS-2000 (manufactured by Konica Minolta, Inc.). The external quantum efficiency EQE (unit: %) was calculated based on the obtained spectral-radiance spectra, assuming that the spectra was provided under a Lambertian radiation.

TABLE 1

| | | | | | | | Evaluation Results of Devices | | |
|---|---|---|---|---|---|---|---|---|---|
| | Compound M3 | | Compound M2 | | Compound M1 | | (@10 mA/cm²) | | |
| | | S₁ | | S₁ | | S₁ | λ p | Voltage V | EQE |
| | Type | [eV] | Type | [eV] | Type | [eV] | [nm] | [V] | [%] |
| Example 1 | M3a | 3.42 | TADF | 2.38 | RD | 2.02 | 621 | 4.41 | 18.2 |
| Comparative 1 | Ref-1 | 3.52 | TADF | 2.38 | RD | 2.02 | 621 | 4.41 | 15.6 |

In Tables 1 and 2 above, S₁[eV] shows values of physical properties of the compounds used in the emitting layer of each of Examples and Comparatives. For instance, for Example 1, it is shown that S₁ of the compound M3a is 3.42 eV and S₁ of the compound TADF is 2.38 eV.

Although Ref-1 of Comparative 1 does not fall under the formula of the compound M3, Ref-1 is shown in the same column as M3a used in Example 1 for convenience.

The organic EL device in Example 1 emitted light with a higher efficiency than the organic EL device in Comparative 1.

### Manufacture of Organic EL Devices

### Example 2

A glass substrate (size: 25 mm×75 mm×1.1 mm thick, manufactured by Geomatec Co., Ltd.) having an ITO transparent electrode (anode) was ultrasonic-cleaned in isopropyl alcohol for five minutes, and then UV/ozone-cleaned for one minute. A film of ITO was 130 nm thick.

After the glass substrate having the transparent electrode line was cleaned, the glass substrate was mounted on a substrate holder of a vacuum evaporation apparatus. Firstly, the compound HT and the compound HA were co-deposited on a surface of the glass substrate where the transparent electrode line was provided in a manner to cover the transparent electrode, thereby forming a 10-nm-thick hole injecting layer. The concentrations of the compound HT and the compound HA in the hole injecting layer were 97 mass % and 3 mass %, respectively.

Next, the compound HT was vapor-deposited on the hole injecting layer to form a 200-nm-thick hole transporting layer.

Next, the compound EBL-2 was vapor-deposited on the hole transporting layer to form a 10-nm-thick electron blocking layer.

Next, the compound M3a (the compound M3), a compound TADF-2 (the compound M2) and the compound RD (the compound M1) were co-deposited on the electron blocking layer to form a 25-nm-thick emitting layer. The concentrations of the compound M3a, the compound TADF-2, and the compound RD in the emitting layer were 74 mass %, 25 mass %, and 1 mass %, respectively.

Next, the compound HBL was vapor-deposited on the emitting layer to form a 10-nm-thick hole blocking layer.

Next, the compound ET was vapor-deposited on the hole blocking layer to form a 30-nm-thick electron transporting layer.

Lithium fluoride (LiF) was vapor-deposited on the electron transporting layer to form a 1-nm-thick electron injectable electrode (cathode).

Subsequently, metal aluminum (Al) was vapor-deposited on the electron injectable electrode to form an 80-nm-thick metal Al cathode.

A device arrangement of the organic EL device of Example 1 is roughly shown as follows. ITO(130)/HT:HA(10,97%:3%)/HT(200)/EBL-2(10)/M3a:TADF-2:RD(25,74%:25%:1%)/HBL(10)/ET(30)/LiF(1)/Al(80)

Numerals in parentheses represent a film thickness (unit: nm).

The numerals (97%:3%) represented by percentage in the same parentheses indicate a ratio (mass %) between the compound HT and the compound HA in the hole injecting layer, and the numerals (74%:25%:1%) represented by percentage in the same parentheses indicate a ratio (mass %) between the compound M3a, the compound TADF-2, and the compound RD in the emitting layer. Similar notations apply to the description below.

### Examples 3 and 4

The organic EL devices in Examples 3 and 4 were each manufactured in the same manner as in Example 2 except that a compound shown in Table 2 was used in place of the compound M3a used in the emitting layer of the organic EL device of Example 2.

### Comparatives 2 and 3

The organic EL devices in Comparatives 2 and 3 were each manufactured in the same manner as in Example 2 except that a compound shown in Table 2 was used in place of the compound M3a used in the emitting layer of the organic EL device of Example 2.

### Evaluation of Organic EL Devices

The manufactured organic EL devices were evaluated by the same method as described above in terms of the external quantum efficiency EQE and main peak wavelength λp. Evaluation results are shown in Table 2.

TABLE 2

| | | | | | | | Evaluation Results | |
|---|---|---|---|---|---|---|---|---|
| | | | | | | | of Devices | |
| | Compound M3 | | Compound M2 | | Compound M1 | | (@10 mA/cm²) | |
| | | S₁ | | S₁ | | S₁ | λ p | EQE |
| | Type | [eV] | Type | [eV] | Type | [eV] | [nm] | [%] |
| Example 2 | M3a | 3.42 | TADF-2 | 2.34 | RD | 2.02 | 621 | 19.5 |
| Example 3 | M3b | 3.39 | TADF-2 | 2.34 | RD | 2.02 | 621 | 18.9 |
| Example 4 | M3d | 3.42 | TADF-2 | 2.34 | RD | 2.02 | 621 | 17.7 |
| Comparative 2 | Ref-1 | 3.52 | TADF-2 | 2.34 | RD | 2.02 | 621 | 16.0 |
| Comparative 3 | Ref-2 | 3.34 | TADF-2 | 2.34 | RD | 2.02 | 621 | 12.4 |

Although Ref-1 of Comparative 2 and Ref-2 of Comparative 2 do not fall under the formula of the compound M3, Ref-1 and Ref-2 are shown in the same column as the compound M3 used in Examples 2 to 4 for convenience.

The organic EL devices in Examples 2 to 4 emitted light with a higher efficiency than the organic EL devices in Comparatives 2 to 3.

### Evaluation of Compounds

Values of physical properties of the compounds used in Examples 1 to 4 and Comparatives 1 to 3 were measured by the following method.

### Delayed Fluorescence

### Delayed Fluorescence of Compound TADF

Delayed fluorescence properties were checked by measuring transient photoluminescence (PL) using a device shown in FIG. 2. The compound TADF was dissolved in toluene to prepare a dilute solution with an absorbance of 0.05 or less at the excitation wavelength to eliminate the contribution of self-absorption. In order to prevent quenching due to oxygen, the sample solution was frozen and degassed and then sealed in a cell with a lid under an argon atmosphere to obtain an oxygen-free sample solution saturated with argon.

The fluorescence spectrum of the above sample solution was measured with a spectrofluorometer FP-8600 (manufactured by JASCO Corporation), and the fluorescence spectrum of a 9,10-diphenylanthracene ethanol solution was measured under the same conditions. Using the fluorescence area intensities of both spectra, the total fluorescence quantum yield was calculated by an equation (1) in Morris et al. J. Phys. Chem. 80 (1976) 969.

Prompt emission was observed immediately when the excited state was achieved by exciting the compound TADF with a pulse beam (i.e., a beam emitted from a pulse laser) having a wavelength to be absorbed by the compound TADF, and Delay emission was observed not immediately when the excited state was achieved but after the excited state was achieved. The delayed fluorescence in Examples means that an amount of Delay Emission is 5% or more with respect to an amount of Prompt Emission. Specifically, provided that the amount of Prompt emission is denoted by X_{P} and the amount of Delay emission is denoted by X_{D}, the delayed fluorescence means that a value of X_{D}/X_{P} is 0.05 or more.

An amount of Prompt emission, an amount of Delay emission and a ratio between the amounts thereof can be obtained according to the method as described in “Nature 492, 234-238, 2012” (Reference Document 1). The amount of Prompt emission and the amount of Delay emission may be calculated using a device different from one described in Reference Document 1 or one shown in FIG. 2.

It was confirmed that the amount of Delay emission was 5% or more with respect to the amount of Prompt emission in the compound TADF.

The compound TADF-2 was measured in the same manner as the compound TADF, so that it was confirmed that the amount of Delay emission was also 5% or more with respect to the amount of Prompt emission in the compound TADF-2.

Specifically, it was found that a value of X_{D}/X_{P} was 0.05 or more in both the compounds TADF and TADF-2.

### Singlet Energy S₁

Singlet energy S₁ of each of the compounds M3a, M3b, M3d, TADF, TADF-2 and RD, and the comparative compounds Ref-1 and Ref-2 was measured according to the above-described solution method. Measurement results are shown in Table 1 or 2.

Singlet energies of compounds M3c and M3e of later-described synthesis examples were measured according to the same method, and were 3.35 eV and 3.46 eV, respectively.

### Energy Gap at 77K

An energy gap T_{77K} at 77K of each of the compounds TADF and TADF-2 was measured by the above-described method.

### ΔST

ΔST was calculated based on the measured singlet energy S₁ and energy gap T_{77K} at 77K. It was confirmed that ΔST of each of the compounds TADF and TADF-2 was less than 0.01.

### Synthesis Example 1: Synthesis of Compound M3a

### (1-1) Synthesis of Compound M3a

A synthesis scheme of the compound M3a is shown below.

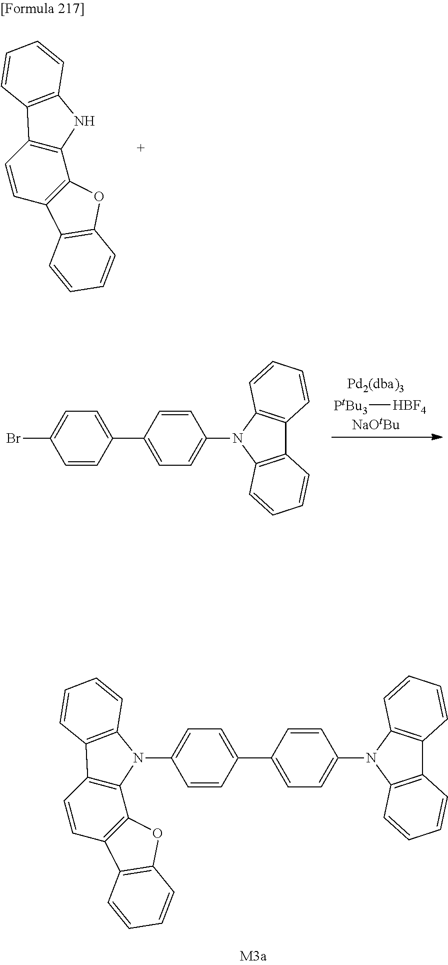

Under nitrogen atmosphere, xylene (675 mL) was added into a mixture of 12H-benzofuro[2,3-a]carbazole (26.6 g, 103 mmol), 9-(4′-bromo-[1,1′-biphenyl]-4-yl)-9H-carbazole (41.2 g, 103 mmol), tris(dibenzylideneacetone)dipalladium (1.90 g, 2.07 mmol), tri-tert-butylphosphonium tetrafluoroborate (1.20 g, 4.14 mmol), and sodium tert-butoxide (11.9 g, 124 mmol), and stirred at 130 degrees C. for eight hours. After the reaction, a solid was collected by filtration. The solid collected by filtration was recrystallized with toluene to obtain the compound M3a (51.5 g, a yield of 87%). The obtained compound was identified as the compound M3a by analysis according to LC-MS (Liquid chromatography mass spectrometry).

### Synthesis Example 2: Synthesis of Compound M3b

### (1-2) Synthesis of Compound M3b

A synthesis scheme of the compound M3b is shown below.

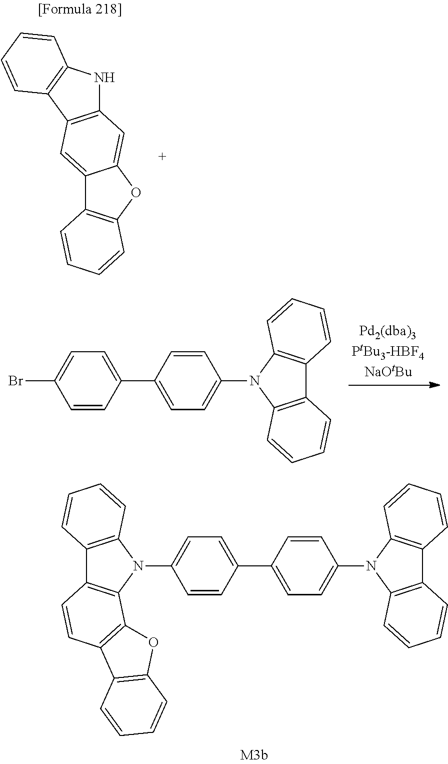 [SMILES: c1ccc2c(c1)[nH]c1cc3oc4ccccc4c3cc12 ; Brc1ccc(-c2ccc(-n3c4ccccc4c4ccccc43)cc2)cc1 ; c1ccc2c(c1)oc1c2ccc2c3ccccc3n(-c3ccc(-c4ccc(-n5c6ccccc6c6ccccc65)cc4)cc3)c21]

Under nitrogen atmosphere, xylene (650 mL) was added into a mixture of 7H-benzofuro[2,3-b]carbazole (25.8 g, 100 mmol), 9-(4′-bromo-[1,1′-biphenyl]-4-yl)-9H-carbazole (40.0 g, 100 mmol), tris(dibenzylideneacetone)dipalladium (1.83 g, 2.00 mmol), tri-tert-butylphosphonium tetrafluoroborate (1.16 g, 4.00 mmol), and sodium tert-butoxide (11.5 g, 120 mmol), and stirred at 130 degrees C. for eight hours. After the reaction, a solid was collected by filtration. The solid collected by filtration was recrystallized with toluene to obtain the compound M3b (45.7 g, a yield of 79%). The obtained compound was identified as the compound M3b by analysis according to LC-MS (Liquid chromatography mass spectrometry).

### Synthesis Example 3: Synthesis of Compound M3c

### (1-3) Synthesis of Compound M3c

A synthesis scheme of the compound M3c is shown below.

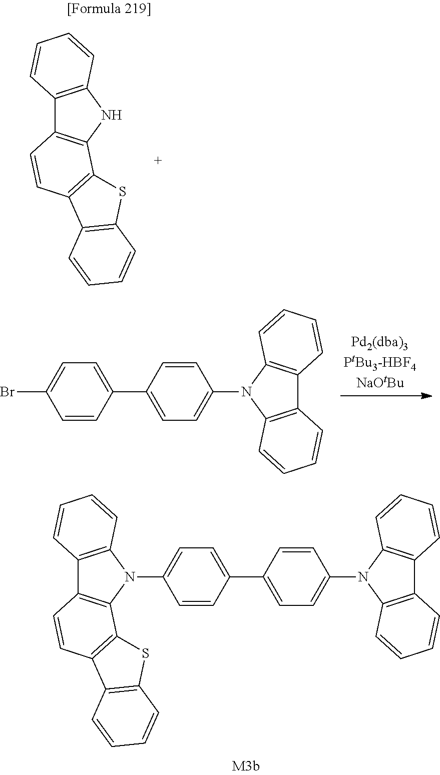 [SMILES: c1ccc2c(c1)[nH]c1c2ccc2c3ccccc3sc21 ; Brc1ccc(-c2ccc(-n3c4ccccc4c4ccccc43)cc2)cc1 ; c1ccc2c(c1)sc1c2ccc2c3ccccc3n(-c3ccc(-c4ccc(-n5c6ccccc6c6ccccc65)cc4)cc3)c21]

Under nitrogen atmosphere, xylene (650 mL) was added into a mixture of 12H-[1]benzothieno[2,3-a]carbazole (27.3 g, 100 mmol), 9-(4′-bromo-[1,1′-biphenyl]-4-yl)-9H-carbazole (40.0 g, 100 mmol), tris(dibenzylideneacetone)dipalladium (1.83 g, 2.00 mmol), tri-tert-butylphosphonium tetrafluoroborate (1.16 g, 4.00 mmol), and sodium tert-butoxide (11.5 g, 120 mmol), and stirred at 130 degrees C. for eight hours. After the reaction, a solid was collected by filtration. The solid collected by filtration was recrystallized with toluene to obtain the compound M3c (42.5 g, a yield of 72%). The obtained compound was identified as the compound M3c by analysis according to LC-MS (Liquid chromatography mass spectrometry).

### Synthesis Example 4: Synthesis of Compound M3d

### (1-4) Synthesis of Compound M3e

A synthesis scheme of the compound M3e is shown below.

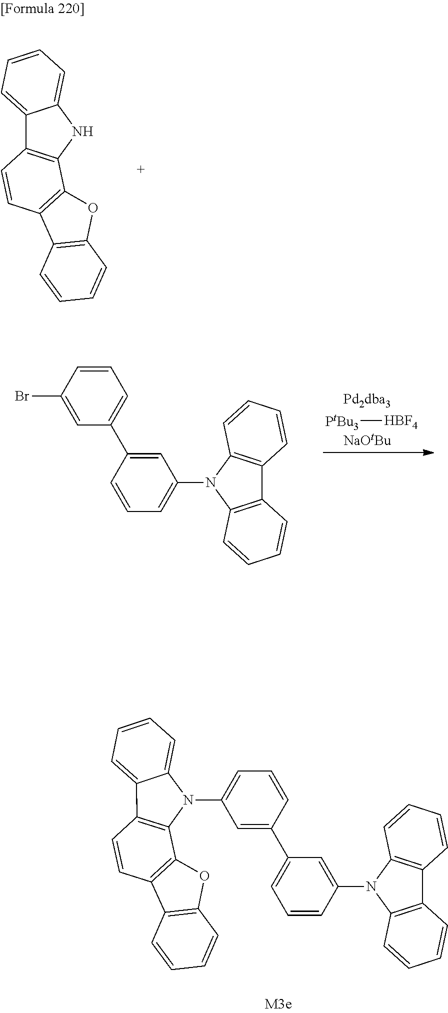

The compound M3e was obtained in the same manner as in Synthesis Example 1 except for using 9-(3′-bromo-[1,1′-biphenyl]-3-yl)-9H-carbazole in place of 9-(4′-bromo-[1,1′-biphenyl]-4-yl)-9H-carbazole in Synthesis Example 1. A yield was 63%. The obtained compound was identified as the compound M3e by analysis according to LC-MS (Liquid chromatography mass spectrometry).

### EXPLANATION OF CODE(S)

1 . . . organic EL device, 2 . . . substrate, 3 . . . anode, 4 . . . cathode, 5 . . . emitting layer, 6 . . . hole injecting layer, 7 . . . hole transporting layer, 8 . . . electron transporting layer, 9 . . . electron injecting layer

1. An organic electroluminescence device, comprising:
an anode;
a cathode; and
an emitting layer provided between the anode and the cathode,
wherein the emitting layer comprises a delayed fluorescent compound M2 and a compound M3 represented by formula (3) below,
wherein a singlet energy S₁(M2) of the compound M2 and a singlet energy S₁(M3) of the compound M3 satisfy the relationship S₁(M3)>S₁(M2), 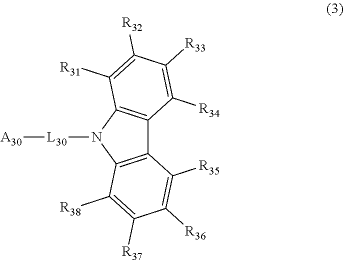
wherein, in formula (3), A₃₀ is a group represented by the formula (3a), (3b), (3c), (3d), (3e), or (3f) below, 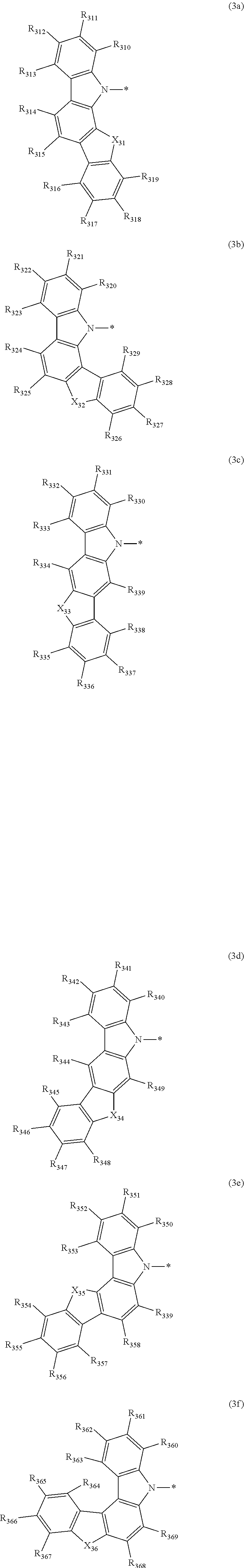
wherein, in the formulae (3a), (3b), (3c), (3d), (3e), and (3f):
X₃₁, X₃₂, X₃₃, X₃₄, X₃₅, and X₃₆ are each independently selected from the group consisting of an oxygen atom, a sulfur atom, NR₃₇₁, and SiR₃₇₂R₃₇₃;
R₃₇₁, R₃₇₂, and R₃₇₃ are each independently a hydrogen atom or a substituent;
R₃₁₀ to R₃₁₉ are each independently a hydrogen atom or a substituent, or at least one pair mutually bonded to form a ring and selected from the group consisting of a pair of R₃₁₀ and R₃₁₁, a pair of R₃₁₁ and R₃₁₂, a pair of R₃₁₂ and R₃₁₃, a pair of R₃₁₄ and R₃₁₅, a pair of R₃₁₆ and R₃₁₇, a pair of R₃₁₇ and R₃₁₈, and a pair of R₃₁₈ and R₃₁₉;
R₃₂₀ to R₃₂₉ are each independently a hydrogen atom or a substituent, or at least one pair mutually bonded to form a ring and selected from the group consisting of a pair of R₃₂₀ and R₃₂₁, a pair of R₃₂₁ and R₃₂₂, a pair of R₃₂₂ and R₃₂₃, a pair of R₃₂₄ and R₃₂₅, a pair of R₃₂₆ and R₃₂₇, a pair of R₃₂₇ and R₃₂₈, and a pair of R₃₂₈ and R₃₂₉;
R₃₃₀ to R₃₃₉ are each independently a hydrogen atom or a substituent, or at least one pair mutually bonded to form a ring and selected from the group consisting of a pair of R₃₃₀ and R₃₃₁, a pair of R₃₃₁ and R₃₃₂, a pair of R₃₃₂ and R₃₃₃, a pair of R₃₃₅ and R₃₃₆, a pair of R₃₃₆ and R₃₃₇, and a pair of R₃₃₇ and R₃₃₈;
R₃₄₀ to R₃₄₉ are each independently a hydrogen atom or a substituent, or at least one pair mutually bonded to form a ring and selected from the group consisting of a pair of R₃₄₀ and R₃₄₁, a pair of R₃₄₁ and R₃₄₂, a pair of R₃₄₂ and R₃₄₃, a pair of R₃₄₅ and R₃₄₆, a pair of R₃₄₆ and R₃₄₇, and a pair of R₃₄₇ and R₃₄₈;
R₃₅₀ to R₃₅₉ are each independently a hydrogen atom or a substituent, or at least one pair mutually bonded to form a ring and selected from the group consisting of a pair of R₃₅₀ and R₃₅₁, a pair of R₃₅₁ and R₃₅₂, a pair of R₃₅₂ and R₃₅₃, a pair of R₃₅₄ and R₃₅₅, a pair of R₃₅₅ and R₃₅₆, a pair of R₃₅₆ and R₃₅₇, and a pair of R₃₅₈ and R₃₅₉;
R₃₆₀ to R₃₆₉ are each independently a hydrogen atom or a substituent, or at least one pair mutually bonded to form a ring and selected from the group consisting of a pair of R₃₆₀ and R₃₆₁, a pair of R₃₆₁ and R₃₆₂, a pair of R₃₆₂ and R₃₆₃, a pair of R₃₆₄ and R₃₆₅, a pair of R₃₆₅ and R₃₆₆, a pair of R₃₆₆ and R₃₆₇, and a pair of R₃₆₈ and R₃₆₉;
R₃₇₁, R₃₇₂, R₃₇₃, R₃₁₀ to R₃₁₉, R₃₂₀ to R₃₂₉, R₃₃₀ to R₃₃₉, R₃₄₀ to R₃₄₉, R₃₅₀ to R₃₅₉, and R₃₆₀ to R₃₆₉ as a substituent are each independently selected from the group consisting of a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, and a substituted or unsubstituted arylthio group having 6 to 30 ring carbon atoms;
* represents a bonding position to L₃₀;
wherein, in formula (3):
L₃₀ is selected from the group consisting of a substituted or unsubstituted arylene group having 6 to 22 ring carbon atoms, a group formed by bonding two substituted or unsubstituted arylene groups having 6 to 22 ring carbon atoms, and a group formed by bonding three substituted or unsubstituted arylene groups having 6 to 22 ring carbon atoms;
R₃₁ to R₃₈ are each independently a hydrogen atom or a substituent, or at least one pair mutually bonded to form a ring and selected from the group consisting of a pair of R₃₁ and R₃₂, a pair of R₃₂ and R₃₃, a pair of R₃₃ and R₃₄, a pair of R₃₅ and R₃₆, a pair of R₃₆ and R₃₇, and a pair of R₃₇ and R₃₈; and
L₃₀, and R₃₁ to R₃₈ as a substituent are each independently selected from the group consisting of a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, and a substituted or unsubstituted arylthio group having 6 to 30 ring carbon atoms.

2. The organic electroluminescence device according to claim 1, wherein:
the emitting layer further comprises a fluorescent compound M1, and
the singlet energy S₁(M2) of the compound M2 and a singlet energy S₁(M1) of the compound M1 satisfy the relationship S₁(M2)>S₁(M1).

3. The organic electroluminescence device according to claim 1, wherein
in the compound M3, a pair of R₃₁ and R₃₂, a pair of R₃₂ and R₃₃, a pair of R₃₃ and R₃₄, a pair of R₃₅ and R₃₆, a pair of R₃₆ and R₃₇, and a pair of R₃₇ and R₃₈ are not mutually bonded, or
the compound M3 is represented by the formula (31), (32), (33), (34), (35), or (36) below, 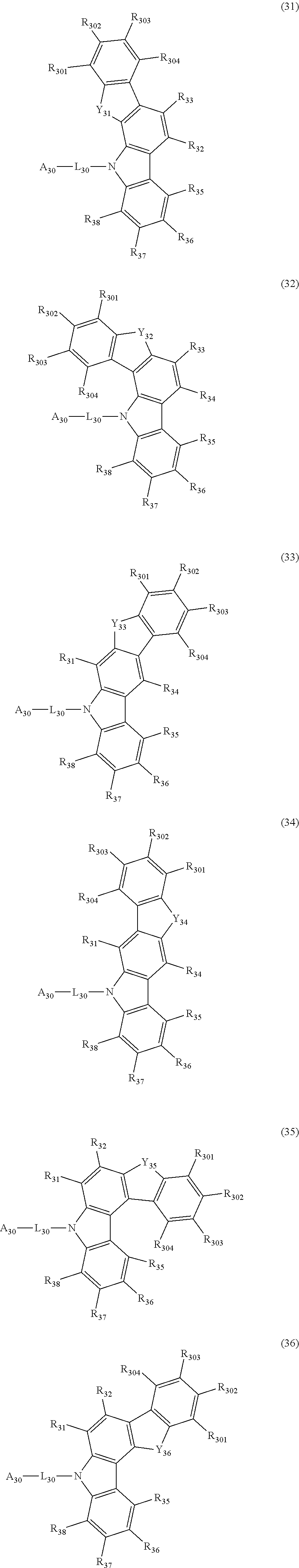
wherein, in formulae (31) to (36):
Y₃₁, Y₃₂, Y₃₃, Y₃₄, Y₃₅, and Y₃₆ are each independently selected from the group consisting of an oxygen atom, a sulfur atom, NR₃₇₄, and SiR₃₇₅R₃₇₆;
R₃₇₄, R₃₇₅, and R₃₇₆ are each independently a hydrogen atom or a substituent;
A₃₀, L₃₀, and R₃₁ to R₃₈ respectively represent the same as A₃₀, L₃₀, and R₃₁ to R₃₈ in the formula (3);
R₃₀₁ to R₃₀₄ are each independently a hydrogen atom or a substituent, or at least one pair mutually bonded to form a ring and selected from the group consisting of a pair of R₃₀₁ and R₃₀₂, a pair of R₃₀₂ and R₃₀₃, and a pair of R₃₀₃ and R₃₀₄; and
R₃₀₁ to R₃₀₄, R₃₇₄, R₃₇₅, and R₃₇₆ as a substituent are each independently selected from the group consisting of a halogen atom, a cyano group, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, and a substituted or unsubstituted arylthio group having 6 to 30 ring carbon atoms.

4. The organic electroluminescence device according to claim 3, wherein Y₃₁, Y₃₂, Y₃₃, Y₃₄, Y₃₅, and Y₃₆ are each independently an oxygen atom or a sulfur atom.

5. The organic electroluminescence device according to claim 3, wherein R₃₁ to R₃₈ are each independently selected from the group consisting of a hydrogen atom, a substituted or unsubstituted aryl group having 6 to 30 ring carbon atoms, a substituted or unsubstituted heterocyclic group having 5 to 30 ring atoms, and a substituted or unsubstituted alkyl group having 1 to 30 carbon atoms.

6. The organic electroluminescence device according to claim 3, wherein R₃₁ to R₃₈ are each independently selected from the group consisting of a hydrogen atom, a substituted or unsubstituted aryl group having 6 to 30 ring carbon atoms and a substituted or unsubstituted heterocyclic group having 5 to 30 ring atoms.

7. The organic electroluminescence device according to claim 3, wherein R₃₁ to R₃₈ are each independently selected from the group consisting of a hydrogen atom and a substituted or unsubstituted aryl group having 6 to 30 ring carbon atoms.

8. The organic electroluminescence device according to claim 3, wherein R₃₁ to R₃₈ are each a hydrogen atom.

9. The organic electroluminescence device according to claim 3, wherein R₃₁ to R₃₈ and R₃₀₁ to R₃₀₄ are each independently selected from the group consisting of a hydrogen atom and a substituted or unsubstituted aryl group having 6 to 30 ring carbon atoms.

10. The organic electroluminescence device according to claim 3, wherein R₃₁ to R₃₈ and R₃₀₁ to R₃₀₄ are each a hydrogen atom.

11. The organic electroluminescence device according to claim 1, wherein at least one pair is mutually bonded to form a ring and selected from the group consisting of a pair of R₃₁ and R₃₂, a pair of R₃₂ and R₃₃, a pair of R₃₃ and R₃₄, a pair of R₃₅ and R₃₆, a pair of R₃₆ and R₃₇, and a pair of R₃₇ and R₃₈.

12. The organic electroluminescence device according to claim 1, wherein L₃₀ is selected from the group consisting of a substituted or unsubstituted phenylene group, a substituted or unsubstituted biphenylene group, and a substituted or unsubstituted terphenylene group.

13. The organic electroluminescence device according to claim 1, wherein L₃₀ is a substituted or unsubstituted biphenylene group.

14. The organic electroluminescence device according to claim 1, wherein a substituent for substituting L₃₀ is selected from the group consisting of a substituted or unsubstituted aryl group having 6 to 30 ring carbon atoms, and a substituted or unsubstituted alkyl group having 1 to 30 carbon atoms.

15. The organic electroluminescence device according to claim 1, wherein X₃₁, X₃₂, X₃₃, X₃₄, X₃₅, and X₃₆ are each independently selected from the group consisting of an oxygen atom, NR₃₀₁, and SiR₃₀₂R₃₀₃.

16. The organic electroluminescence device according to claim 1, wherein X₃₁, X₃₂, X₃₃, X₃₄, X₃₅, and X₃₆ are each independently an oxygen atom or a sulfur atom.

17. The organic electroluminescence device according to claim 1, wherein X₃₁, X₃₂, X₃₃, X₃₄, X₃₅, and X₃₆ are each an oxygen atom.

18. The organic electroluminescence device according to claim 1, wherein:
a pair of R₃₁₀ and R₃₁₁, a pair of R₃₁₁ and R₃₁₂, a pair of R₃₁₂ and R₃₁₃, a pair of R₃₁₄ and R₃₁₅, a pair of R₃₁₆ and R₃₁₇, a pair of R₃₁₇ and R₃₁₈, and a pair of R₃₁₈ and R₃₁₉ are not mutually bonded;
a pair of R₃₂₀ and R₃₂₁, a pair of R₃₂₁ and R₃₂₂, a pair of R₃₂₂ and R₃₂₃, a pair of R₃₂₄ and R₃₂₅, a pair of R₃₂₆ and R₃₂₇, a pair of R₃₂₇ and R₃₂₈, and a pair of R₃₂₈ and R₃₂₉ are not mutually bonded;
a pair of R₃₃₀ and R₃₃₁, a pair of R₃₃₁ and R₃₃₂, a pair of R₃₃₂ and R₃₃₃, a pair of R₃₃₅ and R₃₃₆, a pair of R₃₃₆ and R₃₃₇, and a pair of R₃₃₇ and R₃₃₈ are not mutually bonded;
a pair of R₃₄₀ and R₃₄₁, a pair of R₃₄₁ and R₃₄₂, a pair of R₃₄₂ and R₃₄₃, a pair of R₃₄₅ and R₃₄₆, a pair of R₃₄₆ and R₃₄₇, and a pair of R₃₄₇ and R₃₄₈ are not mutually bonded;
a pair of R₃₅₀ and R₃₅₁, a pair of R₃₅₁ and R₃₅₂, a pair of R₃₅₂ and R₃₅₃, a pair of R₃₅₄ and R₃₅₅, a pair of R₃₅₅ and R₃₅₆, a pair of R₃₅₆ and R₃₅₇, and a pair of R₃₅₈ and R₃₅₉ are not mutually bonded; and
a pair of R₃₆₀ and R₃₆₁, a pair of R₃₆₁ and R₃₆₂, a pair of R₃₆₂ and R₃₆₃, a pair of R₃₆₄ and R₃₆₅, a pair of R₃₆₅ and R₃₆₆, a pair of R₃₆₆ and R₃₆₇, and a pair of R₃₆₈ and R₃₆₉ are not mutually bonded.

19. The organic electroluminescence device according to claim 1, wherein R₃₁₀ to R₃₁₉, R₃₂₀ to R₃₂₉, R₃₃₀ to R₃₃₉, R₃₄₀ to R₃₄₉, R₃₅₀ to R₃₅₉, and R₃₆₀ to R₃₆₉ are each independently selected from the group consisting of a hydrogen atom, a substituted or unsubstituted aryl group having 6 to 30 ring carbon atoms, a substituted or unsubstituted heterocyclic group having 5 to 30 ring atoms, and a substituted or unsubstituted alkyl group having 1 to 30 carbon atoms.

20. The organic electroluminescence device according to claim 18, wherein R₃₁₀ to R₃₁₉, R₃₂₀ to R₃₂₉, R₃₃₀ to R₃₃₉, R₃₄₀ to R₃₄₉, R₃₅₀ to R₃₅₉, and R₃₆₀ to R₃₆₉ are each independently selected from the group consisting of a hydrogen atom, a substituted or unsubstituted aryl group having 6 to 30 ring carbon atoms, and a substituted or unsubstituted heterocyclic group having 5 to 30 ring atoms.

21. The organic electroluminescence device according to claim 18, wherein R₃₁₀ to R₃₁₉, R₃₂₀ to R₃₂₉, R₃₃₀ to R₃₃₉, R₃₄₀ to R₃₄₉, R₃₅₀ to R₃₅₉, and R₃₆₀ to R₃₆₉ are each independently selected from the group consisting of a hydrogen atom and a substituted or unsubstituted aryl group having 6 to 30 ring carbon atoms.

22. The organic electroluminescence device according to claim 18, wherein R₃₁₀ to R₃₁₉, R₃₂₀ to R₃₂₉, R₃₃₀ to R₃₃₉, R₃₄₀ to R₃₄₉, R₃₅₀ to R₃₅₉, and R₃₆₀ to R₃₆₉ are each a hydrogen atom.

23. The organic electroluminescence device according to claim 1, wherein A₃₀ is the group represented by the formula (3a) or (3d).

24. The organic electroluminescence device according to claim 1, wherein A₃₀ is the group represented by the formula (3a).

25. An electronic device comprising the organic electroluminescence device according to claim 1.

26. A compound represented by the formula (301) below, 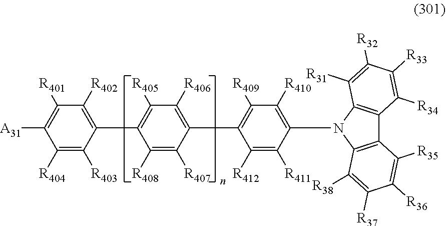
wherein, in the formula (301), A₃₁ is a group represented by the formula (31a), (31b), (31c), (31d), (31e), or (31f) below, 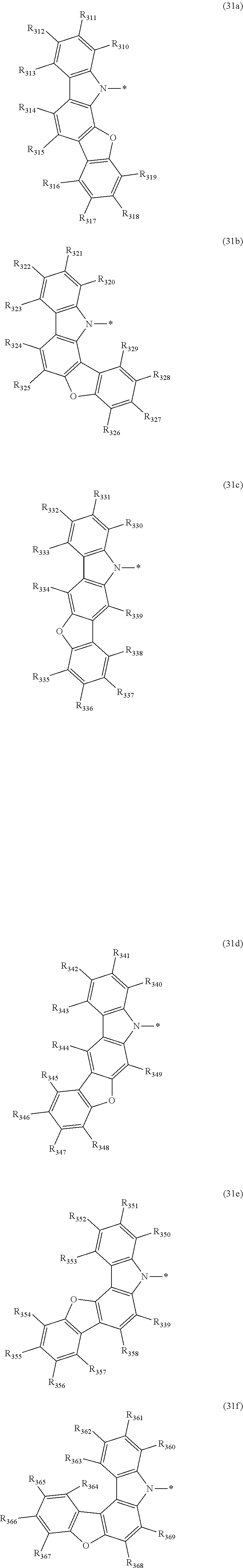
wherein, in the formulae (31a), (31b), (31c), (31d), (31e), and (31f):
R₃₁₀ to R₃₁₉,
R₃₂₀ to R₃₂₉,
R₃₃₀ to R₃₃₉,
R₃₄₀ to R₃₄₉,
R₃₅₀ to R₃₅₉,
R₃₆₀ to R₃₆₉, are each independently a hydrogen atom or a substituent
selected from the group consisting of a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, and a substituted or unsubstituted arylthio group having 6 to 30 ring carbon atoms; and
* represents a bonding position to a benzene ring bonded with R₄₀₁ to R₄₀₄, and wherein, in the formula (301):
R₃₁ to R₃₈,
and R₄₀₁ to R₄₁₂ are each independently a hydrogen atom or a substituent
selected from the group consisting of a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, and a substituted or unsubstituted arylthio group having 6 to 30 ring carbon atoms;
n is 0, 1, 2, or 3;
when a plurality of R₄₀₅ are present, the plurality of R₄₀₅ are mutually the same or different;
when a plurality of R₄₀₆ are present, the plurality of R₄₀₆ are mutually the same or different;
when a plurality of R₄₀₇ are present, the plurality of R₄₀₇ are mutually the same or different; and
when a plurality of R₄₀₈ are present, the plurality of R₄₀₈ are mutually the same or different.

27. The compound according to claim 26, which is represented by the formula (302) below, 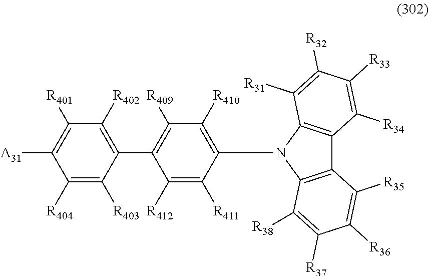
wherein A₃₁, R₄₀₁ to R₄₀₄, R₄₀₉ to R₄₁₂, and R₃₁ to R₃₈ in the formula (302) respectively represent the same as A₃₁, R₄₀₁ to R₄₀₄, R₄₀₉ to R₄₁₂, and R₃₁ to R₃₈ in the formula (301).

28. The compound according to claim 26, wherein R₄₀₁ to R₄₁₂, and R₃₁ to R₃₈ are each a hydrogen atom.

29. The compound according to claim 26, wherein R₃₁₀ to R₃₁₉, R₃₂₀ to R₃₂₉, R₃₃₀ to R₃₃₉, R₃₄₀ to R₃₄₉, R₃₅₀ to R₃₅₉, and R₃₆₀ to R₃₆₉ are each a hydrogen atom.

30. A compound represented by the formula (310) below, 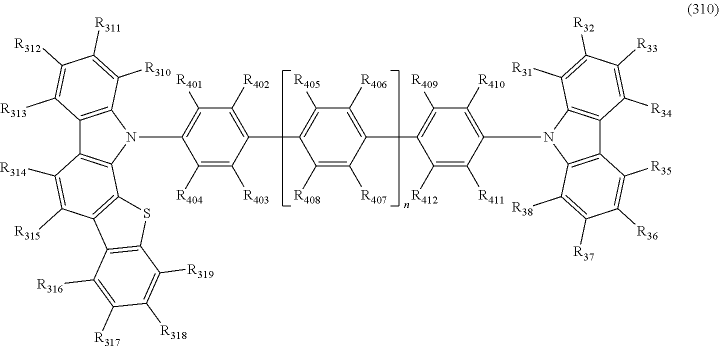
wherein, in the formula (310):
R₃₁₀ to R₃₁₉,
R₃₁ to R₃₈,
R₄₀₁ to R₄₁₂ are each independently a hydrogen atom or a substituent
selected from the group consisting of a halogen atom, a substituted or unsubstituted aryl group having 6 to 30 ring carbon atoms, a substituted or unsubstituted heterocyclic group having 5 to 30 ring atoms, a substituted or unsubstituted alkyl group having 1 to 30 carbon atoms, a substituted or unsubstituted alkyl halide group having 1 to 30 carbon atoms, a substituted or unsubstituted alkenyl group having 2 to 30 carbon atoms, a substituted or unsubstituted alkynyl group having 2 to 30 carbon atoms, a substituted or unsubstituted alkylsilyl group having 3 to 30 carbon atoms, a substituted or unsubstituted arylsilyl group having 6 to 60 ring carbon atoms, a substituted or unsubstituted arylphosphoryl group having 6 to 60 ring carbon atoms, a hydroxy group, a substituted or unsubstituted alkoxy group having 1 to 30 carbon atoms, a substituted or unsubstituted aryloxy group having 6 to 30 ring carbon atoms, an amino group, a substituted or unsubstituted alkylamino group having 2 to 30 carbon atoms, a substituted or unsubstituted arylamino group having 6 to 60 ring carbon atoms, a thiol group, a substituted or unsubstituted alkylthio group having 1 to 30 carbon atoms, and a substituted or unsubstituted arylthio group having 6 to 30 ring carbon atoms;
n is 0, 1, 2, or 3;
when a plurality of R₄₀₅ are present, the plurality of R₄₀₅ are mutually the same or different;
when a plurality of R₄₀₆ are present, the plurality of R₄₀₆ are mutually the same or different;
when a plurality of R₄₀₇ are present, the plurality of R₄₀₇ are mutually the same or different; and
when a plurality of R₄₀₈ are present, the plurality of R₄₀₈ are mutually the same or different.

31. The compound according to claim 30, which is represented by the formula (311) below, 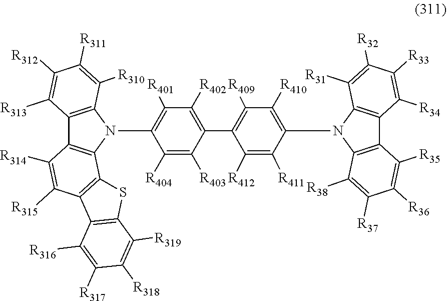
wherein R₃₁₀ to R₃₁₉, R₄₀₁ to R₄₀₄, R₄₀₉ to R₄₁₂, and R₃₁ to R₃₈ in the formula (311) respectively represent the same as R₃₁₀ to R₃₁₉, R₄₀₁ to R₄₀₄, R₄₀₉ to R₄₁₂, and R₃₁ to R₃₈ in the formula (310).

32. The compound according to claim 30, wherein R₄₀₁ to R₄₁₂, and R₃₁ to R₃₈ are each a hydrogen atom.

33. The compound according to claim 30, wherein R₃₁₀ to R₃₁₉ are each a hydrogen atom.

34. An organic electroluminescence device, comprising:
an anode;
a cathode; and
an emitting layer provided between the anode and the cathode,
wherein the emitting layer comprises a delayed fluorescent compound M2 and the compound according to claim 26.

35. An electronic device comprising the organic electroluminescence device according to claim 34.

36. An organic electroluminescence device, comprising:
an anode;
a cathode; and
an emitting layer provided between the anode and the cathode,
wherein the emitting layer comprises a delayed fluorescent compound M2 and the compound according to claim 30.
